(19) United States
(12) Patent Application Publication
(10) Pub. No.: US 2026/0315556 A1
(43) Pub. Date: Oct. 15, 2026

(54) PHAGE THERAPY FOR ALCOHOL-ASSOCIATED HEPATITIS

(71) Applicants: Bernd Schnabl, San Diego, CA (US); Jumpei Fujiki, San Diego, CA (US); THE REGENTS OF THE UNIVERSITY OF CALIFORNIA, Oakland, CA (US)

(72) Inventors: Bernd Schnabl, San Diego, CA (US); Jumpei Fujiki, San Diego, CA (US)

(73) Assignee: THE REGENTS OF THE UNIVERSITY OF CALIFORNIA, Oakland, CA (US)

(21) Appl. No.: 19/483,909

(22) PCT Filed: May 17, 2024
(86) PCT No.: PCT/US2024/030108

§ 371 Date: Nov. 12, 2025

Related U.S. Application Data
(60) Provisional application No. 63/467,265, filed on May 17, 2023.
(60) Provisional application No. 63/467,525, filed on May 18, 2023.

(51) Int. Cl. A61K 35/76; C12N 7/00

(52) U.S. Cl. CPC A61K 35/76; C12N 7/00; C12N 2795/00021; C12N 2795/00032; C12N 2795/00052; C12N 2795/10121; C12N 2795/10132; C12N 2795/10152; C12N 2795/10221; C12N 2795/10232; C12N 2795/10252; C12N 2795/10321; C12N 2795/10332; C12N 2795/10352

(57) ABSTRACT

Provided herein are therapeutic agents and compositions for the treatment of infections associated with alcohol-associated hepatitis. Also provided herein are methods for extending the host range of Enterococcus faecalis (E. faecalis) phages for treatment of alcohol-associated hepatitis related infections.

### PRIORITY

This application claims the benefit of priority of U.S. Provisional Patent Application No. 63/467,265, filed on May 17, 2023, and U.S. Provisional Patent Application No. 63/467,525, filed on May 18, 2023, the benefit of priority to each is claimed hereby, and are incorporated by reference herein in their entireties.

### INCORPORATION BY REFERENCE OF SEQUENCE LISTING

This application contains a Sequence Listing which has been submitted electronically in ST26 format and hereby incorporated by reference in its entirety. Said ST26 file, created on May 12, 2024, is named 1133117WO1.xml and is 317,597 bytes in size.

### FIELD OF THE INVENTION

The present invention relates generally to therapeutic agents and compositions for the treatment of infections associated with alcohol-associated hepatitis. Additionally, the invention relates to methods for extending the host range of Enterococcus faecalis (E. faecalis) phages for treatment of alcohol-associated hepatitis related infections.

### BACKGROUND

Chronic liver disease due to alcohol use disorder contributes markedly to the global burden of disease and mortality (Lozano et al., 2010; Lee et al., 2019; Rehm et al., 2014). Alcoholic liver disease affects several million people in the United States. Chronic alcoholic liver disease may progress from simple steatosis to steatohepatitis, liver fibrosis and to cirrhosis in 15-40% of patients, an end-stage disease, a major cause of morbidity and mortality worldwide.

Alcoholic hepatitis is a severe and life-threatening form of alcohol-related liver disease. Alcoholic hepatitis (AH) is a distinct clinical entity characterized by cholestasis, jaundice and liver failure that generally occurs after decades of heavy alcohol use. Recovery from AH is dictated by abstinence from alcohol, the presence of mild clinical symptoms and the implementation of appropriate therapy. Unfortunately, patients with severe AH have a high mortality rate of about 40% to 50% despite optimal medical management. A significant percentage of patients succumb to infections, with infection attributed mortality of 12% to 54%, underscoring a disrupted gut barrier with subsequent translocation of bacteria to extraintestinal sites.

### SUMMARY

Evolved phages were identified that extend the biological activity of E. faecalis phages as determined by their host range against a well-characterized E. faecalis reference collection (EF01-EF16) and phages were also identified with genetic differences as compared with the parent phage. For example, it was found that the evolved phage Φ2.3N15D5 was infectious to EF06 as well as EF15, indicating an expansion of the host range against E. faecalis strains.

The disclosure provides a method to evolve E. faecalis phages comprising infecting one or more E. faecalis strains with one or more parental phage and determining whether one or more evolved phage are produced. In some embodiments, infecting one or more E. faecalis strains with one or more parental phage comprises co-culturing the one or more parental phage with one or more E. faecalis strains. In other embodiments, determining whether one or more evolved phage are produced comprises monitoring the optical density (OD) value of the co-culture. In other embodiments, the method further comprises obtaining the one or more evolved phages and repeating the steps of infecting one or more E. faecalis strains with the one or more evolved phages and determining whether one or more further evolved phages are produced. In certain embodiments, determining whether one or more evolved phage are produced comprises detecting a mutation in the evolved phage genomic sequence. In further embodiments, the method comprises detecting one, two, three, four, five, six, seven, eight, nine, ten, or more mutations in the evolved phage genomic sequence. In other embodiments, determining whether one or more evolved phage are produced comprises detecting a mutation in the evolved phage genomic sequence encoding a tail fiber, an endolysin, or a single stranded DNA binding protein as compared to the parent phage sequence. In particular embodiments, the mutation comprises an E to K amino acid substitution at position 1287 of the tail fiber protein encoded by SEQ ID NO:2, the mutation comprises an E to K amino acid substitution at position 1559 of the tail fiber protein encoded by SEQ ID NO:2, the mutation comprises a V to A amino acid substitution at position 226 of the endolysin protein encoded by SEQ ID NO:2, the mutation comprises a F to S amino acid substitution at position 260 of the endolysin protein encoded by SEQ ID NO:2, the mutation comprises a P to L amino acid substitution at position 25 of the single stranded DNA binding protein encoded by SEQ ID NO:2, the mutation comprises a N to D amino acid substitution at position 83 of the single stranded DNA binding protein encoded by SEQ ID NO:2, the mutation comprises a M to T amino acid substitution at position 150 of the single stranded DNA binding protein encoded by SEQ ID NO:2, the mutation comprises a Q to R amino acid substitution at position 240 of the single stranded DNA binding protein encoded by SEQ ID NO:2, or a combination of any thereof. In one embodiment, the E. faecalis is cytolysin-positive. In one embodiment, the host range of the E. faecalis includes the one or more phage. In one embodiment, the host range of the E. faecalis does not include the one or more phage. In one embodiment, at least one of the strains is susceptible to infection by the parental phage. In one embodiment, at least one of the strains is not susceptible to infection by the parental phage. In some embodiments, the one or more parental phage comprises bacteriophage strain Φ2.3 or Φ2.3N13D5, wherein representative samples of said phage have been deposited under IDAC Accession No. 060423-03 and 060423-04, respectively. In other embodiments, the one or more parental phage comprises phiEF24C, ECP3, IME-EF1, SAP6, BC611, EfaCPT1, EFDG1, EFLK1, Q69 Phi4D, IME_EF3, EFRM31, EFRM42, EFRM54, PhiFL1A, PhiFL1B, PhiFL1C, PhiFL2A, PhiFL2B, PhiFL3A, PhiFL3B, PhiFL4A, EFC-1, or a combination of any thereof.

Further provided is isolated phage produced by the methods provided herein that can infect multiple E. faecalis strains, thereby having a wider host range than the one or more parental phage or isolated phage produced by the methods provided herein that can infect phage resistant E. faecalis strains. In one embodiment, an isolated infectious phage is provided comprising SEQ ID NO: 2 or a nucleotide sequence with at least 80%, 82%, 84% 85%, 86%, 88%, 90%, 92%, 94% 95%, 96%, 97%, 98% or 99% nucleic acid sequence identity thereto. In certain embodiments, the isolated phage comprises a mutation in its genomic sequence encoding a tail fiber, an endolysin, or a single stranded DNA binding protein as compared to the parent phage sequence. In particular embodiments, the mutation comprises an E to K amino acid substitution at position 1287 of the tail fiber protein encoded by SEQ ID NO:2, the mutation comprises an E to K amino acid substitution at position 1559 of the tail fiber protein encoded by SEQ ID NO:2, the mutation comprises a V to A amino acid substitution at position 226 of the endolysin protein encoded by SEQ ID NO:2, the mutation comprises a F to S amino acid substitution at position 260 of the endolysin protein encoded by SEQ ID NO:2, the mutation comprises a P to L amino acid substitution at position 25 of the single stranded DNA binding protein encoded by SEQ ID NO:2, the mutation comprises a N to D amino acid substitution at position 83 of the single stranded DNA binding protein encoded by SEQ ID NO:2, the mutation comprises a M to T amino acid substitution at position 150 of the single stranded DNA binding protein encoded by SEQ ID NO:2, the mutation comprises a Q to R amino acid substitution at position 240 of the single stranded DNA binding protein encoded by SEQ ID NO:2, or a combination of any thereof. In other embodiments, the isolated phage comprises bacteriophage strain 02.3, Φ2.3N13D5, or a bacteriophage strain derived therefrom, wherein representative samples of bacteriophage strain 02.3 and Φ2.3N13D5 have been deposited under IDAC Accession No. 060423-03 and 060423-04, respectively.

The disclosure also provides for a method to treat alcoholic hepatitis, NASH, liver cirrhosis, or liver failure in a mammal. The method includes administering to the mammal an effective amount of a composition comprising one or more lytic evolved phage specific for Entercoccus faecalis. In one embodiment, the mammal is a human. In one embodiment, the composition comprises a Myoviridae, Podoviridae, Spounaviridae or Siphoviridae phage, or any combination thereof. In one embodiment, one or more of the phage are isolated from the mammal, and optionally amplified, prior to administration. In one embodiment, one or more of the phages have broad host specificity, and/or is a genetically modified phage. In one embodiment, levels of Enterococcus cytolysin or cytolytic E. faecalis are monitored after administration of the composition. In one embodiment, a cocktail of lytic phages is administered. In one embodiment, the composition is orally administered. In one embodiment, the composition is a tablet. In one embodiment, the composition is a sustained release dosage form.

### BRIEF DESCRIPTION OF THE FIGURES

FIGS. 1A-1G. Phage therapy against cytolytic E. faecalis reduces ethanol-induced liver disease in gnotobiotic mice. (A-G) C57BL/6 germfree mice were colonized with feces from two different cytolysin-positive patients with alcoholic hepatitis. The mice were placed on isocaloric (control) or chronic-binge ethanol diets, and gavaged with vehicle (PBS), control phages against C. crescentus (10¹⁰ PFUs), or a cocktail of 3 or 4 different phages targeting cytolytic E. faecalis (10¹⁰ PFUs) 1 day before an ethanol binge. (A) Serum levels of ALT. (B) Hepatic triglyceride content. (C) Representative oil red O-stained liver sections. (D-F) Hepatic levels of mRNAs encoding inflammatory cytokines. (G) Proportions of cylL_{S} detected in liver, measured by qPCR (Control diet: Patient #5: C. crescentus phages, n=5; E. faecalis phages, n=5; Patient #2: C. crescentus phages, n=5; E. faecalis phages, n=5; Ethanol diet: Patient #5: C. crescentus phages, n=14; E. faecalis phages, n=16; Patient #2: C. crescentus phages, n=16; E. faecalis phages, n=15). Scale bar=100 μm. Results are expressed as mean+s.e.m (A-F). P values are determined by One-way ANOVA with Tukey's post-hoc test (A-F) or Fisher's exact test followed by FDR procedures (G). *P<0.05, **P<0.01, *** P<0.001.

FIGS. 2A-2K. Phages reduce translocation of cytolysin to the liver and reduce ethanol-induced liver disease in Atp4α^{Sl/Sl} mice. (A-K) Wild-type (WT) and their Atp4α^{Sl/Sl} littermates were fed oral isocaloric (control) or chronic-binge ethanol diets and gavaged with vehicle (PBS), control phages against C. crescentus (10¹⁰ PFUs), or a cocktail of 4 different phages targeting cytolytic E. faecalis (10¹⁰ PFUs) 1 day before ethanol binge. (A) Serum levels of ALT. (B) Hepatic triglyceride content. (C) Representative oil red O-stained liver sections. (D-F) Hepatic expression of mRNAs encoding inflammatory cytokines. (G) Proportions of cylL_{S} detected in liver, measured by qPCR (Control diet: WT mice: PBS, n=7; C. crescentus phages, n=6; E. faecalis phages, n=5; Atp4α^{Sl/Sl} mice: PBS, n=6; C. crescentus phages, n=8; E. faecalis phages, n=7; Ethanol diet: WT mice: PBS, n=14; C. crescentus phages, n=12; E. faecalis phages, n=13; Atp4α^{Sl/Sl} mice: PBS, n=15; C. crescentus phages, n=15; E. faecalis phages, n=15). (H) Fecal CFUs of Enterococcus. (I) Fecal samples were collected and 16S rRNA genes were sequenced. Principal coordinate analysis based on Jaccard dissimilarity matrices found no significant difference in fecal microbiota among mice given PBS, control phage, or phages targeting cytolytic E. faecalis in each group (Control diet: WT mice: PBS, n=6; C. crescentus phages, n=6; E. faecalis phages, n=5; Atp4α^{Sl/Sl} mice: PBS, n=6; C. crescentus phages, n=8; E. faecalis phages, n=7; Ethanol diet: WT mice: PBS, n=14; C. crescentus phages, n=12; E. faecalis phages, n=13; Atp4α^{Sl/Sl} mice: PBS, n=9; C. crescentus phages, n=9; E. faecalis phages, n=14). (J and K) Serum levels of ethanol and hepatic levels of 1 and Cyp2e1 mRNAs did not differ significantly among colonized mice after ethanol feeding. Scale bar=100 μm. Results are expressed as mean±s.e.m (A-F, H, J, K). P values are determined by One-way ANOVA with Tukey's post-hoc test (A-F, H, J, K), Fisher's exact test followed by FDR procedures (G), or PERMANOVA followed by FDR procedures (i). *P<0.05, **P<0.01, ***P<0.001.

FIGS. 3A-3B. Isolation and amplification of bacteriophages against cytolytic E. faecalis isolated from patients with alcoholic hepatitis. (A) BHI agar plates showing bacteriophage plaque morphology. (B) Transmission electron microscopy revealed that bacteriophages isolated were either siphophages (5_2, 5_4, 5_5, 5_6 and 2_6) or myophages (2_1 and 2_8). Phages specific to cytolytic E. faecalis strains isolated from feces of cytolysin-positive patients with alcoholic hepatitis were named as Efφ with patient number plus a digit (Ef for E. faecalis, φ for phage, last digit for isolation order). (Transmission electron microscopy graphs of bacteriophage 2_1 and 2_8, stained with phosphotungstic acid showing contracted tails. Scale bar=50 nm.

FIGS. 4A-4D. Phages that target cytolytic E. faecalis reduce ethanol-induced liver disease in gnotobiotic mice. (A-D) C57BL/6 germ-free mice were colonized with feces from two different cytolysin-positive patients with alcoholic hepatitis. The mice were then fed oral isocaloric (control) or chronic-binge ethanol diets, and gavaged with vehicle (PBS), control phages against C. crescentus (10¹⁰ PFUs), or a cocktail of 3 or 4 different phages targeting cytolytic E. faecalis (10¹⁰ PFUs) 1 day before an ethanol binge. (A) Fecal CFUs of Enterococcus. (B) Fecal samples were collected and 16S rRNA genes were sequenced. Principal coordinate analysis based on Jaccard dissimilarity matrices shows no significant differences in fecal microbiota of mice gavaged with control phage and phages targeting cytolytic E. faecalis in each group (Control diet: Patient #5: C. crescentus phages, n=5; E. faecalis phages, n=5; Patient #2: C. crescentus phages, n=4; E. faecalis phages, n=5; Ethanol diet: Patient #5: C. crescentus phages, n=14; E. faecalis phages, n=16; Patient #2: C. crescentus phages, n=16; E. faecalis phages, n=15). (C and D) Serum levels of ethanol and hepatic levels of Adh1 and Cyp2e1 mRNAs did not differ significantly among colonized mice after ethanol feeding. Results are expressed as mean±s.e.m (A, C, D). P values are determined by One-way ANOVA with Tukey's post-hoc test (A, C, D), or PERMANOVA followed by FDR procedures (b). *P<0.05.

FIGS. 5A-5C. Isolation and amplification of bacteriophages against non-cytolytic E. faecalis isolated from patients with alcoholic hepatitis. (A) BHI agar plates showing bacteriophage plaque morphology. (B) Transmission electron microscopy revealed that bacteriophages isolated were either podophages (6_1, 6_2, 6_3, 7_2, 7_3 and 7_4) or siphophages (6_4 and 7_1). Phages specific to non-cytolytic E. faecalis strains isolated from feces of cytolysin negative alcoholic hepatitis patients were named as Efφ with patient number plus a digit (Ef for E. faecalis, φ for phage, last digit for isolation order). Scale bar=50 nm. (C) Phylogenetic tree of all bacteriophages used in this study.

FIGS. 6A-6K. Phages that target non-cytolytic E. faecalis do not reduce ethanol-induced liver disease in gnotobiotic mice. (A-K) C57BL/6 germfree mice were colonized with feces from two different cytolysin-negative patients with alcoholic hepatitis. Transplanted gnotobiotic mice were fed oral isocaloric (control) or chronic-binge ethanol diets and gavaged with vehicle (PBS), control phages against C. crescentus (10¹⁰ PFUs), or a cocktail of 4 different phages targeting non-cytolytic E. faecalis (10¹⁰ PFUs) 1 day before an ethanol binge (A) Serum levels of ALT. (B) Hepatic triglyceride content. (C) Representative oil red O-stained liver sections. (D-F) Hepatic levels of mRNAs encoding inflammatory cytokines. (G) Proportions of cylL_{S} detected in liver, measured by qPCR (Control diet: Patient #6: C. crescentus phages, n=6; E. faecalis phages, n=6; Patient #7: C. crescentus phages, n=5; E. faecalis phages, n=6; Ethanol diet: Patient #6: C. crescentus phages, n=14; E. faecalis phages, n=15; Patient #7: C. crescentus phages, n=13; E. faecalis phages, n=13). (H) Fecal CFUs of Enterococcus. (I) Fecal samples were collected and 16S rRNA genes were sequenced. Principal coordinate analysis based on Jaccard dissimilarity matrices found no significant difference in fecal microbiota among mice gavaged with control phages and phages targeting cytolytic E. faecalis in each group (Control diet: Patient #6: C. crescentus phages, n=5; E. faecalis phages, n=6; Patient #7: C. crescentus phages, n=4; E. faecalis phages, n=6; Ethanol diet: Patient #6: C. crescentus phages, n=13; E. faecalis phages, n=14; Patient #7: C. crescentus phages, n=13; E. faecalis phages, n=13). (J and K) Serum levels of ethanol and hepatic levels of Adh1 and Cyp2e1 mRNAs did not differ significantly among colonized mice after ethanol feeding. Scale bar=100 μm. Results are expressed as mean±s.e.m (A-F, H, J, K). P values are determined by One-way ANOVA with Tukey's post-hoc test (A-F, H, J, K), Fisher's exact test followed by FDR procedures (G), or PERMANOVA followed by FDR procedures (i). *P<0.0.

FIG. 7. OD₆₀₀ over time for the reference collection of 16 different clinical E. faecalis isolates (susceptible and non-susceptible strains) infected with a parental phage.

FIG. 8. Plaque Forming Units per milliliter (PFU/mL) in co-cultures.

FIG. 9. Sensitivity of the reference collection 16 different clinical E. faecalis isolates against evolved phages with single mutation.

FIG. 10. An exemplary parental phage sequence (SEQ ID NO: 1). SEQ ID NO: 1—The genomic sequence of Enterococcus faecalis bacteriophage Ef2.3, also known as vB_EfaM_Ef2.3.

FIG. 11. An exemplary evolved phage sequence (SEQ ID NO:2). SEQ ID NO:2—The genomic sequence of Enterococcus faecalis bacteriophage Ef2.3v1, also known as Φ2.3N13D5.

FIG. 12. Activity of evolved phages against bacteriophage insensitive mutants (BIMs).

FIG. 13. Mutations in evolved phages obtained from the experiments described in FIG. 7. * Φ2.3N03Φ5=Ev03, Φ2.3N05Φ5=Ev05, Φ2.3N06D5=Ev06, Φ2.3N08D5=Ev08, Φ2.3N10D5=Ev10, Φ2.3N11D5=Ev11, Φ2.3N12D5=Ev12, Φ2.3N13D5=Ev13=Ef2.3v01, Φ2.3N14D5=Ev14 and Φ2.3N15D5=Ev15. (SEQ ID NOS: 14-17)

FIGS. 14A-14H. (A) Scheme for insertion of double or triple mutations to the Ef2.3. We used the same method described in FIG. 7 for co-cultures. (B-D) OD₆₀₀ over time for the reference collection of different clinical E. faecalis isolates (susceptible=EF13 and non-susceptible strains=EF06 and EF12) infected with evolved phages which have single mutation. (E) Plaque Forming Units per milliliter (PFU/mL) in co-cultures described in 14B-D. (F) OD₆₀₀ over time for the reference collection of the clinical E. faecalis isolate (EF06) infected with evolved phages which have double mutation. (G) Plaque Forming Units per milliliter (PFU/mL) in co-cultures described in 14F. (H) Sensitivity of the reference collection 16 different clinical E. faecalis isolates against evolved phages with double and triple mutations.

FIG. 15. Mutations in evolved phages obtained from the experiments described in FIG. 14.

FIGS. 16A-16F. Comparative analysis of mutations in all evolved phages. (A-C) Location of all mutations in the genes of Ef2.3. (D-F) BLAST analysis of these mutations (SEQ ID NOS: 18-35).

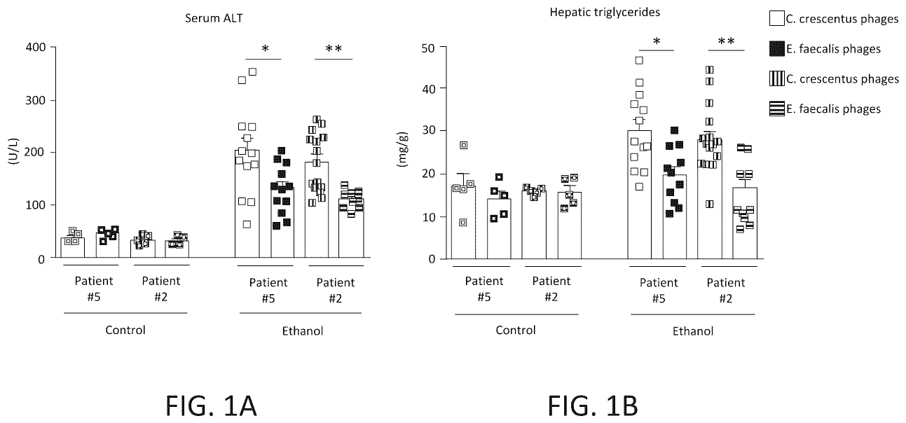

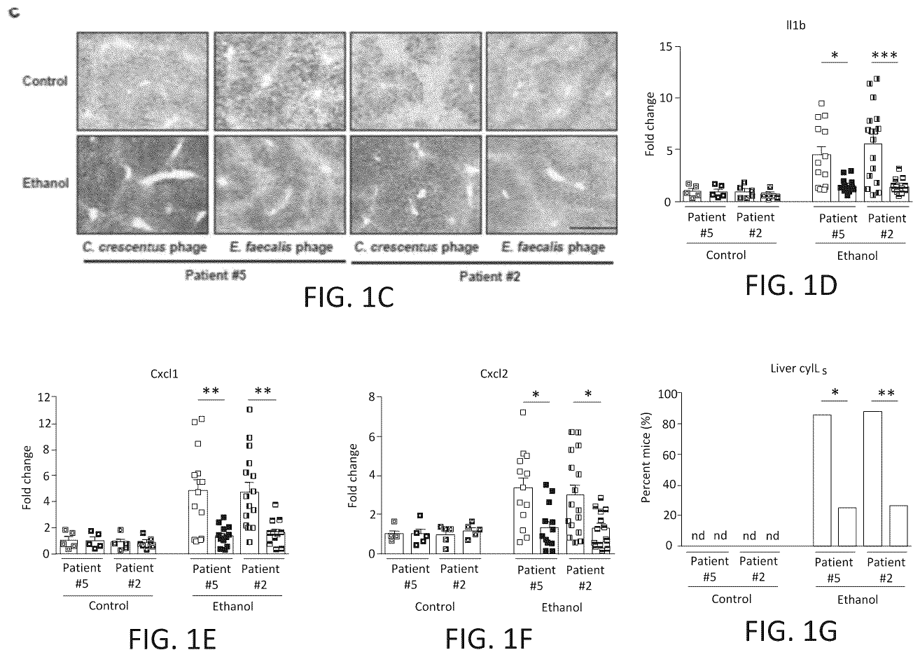

### DETAILED DESCRIPTION

The number of patients with chronic liver disease is increasing rapidly-liver cirrhosis is now the 12′ leading cause of death worldwide (Lozano et al., 2012) and more than 50% of cases are associated with chronic alcohol abuse (Rehm et al., 2013). Furthermore, alcohol-associated liver disease recently became the leading cause for liver transplantation in the United States (Lee et al., 2019). The most severe form of alcohol-related liver disease is alcoholic hepatitis; where mortality ranges from 20% to 40% at 1-6 months, and as many as 75% of patients die within 90 days of a diagnosis of severe alcoholic hepatitis (Thursz et al., 2015; Maddrey et al., 1978; Dominguez et al., 2008). Therapy with corticosteroids is only marginally effective (Thursz et al., 2015) with early liver transplantation representing the only curative therapy. However, liver transplantation is only offered at some centers, to a select group of patients (Mathurin and Lucey, 2012). Therefore, a continuing need exists in the art to develop novel compositions and methods to improve outcomes for patients with severe AH.

The present disclosure overcomes the limitations of the prior art by providing novel bacteriophage as well as compositions and methods producing and using the same. These novel bacteriophages exhibit broader E. faecalis host specificity; and provide the ability to protect against E. faecalis clinical isolates resistant to known E. faecalis bacteriophages. As such, the compositions and methods provided herein can be helpful in reducing the number of patients that succumb to AH-related infections.

The disclosure provides, for the first time, bacteriophage capable of infecting E. faecalis clinical isolates resistant to ΦEf2.3 infection as well as methods allowing efficient identification and isolation of bacteriophage (phage) capable of infecting, replicating within, and lysing E. faecalis resistant to ΦEf2.3 infection. The disclosure also provides a method for controlling E. faecalis infection. The bacteriophages described herein can precisely edit the intestinal microbiota and selectively target and eradicate specific strains of bacteria, including cytolytic E. faecalis.

The disclosure also provides a method of evolving a virus (bacteriophage) virulent to E. faecalis. Methods of evolving bacteriophages can encompass, e.g., any method capable of producing bacteriophage having increased infectivity against E. faecalis clinical isolates sufficient for treating AH-related bacterial infections. In one embodiment, evolving bacteriophage virulent to E. faecalis can comprise growing bacteriophage in the presence of one or more E. faecalis clinical isolates, allowing the bacteriophage to propagate, and isolating bacteriophage particles from the culture. In specific embodiments, the method can comprise monitoring the optical density over time, e.g., over a 5-day period.

As described herein, mutations observed in the evolved phage were identified in genomic regions encoding a tail fiber protein, an endolysin protein, a single stranded DNA binding protein, and combinations thereof. For example, with reference to SEQ ID NO:2, such mutations may be further defined as a lysine (K) at the position corresponding to amino acid position 1287 of the tail fiber protein encoded by SEQ ID NO:2; a lysine (K) at the position corresponding to amino acid position 1559 of the tail fiber protein encoded by SEQ ID NO:2; an alanine (A) at the position corresponding to amino acid position 226 of the endolysin protein encoded by SEQ ID NO:2; a serine (S) at the position corresponding to amino acid position 260 of the endolysin protein an endolysin protein encoded by SEQ ID NO:2; a leucine (L) at the position corresponding to amino acid position 25 of the single stranded DNA binding protein encoded by SEQ ID NO:2; an aspartate (D) at the position corresponding to amino acid position 83 of the single stranded DNA binding protein encoded by SEQ ID NO:2; a threonine (T) at the position corresponding to amino acid position 150 of the single stranded DNA binding protein encoded by SEQ ID NO:2; an arginine (R) at the position corresponding to amino acid position 240 of the single stranded DNA binding protein encoded by SEQ ID NO:2; or a combination of any thereof. As described herein, such amino acid substitutions are not known in the art and are observed to be associated with the expansion of phage host range.

Because of the degeneracy of the genetic code, a variety of different polynucleotide sequences can encode proteins, such as the tail fiber protein, endolysin protein, and single stranded DNA binding proteins disclosed herein. Polynucleotide sequences encoding such proteins with the amino acid substitutions described herein can be produced by introducing mutations into a polynucleotide sequence encoding a parental tail fiber protein, endolysin protein, and/or single stranded DNA binding protein using methods known in the art. It is well within the capability of one of skill in the art to create alternative polynucleotide sequences encoding the same, or essentially the same, mutated proteins as described herein. These variant or alternative polynucleotide sequences are within the scope of the embodiments described herein.

The following definitions and methods are provided to better define the present invention and to guide those of ordinary skill in the art in the practice of the present invention. Unless otherwise noted, terms are to be understood according to conventional usage by those of ordinary skill in the relevant art.

### DEFINITIONS

References in the specification to “one embodiment,” “an embodiment,” etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrase “one or more” is readily understood by one of skill in the art, particularly when read in context of its usage. For example, one or more substituents on a phenyl ring refers to one to five, or one to four, for example if the phenyl ring is di-substituted.

As used herein, “or” should be understood to have the same meaning as “and/or” as defined above. For example, when separating a listing of items, “and/or” or “or” shall be interpreted as being inclusive, e.g., the inclusion of at least one, but also including more than one of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including,” “includes,” “having,” “has,” “with,” or variants thereof, are intended to be inclusive similar to the term “comprising.”

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. The term about can also modify the endpoints of a recited range as discuss above in this paragraph.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The term “standard,” as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

As used herein, the term “isolated” in the context of phage or cytolysin, is intended to mean that the phage or cytolysin is one that is present alone or in combination with other compositions, but not within its natural environment. As used herein, the term “isolation” is defined as separation and identification of an organism from a solution containing a mixed culture of organisms. Organisms able to be isolated can include viruses, bacteria, mammalian cells, or the like. Bacteriophages can be isolated as described herein and known in the art. In one embodiment, general laboratory methods for isolating bacteriophages may include but are not limited to growth in cultured cells, bacteriophage assay, double agar method, and plaque assay, among others. The present disclosure provides a method of isolating bacteriophages by a method involving infecting one or more E. faecalis strains with one or more parental phage; and determining whether one or more evolved phages are produced; and isolating the evolved bacteriophages. In some embodiments, an evolved phage, e.g., an isolated phage produced by the methods described herein, may be further defined as having broad host specificity. As used herein, “broad host specificity,” “wider host range,” and similar terms, refer to a phage that is capable of infecting one or more additional strains of bacteria as compared to the corresponding parental phage.

As used herein, the term “virulent” refers to a virus, particularly a bacteriophage, that is able to infect, replicate within, and lyse (kill) a host cell. The term “temperate” refers to a bacteriophage that can integrate into the host genome (lysogenize) or lyse the host cell.

The term “purified” with respect to a bacteriophage means that the phage has been measurably increased in concentration by any purification process, including but not limited to, isolation from the environment or culture, e.g., isolation from culture following propagation and/or amplification, centrifugation, etc., thereby partially, substantially, nearly completely, or completely removing impurities, such as host cells and host cell components.

As used herein, the terms “therapeutic agent” and “therapeutic agents” refer to an agent, such as a bacteriophage or bacteriophage cocktail, that can be used in the treatment, management, or control of one or more symptoms of a disease or disorder.

As used herein, the terms “treat”, “treatment” and “treating” refer to obtaining a therapeutic benefit in a subject (e.g., a mammal, such as a human, companion animal (e.g., dog, or cat) or livestock (e.g., cow, horse, pig, goat or chicken) receiving a pharmaceutical composition. With respect to achieving a therapeutic benefit, the object is to eliminate, lessen, decrease the severity of, ameliorate, or slow the progression of the symptoms or underlying cause (e.g., bacterial infection) associated with the pathological condition or disorder. A “therapeutically effective amount” refers to that amount of a therapeutic agent, such as a phage cocktail pharmaceutical composition, sufficient to achieve at least one therapeutic benefit in a subject receiving the composition. In one embodiment, the composition, therapeutic agent, or formulation of the present disclosure decreases the viability and/or replicative capacity of cytolytic Enterococcus faecalis in a mammal by at least about 0.5%, or by at least about 1%, or by at least about 5%, or by at least about 10%, or by at least about 15%, or by at least about 20%, or by at least about 25%, or by at least about 30%, or by at least about 40%, or by at least about 50%, or by at least about 75%, or by at least about 90% when compared to mammals under the same conditions but without treatment.

As used herein, the terms “prevent”, “prevention” and “preventing” refer to obtaining a prophylactic benefit in a subject receiving a pharmaceutical composition. With respect to achieving a prophylactic benefit, the object is to delay or prevent the symptoms or underlying cause (e.g., bacterial infection) associated with the pathological condition or disorder. A “prophylactically effective amount” refers to that amount of a prophylactic agent, such as a phage cocktail composition, sufficient to achieve at least one prophylactic benefit in a subject receiving the composition. In some embodiments, the composition, therapeutic agent, or formulation of the present disclosure reduces translocation of cytolytic E. faecalis from the intestine to the liver by at least about 0.5%, or by at least about 1%, or by at least about 5%, or by at least about 10%, or by at least about 15%, or by at least about 20%, or by at least about 25%, or by at least about 30%, or by at least about 40%, or by at least about 50%, or by at least about 75%, or by at least about 90% when compared to mammals under the same conditions but without treatment.

### Diagnostic, Predictive and Detection Methods

The presence or amount of E. faecalis cytolysin or cytolytic Enterococcus faecalis in a physiological sample, such as a fecal sample, was found to be useful to predict whether a mammal has a short life expectancy, e.g., due to the severity of disease in the mammal, including liver disease. The presence or amount of E. faecalis cytolysin or cytolytic Enterococcus faecalis in a physiological sample, such as a fecal sample, may also be useful to determine whether a mammal is in liver failure, whether liver disease is progressing, whether intervention is warranted or whether a therapy is effective.

The presence or amount of E. faecalis cytolysin or cytolytic Enterococcus faecalis in a physiological sample may be detected by any means, direct or indirect. For example, the presence or amount of the large subunit of E. faecalis cytolysin or the small subunit of E. faecalis cytolysin, or both, may be detected using an antibody to cytolysin. The presence or amount of E. faecalis cytolysin or cytolytic Enterococcus faecalis in a physiological sample may be detected by detecting lanthionine. The presence or amount of E. faecalis cytolysin or cytolytic Enterococcus faecalis in a physiological sample may be detected by detecting RNA that encodes the large subunit, the small subunit, or both, of E. faecalis cytolysin, or detecting genomic DNA that encodes the large subunit, the small subunit, or both, E. faecalis cytolysin. In one embodiment, the method detects E. faecalis cytolysin or cytolytic Enterococcus faecalis, e.g., in bacteria found in human intestines, but not the cytolysin of closely related organisms, e.g., E. lactis, E. caccae, E. avium, E. canis, E. durans, E. ratti or other bacterial species found in the gut.

In one embodiment, any primer may be employed to detected E. faecalis cytolysin nucleic acid, e.g., that encodes the large subunit or the small submit. In one embodiment, one or more oligonucleotides are employed as primers in a nucleic acid amplification reaction. In one embodiment, at least one of the oligonucleotides has a length of about 5 to about 50, about 10 to about 25, about 15 to about 40, or about 15 to about 25 nucleotides. In one embodiment, at least one of the oligonucleotides has about 70%, 72&, 75%, 77%, 80%, 82%, 85%, 88%, 89%, 90%, 92%, 95%, 98% or more nucleic acid identity to one of GTAAAATAAGTAAAATCAAGAAAACTATTACTC (SEQ ID NO: 10), CAAAAGAAGGACCAACAAGTTCTAATT (SEQ ID NO: 11), CTGTTGCGGCGACAGCT (SEQ ID NO: 12), CCACCAACCCAGCCACAA (SEQ ID NO: 13). Thus, oligonucleotides within the scope of this disclosure include those having 1, 2, 3, 4, 5, or 6 nucleotide substitutions, lengths that are shorter, e.g., by 1, 2, 3, 4, 5, 6, 7, 8, 9 or 10 nucleotides, or lengths greater than, one of SEQ ID NOs: 10-13, or a combination thereof.

Mammals having cytolytic E. faecalis detected in a physiological sample may be subjected to therapy, including phage therapy, to reduce the number of E. faecalis.

### Phage Therapy

Bacteriophages, or phages for short, are viruses infecting bacteria that usually have a narrow host range. During infection of a bacterium, a phage has two principal life cycles it can enter—the lytic cycle and the lysogenic cycle. Both cycles are initiated by the attachment of the phage to a surface structure, which usually is species- and even strain-specific. After attachment, the phage injects its genetic material, which could be either DNA or RNA. After this injection, the phage can enter several different life cycles, with the lytic and lysogenic life cycle being the most common.

While all phages are capable of entering the lytic cycle (virulent phages), some phages (temperate phages) can also enter the lysogenic cycle. The lytic cycle results in production of phage particles; at the end of the cycle, the lysis cassette of the phage is expressed; this results in bacterial lysis and eventually in the release of new mature phage progeny.

Naturally occurring lytic phages are known for their antibacterial potential. To use bacteriophages as a therapeutic agent, naturally occurring bacteriophages that are able to lyse/kill Enterococcus faecalis were isolated. Germ-free mice were colonized with feces from a patient with AH. Using a chronic-plus-binge model of alcoholic liver disease, AH can be ameliorated using a personalized medicine approach with bacteriophages targeting Enterococcus faecalis. Phage therapy is safe and has been used in many clinical trials.

Exemplary phage useful in the therapeutic method include but are not limited to: those in any phage family that infects and optionally lyses or otherwise decreases the viability and/or replicative capacity of Enterococcus faecalis, e.g., EFDG1 (Khalifa et al., Appl. Environ. Microbiol., DOI: 10.1128/AEM.00096.15), φEf11 or variants thereof (see Zhang et al., Microbiology, 159:1023 (2013), IME-EF1 (Zhang et al., PloS One, doi.org/10.1371/journal.pone.0080435), including Myoviridae (non-enveloped, with head-tail (with a neck) geometries, and genomes are linear, double-stranded DNA, around 33-244 kb in length), Podoviridae (non-enveloped, with icosahedral and head-tail geometries where the double stranded DNA genome is linear, around 40-42 kb in length), Siphoviridae (non-enveloped, with icosahedral and head-tail geometries or a prolate capsid; genomes are double stranded and linear, around 50 kb in length).

The table below lists exemplary phages for use in the therapeutic methods:

| Phages of | Lytic/lysogenic | Accession | |
|---|---|---|---|
| E. faecalis | phage | number | Family |
| phiEF24C | Lytic | AP009390.1 | Myoviridae |
| ECP3 | Lytic | KJ801817.1 | Myoviridae |
| IME-EF1 | Lytic | KF1920531 | Siphoviridae |
| SAP6 | | JF731128.1 | Siphoviridae |
| BC611 | | AB712291.1 | Siphoviridae |
| EfaCPT1 | | JX193904.1 | Siphoviridae |
| EFDG1 | Lytic | KP339049.1 | Myoviridae |
| EFLK1 | Lytic | KR049063.1 | Myoviridae |
| Q69 | | | |
| Phi4D | | | Myoviridae |
| IME_EF3 | Lytic | KF728385 | Siphoviridae |
| EFRM31 | Lytic | GU815339 | Siphoviridae |
| EFRM42 | Lytic | | Siphoviridae |
| EFRM54 | Lytic | | Siphoviridae |
| PhiFL1A | Lysogenic | GQ478081 | Siphoviridae |
| PhiFL1B | Lysogenic | GQ478082 | Siphoviridae |
| PhiFL1C | Lysogenic | GQ478083 | Siphoviridae |
| PhiFL2A | Lysogenic | GQ478084 | Siphoviridae |
| PhiFL2B | Lysogenic | GQ478085 | Siphoviridae |
| PhiFL3A | Lysogenic | GQ478086 | Siphoviridae |
| PhiFL3B | Lysogenic | GQ478087 | Siphoviridae |
| PhiFL4A | Lysogenic | GQ478088 | Siphoviridae |
| EFC-1 | Lysogenic | KJ608188 | Siphoviridae |

### Exemplary Compositions and Therapeutic Methods

Thus, phages with the capacity of infecting bacteria and specifically, lytic phages, through infection of host bacteria, and are capable of decreasing populations of its host (target) bacteria without affecting other non-target bacterial strains, are useful in compositions and the methods described herein. By using more than one phage isolate or strain with a different specificity, resistance to the combination of phages is less likely. In one embodiment, the phages are employed that bind to different receptors (e.g., using a phage cocktail), to lower the risk of developing resistance.

In the present disclosure, the choice of the lytic phage specific for E. faecalis decreases the population of target bacteria. Thus, phages can be employed to treat disorders associated with E. faecalis, such as liver disease. The phage or phages are administered using, for instance, a delivery vehicle, such as a tablet, systemically or locally.

In one aspect, a composition comprising one phage isolate is employed. In one aspect, the composition comprises phage cocktails. In some embodiments, the composition comprises at least two different isolated strains of phage. In another aspect, the composition comprises a phage cocktail and a pharmaceutically acceptable carrier. In some embodiments, the pharmaceutical composition is formulated for systemic or local application. In some embodiments, the pharmaceutical composition comprises a sterile buffer, e.g., a buffer comprising about 0.05 M Tris-HCl, about 0.1M NaCl, and about 10 mM MgSO₄. In some embodiments, the composition further comprises an additional agent, e.g., an agent selected from the group consisting of an antibiotic agent, an anti-inflammatory agent, an antiviral agent, a local anesthetic agent, and a corticosteroid. In some embodiments, the composition is for use in treating a bacterial infection, and each of the phage strains is present in the composition in an amount corresponding to 103 to 10¹⁴ phage particles. In one embodiment, the daily dosage may be from 103 to 10¹⁰ phage particles, 10⁵ to 10¹⁰ phage particles, 10¹⁰ to 10²⁰ phage particles, 10¹⁵ to 10²⁰ phage particles, or 10²⁰ to 10²⁵ phage particles. The daily dosage may be administered in one or more doses. In some embodiments, each of the phage strains is present in the composition in an amount corresponding to 103 to 10⁵ phage, 10⁵ to 10⁸ phage, 10⁵ to 10¹⁰ phage, or 107 to 109 phage, or 109 to 10¹¹ phage, or 10¹¹ to 10¹³ phage. In some embodiments, the subject is a mammal, e.g., a human. In some embodiments, the treatment comprises administering a tablet or other orally compatible delivery vehicle having the composition.

In one aspect, cocktail compositions of different phage strains are administered to a human with liver disease, e.g., associated with alcohol use. The “cocktail” may comprise at least two different isolated strains of phage, for example, two, three, four, five, six, seven, eight, nine, ten, or more different isolated bacteriophage strains. The cocktail may be used alone or in further combination with other therapies, e.g., antibiotic agents and/or growth factors. In some embodiments, the phage cocktail comprises at least 2 phage strains, at least 3 phage strains, at least 4 phage strains, at least 5 phage strains, at least 6 phage strains, at least 7 phage strains, at least 8 phage stains, at least 9 phage strains, at least 10 phage strains, or more. In some embodiments, the phage cocktail comprises 2-20 phage strains, 2-15 phage strains, 2-10 phage strains, 3-8 phage strains, or 4-6 phage strains. In more embodiments, the combination does not impair or reduce (or does not substantially or significantly impair or reduce) infecting ability and/or lytic activity of the individual bacteriophage in the presence of distinct bacteriophage strains.

The phage or phage cocktails are incorporated into a composition for the use in treatment of a disease. A cocktail of different phage strains, or a single phage isolate, may be combined with a pharmaceutically acceptable carrier, such as an excipient or stabilizer, e.g., to form a tablet. Examples of pharmaceutically acceptable carriers, excipients, and stabilizers include, but are not limited to, buffers such as phosphate, citrate, and other organic acids; antioxidants including ascorbic acid; low molecular weight polypeptides; proteins, such as serum albumin and gelatin; hydrophilic polymers such as polyvinylpyrrolidone; amino acids such as glycine, glutamine, asparagine, arginine or lysine; monosaccharides, disaccharides, and other carbohydrates including glucose, mannose, or dextrins; sugar alcohols such as mannitol or sorbitol; salt-forming counterions such as sodium.

The bacteriophage or cocktail compositions may also be combined with one or more non-phage therapeutic and/or prophylactic agents, useful for the treatment and/or prevention of bacterial infections, as known in the art (e.g., one or more antibiotic agents). Other therapeutic and/or prophylactic agents that may be used in combination with the phage or phage cocktails include, but are not limited to, antibiotic agents, anti-inflammatory agents, antiviral agents, and corticosteroids. In some embodiments, the phage or phage cocktail is administered in the absence of a non-phage based antibiotic agent.

Standard antibiotics that may be used with pharmaceutical compositions comprising a phage cocktail include, but are not limited to, amikacin, gentamicin, kanamycin, neomycin, netilmicin, paromomycin, rhodostreptomycin, streptomycin, tobramycin, apramycin, rifamycin, naphthomycin, mupirocin, geldanamycin, ansamitocin, carbacephems, imipenem, meropenem, ertapenem, faropenem, doripenem, panipenem/betamipron, biapenem, PZ-601, cephalosporins, cefacetrile, cefadroxil, cefalexin, cefaloglycin, cefalonium, cefaloridine, cefalotin, cefapirin, cefatrizine, cefazaflur, cefazedone, cefazolin, cefradine, cefroxadine, ceftezole, cefaclor, cefonicid, cefprozil, cefuroxime, cefuzonam, cefmetazole, cefotetan, cefoxitin, cefcapene, cefdaloxime, cefdinir, cefditoren, cefetamet, cefixime, cefmenoxime, cefteram, ceftibuten, ceftiofur, ceftiolene, ceftizoxime, ceftriaxone, cefoperazone, ceftazidime latamoxef, cefclidine, cefepime, cefluprenam, cefoselis, cefozopran, cefpirome, cefquinome, flomoxef ceftobiprole, azithromycin, clarithromycin, dirithromycin, erythromycin, roxithromycin, aztreonam, pencillin and penicillin derivatives, actinomycin, bacitracin, colistin, polymyxin B, cinoxacin, flumequine, nalidixic acid, oxolinic acid, piromidic acid, pipemidic acid, rosoxacin, ciprofloxacin, enoxacin, fleroxacin, lomefloxacin, nadifloxacin, norfloxacin, ofloxacin, pefloxacin, rufloxacin, balofloxacin, gatifloxacin, grepafloxacin, levofloxacin, moxifloxacin, pazufloxacin, sparfioxacin, temafloxacin, tosufloxacin, clinafloxacin, garenoxacin, gemifloxacin, stifloxacin, trovalfloxacin, prulifloxacin, acetazolamide, benzolamide, bumetanide, celecoxib, chlorthalidone, clopamide, dichlorphenamide, dorzolamide, ethoxyzolamide, furosemide, hydrochlorothiazide, indapamide, mafendide, mefruside, metolazone, probenecid, sulfacetamide, sulfadimethoxine, sulfadoxine, sulfanilamides, sulfamethoxazole, sulfasalazine, sultiame, sumatriptan, xipamide, tetracycline, chlortetracycline, oxytetracycline, doxycycline, lymecycline, meclocycline, methacycline, minocycline, rolitetracycline, methicillin, nafcillin, oxacilin, cloxacillin, vancomycin, teicoplanin, clindamycin, co-trimoxazole, flucloxacillin, dicloxacillin, ampicillin, amoxicillin and any combination thereof in amounts that are effective to additively or synergistically enhance the therapeutic effect of a composition having phage for a given infection.

In one embodiment, the compositions generally may include a sterile buffer, such as a sterile PBS, water, or saline buffer. One particular buffer comprises Tris-HCl, NaCl, and/or MgSO₄7H₂O, e.g., about 0.05 M Tris-HCl (pH 7.4-7.5), about 0.1 M NaCl, and/or about 10 mM MgSO₄7H₂O. In other embodiments, the formulation further comprises a buffer and 10 mM MgCl₂. In other embodiments, the phage containing formulation further comprises a buffer having about 5 mM to about 15 mM CaCl₂, e.g., about 10 mM CaCl₂.

In some embodiments, compositions are provided in a hermetically sealed container.

In one embodiment, the phages are formulated as an aqueous solution or gel. The composition may include water; esters, isopropyl myristate and isopropyl palmitate; ethers such as dicapryl ether and dimethyl isosorbide; alcohols such as ethanol and isopropanol; fatty alcohols such as cetyl alcohol, cetearyl alcohol, stearyl alcohol and biphenyl alcohol; isoparaffins such as isooctane, isododecane and is hexadecane; silicone oils such as cyclomethicone, dimethicone, dimethicone cross-polymer, polysiloxanes and their derivatives, e.g., organomodified derivatives; polyols such as propylene glycol, glycerin, butylene glycol, pentylene glycol and hexylene glycol; or any combinations or mixtures of the foregoing. Aqueous vehicles may include one or more solvents miscible with water, including lower alcohols, such as ethanol, isopropanol, and the like.

### Formulations/Compositions

The phage or cocktails thereof can be formulated as pharmaceutical compositions and administered to a mammalian host, such as a human patient in a variety of forms adapted to the chosen route of administration, e.g., orally or parenterally, by intravenous, intramuscular, or subcutaneous routes. In one embodiment, the phage or cocktails thereof may be administered as a tablet.

In one embodiment, the phage or cocktails thereof may be administered by infusion or injection. Solutions of the phage or cocktails thereof can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion may include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it may include isotonic agents, for example, sugars, buffers or sodium chloride. Prolonged absorption of the injectable compositions can be brought about by the use in the compositions of agents delaying absorption, for example, aluminum monostearate and gelatin.

In one embodiment, the combination of phages does not impair or reduce (or does not substantially or significantly impair or reduce) infecting ability and/or lytic activity of the individual bacteriophage in the presence of distinct bacteriophage strains.

The phage or cocktails thereof optionally in combination with another active compound may be administered parenterally, for example, intravenously, orally, intraperitoneally, intramuscularly, or subcutaneously. Such administration may be as a single bolus injection, multiple injections, or as a short- or long-duration infusion. Implantable devices (e.g., implantable infusion pumps) may also be employed for the periodic parenteral delivery over time of equivalent or varying dosages of the particular formulation. For such parenteral administration, the compounds (a conjugate or other active agent) may be formulated as a sterile solution in water or another suitable solvent or mixture of solvents. The solution may contain other substances such as salts, sugars (particularly glucose or mannitol), to make the solution isotonic with blood, buffering agents such as acetic, citric, and/or phosphoric acids and their sodium salts, and preservatives.

Thus, the phage or cocktails thereof or in combination with another active agent, may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the phage or cocktails thereof optionally in combination with an active compound may be combined with one or more excipients and used in the form of ingestible tablets, buccal tablets, troches, capsules, elixirs, suspensions, syrups, wafers, and the like. Such compositions and preparations should contain at least 0.10% of active compound. The percentage of the compositions and preparations may, of course, be varied and may conveniently be between about 2 to about 60% of the weight of a given unit dosage form. The amount of conjugate and optionally other active compound in such useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the phage or cocktails thereof optionally in combination with another active compound may be incorporated into sustained-release preparations and devices.

The phage or cocktails thereof optionally in combination with another active compound may also be administered intravenously or intraperitoneally by infusion or injection. Solutions of the phage or cocktails thereof optionally in combination with another active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms during storage can be brought about by various antibacterial and antifungal agents, for example, parabens, chlorobutanol, phenol, sorbic acid, thimerosal, and the like. In many cases, it may be useful to include isotonic agents, for example, sugars, buffers or sodium chloride. Prolonged absorption of the injectable compositions can be brought about by the use in the compositions of agents delaying absorption, for example, aluminum monostearate and gelatin.

### Deposit Information

A deposit of representative bacteriophage of each of strains Φ2.3 and Φ2.3N13D5 (Φ2.3v1), which are disclosed herein above and referenced in the claims, has been made with the NRRL culture collection (USDA NRRL Agricultural Research Service Culture Collection, 1815 N. University St., Peoria, IL 61604). The date of deposit was Apr. 6, 2023. Upon issuance of a patent, all restrictions upon the deposit will be removed, and the deposit is intended to meet all of the requirements of 37 C.F.R. § 1.801-1.809. The accession numbers for the deposited strains are IDAC Accession No. 060423-03 and 060423-04 respectively. The deposit will be maintained in the depository for a period of 30 years, or 5 years after the last request, or for the effective life of the patent, whichever is longer, and will be replaced if necessary, during that period.

### EXAMPLES

The following examples are included to demonstrate embodiments of the invention. It should be appreciated by those of skill in the art that the techniques disclosed in the examples which follow represent techniques discovered by the inventor to function well in the practice of the invention, and thus can be considered to constitute exemplary modes for its practice. However, those of skill in the art should, in light of the present disclosure, appreciate that many changes can be made in the specific embodiments that are disclosed and still obtain a like or similar result without departing from the spirit and scope of the invention.

### Example 1

### Materials and Methods for Investigating Microbes and Microbial Factors Responsible for Progression of Alcohol-Related Liver Disease

### Patient Cohorts

26 subjects without alcohol use disorder (controls), 44 patients with alcohol use disorder, and 88 patients with alcoholic hepatitis were evaluated. Patients fulfilling the DSM IV criteria (Ball et al., 1997) of alcohol dependence and with active alcohol consumption (self-reported >60 g/day) were recruited in a clinic with an alcohol treatment program and compared to individuals without alcohol dependency (non-alcoholic controls; social drinkers consuming less than 20 g/day). Non-alcoholic controls or patients with alcohol use disorder did not take antibiotics or immunosuppressive medication during the two months preceding enrollment. Other exclusion criteria were diabetes, inflammatory bowel disease, known liver disease of any other etiology, and clinically significant cardio-vascular, pulmonary or renal co-morbidities. Alcoholic hepatitis patients were enrolled from the InTeam Consortium (ClinicalTrials.gov identifier number: NCT02075918) from 12 participating centers in the US, Mexico, Canada, United Kingdom, France and Spain. Inclusion criteria were: 1. Age >18 years and ≤70 years, 2. Active alcohol abuse (>50 g/day for men and >40 g/day for women) in the last 3 months, 3. Aspartate aminotransferase (AST)>alanine aminotransferase (ALT) and total bilirubin >3 mg/dl in the past 3 months, 4. Liver biopsy and/or clinical picture consistent with alcoholic hepatitis. Exclusion criteria were: 1. Autoimmune liver disease (ANA> 1/320), 2. Chronic viral hepatitis, 3. Hepatocellular carcinoma, 4. Complete portal vein thrombosis, 5. Extrahepatic terminal disease, 6. Pregnancy, and 7. Lack of signed informed consent (Brandl et al., 2018). In all patients, the clinical picture was consistent with alcoholic hepatitis and in patients who underwent liver biopsy, the histology was in line with the diagnosis of alcoholic hepatitis. For 3 patients who underwent liver transplantation, the transplantation date was considered as date of death. The baseline characteristics of the cohort with alcoholic hepatitis are shown in FIGS. 18A-B. Fecal 16S rRNA sequencing, Enterococcus culture and qPCR for cytolysin were performed. The MELD score was calculated from all patients from whom bilirubin level, INR, and creatinine level was available. The protocol was approved by the Ethics Committee of each participating center and patients were enrolled after written informed consent was obtained from each patient.

### Mice

C57BL/6 mice were purchased from Charles River. C57BL/6 germ-free mice were bred at UCSD. Sublytic Atp4α^{Sl/Sl} mice on a C57BL/6 background have been described (Llorente et al., 2017; Krieg et al., 2011) and heterozygous mice were used for breeding; sublytic Atp4α^{Sl/Sl} littermate mice and their wild-type littermates were used.

Female and male mice (age, 9-12 weeks) were placed on a chronic-binge ethanol diet (NIAAA model) as described (Bertola et al., 2013). Mice were fed with Lieber-DeCarli and the caloric intake from ethanol was 0% on days 1-5 and 36% from day 6 until the end of the study period. At day 16, mice were gavaged with a single dose of ethanol (5 g/kg body weight) in the early morning and sacrificed 9 hours later. Pair-fed control mice received a diet with an isocaloric substitution of dextrose.

Stool samples from patients with alcoholic hepatitis were used for fecal transplantation in germ-free mice. Mice were gavaged with 100 μl of stool samples (1 g stool dissolved in 30 ml Luria-Bertani (LB) medium containing 15% glycerol under anaerobic conditions), starting at an age of 5-6 weeks and repeated two weeks later. Two weeks after the second gavage, mice were placed on the ethanol or control (isocaloric) diet.

In studies of the effects of cytolysin, 5×10⁸ colony forming units (CFUs) of an isogenic cytolytic E. faecalis strain (FA2-2(pAM714)), a non-cytolytic E. faecalis strain that lacks cytolysin (FA2-2(pAM771)) (Ike et al., 1990) (E. faecalis Dcytolysin), or PBS (vehicle control) were fed to mice by gavage every third day, starting from day 6 through day 15 of ethanol feeding (see above). To determine the effect of bacteriophage treatment, 10¹⁰ plaque forming units (PFUs) E. faecalis phages (or C. crescentus phage phiCbK as control) (Gill et al., 2012) were gavaged to the mice 24 hours before the ethanol binge (at day 16). All animal studies were reviewed and approved by the Institutional Animal Care and Use Committee of the University of California, San Diego.

### Bacteriophages Isolation and Amplification

E. faecalis strain from mice feces was isolated before (Llorente et al., 2017) and E. faecalis strains from human stool samples were isolated using methods described below. All E. faecalis strains were grown statically in brain heart infusion (BHI) broth or on BHI agar at 37° C C. crescentus phage phiCbK was purified as previously described (Gill et al., 2012).

E. faecalis phages were isolated from untreated raw sewage water obtained from a North City Water Reclamation Plant in San Diego, California. Fifty milliliter raw sewage water was centrifuged at 8,000×g for 1 minute at room temperature (RT) to pellet large particles. The supernatant was passed through a 0.45 μm and then a 0.2 μm syringe filter (Whatman, PES membrane). One hundred microliter of the clarified sewage was mixed with 100 μl overnight E. faecalis culture and then added to BHI broth top agar (0.5% agar) and poured over a BHI plate (1.5% agar). After overnight growth at 37° C, the resulting plaques were recovered using a sterile pipette tip in 500 μl PBS. Phages were replaqued on E. faecalis three more times to ensure that the phages were clonal isolates.

High-titer phage stocks were propagated by infecting 200 ml of exponentially growing E. faecalis at a multiplicity of infection (MOI) of 0.1 in BHI broth containing 10 mM MgSO₄. Lysis was allowed to proceed for up to six hours at 37° C with shaking. The lysates were centrifuged at 10,000×g for 20 minutes at RT to remove the remaining bacterial cells and debris. Supernatant was then vacuum filtered through a 0.2 μm membrane filter and kept at 4° C until use.

Before mice were gavaged, 10-20 milliliter lysates were concentrated using Corning Spin-X UF Concentrators with 100,000-molecular-weight-cutoff (MWCO) to a volume of approximately 1 ml. Following concentration, the culture medium was replaced with PBS via diafiltration. The resulting lysate was further concentrated to a final volume of 0.5 ml and adjusted to the required PFUs.

### Whole-Genome Sequencing for Bacteriophages

Ten milliliter of lysates were treated with 10 μg/ml each of DNase and RNase at 37° C for 1 hour and phages were precipitated by adding 1 M NaCl and 10% (w/v) polyethylene glycol 8000 (PEG 8000) and incubated at 4° C overnight. Precipitated phages were then pelleted by centrifuging at 10,000×g for 10 minutes at 4° C and resuspended in 500 μl of resuspension buffer (5 mM MgSO₄). Phage DNA was then extracted using Promega Wizard DNA Clean-up kit (Promega).

### Electron Microscopy

Bacteriophage morphology was examined by transmission electron microscopy of negatively stained grids, prepared using the valentine method with 2% uranyl-acetate (Valentine et al., 1968), and examined at an acceleration voltage of 100 KV in the JEOL 1200 EX. Phosphotungstic acid was used to confirm the contractile tails of myophages. Transmission Electron Microscope at the Microscopy and Imaging Center at Texas A&M University.

Bacterial DNA extraction and 16S rRNA sequencing DNA from human stool samples, mouse liver sections or bacterial culture was extracted as described before (Llorente et al., 2017) and DNA from mouse feces were extracted using QIAamp Fast DNA Stool kit (QIAGEN). 16S ribosomal RNA (rRNA) gene sequencing was performed as described (Llorente et al., 2017).

### Real-Time Quantitative PCR

Bacterial genomic DNA was extracted from human stool samples and mouse liver (Llorente et al., 2017). RNA was extracted from mouse liver and cDNAs were generated (Llorente et al., 2017). Primer sequences for mouse genes were obtained from the NIH qPrimerDepot. Primer sequences for E. faecalis 16S rRNA gene, E. faecalis cylL_{S} and cylL_{L} genes were described before (Ryu et al., 2013; Haas et al., 2002). Mouse gene expression and amplification of bacterial genes were determined with Sybr Green (Bio-Rad Laboratories) using ABI StepOnePlus real-time PCR system. The qPCR value of mouse genes was normalized to 18S.

E. faecalis Whole-Genome Sequencing

To isolate E. faecalis strains from human subjects, 50-300 mg of human stool was resuspended in 500 μl PBS, serial dilutions were made, and 100 μl was placed on plates with selective medium, BBL Enterococcosel broth (Becton Dickinson). Enterococci colonies were identified by the production of dark brown or black color generated by hydrolysis of esculin to esculetin that reacts with ferric ammonium citrate. Each Enterococcus colony was then picked, and we performed qPCR to identify E. faecalis, using specific primers against the E. faecalis 16S rRNA gene (Ryu et al., 2013). For each subject, 1-6 E. faecalis colonies were analyzed and bacterial genomic DNA was then extracted as described in above section. DNA sequencing was performed on the Illumina HiSeq X generating paired end reads (2×151 bp). Bacterial genomes were assembled and annotated using the pipeline described previously (Page et al., 2016). For the phylogeny of E. faecalis, the genome assemblies of the UK clinical isolates (Raven et al., 2016) were combined with the assemblies of the study isolates, annotated with Prokka (Seemann, 2014), and a pangenome estimated using Roary (Page et al., 2016). A 95% identity cut-off was used, and core genes were defined as those in 99% of isolates. A maximum likelihood tree of the SNPs in the core genes was created using RaxML (Stamatakis, 2014) and 100 bootstraps. The resulting tree was visualized using Microreact (Argimon et al., 2016).

E. faecalis Culture

All E. faecalis strains were grown statically in brain heart infusion (BHI) broth or on BHI agar plate at 37° C 50 μg/ml erythromycin.

### Fecal Enterococcus Level Determination

To determine fecal Enterococci level in mice, 10-30 mg of mouse feces was resuspended into 500 μl PBS and serial dilutions were made. Five microliter of each dilution from each sample were spotted onto a plate with a selective medium, BBL Enterococcosel broth (Becton Dickinson). Enterococci colonies were identified by the production of dark brown or black color generated by hydrolysis of esculin to esculetin that reacts with ferric ammonium citrate. Colony numbers of each sample were then counted and CFUs were calculated.

### Cytolysin Expression and Purification

To purify bioactive CylL_{L}″ and CylL_{S}″, an E. coli heterologous expression system was used. Briefly, either His×6-CylL_{L} or His×6-CylL_{S} were co-expressed with CylM (enzyme that performs dehydration and cyclization reactions on cytolysin) in E. coli to yield fully dehydrated and cyclized full-length peptides. The His-tag and leader peptide were then cleaved off using recombinant CylA (27-412), the soluble domain of the native peptidase used in cytolysin maturation, to yield bioactive CylL_{L}″ or CylL_{S}″. The resulting core peptides were further purified by reverse-phase HPLC.

The cylL_{L} and cylL_{S} genes were previously cloned into the MCSI of a pRSFDuet-1 backbone vector which contained the cylMgene in MCSII (Tang and van der Donk, 2013). The cylA (27-412) gene was previously cloned into MCSI of a pRSFDuet-1 backbone vector (Tang et al., 2018). E. coli BL21 Star™ (DE3) cells (50 μl) were transformed with 100 ng of either the cylL_{L}_cylM:pRSFDuet, cylL_{S}_cylMpRSFDuet or cylA (27-412):pRSFDuet plasmids via KCM chemical transformation. The cells were plated on LB agar plates supplemented with kanamycin (50 μg/ml) and grown at 37° C overnight. One colony was picked to inoculate 15 ml of LB broth supplemented with kanamycin overnight at 37° C The culture was used to inoculate 1.5 liters of terrific broth supplemented with kanamycin. Cultures were grown with shaking at 37° C to an OD₆₀₀ of 0.8. The temperature of the incubator was lowered to 18° C and expression was induced with the addition of 0.3 mM final concentration of isopropyl 3-D-thiogalactoside. The cultures were allowed to incubate at 18° C for 18 hours. The cells were collected by centrifugation at 5000×g for 12 minutes. The cell paste was collected and frozen at −70° C.

For the purification of the protease CylA (27-412), the cell paste was thawed and resuspended in 50 ml LanP buffer (20 mM HEPES, 1 M NaCl, pH 7.5). The cell suspension was lysed by homogenization. The lysate was clarified by centrifugation at 13,000×g for 45 minutes and filtered through a 0.45 μm centrifugal filter (Thermo Scientific). The clarified lysate was applied to a pre-equilibrated HisTrap HP 5 ml column (GE Healthcare) through a peristaltic pump. The loaded column was connected to an AKTA pure 25 M system. The protein was eluted by a linear gradient of LanP buffer and Elution Buffer (20 mM HEPES, 1 M NaCl, 500 mM imidazole, 10% glycerol, pH 7.5). The purest fractions, as determined by 4%-20% SDS-PAGE, were combined, concentrated to 1 mg/ml by Amicon Ultra Centrifugal Filters (30 kDa MWCO), and buffer exchanged into storage buffer (20 mM HEPES, 300 mM KCl, 10% glycerol, pH 7.5) by PD-10 desalting column (GE Healthcare). Protein concentration was determined by absorbance at 280 nm.

For the purification of CylL_{L} and CylL_{S} peptides, the cell paste was thawed and resuspended in 50 ml of LanA Buffer B1 (6 M guanidine HCl, 20 mM NaH₂PO₄, 500 mM NaCl, 0.5 mM imidazole, pH 7.5). The cell suspension was lysed via sonication (2 sec pulse on, 5 seconds pulse off, 7 minutes total pulse on time). The cell lysate was clarified by centrifugation at 13,000×g for 45 minutes. The clarified cell lysate was filtered through a 0.45 μm centrifugal filter and applied via gravity flow to a pre-equilibrated, 2 ml bed volume of His60 Ni Superflow Resin (Clonetech). After the lysate had been applied, the resin was washed with 15 ml of LanA Buffer B2 (4 M guanidine HCl, 20 mM NaH₂PO₄, 500 mM NaCl, 30 mM imidazole, pH 7.5). The resin was washed again with 15 ml of LanA Wash Buffer (20 mM NaH₂PO₄, 500 mM NaCl, 30 mM imidazole, pH 7.5) to remove the guanidine HCl. The peptides were eluted with 10 ml of LanA Elution Buffer (20 mM NaH₂PO₄, 500 mM NaCl, 500 mM imidazole, pH 7.5). A 0.02 mg/ml final concentration of CylA (27-412) was added to the elution fraction and allowed to incubate at room temperature overnight to remove the leader peptide.

The digestion was quenched by adding 2% (v/v) final concentration of trifluoroacetic acid. The solution was centrifuged at 4500×g for 10 minutes and filtered through a 0.45 μm syringe filter (Thermo Scientific). The core peptides were purified by semi-preparative reverse phase HPLC using a Phenomenex Jupiter Proteo column (10 mm×250 mm, 4 μm, 90 A) connected to an Agilent 1260 Infinity II liquid chromatography system. The peptides were separated using a linear gradient of 3% (v/v) solvent B (acetonitrile+0.1% trifluoroacetic acid) in solvent A (water+0.10% trifluoroacetic acid). The fractions were spotted on a MALDI target plate by mixing 1 μl of sample with 1 μl of a 25 mg/ml solution of Super-DHB (Sigma) in 80% acetonitrile/water+0.1% trifluoroacetic acid. The fractions were analyzed by MALDI-TOF MS on a Bruker UltrafleXtreme MALDI-TOF/TOF operating in positive ionization, reflector mode.

### Primary Mouse Hepatocytes

Hepatocytes were isolated from C57BL/6 female mice fed the chronic-binge ethanol diet (NIAAA model) (Bertola et al., 2013). Livers were perfused in situ with 0.5 mM EGTA containing calcium-free salt solution and then perfused with a solution containing 0.02% (w/v) collagenase D (Roche Applied Science). Livers were then carefully minced and filtered using a 70 μm nylon cell strainer. Hepatocytes were centrifuged at 50×g for 1 minute after three times washing. Hepatocyte viability was assessed by Trypan Blue (Thermo Fisher Scientific). 1.5×10⁵ hepatocytes were seeded on 12-well plates coated with rat collagen type I in DMEM-F12 (Thermo Fisher Scientific) with insulin-transferrin-selenium (1% v/v) (Thermo Fisher Scientific) and 40 ng/ml dexamethasone (MP Biomedicals) containing 10% (v/v) fetal bovine serum (FBS; Gemini Bio-Products) and antibiotics. After 4 hours, the culture was washed with DMEM-F12 media and changed to the same complemented media without FBS (Iwaisako et al., 2012). Then 16 hours later, hepatocytes were cultured with 0 or 25 mM ethanol and stimulated with 0, 200 or 400 nM CylL_{S}″ and/or CylL_{L}″ in the same culture medium without FBS. After 3 hours stimulation, hepatocyte cytotoxicity was assessed using Pierce LDH Cytotoxicity Detection Kit (Thermo Fisher Scientific). After 6 hours stimulation, the viability of hepatocytes was determined by incubation with 0.3 mg/ml 3-(4,5-dimethylthiazol-2-yl)-2,5′-diphenyltetrazolium bromide solution (MTT; Sigma-Aldrich) in DMEM-F12 media with 10% (v/v) FBS and incubated at 37° C for one hour. The medium was then removed, and dimethyl sulfoxide (Sigma-Aldrich) was added to dissolve the formazan. Formazan concentration was determined by absorbance at 550 nm and the survival percentage was calculated accordingly (hepatocytes not stimulated with cytolysin peptides were set as 100% survival).

### Biochemical Analysis

Serum levels of ALT were determined using Infinity ALT kit (Thermo Scientific). Hepatic triglyceride levels were measured using Triglyceride Liquid Reagents kit (Pointe Scientific). Levels of serum LPS and fecal albumin were determined by ELISA kits (Lifeome Biolabs and Bethyl Labs, respectively). Serum levels of ethanol were measured using Ethanol Assay kit (BioVision).

### Staining Procedures

To determine lipid accumulation, liver sections were embedded in OCT compound. 8 μm frozen sections were then cut and stained with Oil Red O (Sigma-Aldrich). Representative pictures from each group of mice were shown in each figure.

### Statistical Analysis

Results are expressed as mean±s.e.m. (except when stated otherwise). Univariate Cox regression analysis was used to detect associations of cytolysin and MELD with overall mortality. Univariate logistic regression analysis of laboratory and clinical parameters associated with cytolysin was performed. A multivariate logistic regression model was performed controlling for MELD and platelet numbers. Multicollinearity was examined using the variance inflation factor (VIF). Kaplan-Meier curves were used to compare survival between cytolysin positive and negative alcoholic hepatitis patients. Fecal E. faecalis, bacterial diversity and richness from controls and patients were compared using Kruskal-Wallis test with Dunn's post-hoc test. Percentage of subjects with fecal samples positive for E. faecalis and cytolysin was compared using Fisher's exact test followed by false discovery rate (FDR) procedures. Jaccard dissimilarity matrices were used for principal coordinate analysis (PCoA) and p-values were determined by permutational multivariate analysis of variance (PERMANOVA) followed by FDR procedures to correct for multiple comparisons.

For mouse and cell culture studies, significance of multiple groups was evaluated using one-way or two-way analysis of variance (ANOVA) with Tukey's post-hoc test. Fisher's exact test was used in the analysis of liver cylL_{S}. FDR correction was applied to the p-values from Fisher's exact test to correct for multiple comparisons. Kaplan-Meier curves were used to compare survival between experimental mouse groups. PCoA based on Jaccard dissimilarity matrices was performed between experimental mouse groups and the p-values were determined by PERMANOVA followed by FDR procedures to correct for multiple comparisons. Statistical analyses were performed using R statistical software, R version 3.5.1, 2018 the R Foundation for Statistical Computing and GraphPad Prism v6.01. A p-value <0.05 was considered to be statistically significant.

### Example 2

### Investigating Mechanism of Cytolysin-Associated Liver Damage

Alcohol-related liver disease can be transmitted via fecal microbiota. Colonization of germ-free mice with feces from patients with alcoholic hepatitis exacerbates ethanol-induced liver disease, compared to feces from conventionally colonized mice (Llopis et al., 2016). Microbes and microbial factors responsible for this transmissible phenotype and for progression of alcohol-related liver disease were investigated.

To determine whether chronic alcohol use and alcoholic hepatitis are associated with an altered composition of the gut microbiota, changes in the fecal microbiota were analyzed by 16S ribosomal RNA (rRNA) gene sequencing. Changes in fecal microbiota composition were noted in patients with alcohol use disorder or alcoholic hepatitis, compared to subjects without alcohol use disorder (controls). One significant change that was observed was an increase in proportion of Enterococcus spp. In patients with alcoholic hepatitis, about 5% fecal bacteria were Enterococcus spp, compared with almost none in patients with alcohol use disorder or controls. Fecal samples from patients with alcoholic hepatitis had about 2700-fold more Enterococcus faecalis (E. faecalis) than samples from controls, measured by quantitative PCR (qPCR), consistent with 16S rRNA sequencing results. However, proportions of E. faecalis did not correlate with disease severity in patients with alcoholic hepatitis (data not shown).

Colonization of mice with E. faecalis induces mild hepatic steatosis and exacerbates ethanol-induced liver disease (Lorente et al., 2017) by unclear mechanisms. Cytolysin is a bacterial exotoxin or bacteriocin produced by E. faecalis (Huycke et al., 1991) that contains two post-translationally modified peptides, CylL_{L}″ and CylL_{S}″ (Tang and van der Donk, 2013). Cytolysin has lytic activity against not only Gram-positive bacteria but also eukaryotic cells (Cox et al., 2005). cylL_{L} and cylL_{S} genomic DNA (cytolysin positive) was detected in fecal samples of 30% of patients with alcoholic hepatitis; none of the fecal samples from controls and the sample from only one patient with alcohol use disorder (out of a total of 38) was cytolysin positive, detected by qPCR. After this patient was enrolled in a physician-supervised abstinence program, their fecal sample became negative for cytolysin (not shown). Importantly, 78% of cytolysin-positive patients with alcoholic hepatitis died within 180 days after admission.

Univariate logistic regression of laboratory and clinical parameters associated detection of cytolysin in feces with international normalized ratio (INR), platelet numbers, model for end-stage liver disease (MELD) score, sodium MELD (MELDNa) score, and death. Detection of cytolysin correlated with overall mortality in alcoholic hepatitis patients with a hazard ratio of 22.24 (95% CI, 5.136-96.3; P=3e-09), whereas MELD score, which is used to predict mortality in clinical practice, had a hazard ratio of 1.068 (95% CI, 1.009-1.13; P=0.02). When we performed receiver operating characteristic (ROC) curve analysis, MELD had an area under the curve (AUC) of 0.7, whereas detection of cytolysin had an AUC of 0.81. Whole-genome sequencing of 148 E. faecalis isolates revealed the phylogenetic diversity of cytolysin-positive E. faecalis from patients with alcohol-associated hepatitis. Based on our findings, detection of cytolysin is a prognostic factor for worse liver-related outcomes and death.

To determine whether cytolysin contributes to liver damage mediated by E. faecalis, mice were gavaged with an isogenic cytolytic E. faecalis strain (FA2-2(pAM714)) or an E. faecalis strain that lacks cytolysin (FA2-2(pAM771)) (Ike et al., 1990) (non-cytolytic); the mice were then placed on a chronic-plus-binge ethanol diet (Bertola et al., 2013). Compared to mice given phosphate-buffed saline (PBS), mice fed ethanol after they were colonized with cytolytic E. faecalis developed more severe liver injury, indicated by higher level of alanine amino-transferase (ALT) and hepatic steatosis. The mice fed ethanol after they were colonized with cytolytic E. faecalis also had more liver inflammation with higher expression levels of mRNAs encoding inflammatory cytokines and chemokines (Il1b, Cxcl1, and Cxcl2) compared to mice given PBS. Mice fed ethanol after they were colonized with non-cytolytic E. faecalis had significantly less ethanol-induced liver injury, steatosis, and inflammation and longer survival times, compared with mice fed ethanol after they were colonized with cytolytic E. faecalis.

To explore the mechanism of cytolysin-associated liver damage, we measured cytolysin in the liver. CylL_{S} was significantly increased in livers of mice colonized with cytolytic E. faecalis but not mice that were not given E. faecalis or mice colonized with non-cytolytic E. faecalis following chronic ethanol administration. Cytolysin was not detectable in livers of mice fed an isocaloric (control) diet, indicating that ethanol-induced changes in the gut barrier are necessary for translocation of cytolytic E. faecalis from the intestine to the liver. Increased intestinal permeability was observed in ethanol-fed mice compared with isocaloric diet-fed mice, but there were no significant differences among mice that were not given E. faecalis and mice colonized with cytolytic or non-cytolytic E. faecalis following chronic ethanol administration, indicating that cytolysin does not affect intestinal barrier function.

Colonization of mice with cytolytic or non-cytolytic E. faecalis did not significantly change the composition of the intestinal microbiota, based on 16S rRNA gene sequencing. Cytolytic E. faecalis did not affect intestinal absorption or hepatic metabolism of ethanol, based on serum levels of ethanol and liver levels of Adh1 or Cyp2e1 mRNAs (encode enzymes that metabolize ethanol in the liver). These results indicate that E. faecalis that produce cytolysin promotes ethanol-induced liver disease in mice.

Germ-free mice were colonized with feces from cytolysin-negative and -positive patients with alcoholic hepatitis. Consistent with the findings from mice colonized with cytolytic E. faecalis, gnotobiotic C57BL/6 mice colonized with feces from two different cytolysin-positive patients developed more severe ethanol-induced liver injury, steatosis, and inflammation than mice given feces from two different cytolysin-negative patients. Transplantation of feces from cytolysin-positive patients reduced survival time of the mice and increased translocation of cytolytic E. faecalis to the liver following ethanol administration. Composition of the intestinal microbiota was not different between mice colonized with feces from cytolysin positive or negative alcoholic hepatitis donors following the control diet, based on 16S rRNA gene sequencing. Mice transplanted with feces from one cytolysin-positive alcoholic hepatitis patient (#2) showed a significantly different microbiota than the other mouse groups following ethanol administration. Numbers of Enterococcus were higher in feces from ethanol-fed mice given feces from cytolysin-positive than cytolysin-negative patients, but there was no significant difference between these mice if they were on the control diet. Interestingly, stool samples from donors with cytolytic E. faecalis did not appear to contain cytolysin-negative E. faecalis. No differences were observed between mice colonized with feces from cytolysin-positive vs cytolysin-negative patients in intestinal absorption or hepatic metabolism of ethanol. These results provide further evidence that cytolysin promotes ethanol-induced liver disease.

To determine the mechanism by which cytolysin increases liver disease, we isolated hepatocytes from mice on ethanol or control diets and stimulated them with pure bioactive cytolysin peptides (CylL_{L} and CylL_{S}) (Tang and van der Donk, 2013). Incubation of the primary mouse hepatocytes with two cytolysin subunits caused a dose-dependent increase in cell death compared to hepatocytes incubated with vehicle or with one subunit alone. Interestingly, when hepatocytes were isolated from ethanol-fed mice and then incubated with ethanol, increased levels of cytolysin-induced cell death were not observed compared to hepatocytes isolated from mice on the control diet. The cytotoxic effects of cytolysin are likely mediated by pore formation, resulting in cell lysis (Van Tyne et al., 2013). The results indicate that ethanol-associated gut barrier dysfunction and subsequent translocation of cytolytic E. faecalis from the intestine to the liver contributes to liver cell damage.

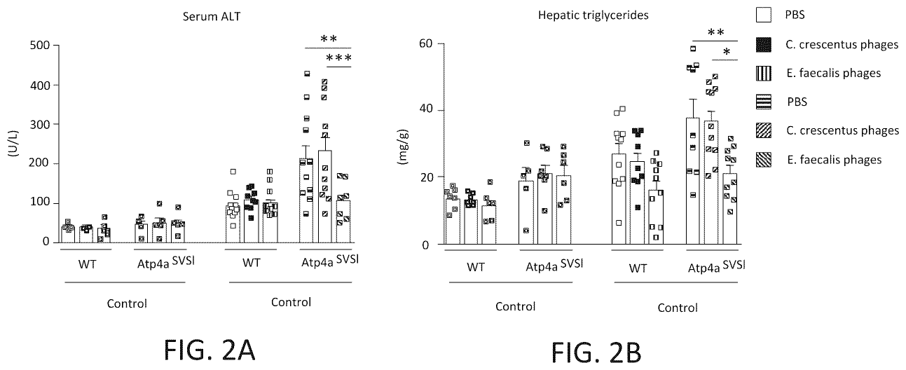

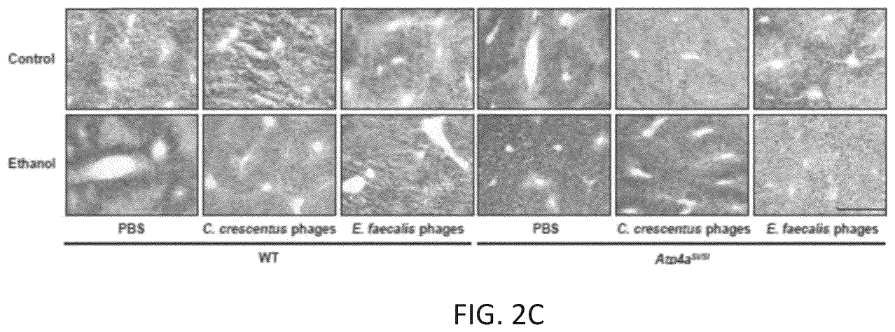

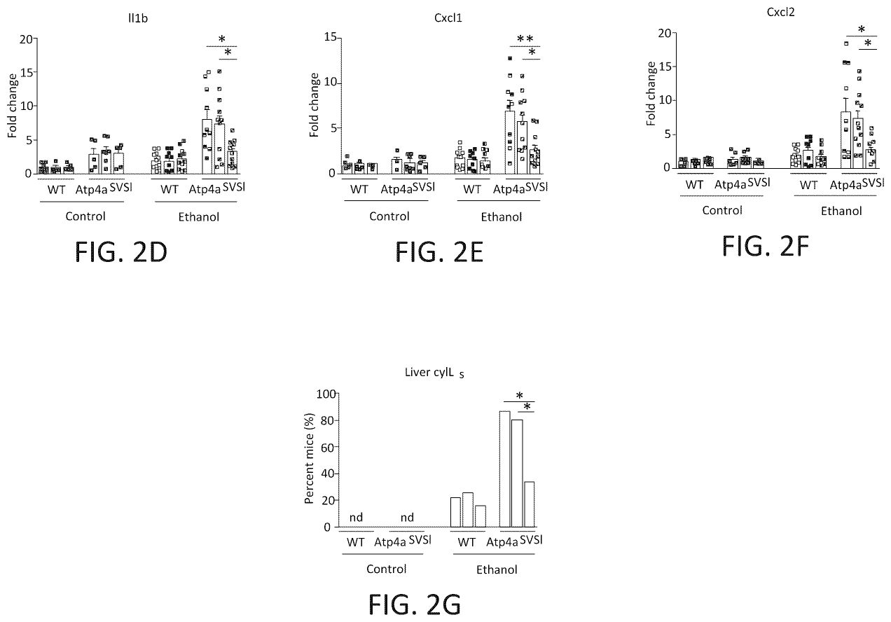

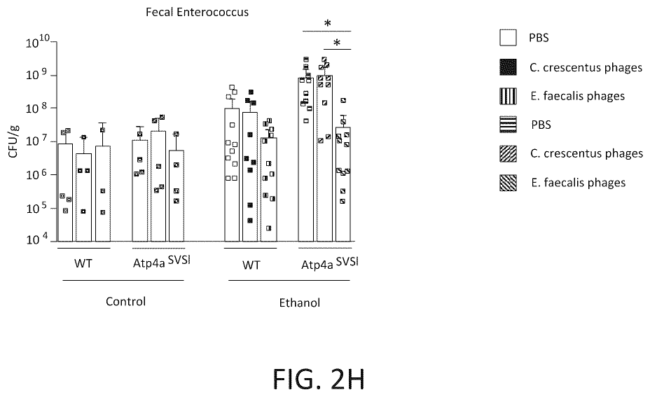

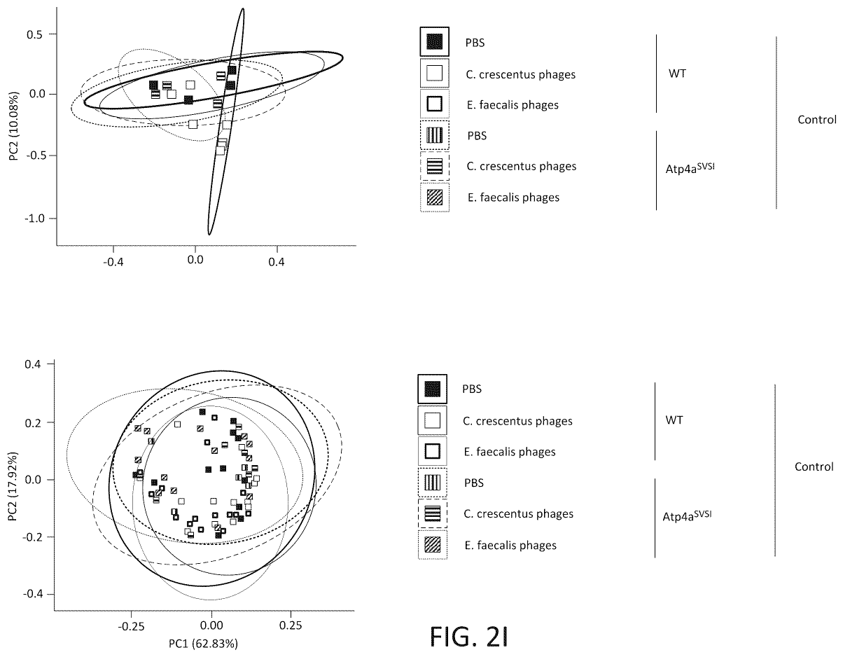

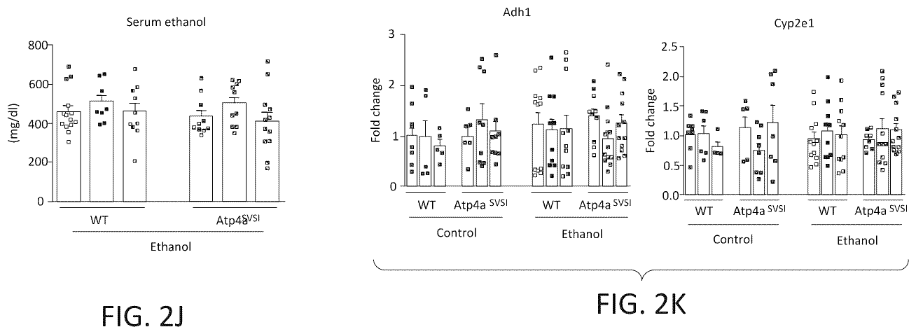

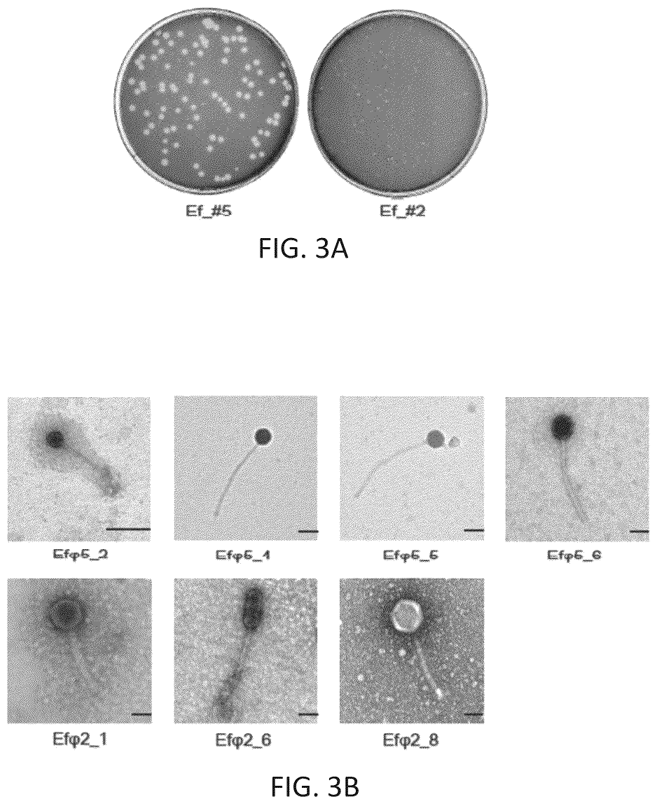

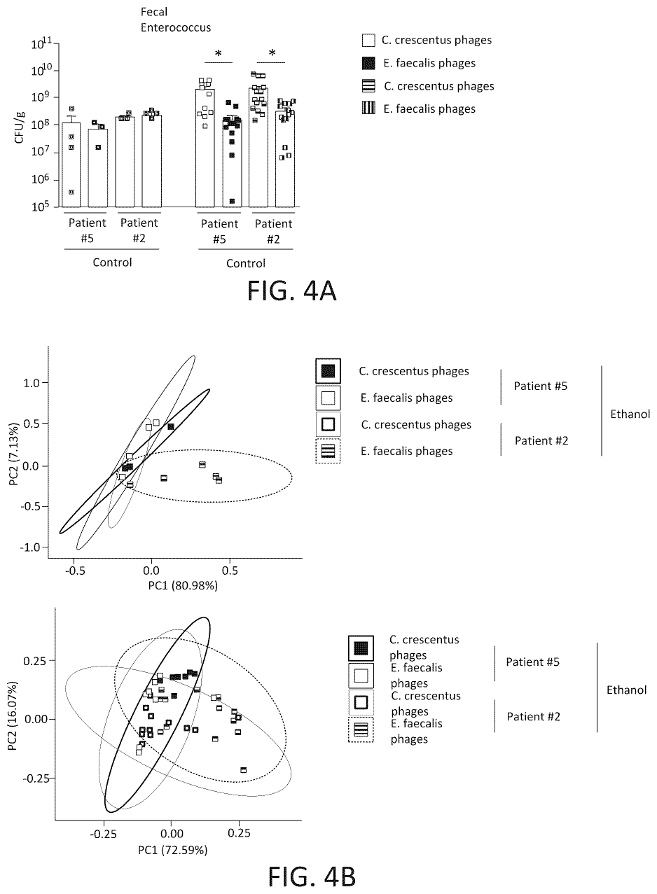

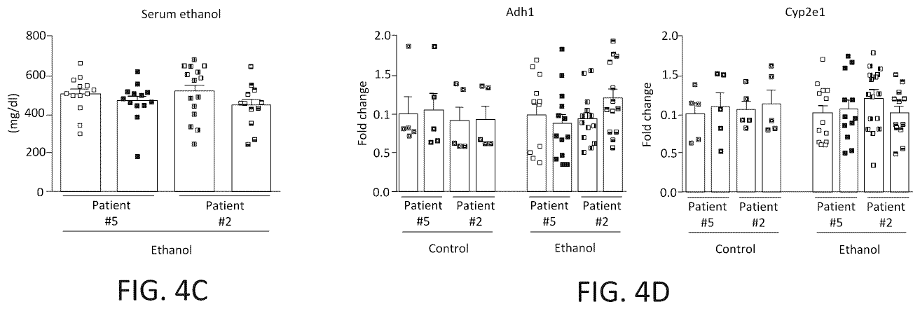

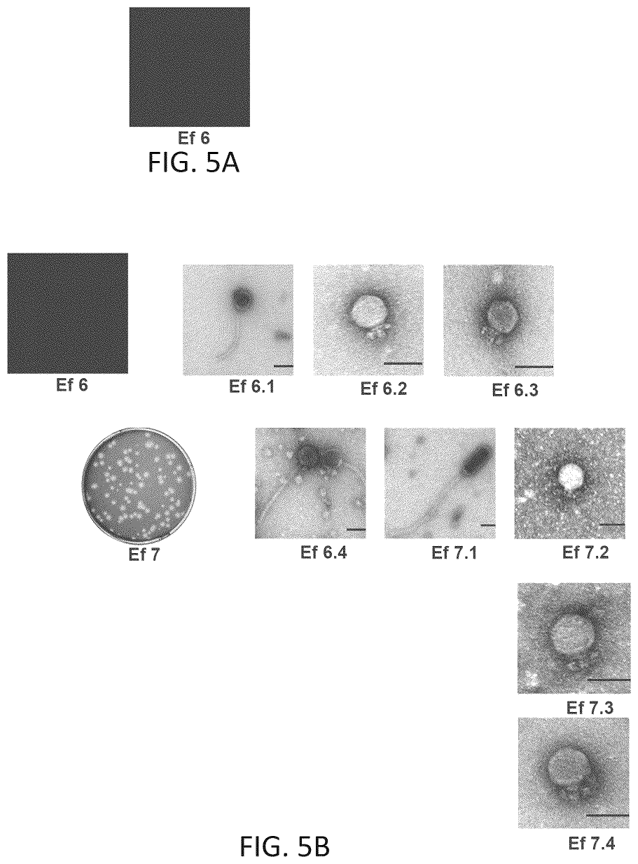

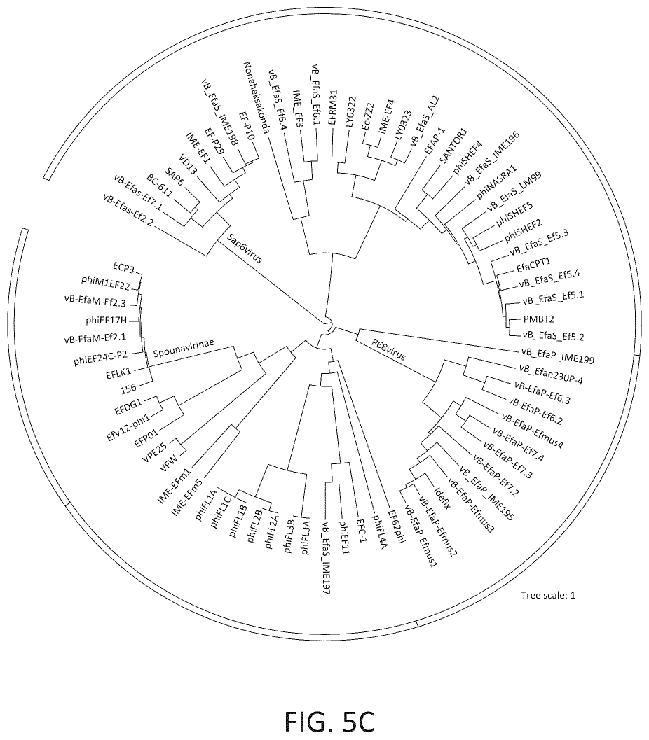

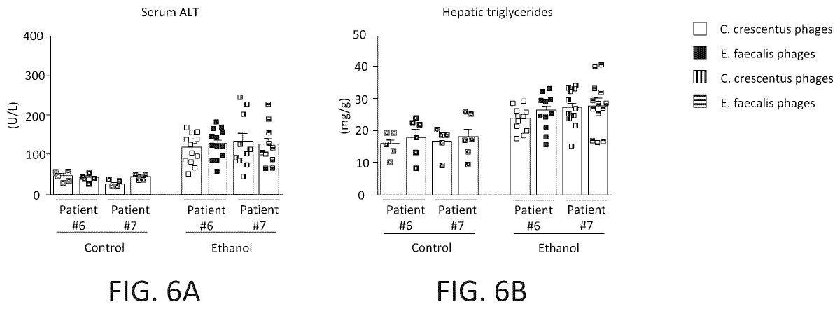

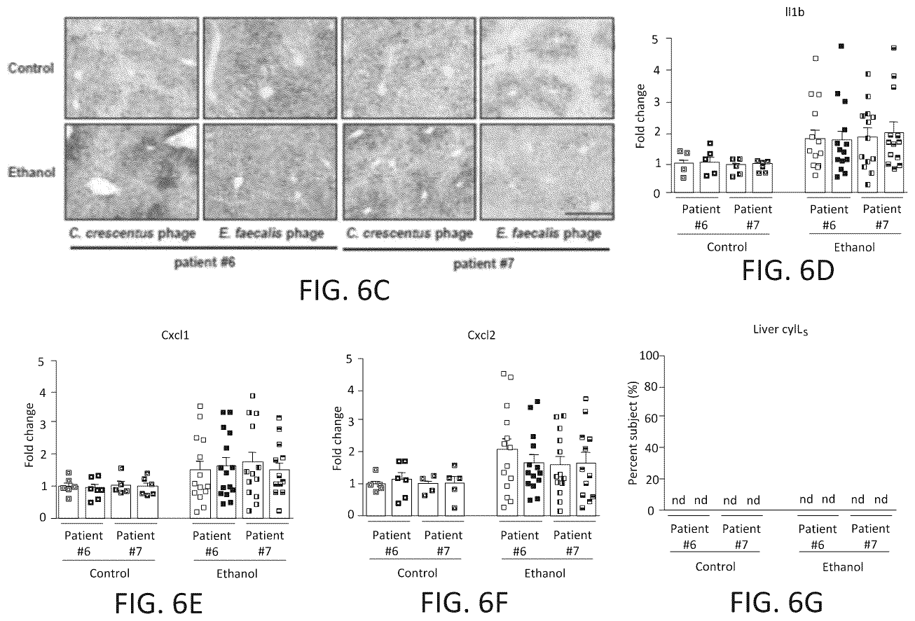

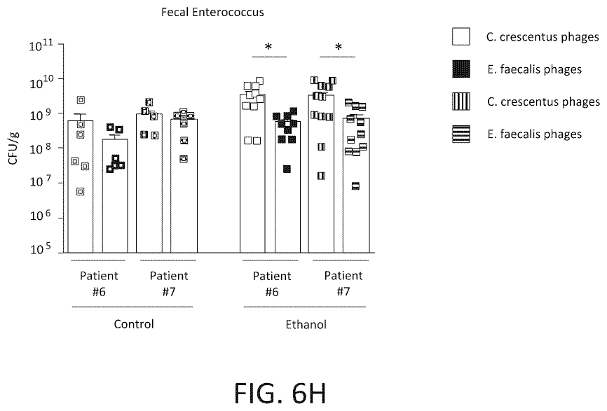

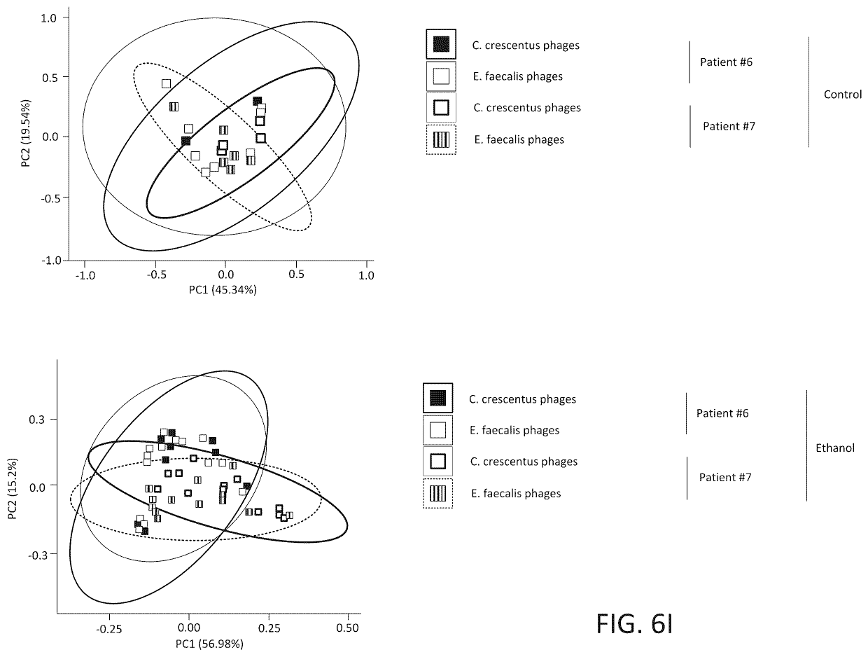

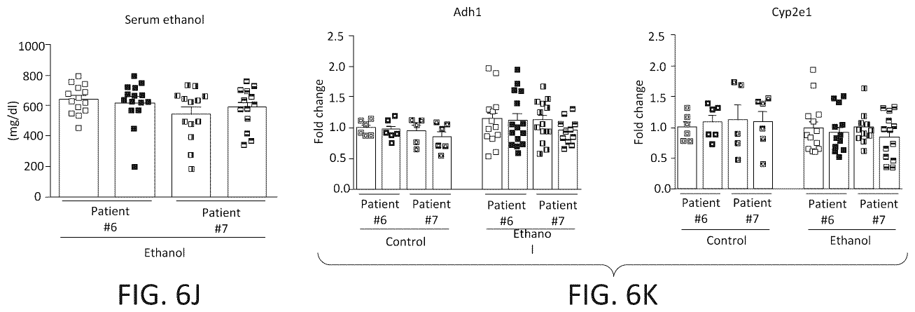

To further demonstrate a causative role of cytolytic E. faecalis for the development of ethanol-induced steatohepatitis, the effects of naturally occurring bacteriophages that reduce cytolytic E. faecalis in the intestine were investigated. Bacteriophages, viruses that infect bacteria, are highly specific for bacterial strains (Nobrega et al., 2018). Atp4α^{sl/sl} mice, which lack gastric acid, have overgrowth of intestinal enterococci, associated with increased susceptibility to alcohol-induced steatohepatitis (Llorente et al., 2017). Gavaging of wild-type mice with an E. faecalis strain isolated from Atp4α^{sl/sl} mice increased ethanol-induced steatohepatitis (Llorente et al., 2017). It was found that this E. faecalis strain expressed cytolysin. Four distinct naturally occurring bacteriophages that lyse the cytolytic E. faecalis strain isolated from Atp4α^{Sl/Sl} mice were isolated. Lytic bacteriophages were from the family of Podoviridae. Atp4α^{Sl/Sl} mice and their wild-type littermates were then placed on the chronic-plus-binge ethanol diet and gavaged with the lytic bacteriophage cocktail. Bacteriophages directed against Caulobacter crescentus (C. crescentus), a bacterium that is present in freshwater lakes and streams (Poindexter, 1964), but does not colonize humans or rodents (Shin et al., 2016), were used as controls. Compared to Atp4α^{Sl/Sl} mice gavaged with control bacteriophage or vehicle, Atp4α^{Sl/Sl} mice gavaged with bacteriophages that target cytolytic E. faecalis had less liver injury, steatosis, and inflammation following chronic ethanol feeding. Administration of E. faecalis bacteriophage significantly reduced levels of cytolysin in the liver (FIG. 2g) and fecal amounts of Enterococcus. Bacteriophage administration did not affect the overall composition of the fecal microbiome or intestinal absorption or hepatic metabolism of ethanol.

To develop a novel therapeutic approach to precisely edit the intestinal microbiota, bacteriophages against cytolytic E. faecalis strain that was isolated from fecal samples of patients with alcoholic hepatitis were isolated and amplified. Lytic bacteriophages against cytolytic E. faecalis were from the family of either Siphoviridae or Myoviridae. These bacteriophages were patient specific, in that they did not lyse cytolytic E. faecalis from another patient with alcoholic hepatitis (data not shown). Gnotobiotic mice were colonized with feces from two different cytolysin-positive patients with alcoholic hepatitis and given 3-4 different lytic bacteriophages against cytolytic E. faecalis. The bacteriophages against cytolytic E. faecalis reduced ethanol-induced liver disease, based on lower levels of ALT and hepatic triglycerides, decreased hepatic levels of Il1b, Cxcl1, and Cxcl2 mRNAs, and reduced hepatic levels of cylL_{S}, compared with mice given control bacteriophages (against C. crescentus). Bacteriophages against cytolytic E. faecalis also reduced fecal amounts of Enterococcus without affecting the overall composition of the gut microbiota. Intestinal absorption of ethanol and hepatic metabolism were similar in all groups.

To demonstrate that the effect of bacteriophage treatment occurs via targeting of cytolytic E. faecalis, rather than reductions in non-cytolytic E. faecalis, gnotobiotic mice were colonized with feces from cytolysin-negative patients with alcoholic hepatitis. Bacteriophages against non-cytolytic E. faecalis from patients were isolated and amplified, and they were from the family of either Siphoviridae or Podoviridae. These bacteriophages did not reduce features of ethanol-induced liver disease compared with control bacteriophages, despite the reduction of fecal Enterococcus. These findings indicate that lytic bacteriophage treatment can reduce features of ethanol-induced liver disease caused by cytolytic E. faecalis.

These findings link an increase in a specific bacterial strain with worse outcomes and mortality in patients with alcoholic hepatitis. Cytolysin, a virulence factor produced by cytolytic E. faecalis, causes direct hepatocyte death and contributes to liver disease. Cytolysin does not affect the intestinal barrier or promote its own translocation from the intestine to the liver. Translocation of cytolytic E. faecalis to the liver is required for development of liver disease during chronic administration of ethanol, which is likely facilitated by ethanol-induced changes in the intestinal barrier (Schnabl and Brenner, 2014).

Bacteriophages that target cytolytic E. faecalis might be used to treat patients with alcoholic hepatitis, a life-threatening disease with no effective treatment. Personalized diagnostics allow identification of patients colonized with cytolytic E. faecalis, who are predicted to have worse clinical outcomes. Phages can precisely edit the intestinal microbiota and selectively target and eradicate specific strains of bacteria. These types of phage-based therapies have been predominantly studied in trials of patients infected with multidrug-resistant bacteria (Schooley et al., 2014) or gastrointestinal diseases (Marcuk et al., 1971; Sarker et al., 2016; Dalmasso et al., 2014). These trials have proven phage-based therapies to be safe-even when bacteriophages are administered intravenously (Schooley et al., 2014). The data herein evidence that eradication of cytolytic E. faecalis in patients with alcoholic hepatitis might produce better outcomes than current treatments.

### Example 3

Bacteriophages Having a Broader Host Range Against Cytolytic E. faecalis and Methods of Producing the Same

To broaden the host range of bacteriophages against cytolytic E. faecalis natural selection of existing phages was used to expand the host range and to identify phages with genetic differences.

Although about 40 Enterococcus faecalis (E. faecalis) phages have been isolated, the host range of the E. faecalis phages for cytolysin-positive E. faecalis clinical isolates (reference bacterial strains), which drive alcoholic hepatitis, is around 60%. On the other hand, phages with a wider host range are needed for clinical application.

### Methods

Among the E. faecalis phages isolated to date, ΦEf2.3, which has the widest host range against the reference bacterial strains, was used. The phage was co-cultured with the reference strains (16 E. faecalis strains) for 5 days for producing evolved phage, and “evolved phages” adapted to the host by co-culturing were isolated from the supernatant of the culture on day 5.

The obtained evolved phages were used to determine the host range. In addition, the obtained evolved phages were further used for second and third rounds to insert additional mutations into their viral genomes. The presence of those mutations in known phages was verified by BLAST.

### Results

### Validation of the Host Range of ΦEf2.3

The infectivity of ΦEf2.3 against reference strains (16 E. faecalis strains) was determined. 3 μL of phage solution was dropped onto a lawn containing each host and infectivity was evaluated by plaque formation activity (spot test). As shown in Table 1, ΦEf2.3 was active against 10 of the 16 strains, but not against 6 strains (EF02, 04, 06, 09, 12 and 15).

TABLE 1
Host range of ΦEf2.3
Sensitivity of reference 16 E. faecalis clinical isolates against ΦEf2.3

| | E. faecalis Reference strains | | | | | | | | | | | | | | | |
|---|---|---|---|---|---|---|---|---|---|---|---|---|---|---|---|---|
| | EF01 | EF02 | EF03 | EF04 | EF05 | EF06 | EF07 | EF08 | EF09 | EF10 | EF11 | EF12 | EF13 | EF14 | EF15 | EF16 |
| [also | 41-2 | A43-1 | A18-3 | 52-2 | A49-2 | 66-9 | A26-3 | 13-36 | A2-7 | 70-6 | A17-1 | 61-1 | 47-1 | 11-1 | A25-8 | 4-1 |
| known as] | | | | | | | | | | | | | | | | |
| ΦEf2.3 | + | − | + | − | + | − | + | + | − | + | + | − | + | + | − | + |
| Infectivity | | | | | | | | | | | | | | | | |

+: Activity detected
−: No activity detected

Co-Culture of ΦEf2.3 with Reference Strains

In order to evolve ΦEf2.3, the phage was co-cultured with reference strains (16 E. faecalis strains). ΦEf2.3 was inoculated at MOI of 0.01 against each reference strain in the log growth phase (OD=0.2-0.4). ΦEf2.3 and reference strains were co-cultured at 37±2° C with shaking, and 10% of co-culture was passaged every 24 hours into fresh BHI medium. This procedure was repeated for 5 days, and the lytic activity of the phage was determined by measuring the OD values as an indicator.

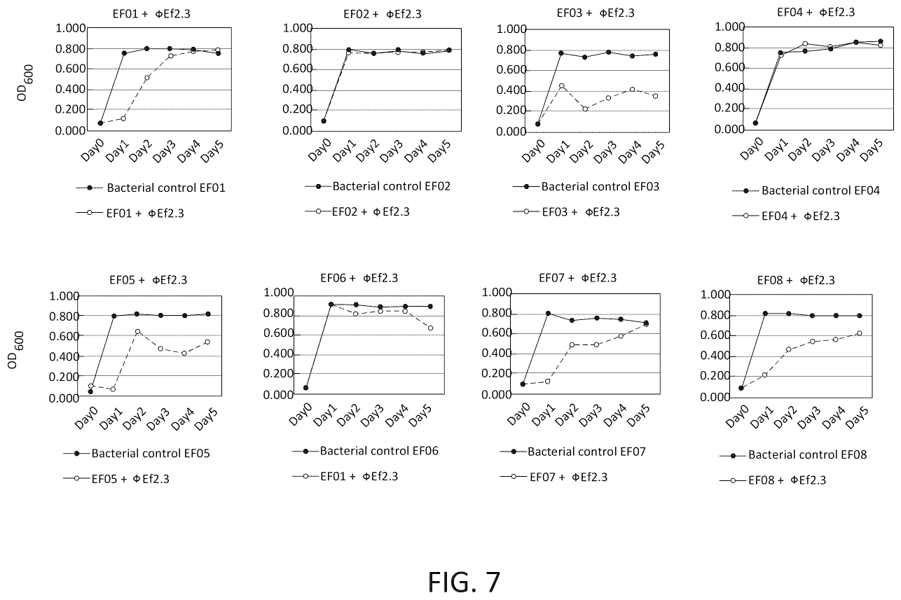

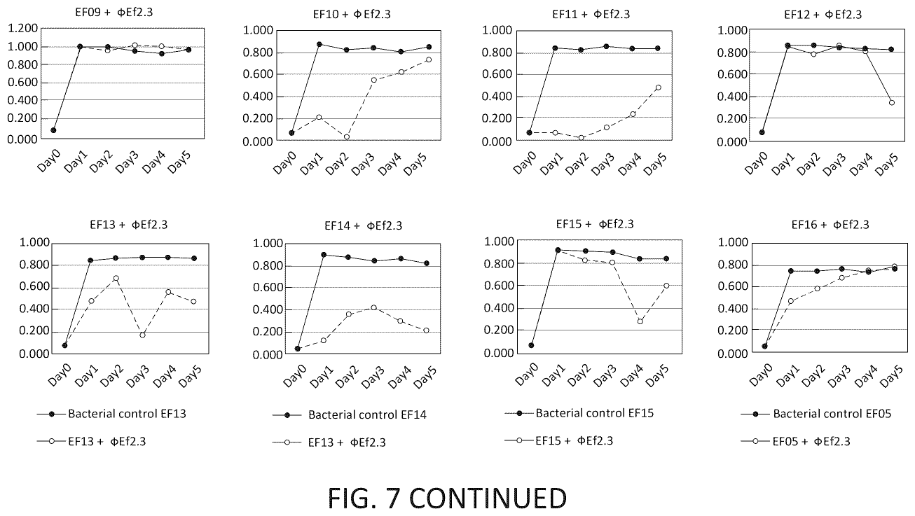

As shown in FIG. 7, in the case of ΦEf2.3-susceptible hosts (EF01, 03, 05, 07, 08, 10, 11, 13, 14, 16), the OD value was lower in phage inoculation compared with non-inoculation until about 1-2 days post inoculation, suggesting that the phage suppressed the growth of reference strains. In the case of EF01, 07, 08, 11, and 16, the OD value increased continuously, suggesting the occurrence of phage-resistant variants; however, in the case of EF03, 05, 10, 13, and 14, a phase in which OD decreased again after an increase in OD was observed, suggesting that the supernatant may contain evolved phages that can reinfect phage-resistant variants.

In the case of ΦEf2.3 non-susceptible hosts (EF02,04,06,09,12,15), the growth curve was similar to that of non-phage inoculation, as in the case of co-cultures with EF02,04,09. On the other hand, in the co-culture with EF06,12,15, a decrease in OD value was observed on the day 4 (EF15) and day 5 (EF06 and 12) of co-culture, suggesting lysis by evolved phages adapted to the host.

### Isolation of Evolved Phages

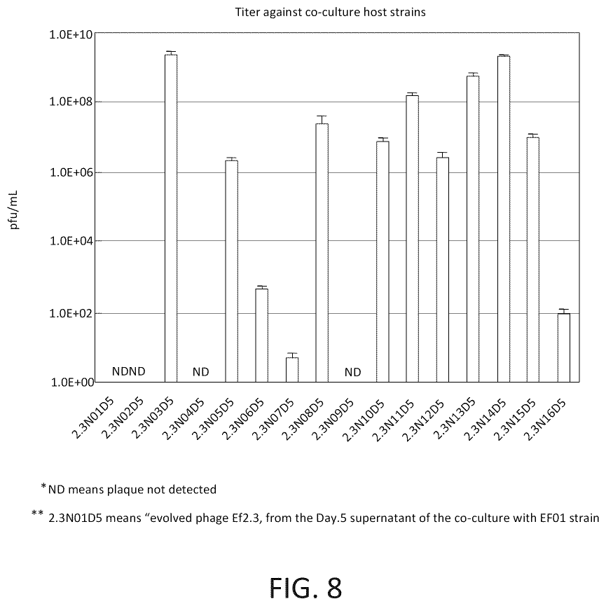

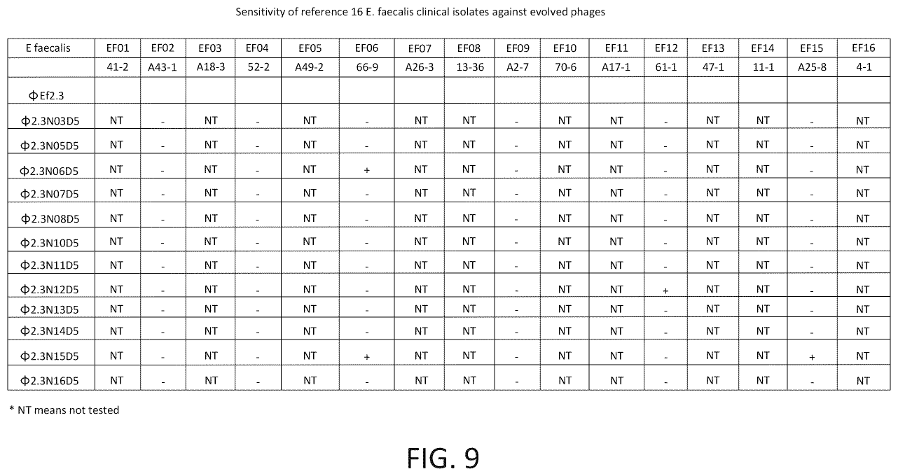

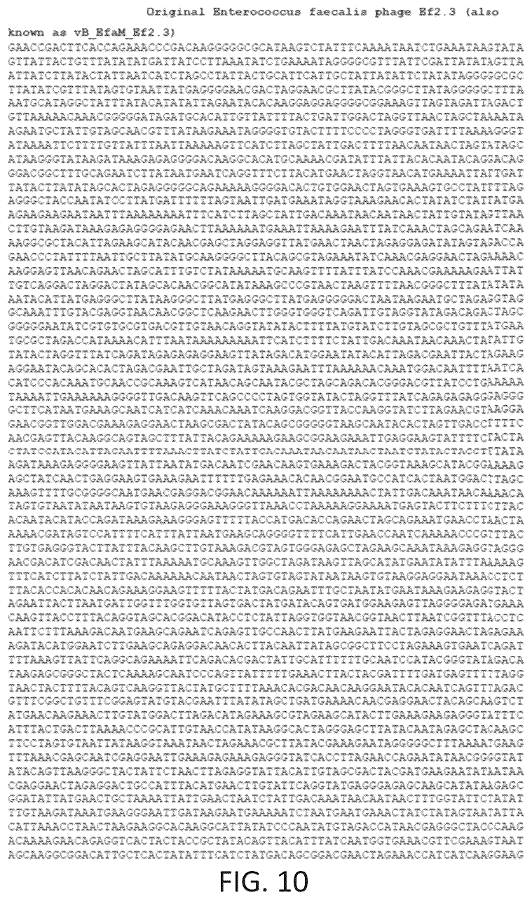

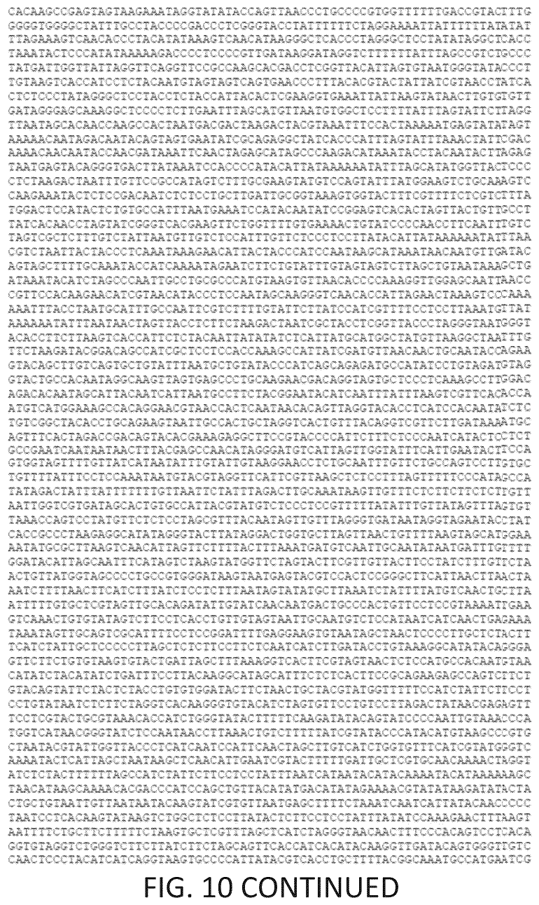

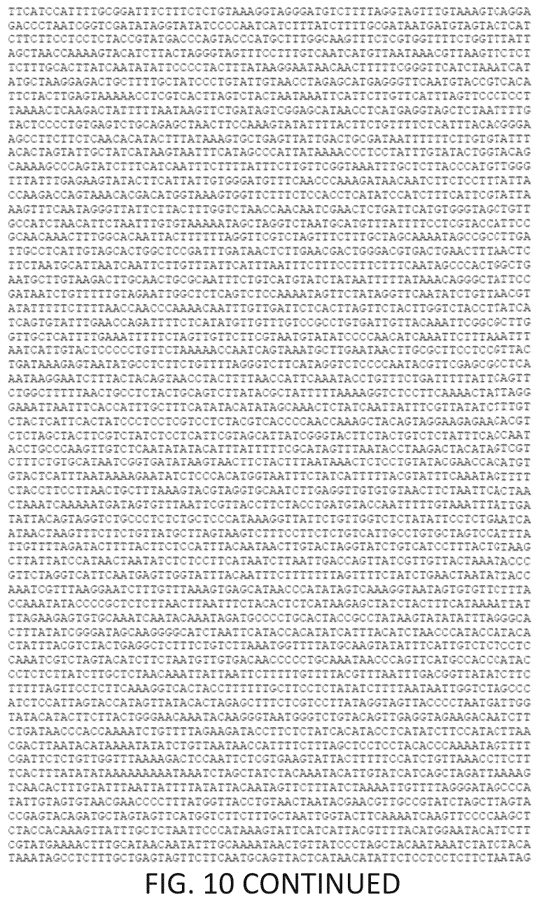

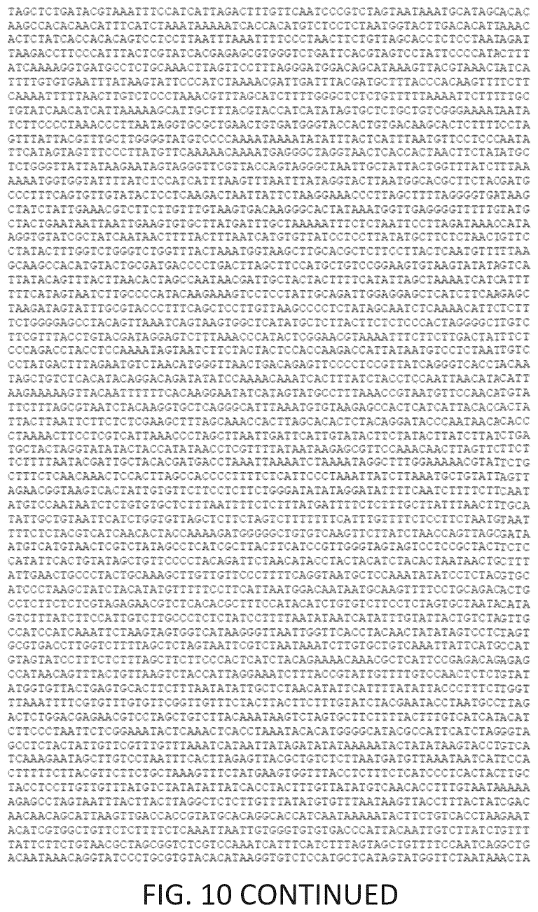

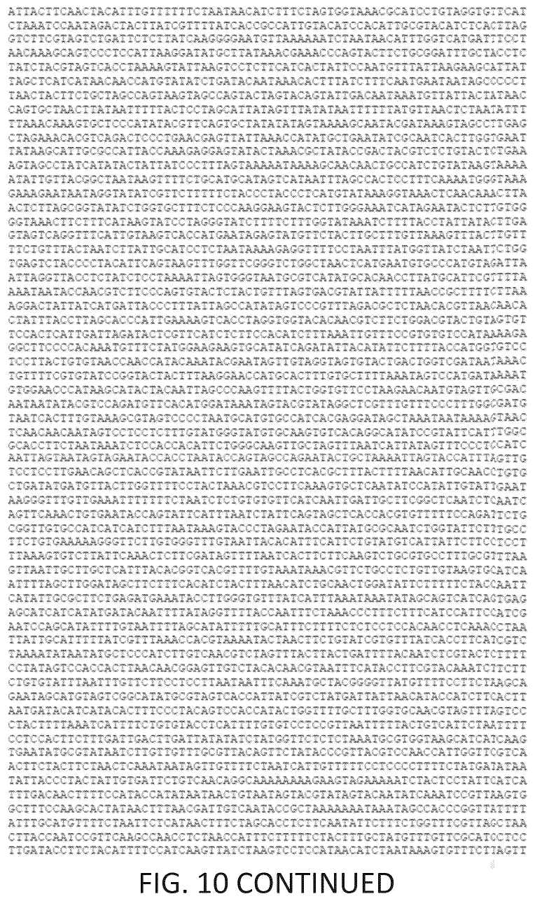

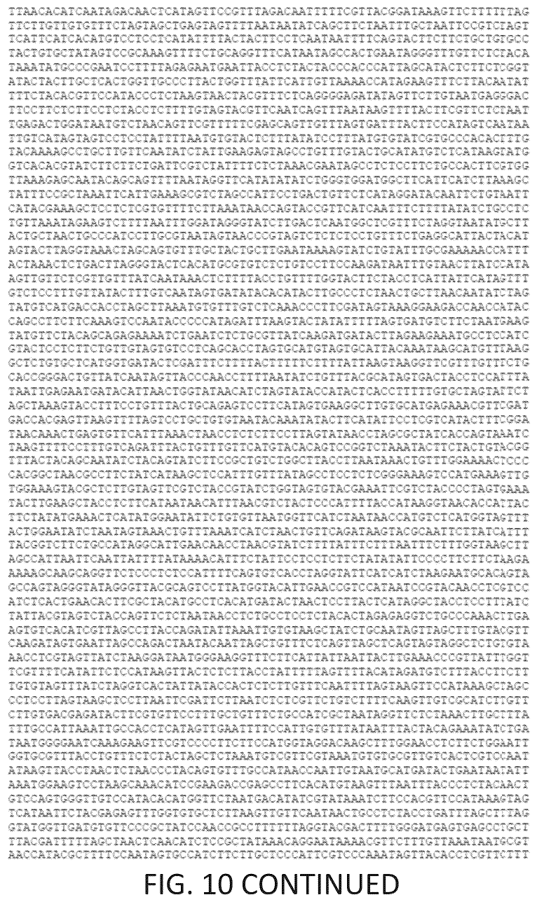

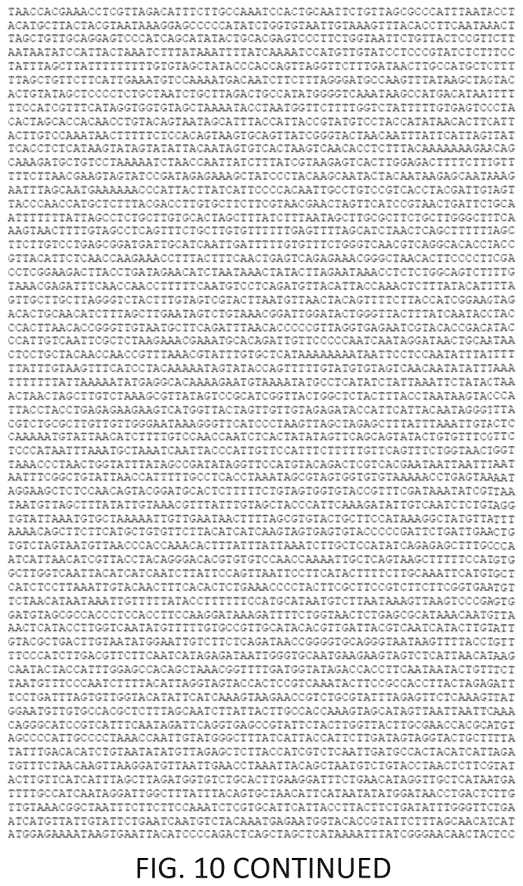

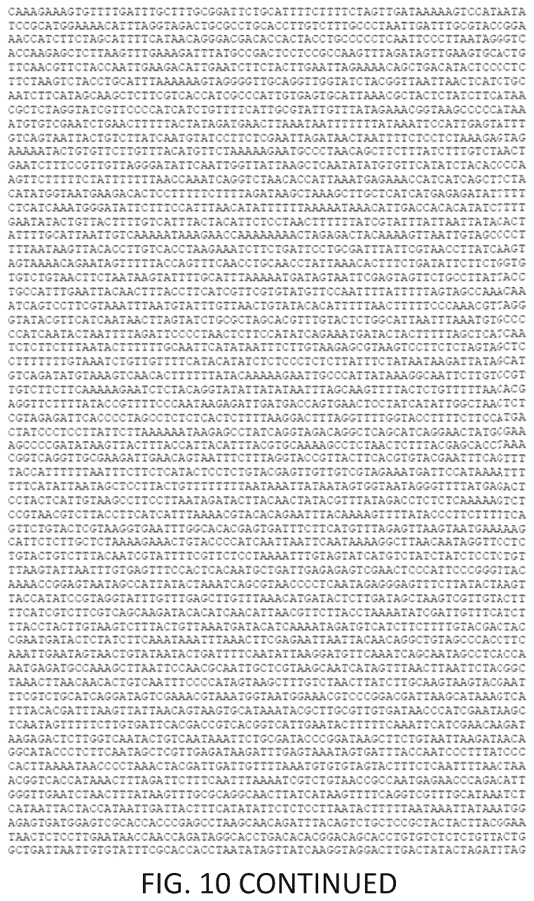

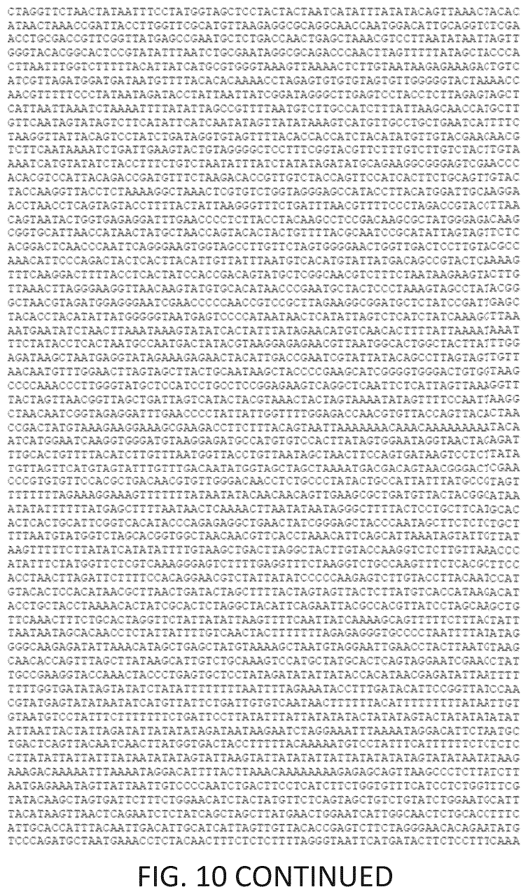

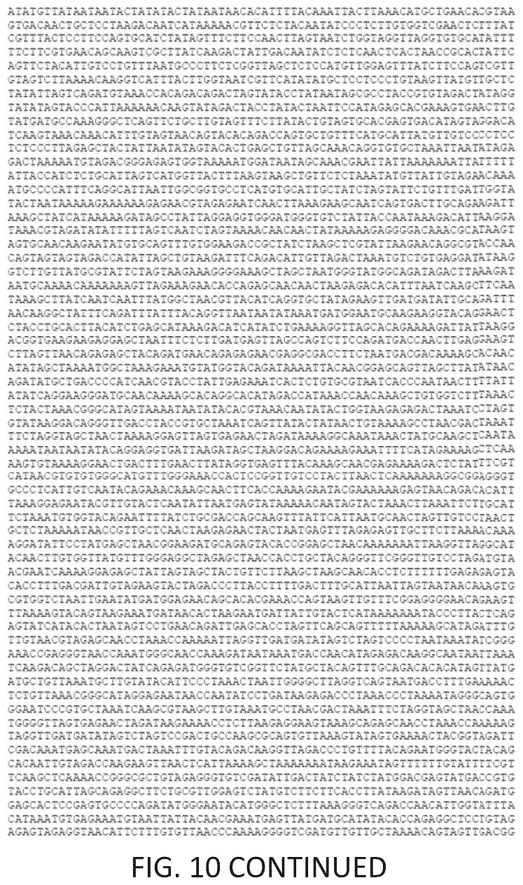

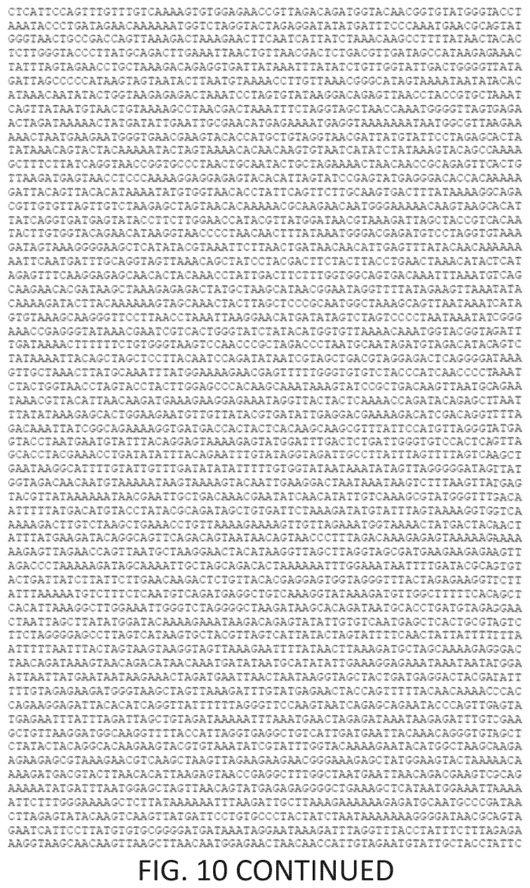

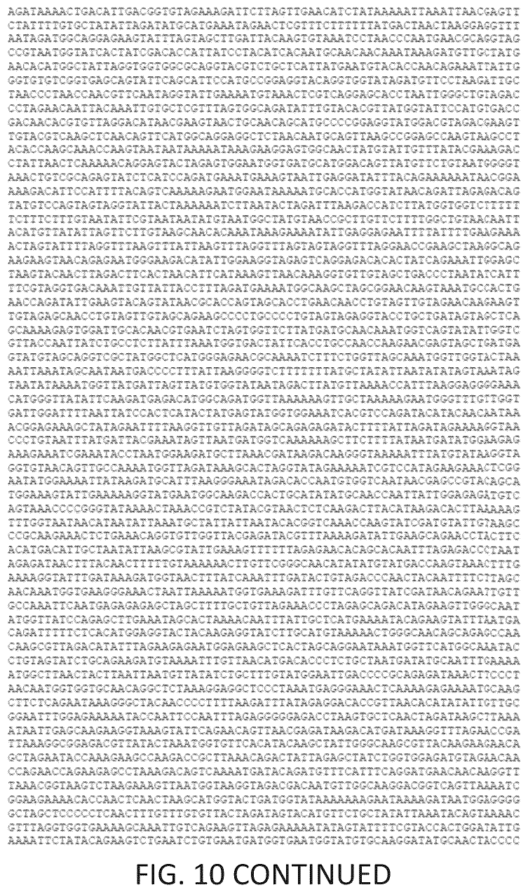

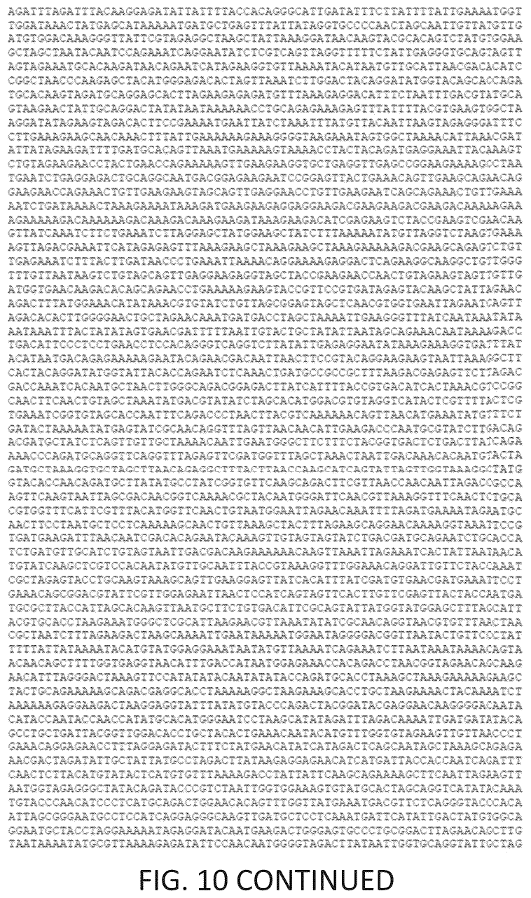

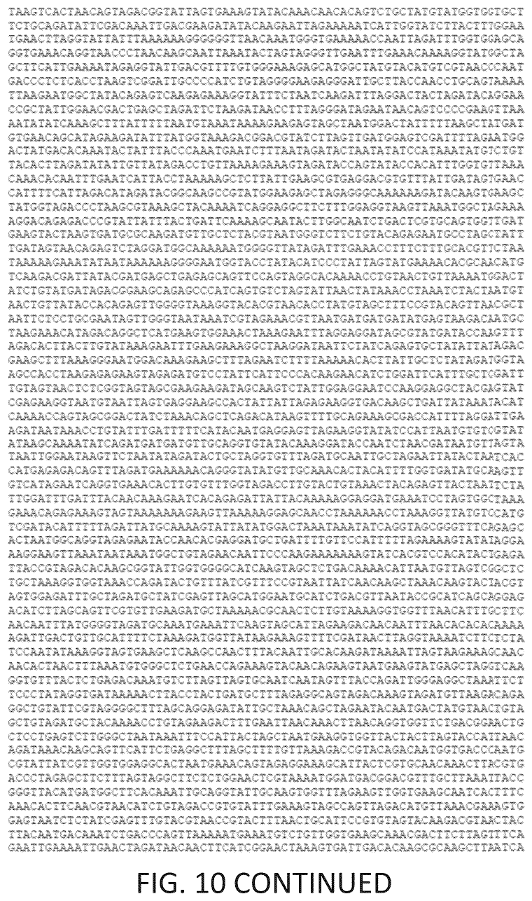

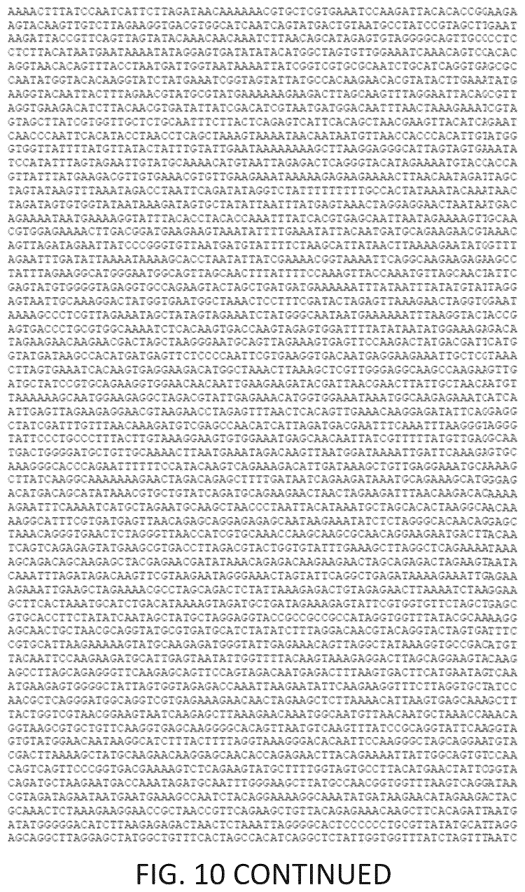

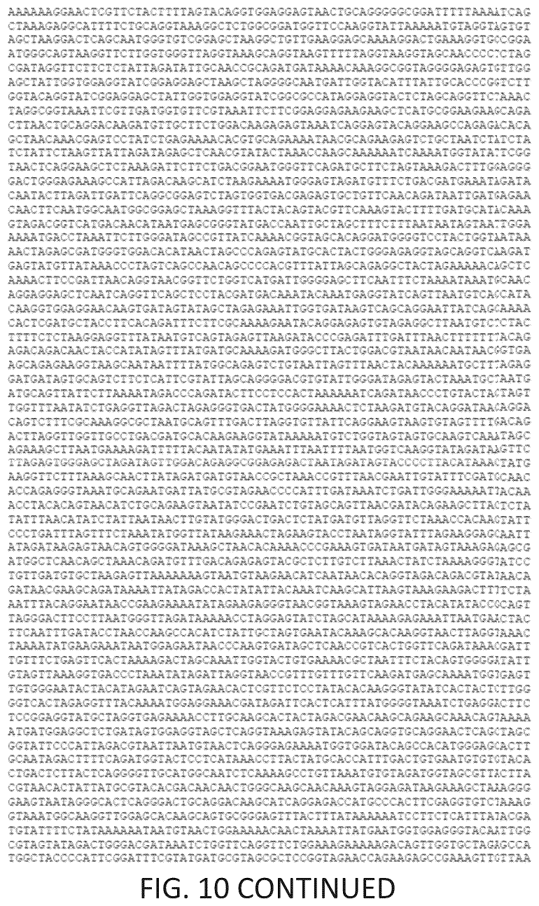

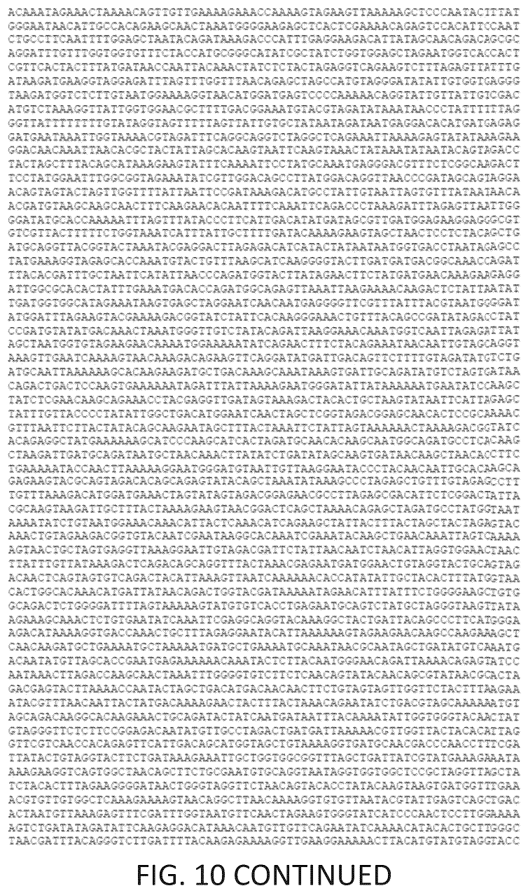

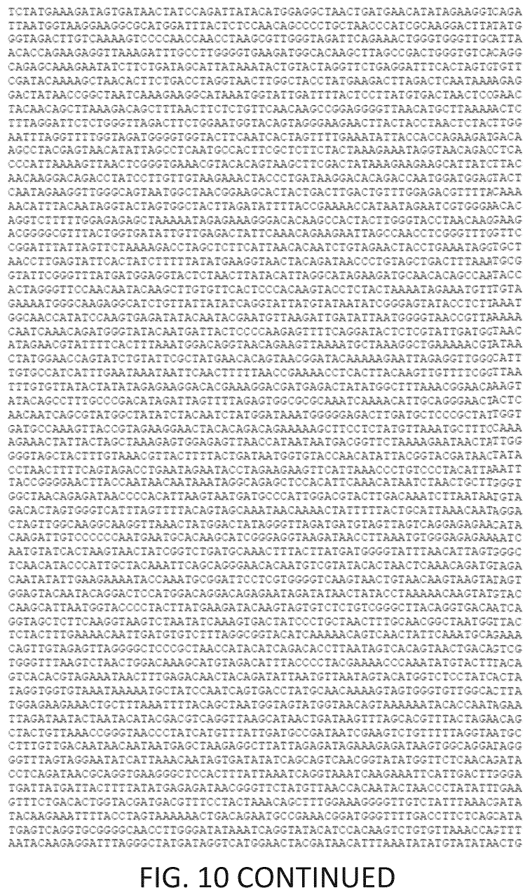

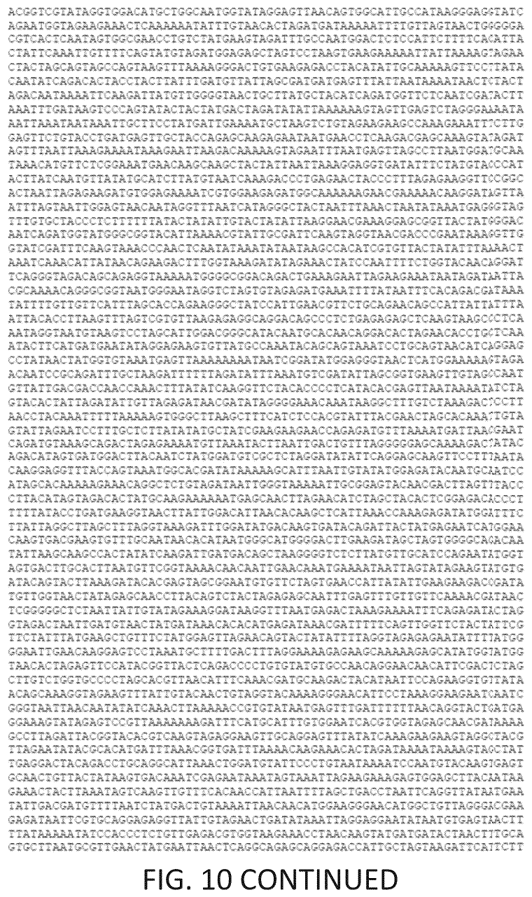

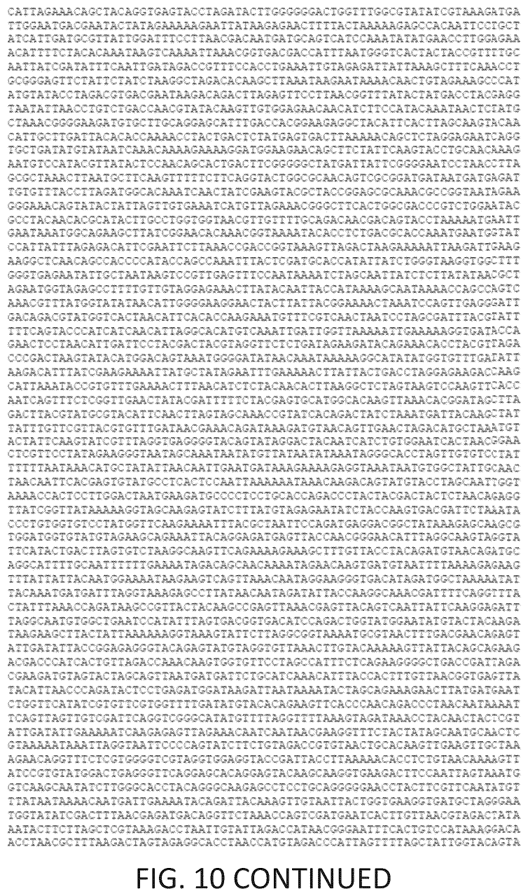

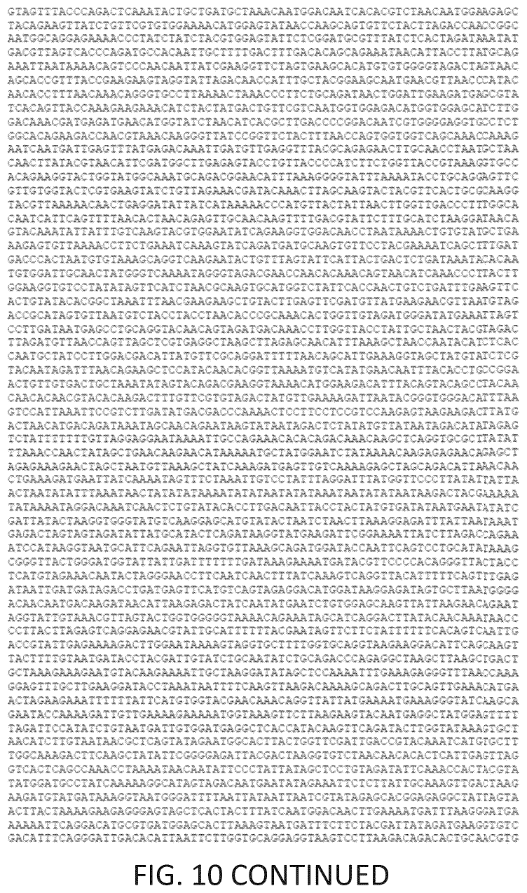

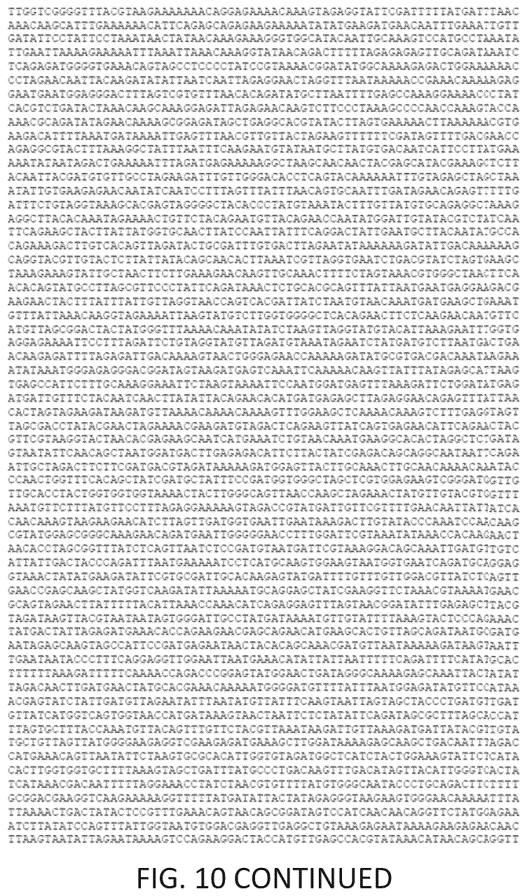

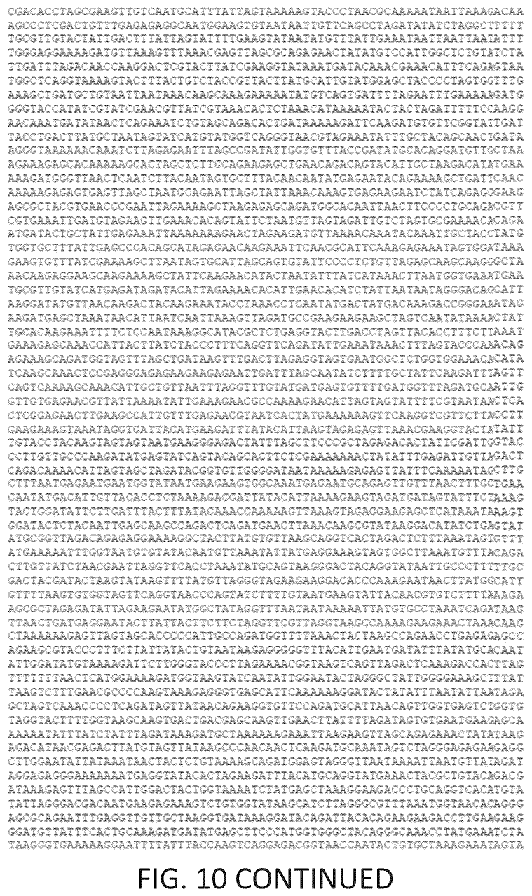

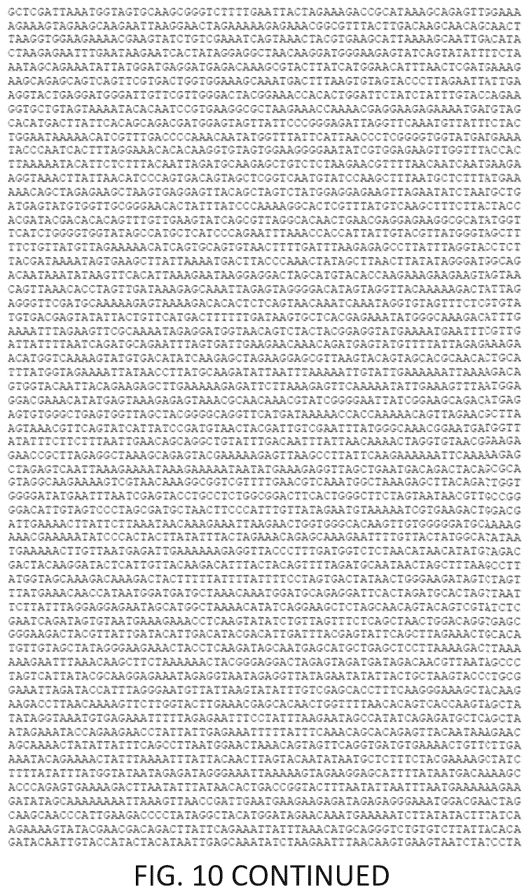

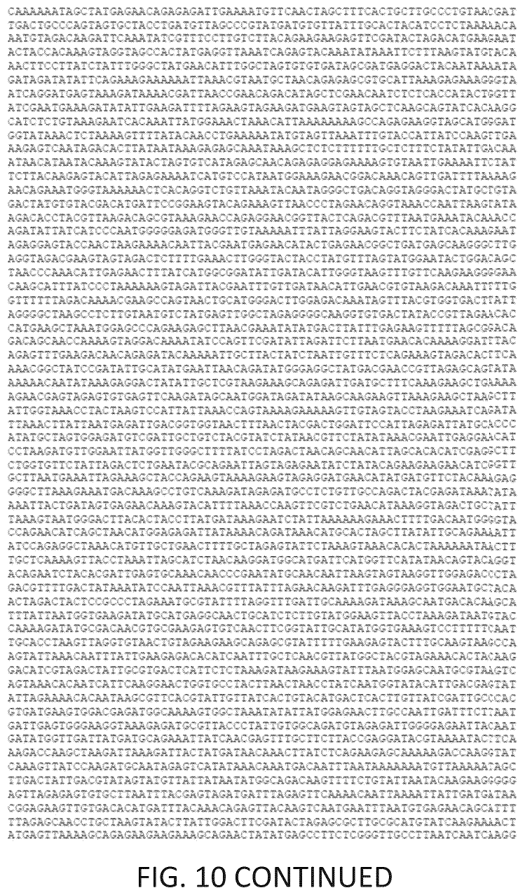

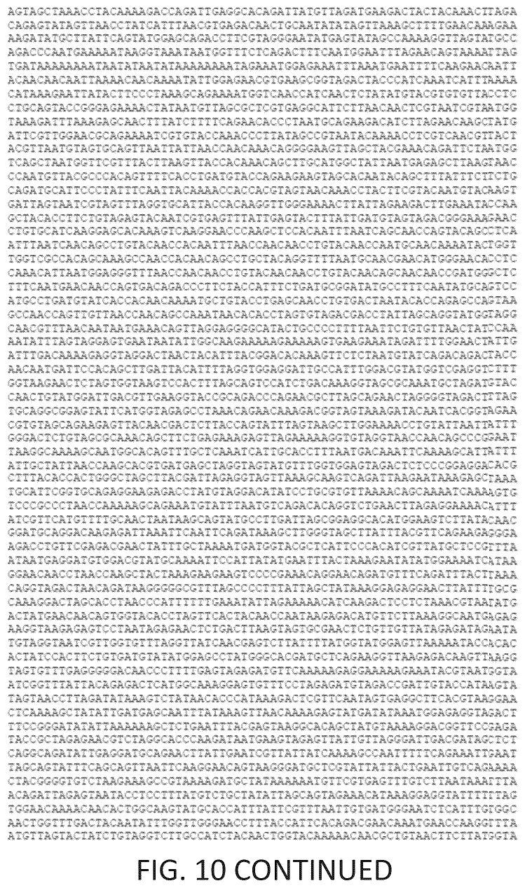

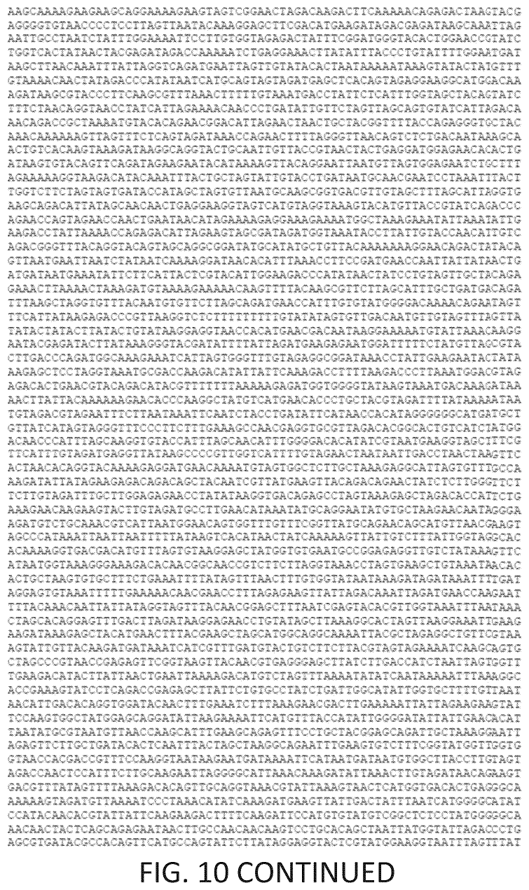

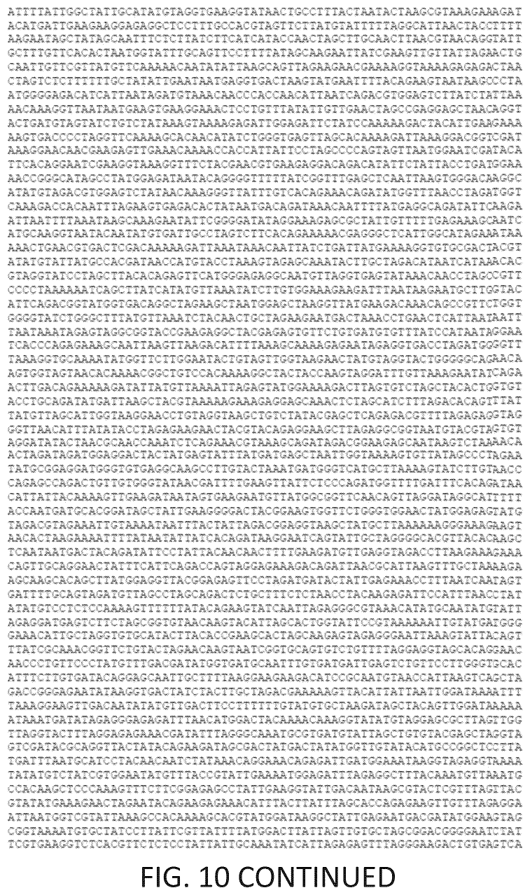

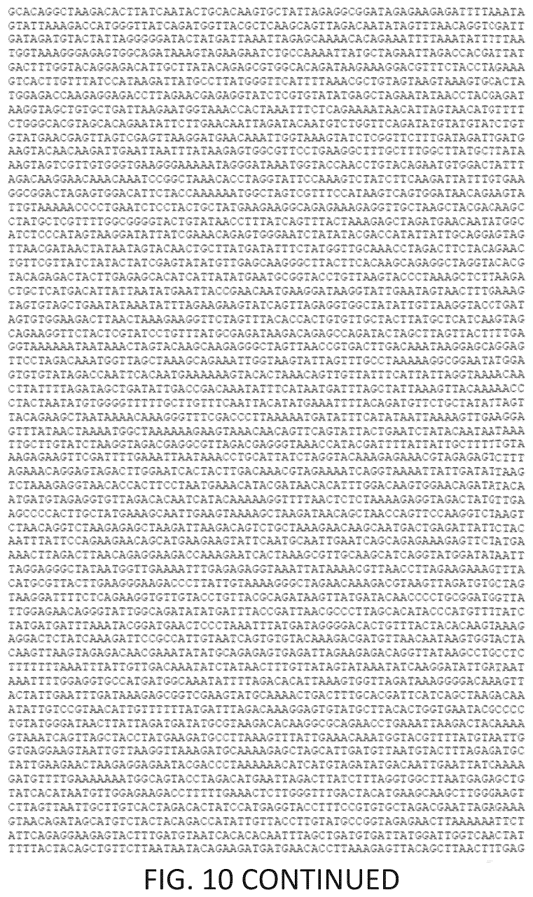

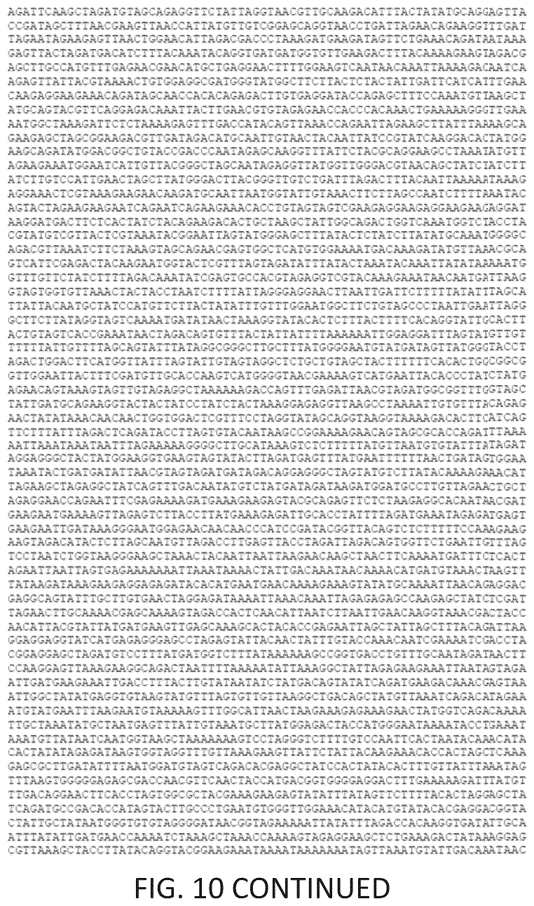

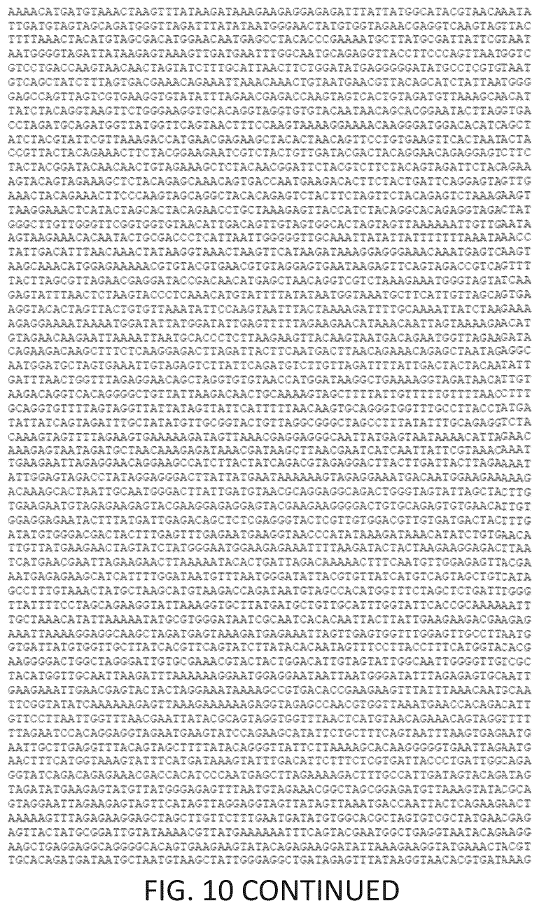

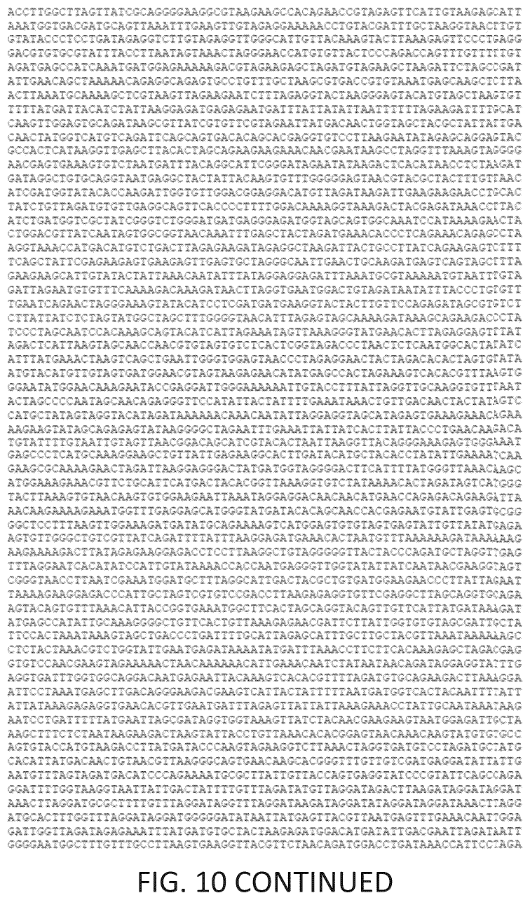

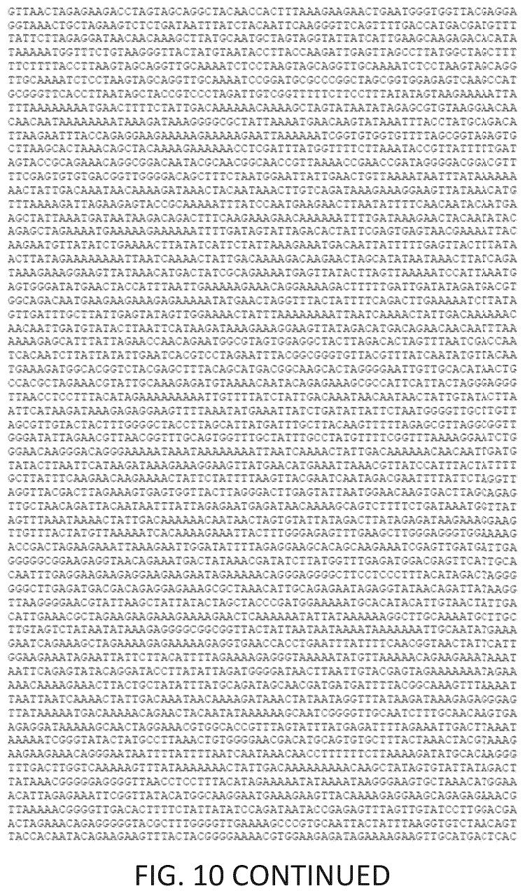

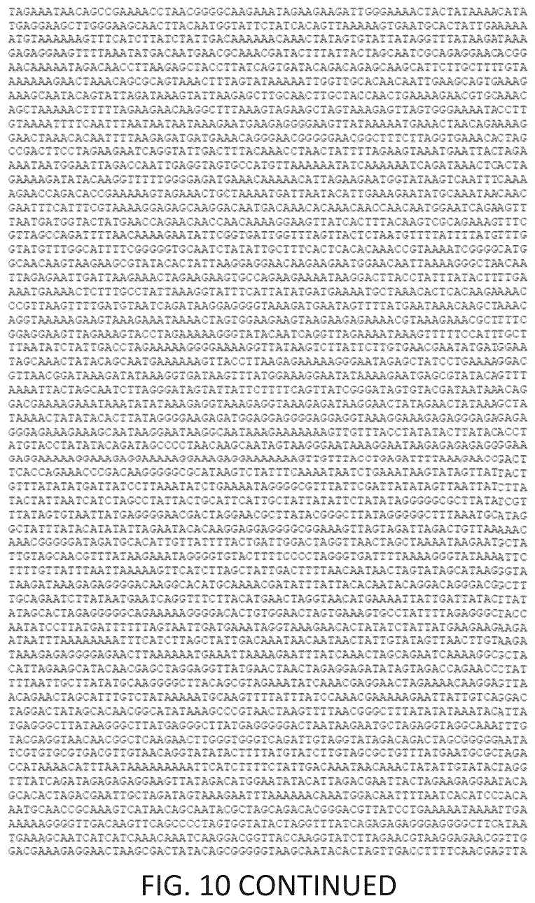

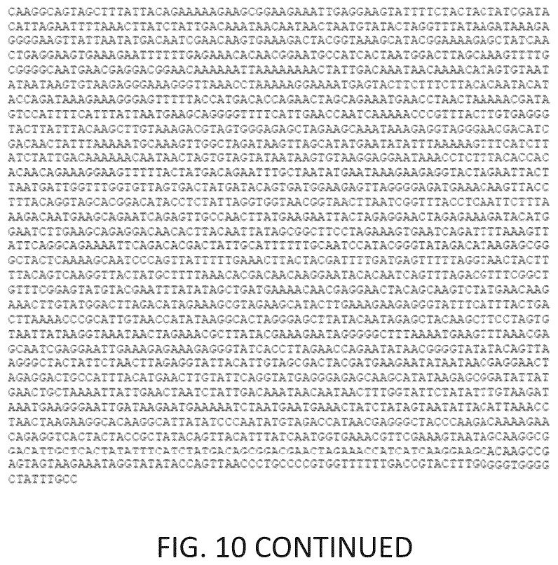

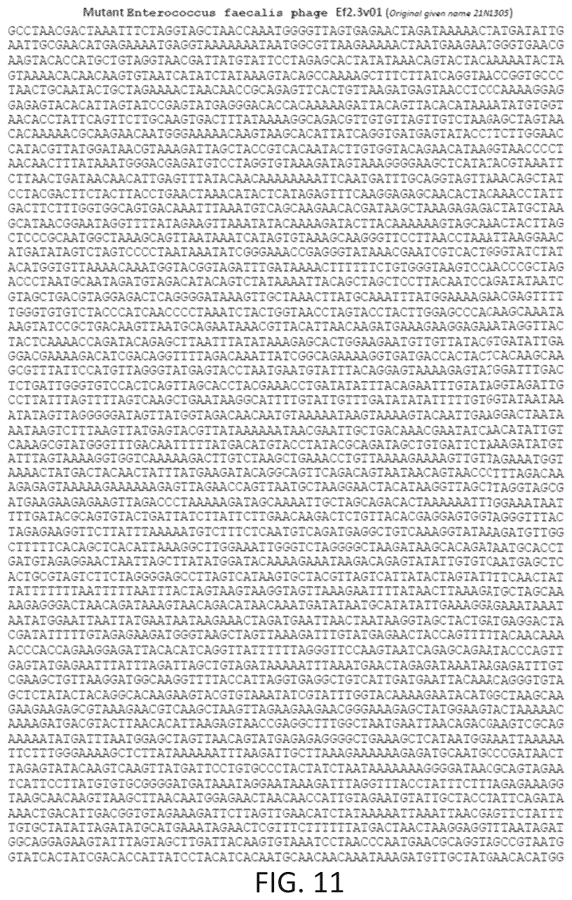

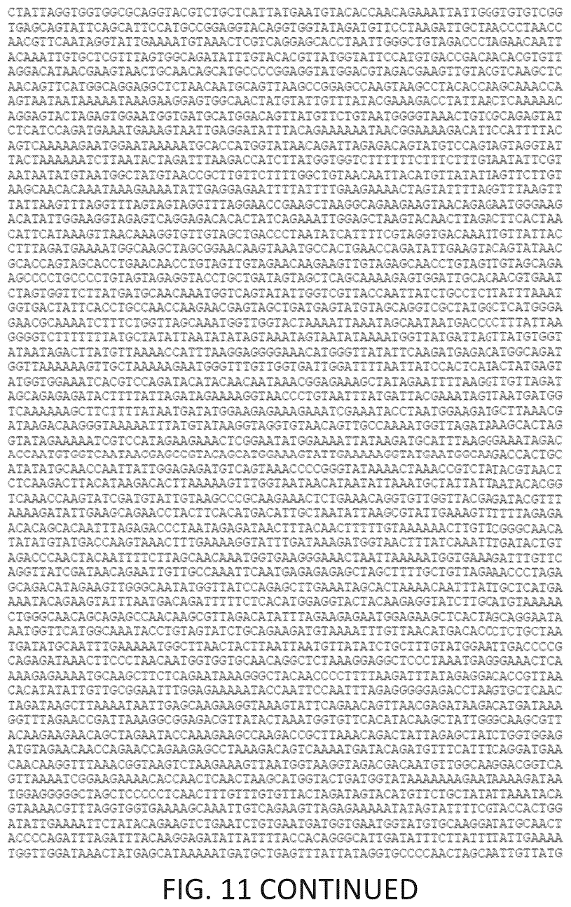

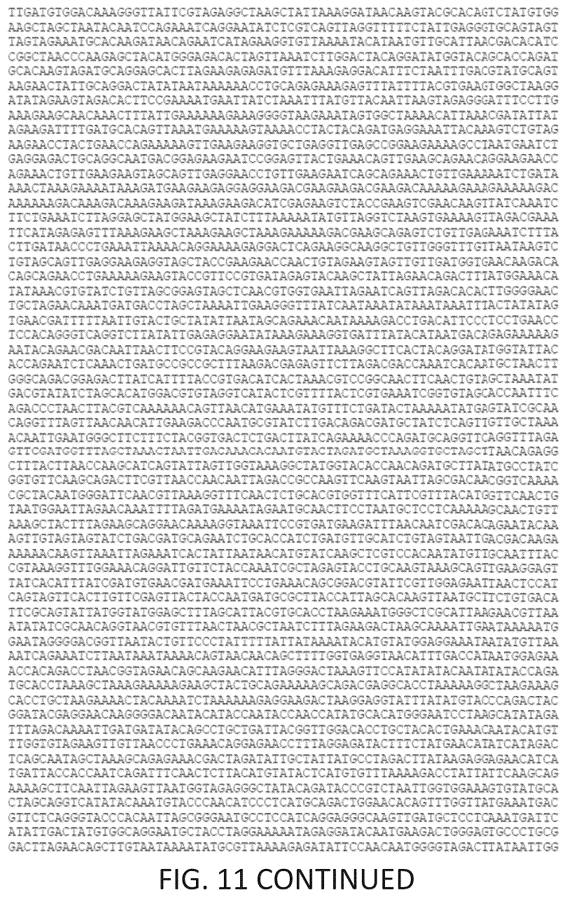

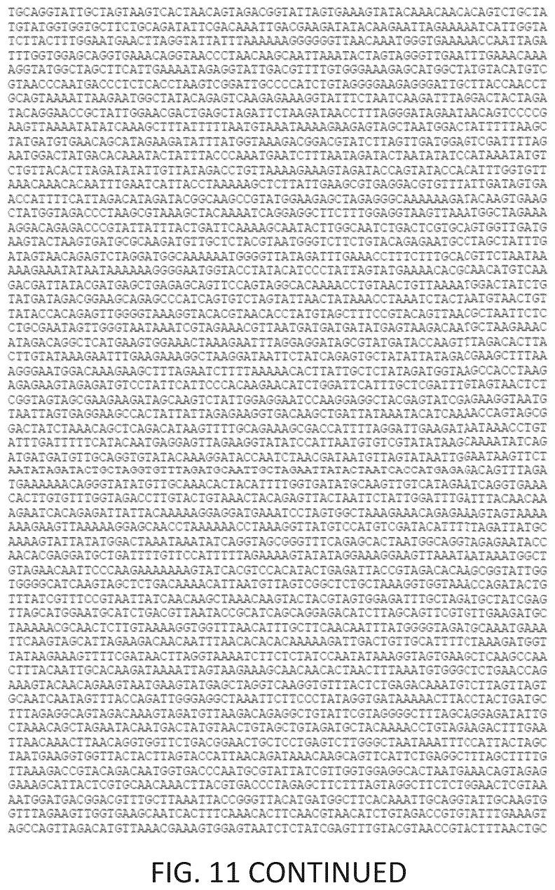

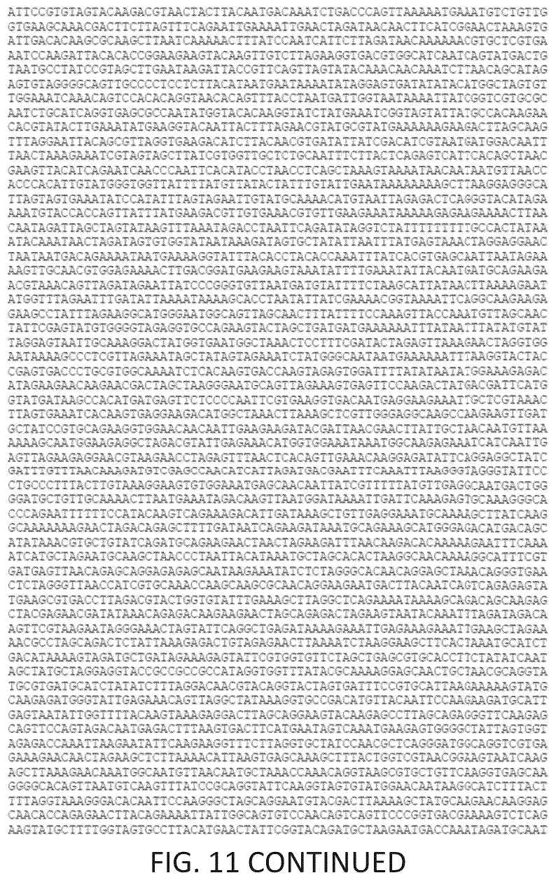

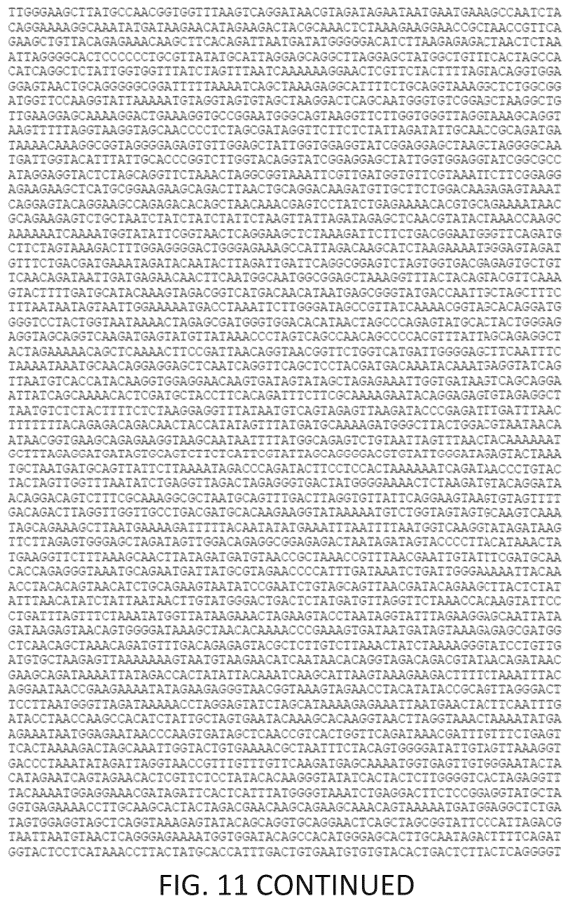

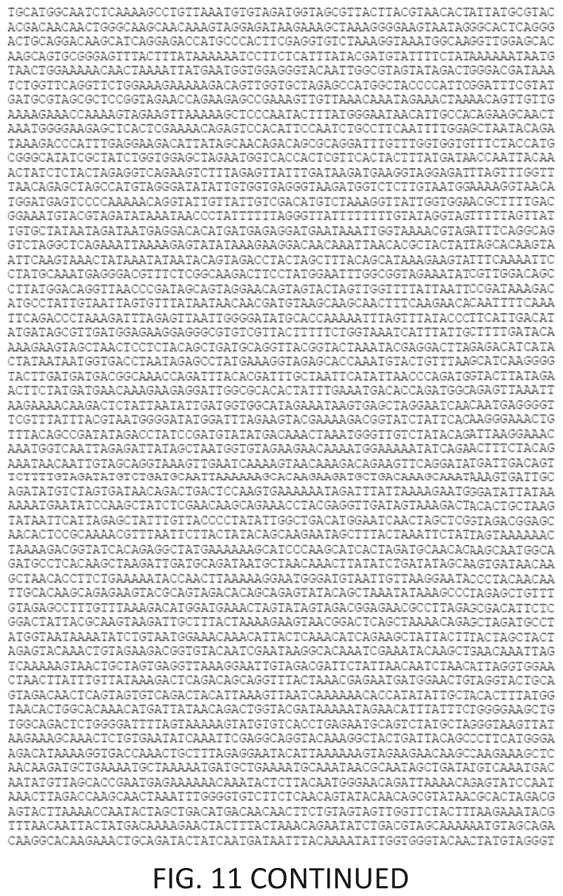

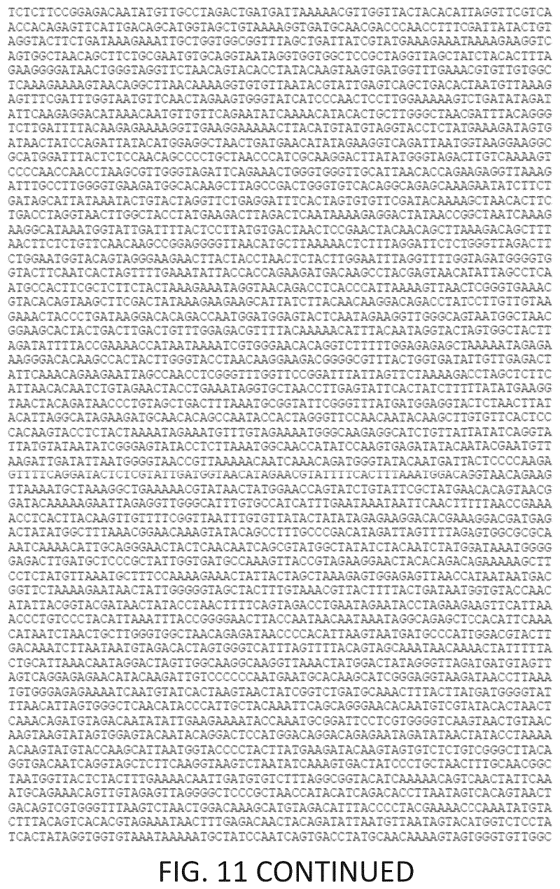

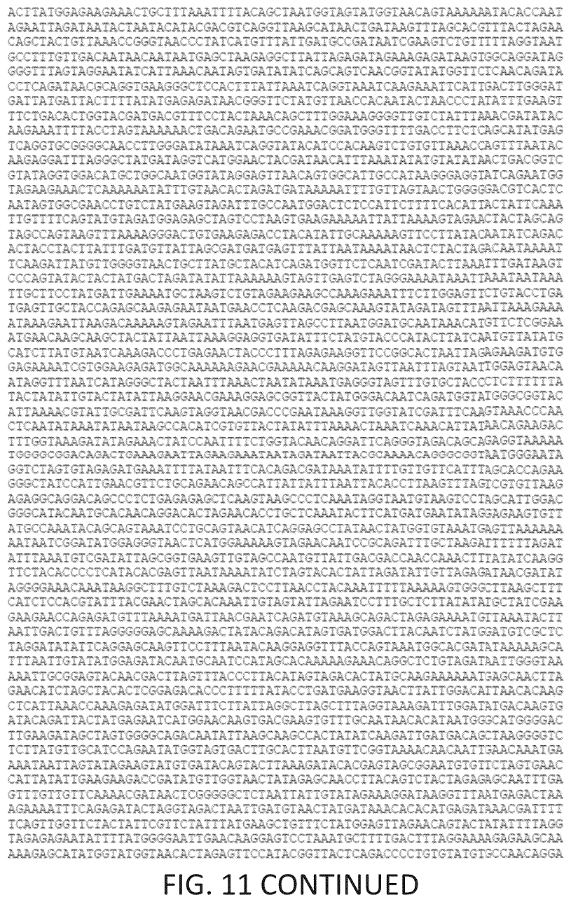

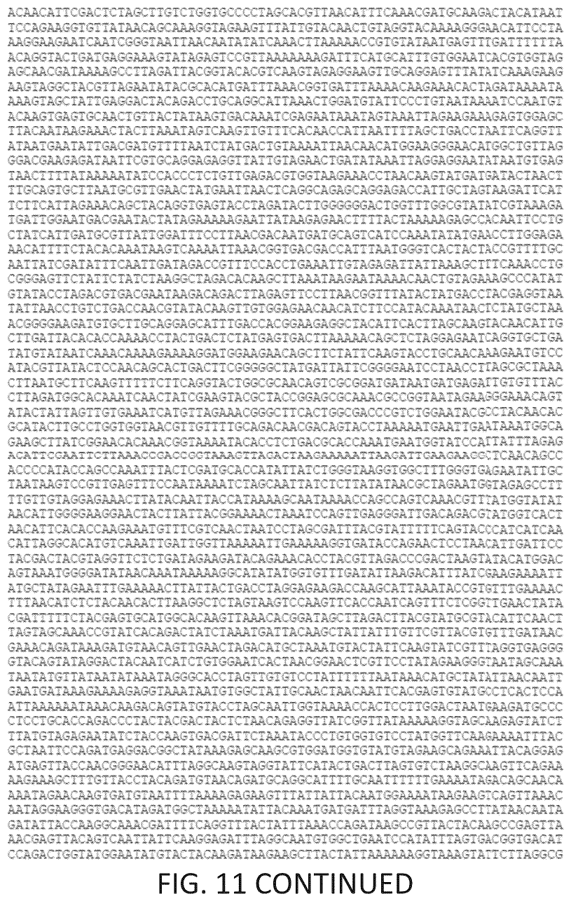

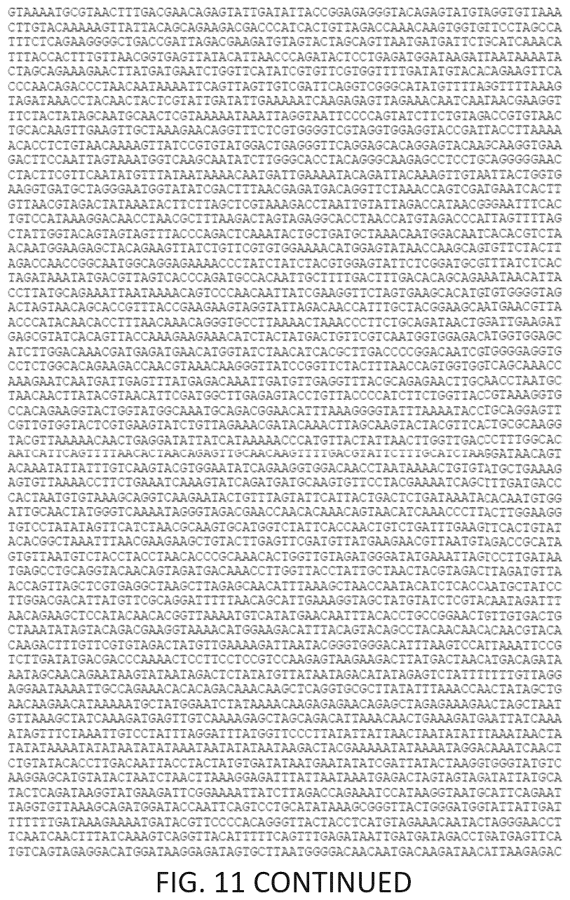

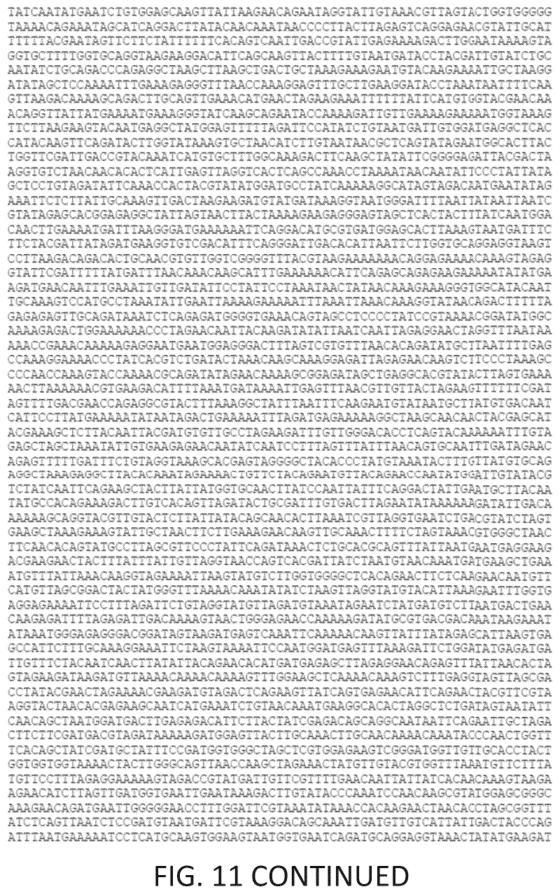

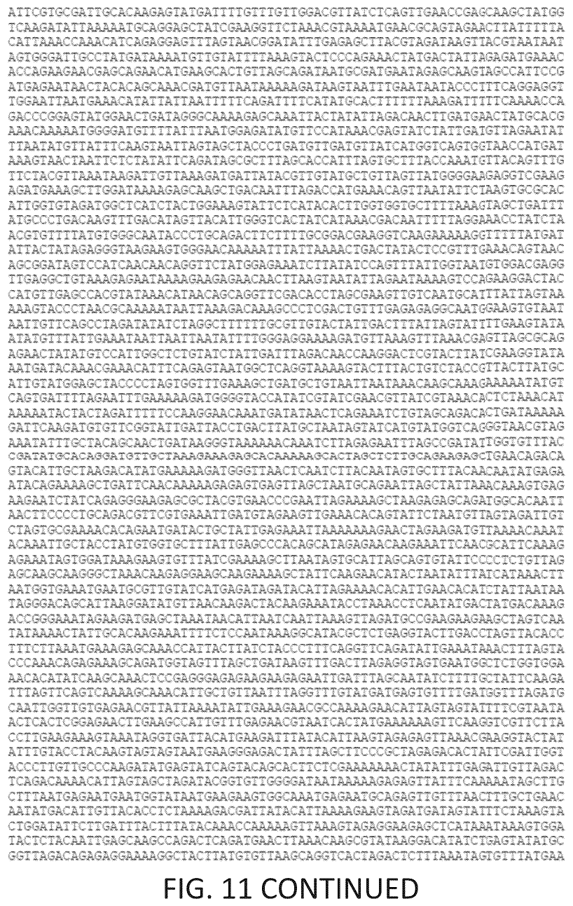

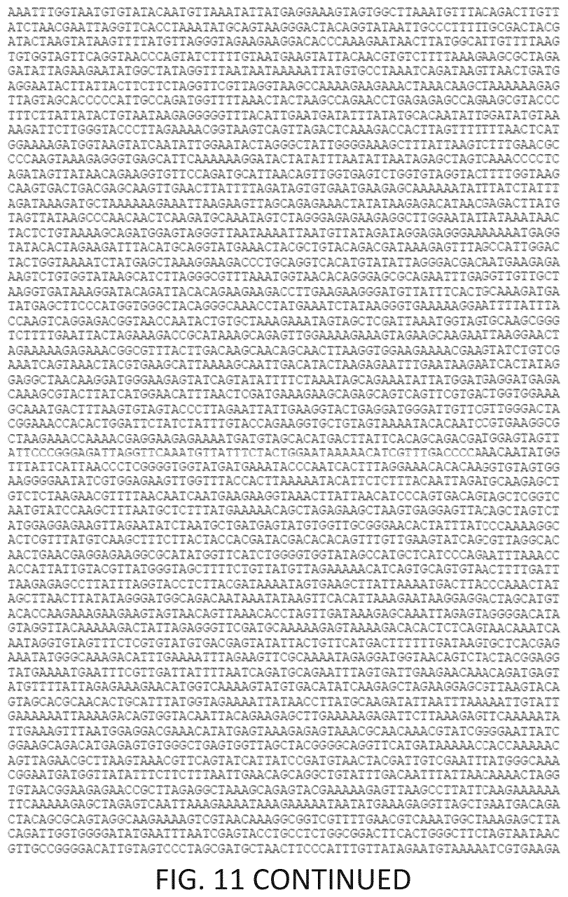

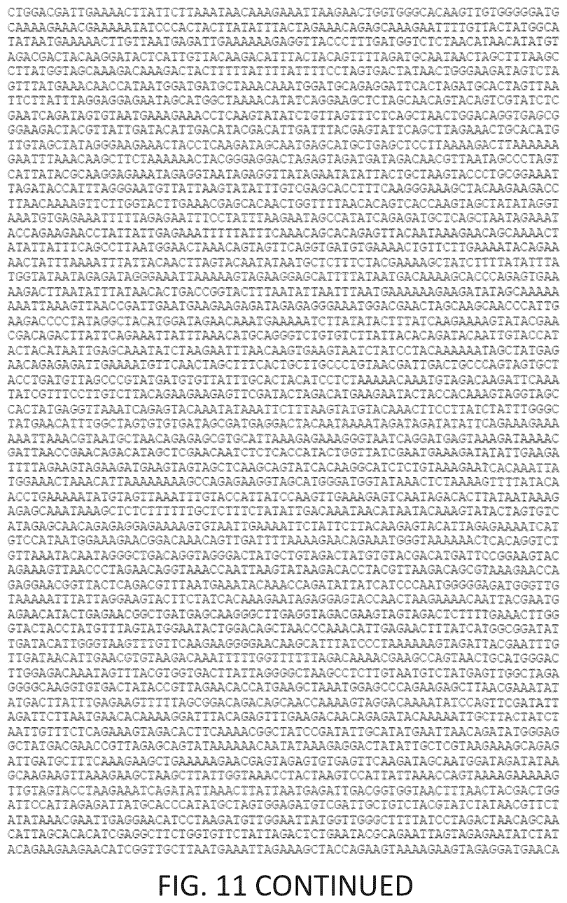

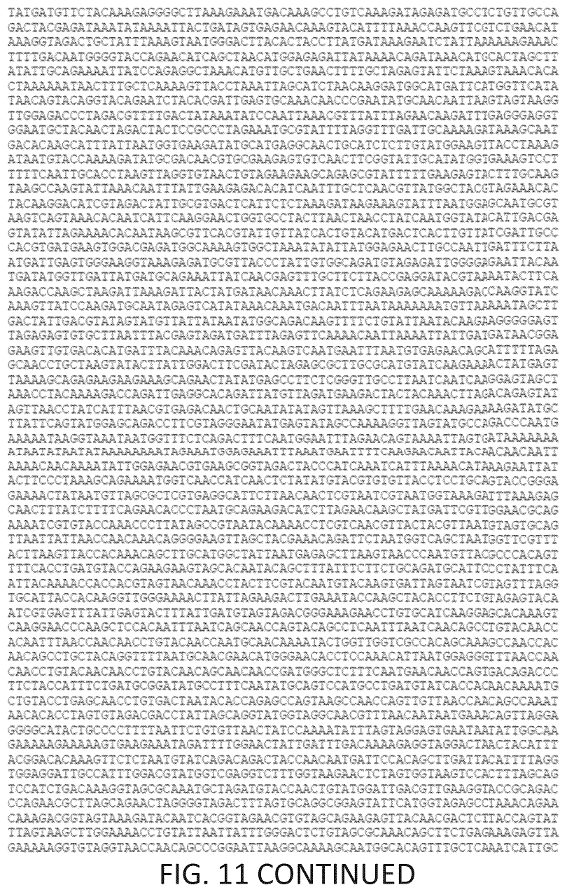

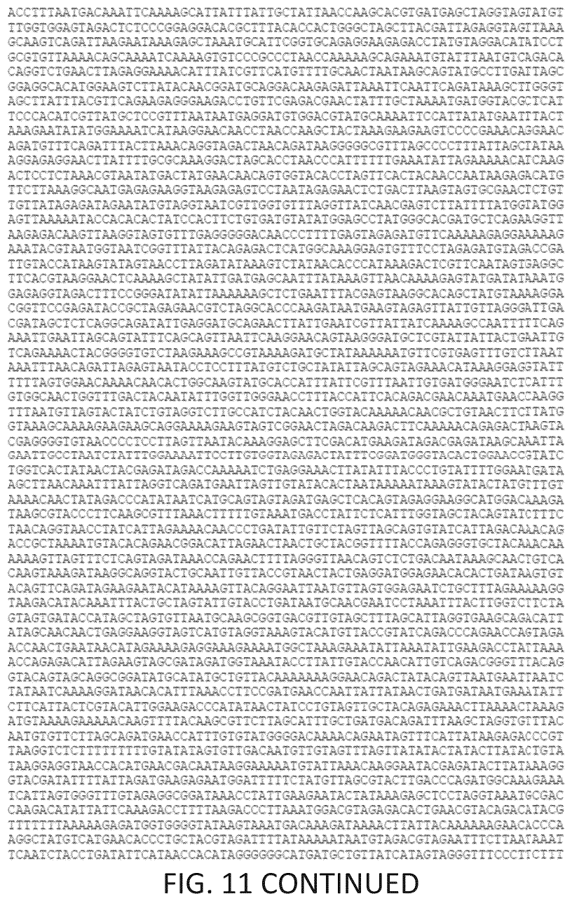

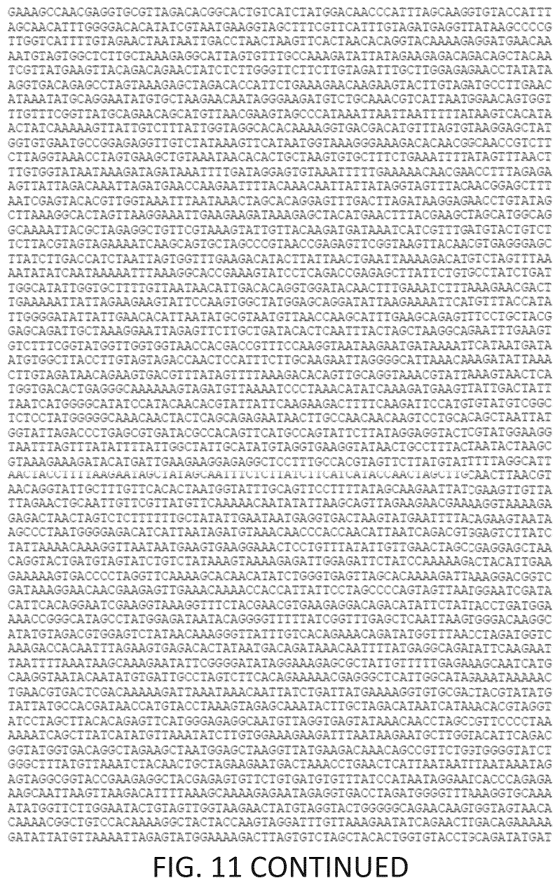

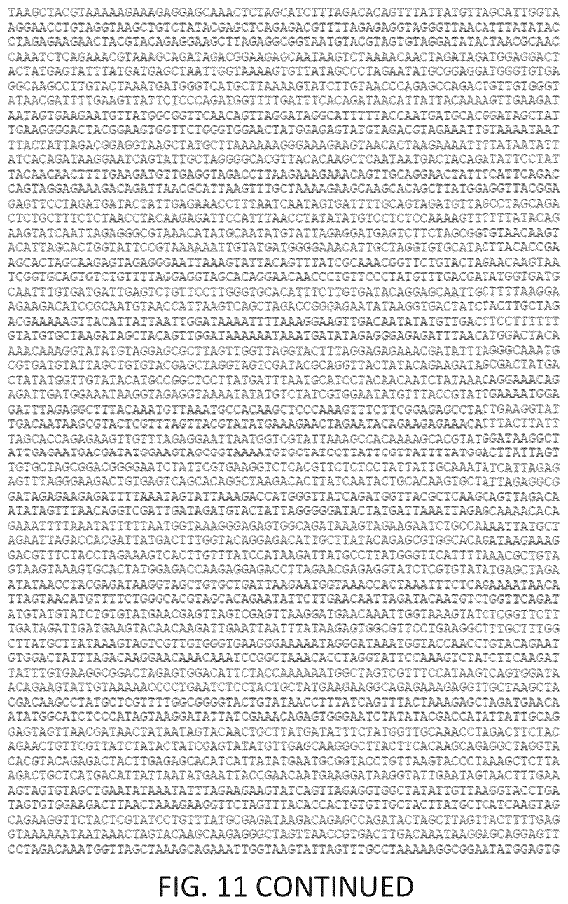

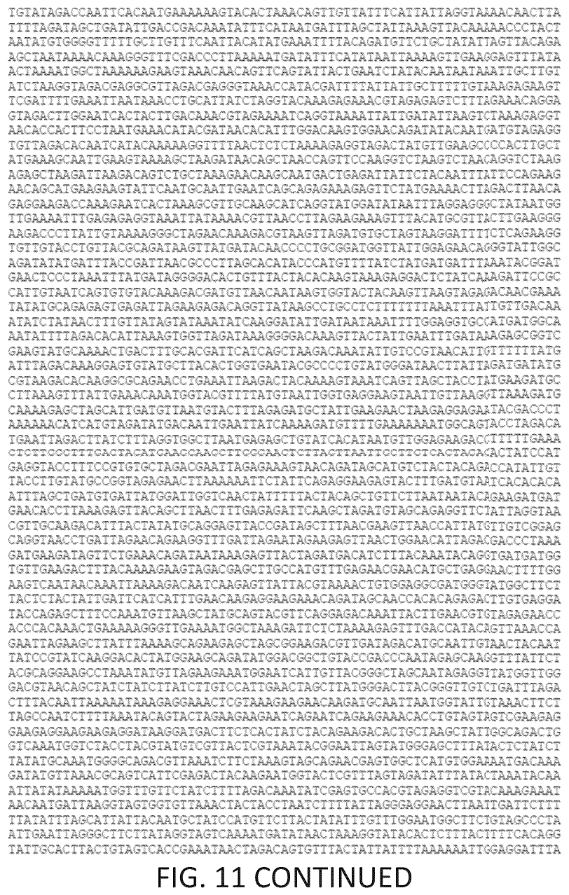

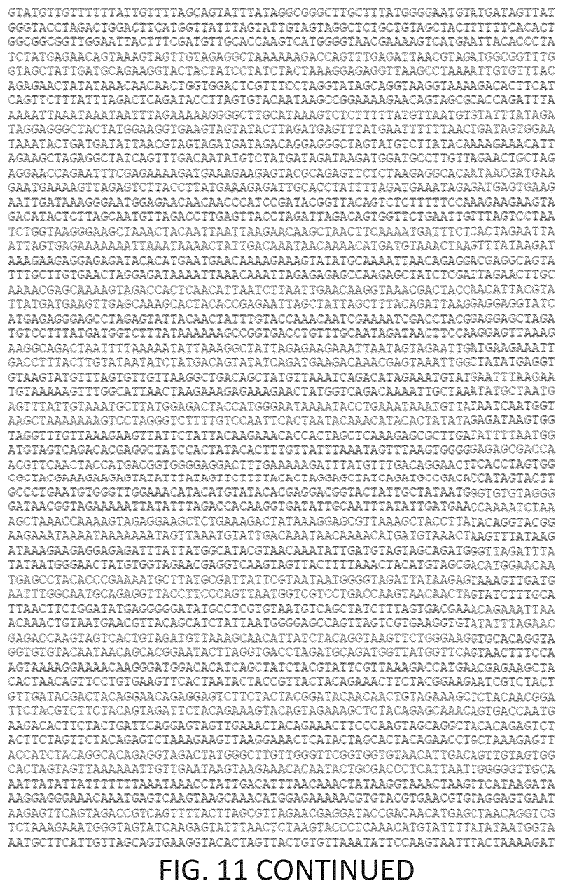

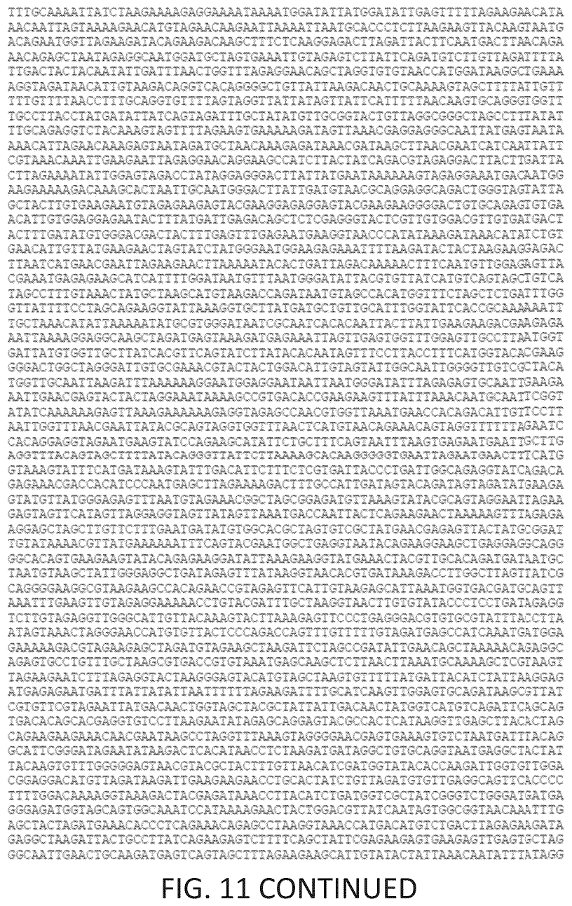

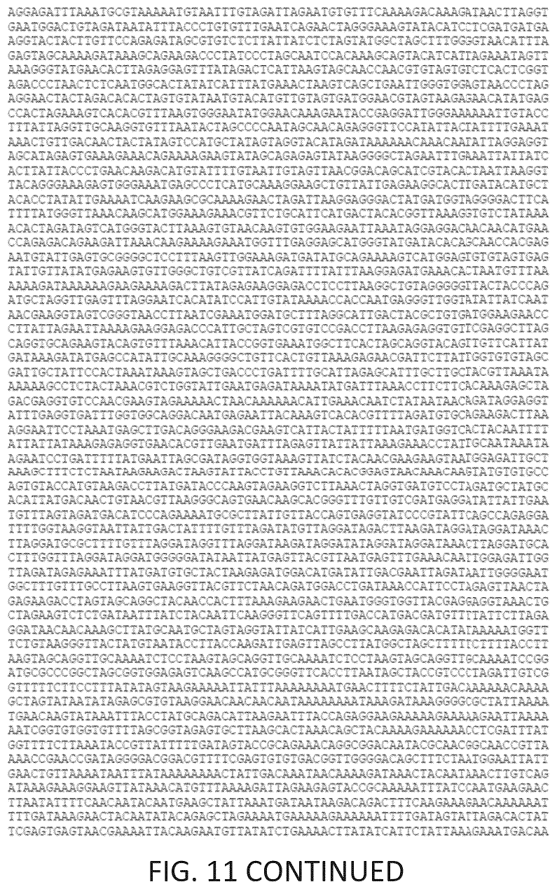

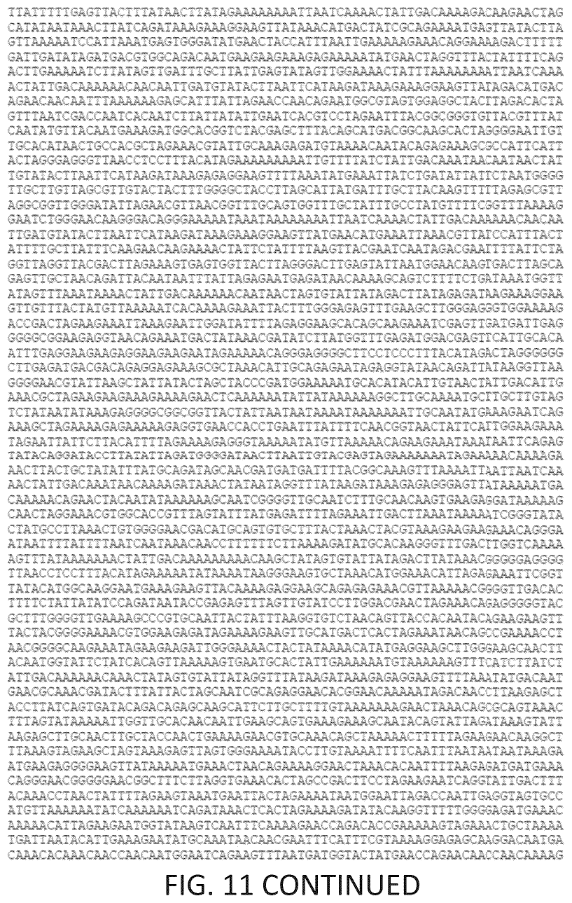

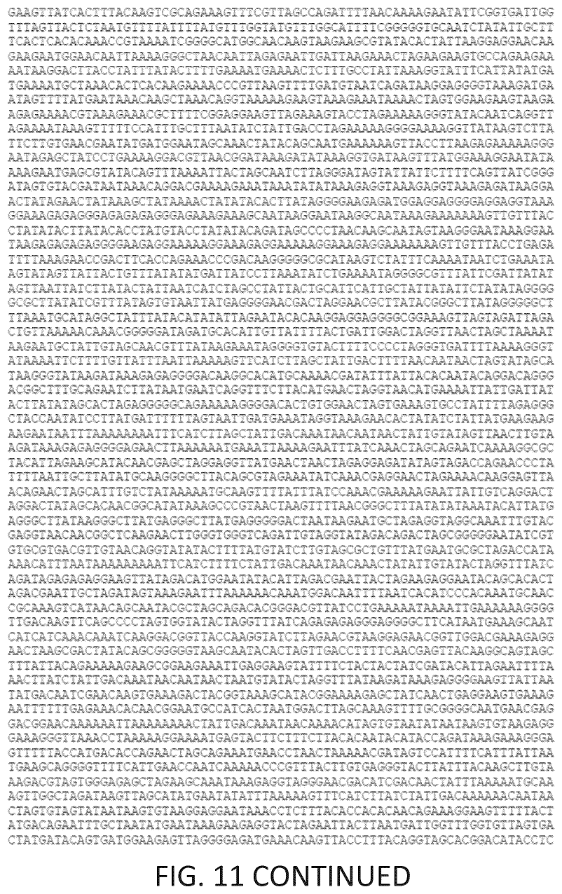

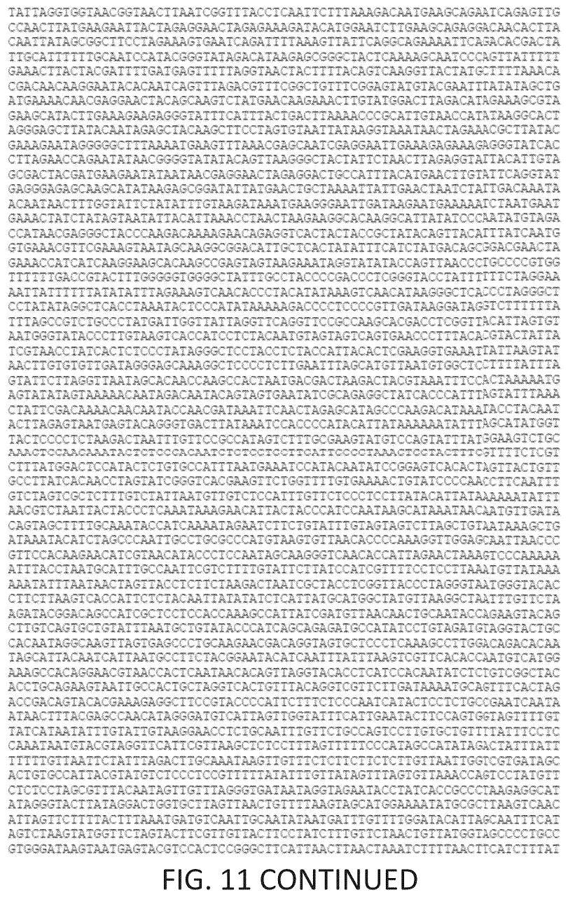

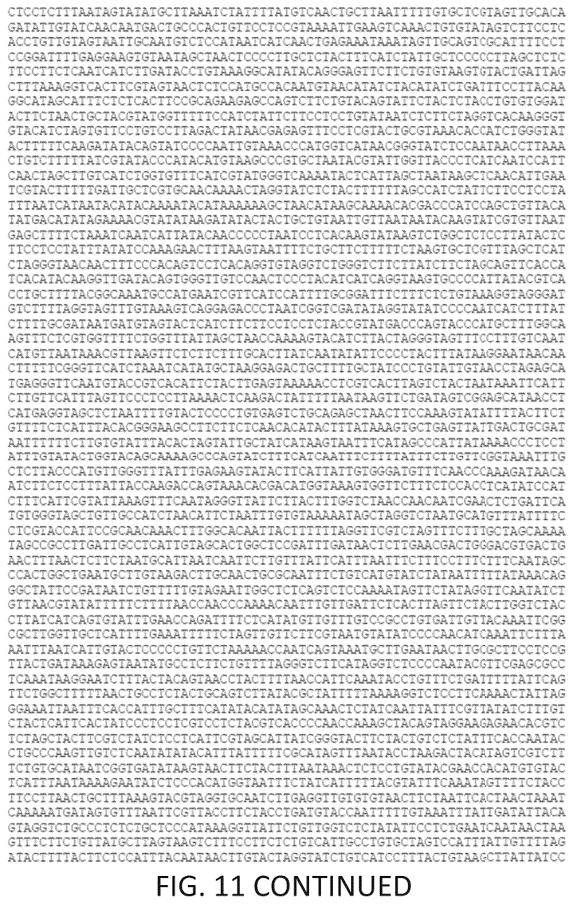

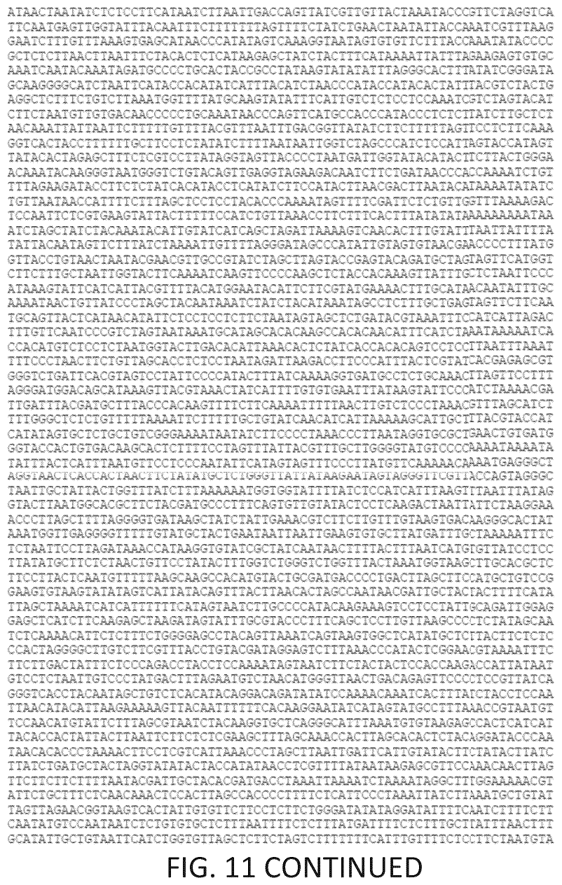

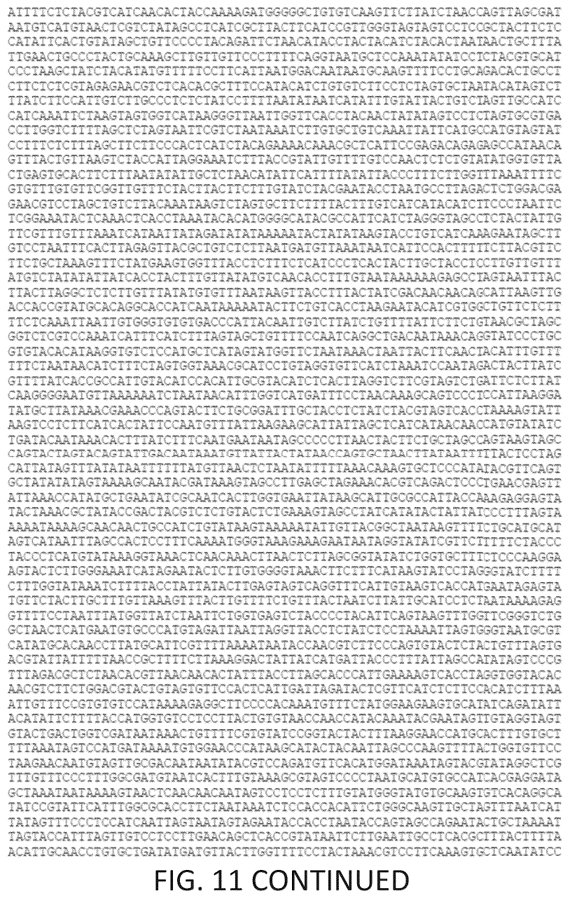

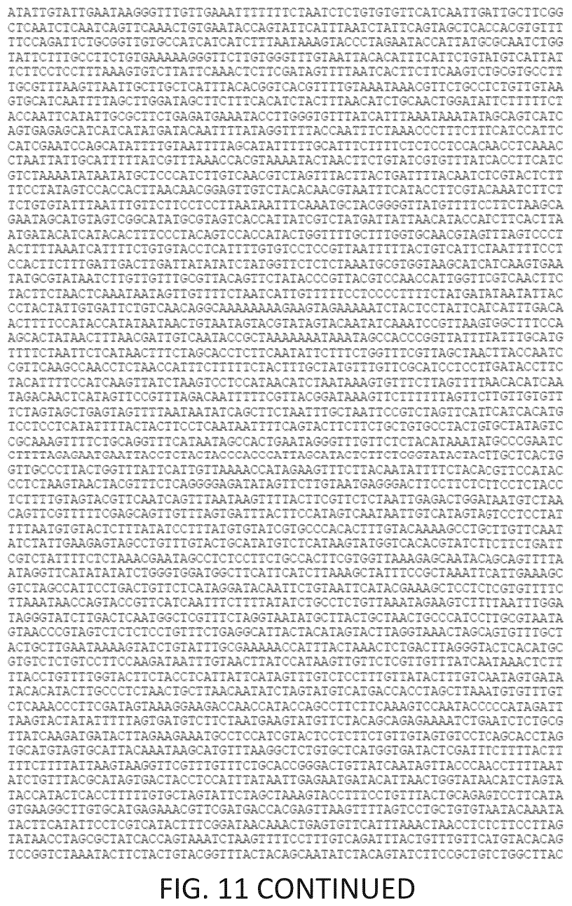

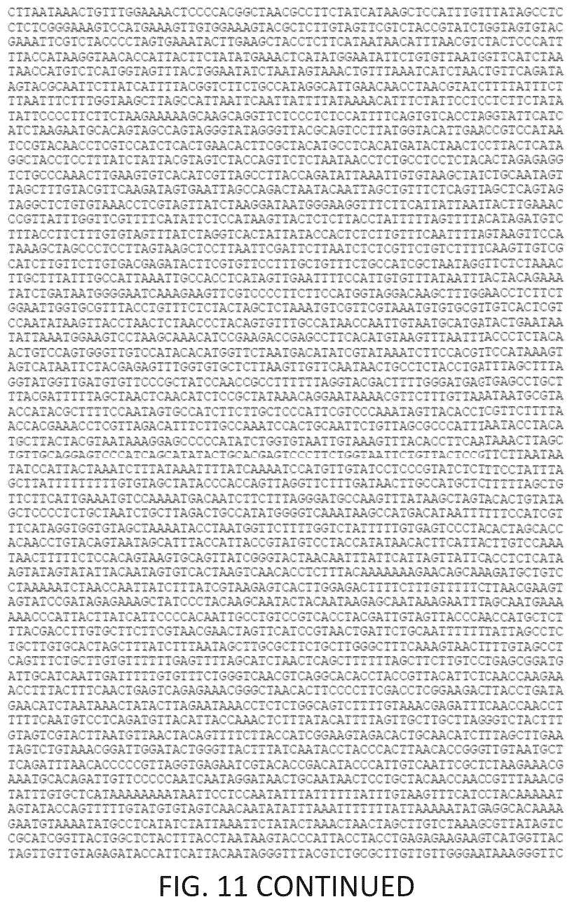

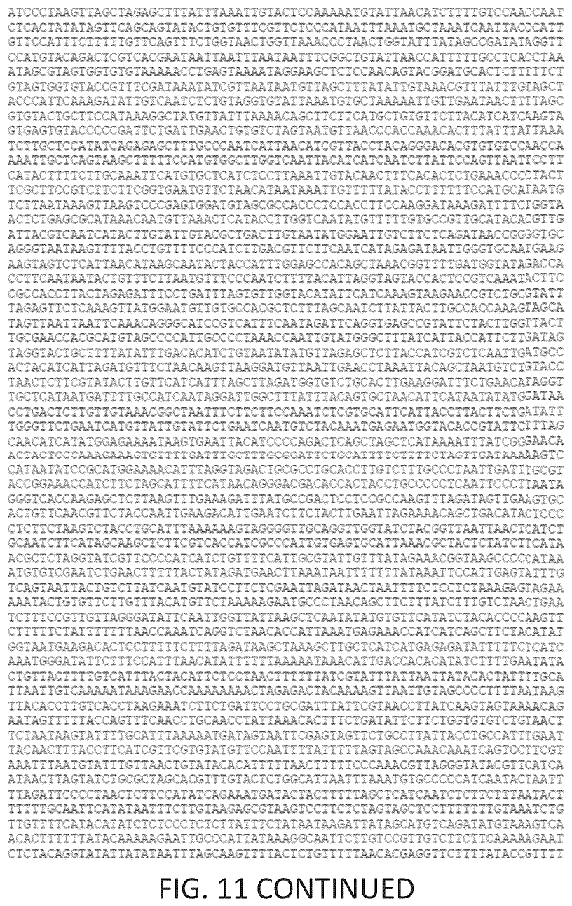

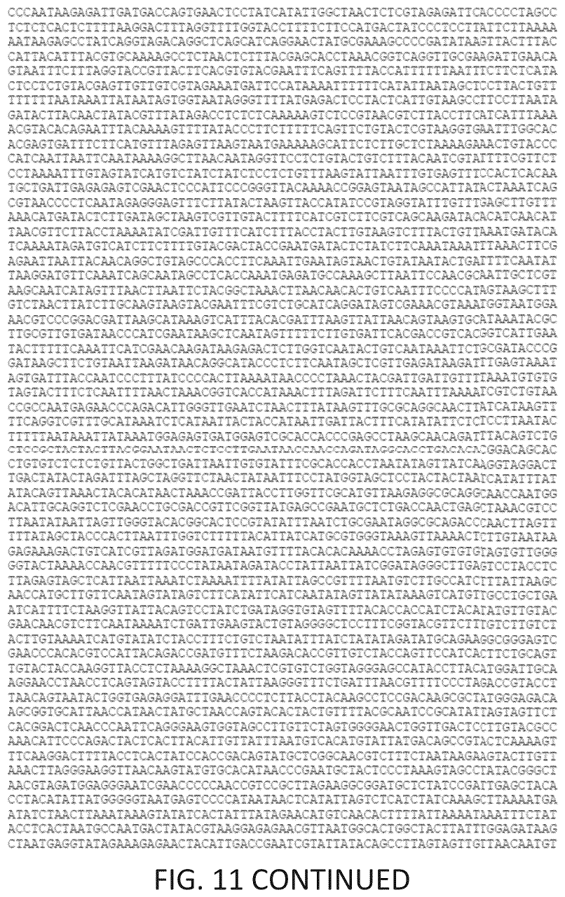

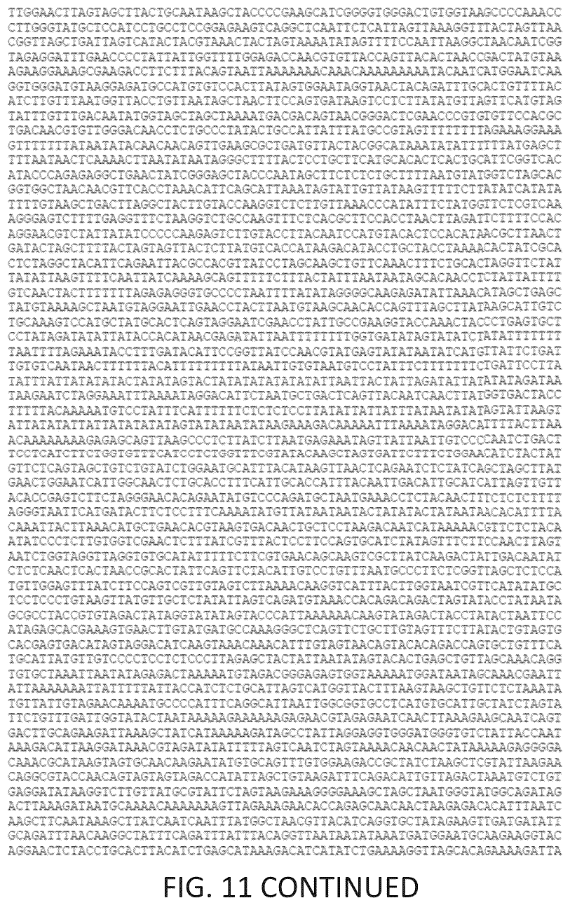

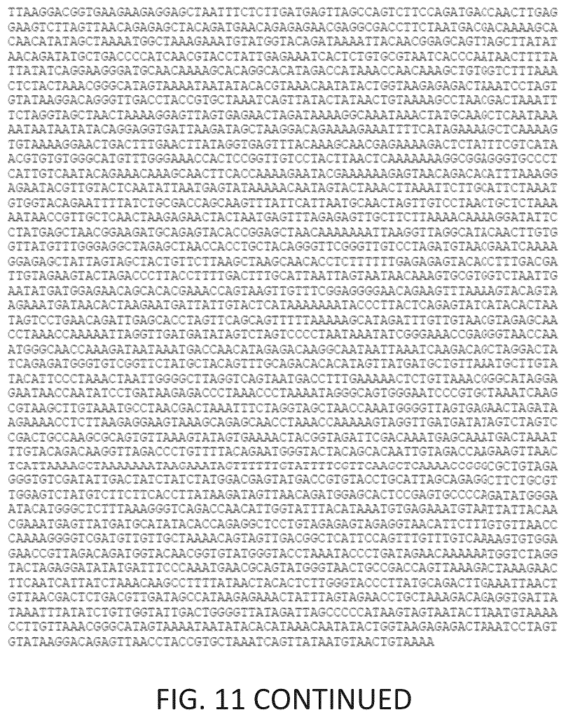

The supernatant from day 5 of the co-culture sample (FIG. 7) and the bacterial strains used in the co-culture were used to isolate evolved phages. As a result, phages were isolated from the supernatants of co-cultures with EF03, 05, 06, 07, 08, 10, 11, 12, 13, 14, and 16. The titer of phages against the co-culture host strain is shown in FIG. 8.

### Validation of Host Range of Evolved Phage

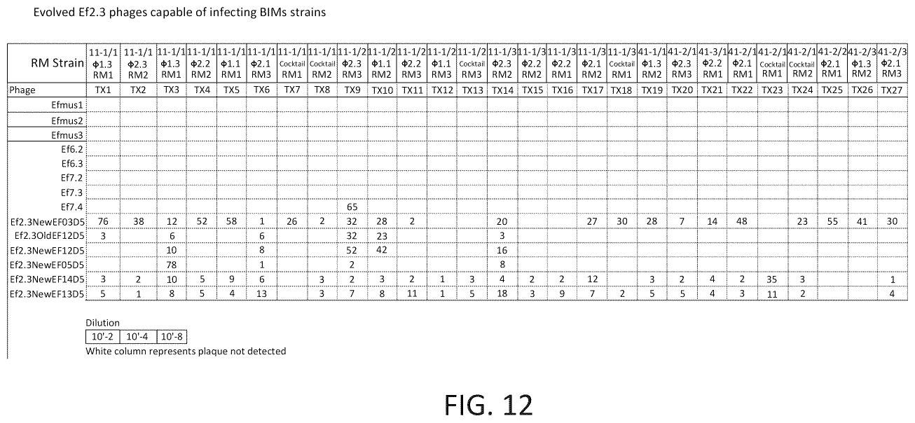

Using the isolated phages, a spot test was performed to assess the host range of evolved phages against non-susceptible reference strains. As shown in FIG. 12, phages derived from co-culture with EF06, 12, and 15 were infectious to EF06, EF12, and EF15, respectively, which were the hosts in the co-culture. In addition, Φ2.3N15D5 was infectious to EF06 as well as EF15, indicating an expansion of the host range. In addition, 02.3N03D5, Φ2.3N13D5 and Φ2.3N14D5 were infectious to phage-resistant variants (RM1 and RM2: isolated from co-culture of EF01 and ΦEf2.3)

Evolved phage Φ2.3N13D5 was sequenced and differences were found in the genome as compared with the parent phage 02.3.

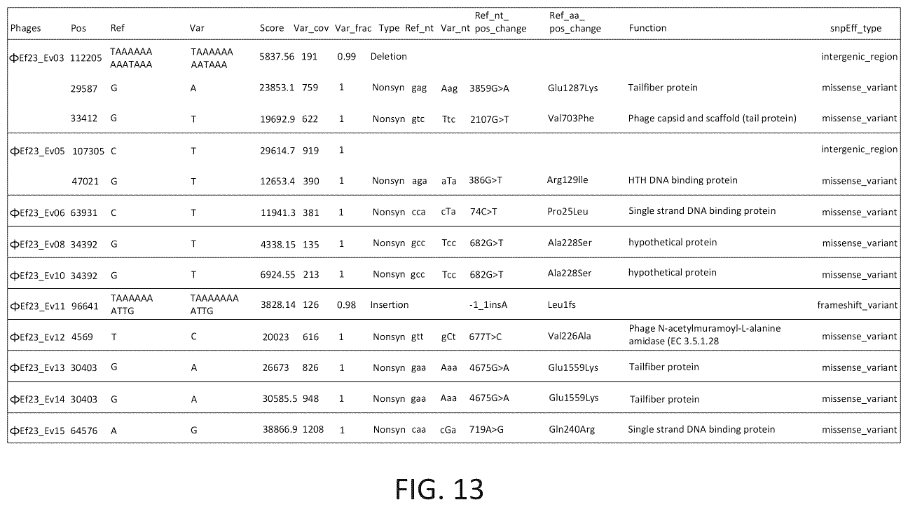

Thus, an evolved phage was identified with an extended host range and differences in the genome (FIG. 13) were identified after co-culturing. This can contribute to a larger number of cases with a single phage in practical applications. This is because the strains used in this study are all clinical isolates derived from patients, especially EF06, EF12 and EF15, which could not be killed by natural phage from environmental origin. Furthermore, in past phage therapy clinical trials, occurrence of phage-resistant variants has been reported, and it would be extremely useful to prepare evolved phages in advance that can kill even phage-resistant variants such as those obtained in this experiment.

Additional Insertion of Mutations into Evolved Phages from the First Round

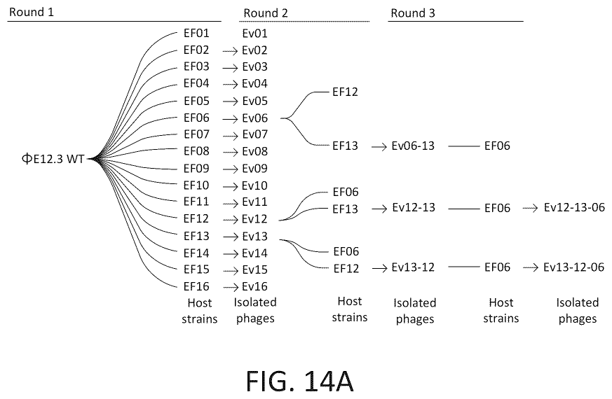

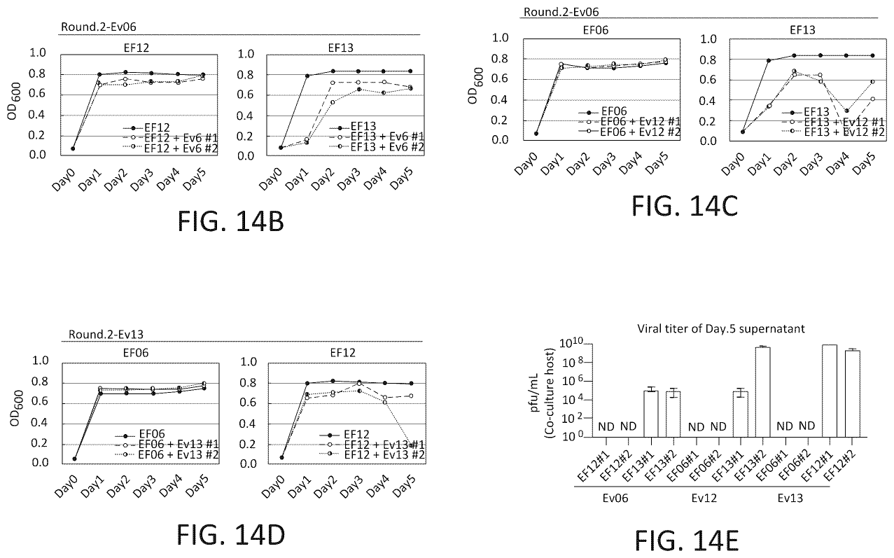

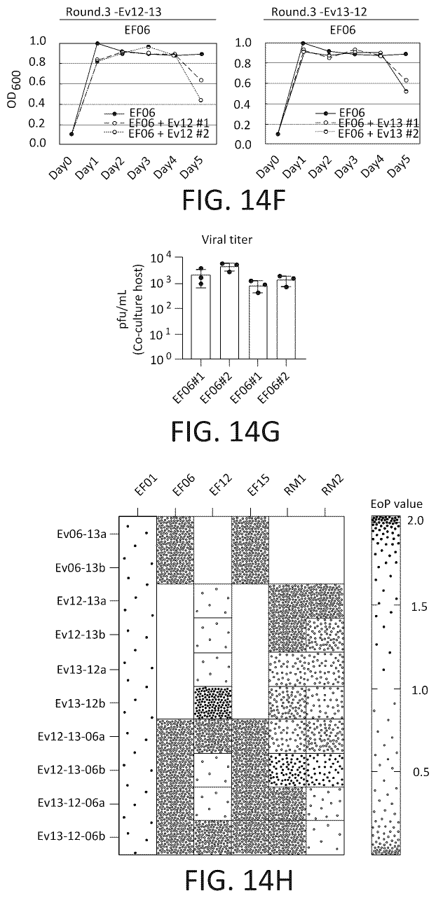

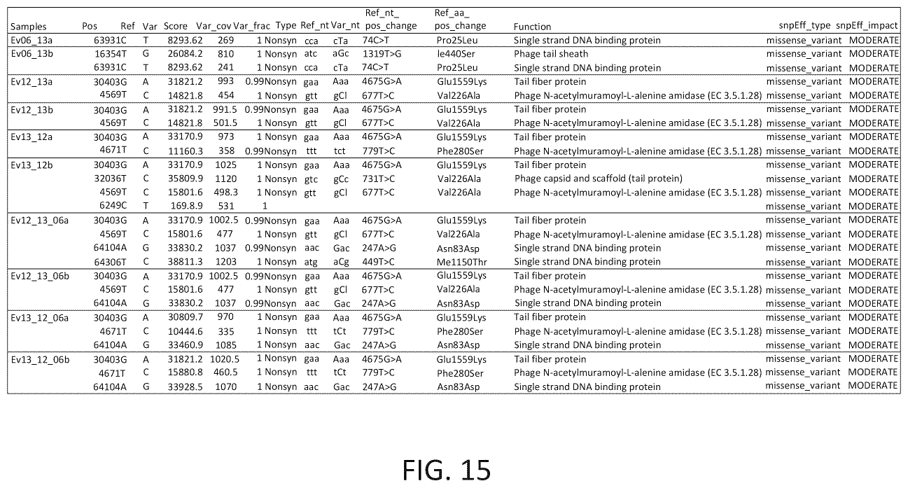

Using the isolated evolved phages Ev06, Ev12 and Ev13, which have single mutation described in FIG. 13, these phages were co-cultured with reference strains (EF06, EF12 and EF13) again as shown in FIG. 14A. Evolved phages were inoculated at MOI of 0.01 against each reference strain in the log growth phase (OD=0.2-0.4). Phages and reference strains were co-cultured at 37±2° C with shaking, and 10% of co-culture was passaged every 24 hours into fresh BHI medium. This procedure was repeated for 5 days, and the lytic activity of the phage was determined by measuring the OD values as an indicator. As shown in FIG. 14B-D, a phase in which OD decreased after an increase in OD was also observed, suggesting that the supernatant may contain further evolved phages that can reinfect phage-resistant variants. The supernatant from day 5 of the co-culture sample (FIG. 14B-D) and the bacterial strains used in the co-culture were used to isolate evolved phages. As a result, phages were isolated from the supernatants of co-cultures of Ev06 with EF13, Ev12 with EF13 and Ev13 with EF12. The titer of phages against the co-culture host strain is shown in FIG. 14E. As shown in FIG. 14A, evolved phages from round 2 were inoculated at MOI of 0.01 against EF06 in the log growth phase (OD=0.2-0.4), and co-cultured as described above (FIG. 14F). The supernatant from day 5 of the co-culture sample (FIG. 14F) and EF06 was used to isolate evolved phages. As a result, phages were isolated from the supernatants of co-cultures of Ev12-13 with EF06, and Ev13-12 with EF06. The titer of phages against the co-culture host strain (EF06) is shown in FIG. 14G. Using the isolated phages from round 2 and 3, a spot test was performed to assess the host range of evolved phages against non-susceptible reference strains. As shown in FIG. 14H, notably phages derived from co-culture of round 3 were infectious to EF06, EF12, and EF15 and phage-resistant variants (RM1 and RM2) respectively. These evolved phages had double or triple mutations in their viral genomes as described in FIG. 15.

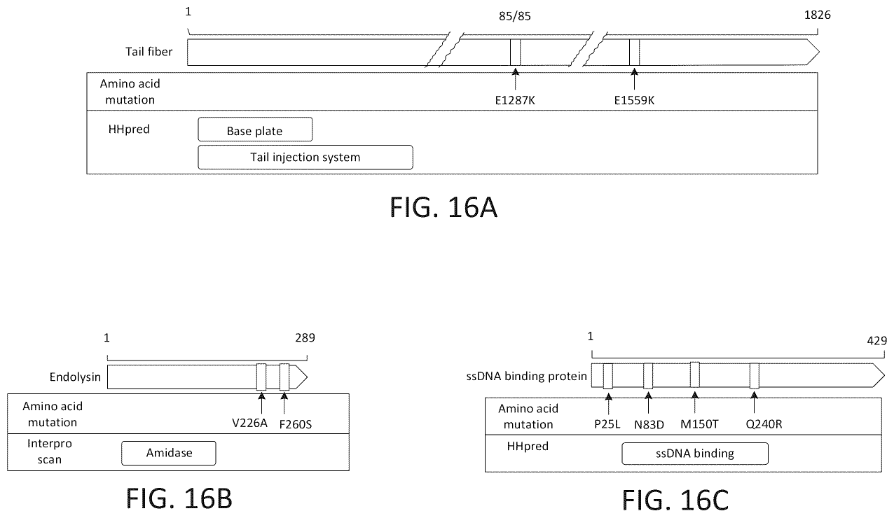

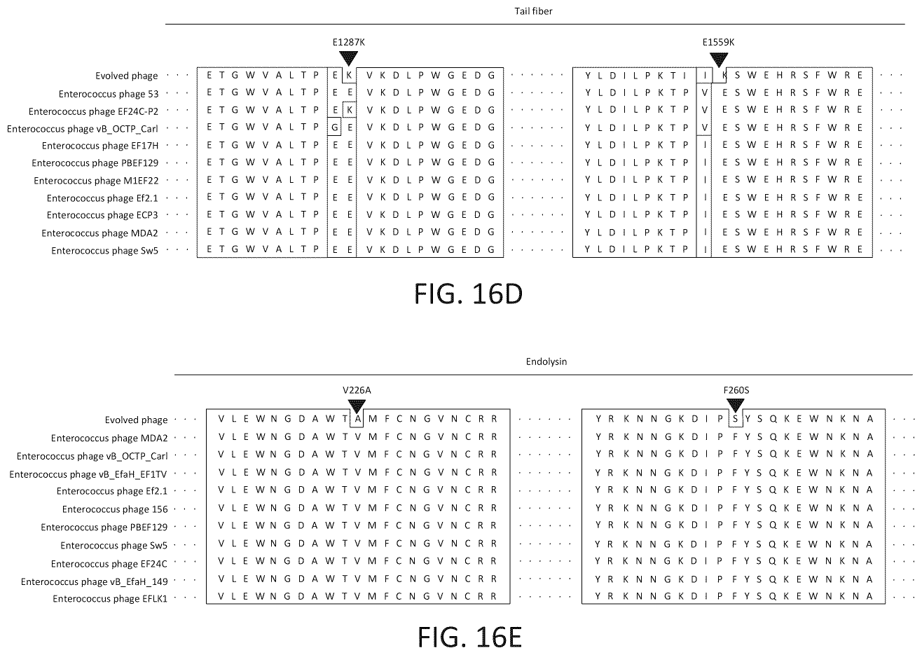

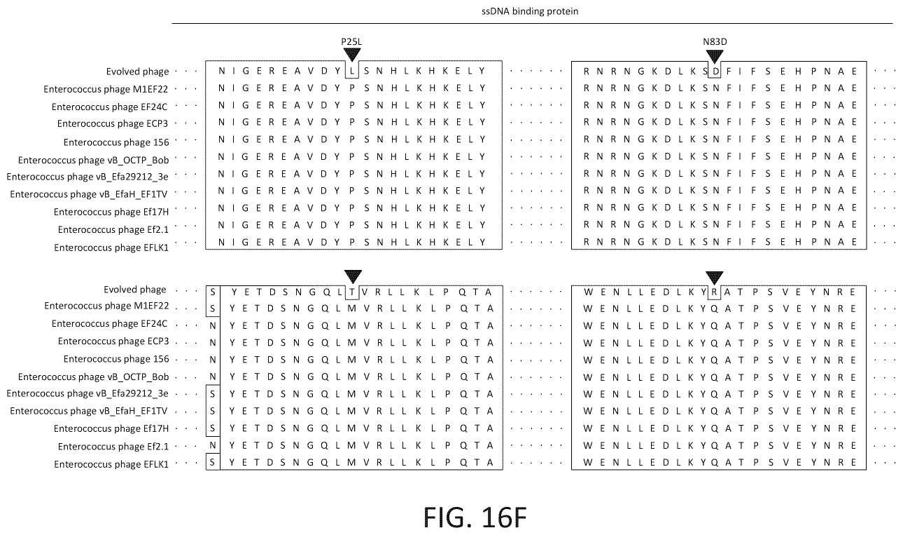

To summarize all these mutations (FIG. 13 and FIG. 15) can be inserted at two sites in the phage tail fiber and endolysin, and at four sites in the ssDNA binding protein, suggesting that these nonsynonymous substitutions contribute to the expansion of the phage host range. Furthermore, comparative BLAST analysis showed that, with the exception of a single amino acid mutation (E1287K) in the tail fiber, there are no identical mutations in known phages (FIG. 16D-F), suggesting that phages with similar mutations are extremely rare. In other words, the obtained evolved phages are phages with an expanded host range that evolved uniquely in the in vitro co-culture system in this experiment. As such, the methods disclosed herein yield evolved phages having broader host specificity as compared to the corresponding parental phage. Moreover, such evolved phage comprise unique mutations described herein conferring broader host specificity.

In addition, mixing too many phages as a cocktail may weaken the action of individual phages. For example, in the case of EF12, EF06, or phage-resistant variants (RMs) that could not be killed by natural phage (parental (Df2.3), if phages with only one mutation are combined, it is necessary to prepare a cocktail of at least three phages, such as Ev06, Ev12, and Ev13. However, if the evolved phages with three mutations is used, it is not necessary to make a phage cocktail in the first place, and it may be possible to handle more clinical cases with only one type of phage. This could also contribute to reducing the number of phages that make up a phage cocktail.

### EMBODIMENTS

1. A method to evolve Entercoccus faecalis (E. faecalis) phage comprising infecting one or more E. faecalis strains with one or more parental phage and determining whether one or more evolved phage are produced.
2. The method of embodiment 1 wherein, the E. faecalis is cytolysin-positive.
3. The method of embodiment 1 or 2, wherein the host range of the E. faecalis includes the one or more phage.
4. The method of embodiment 1 or 2, wherein the host range of the E. faecalis does not include the one or more phage.
5. The method of any one of embodiments 1 to 4, wherein at least one of the strains is susceptible to infection by the parental phage.
6. The method of any one of embodiments 1 to 4, wherein at least one of the strains is not susceptible to infection by the parental phage.
7. Isolated phage produced by the method of any one of embodiments 1 to 4 that can infect multiple E. faecalis strains, thereby having a wider host range than the one or more parental phage.
8. Isolated phage produced by the method of any one of embodiments 1 to 4 that can infect parental phage resistant E. faecalis strains.
9. An isolated infectious phage comprising SEQ ID NO: 2 or a nucleotide sequence with at least 80%, 82%, 84% 85%, 86%, 88%, 90%, 92%, 94% 95%, 96%, 97%, 98% or 99% nucleic acid sequence identity thereto.
10. A composition comprising one or more of the isolated phage of any one of embodiments 7 to 9 or a combination(s) thereof.
11. A method to treat a disease or condition comprising administering an effective amount of the composition of embodiment 10 to mammal in need thereof, wherein the disease or condition is alcoholic hepatitis, nonalcoholic steatohepatitis (NASH), liver cirrhosis and/or liver failure.
12. The method of embodiment 11, wherein the mammal is a human.
13. The method of embodiment 11 or 12, wherein the composition comprises aMyoviridae, Podoviridae, Spounaviridae or Siphoviridae phage, or any combination thereof.
14. The method of any one of embodiments 11 to 13, wherein the phage is isolated from the mammal, and optionally amplified, prior to administration.
15. The method of any one of embodiments 11 to 14, wherein the phage has broad host specificity and/or is a genetically modified phage.
16. The method of any one of embodiments 11 to 15, wherein levels of Enterococcus cytolysin or cytolytic E. faecalis are monitored after administration of the composition.
17. The method of any one of embodiments 11 to 16, wherein a cocktail of lytic phages is administered.
18. The method of any one of embodiments 11 to 17, wherein the composition is orally administered.
19. The method of any one of embodiments 11 to 18, wherein the composition is a tablet.
20. The method of any one of embodiments 11 to 19, wherein the composition is a sustained release dosage form.

### BIBLIOGRAPHY

Argimon et al., Microb. Genom., 2:e000093 (2016).
Ball et al., J. Abnorm. Psychol., 106:545 (1997).
Bertola et al., Nat. Protoc., 8:627 (2013).
Brandl et al., J. Hepatol., 69:396 (2018).
Cox et al., Curr. Protein Pept. Sci., 6:77 (2005).
Dalmasso et al., Trends Microbiol., 22:399 (2014).
Dominguez et al., Am. J. Gastroenterology, 103:2747 (2008).
Gill et al., BMC Genomics, 13:542 (2012).
Haas et al., Nature, 415:84 (2002).
Huycke et al., Antimicrob. Agents Chemother., 35:1626 (1991).
Ike et al., J. Bacteriol., 172:155 (1990).
Iwaisako et al., Proc. Nat. Acad. Sci. USA, 109:E1369 (2012).
Krieg et al., Blood, 118:6418 (2011).
Lee et al., JAMA Intern. Med., 179:340 (2019).
Llopis et al., Gut, 65:830 (2016).
Llorente et al., Nature Commun., 8:837 (2017).
Lozano et. al.., Lancet, 380:2095 (2012).
Maddrey et al., Gastroenterology, 75:193 (1978).
Marcuk et al., Bull. World Health Organ., 45:77 (1971).
Mathurin and Lucey, J. Hepatol., 56:S39 (2012).
Nobrega et al., Nat. Rev. Microbiol., 16:760 (2018).
Page et al., Microb. Genom., 2:e000083 (2016).
Poindexter, Bacteriol. Rev., 28:231 (1964).
Raven et al., Nat. Microbiol., 1:15033 (2016).
Rehm et al., Clin. Exp. Res., 38:1068 (2014).
Rehm et al., J. Hepatol., 59:160 (2013).
Ryu et al., Appl. Environ. Microbiol., 79:196 (2013).
Sarker et al., EbioMedicine, 4:124 (2016).
Schnabl and Brenner, Gastroenterology, 146:1513 (2014).
Schooley et al., Antimicrob. Agents Chemother., 61:10 (2017).
Seemann, Bioinformatics, 30:2068 (2014).
Shin et al., Sci. Rep., 6:29681 (2016).
Stamatakis, Bioinformatics, 30:1312 (2014).
Tang and van der Donk, Nature Chem. Biol., 9:157 (2013).
Tang et al., J. Ind. Microbiol. Biotechnol., 46:537(2018).
The Human Microbiome Consortium, Nature, 486:207 (2012).
Thursz et al., N. Engl. J. Med., 373:282 (2015).
Valentine et al., Biochemistry, 7:2143 (1968).
Van Tyne et al., Toxins (Basel), 5:895 (2013).

All publications, patents and patent applications, Genbank sequences websites and other published materials referred to throughout the disclosure herein are incorporated herein by reference to the same extent as if each individual publication, patent, or patent application, Genbank sequences, websites and other published materials was specifically and individually indicated to be incorporated by reference. In the event that the definition of a term incorporated by reference conflicts with a term defined herein, this specification shall control.

While in the foregoing specification, this invention has been described in relation to certain embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A method to evolve Entercoccus faecalis (E. faecalis) phage comprising infecting one or more E. faecalis strains with one or more parental phage and determining whether one or more evolved phage are produced.

2. The method of claim 1 wherein, the E. faecalis is cytolysin-positive.

3. The method of claim 1, wherein the host range of the E. faecalis includes the one or more phage.

4. The method of claim 1, wherein the host range of the E. faecalis does not include the one or more phage.

5. The method of claim 1, wherein at least one of the strains is susceptible to infection by the parental phage.

6. The method of claim 1, wherein at least one of the strains is not susceptible to infection by the parental phage.

7. Isolated phage produced by the method of claim 1 that can infect multiple E. faecalis strains, thereby having a wider host range than the one or more parental phage.

8. Isolated phage produced by the method of claim 1 that can infect parental phage resistant E. faecalis strains.

9. An isolated infectious phage comprising SEQ ID NO: 2 or a nucleotide sequence with at least 80%, 82%, 84% 85%, 86%, 88%, 90%, 92%, 94% 95%, 96%, 97%, 98% or 99% nucleic acid sequence identity thereto.

10. A composition comprising one or more of the isolated phage of claim 7 or a combination(s) thereof.

11. A method to treat a disease or condition comprising administering an effective amount of the composition of claim 10 to mammal in need thereof, wherein the disease or condition is alcoholic hepatitis, nonalcoholic steatohepatitis (NASH), liver cirrhosis and/or liver failure.

12. The method of claim 11, wherein the mammal is a human.

13. The method of claim 11, wherein the composition comprises a Myoviridae, Podoviridae, Spounaviridae or Siphoviridae phage, or any combination thereof.

14. The method of claim 11, wherein the phage is isolated from the mammal, and optionally amplified, prior to administration.

15. The method of claim 11, wherein the phage has broad host specificity and/or is a genetically modified phage.

16. The method of claim 11, wherein levels of Enterococcus cytolysin or cytolytic E. faecalis are monitored after administration of the composition.

17. The method of claim 11, wherein a cocktail of lytic phages is administered.

18. The method of claim 11, wherein the composition is orally administered.

19. The method of claim 11, wherein the composition is a tablet.

20. The method of claim 11, wherein the composition is a sustained release dosage form.


SEQUENCE LISTING (35 sequences)

SEQ ID NO: 1 - DNA, 147289 bp, Phage Ef2.3
  source 1..147289: mol_type = other DNA; note = Enterococcus faecalis phage Ef2.3 (also known as vB_EfaM_Ef2.3)
gaaccgacttcaccagaaacccgacaagggggcgcataagtctatttcaaaataatctgaaataagtatagttattactgtttatatatgattatccttaaatatctgaaaataggggcgtttattcgattatatagttaattatcttatactattaatcatctagcctattactgcattcattgctattatattctatatagggggcgcttatatcgtttatagtgtaattatgaggggaacgactaggaacgcttatacgggcttatagggggctttaaatgcataggctatttatacatatattagaatacacaaggaggaggggcggaaagttagtagattagactgttaaaaacaaacgggggatagatgcacattgttattttactgattggactaggttaactagctaaaataagaatgctattgtagcaacgtttataagaaataggggtgtacttttcccctagggtgattttaaaagggtataaaattcttttgttatttaattaaaaagttcatcttagctattgacttttaacaataactagtatagcataagggtataagataaagagaggggacaaggcacatgcaaaacgatatttattacacaatacaggacagggacggctttgcagaatcttataatgaatcaggtttcttacatgaactaggtaacatgaaaattattgattatacttatatagcactagagggggcagaaaaaggggacactgtggaactagtgaaagtgcctattttagagggctaccaatatccttatgattttttagtaattgatgaaataggtaaagaacactatatctattatgaagaagaagaataatttaaaaaaaatttcatcttagctattgacaaataacaataactattgtatagttaacttgtaagataaagagaggggagaacttaaaaaatgaaattaaaagaatttatcaaactagcagaatcaaaaggcgctacattagaagcatacaacgagctaggaggttatgaactaactagaggagatatagtagaccagaaccctattttaattgcttatatgcaaggggcttacagcgtagaaatatcaaacgaggaactagaaaacaaggagttaacagaactagcatttgtctataaaaatgcaagttttatttatccaaacgaaaaagaattattgtcaggactaggactatagcacaacggcatataaagcccgtaactaagttttaacgggctttatatataaatacattatgagggcttataagggcttatgagggcttatgagggggactaataagaatgctagaggtaggcaaatttgtacgaggtaacaacggctcaagaacttgggtgggtcagattgtaggtatagacagactagcgggggaatatcgtgtgcgtgacgttgtaacaggtatatacttttatgtatcttgtagcgctgtttatgaatgcgctagaccataaaacatttaataaaaaaaaattcatcttttctattgacaaataacaaactatattgtatactaggtttatcagatagagagaggaagttatagacatggaatatacattagacgaattactagaagaggaatacagcacactagacgaattgctagatagtaaagaatttaaaaaacaaatggacaattttaatcacatcccacaaatgcaaccgcaaagtcataacagcaatacgctagcagacacgggacgttatcctgaaaaataaaattgaaaaaaggggttgacaagttcagcccctagtggtatactaggtttatcagagagagggaggggcttcataatgaaagcaatcatcatcaaacaaatcaaggacggttaccaaggtatcttagaacgtaaggagaacggttggacgaaagaggaactaagcgactatacagcgggggtaagcaatacactagttgaccttttcaacgagttacaaggcagtagctttattacagaaaaagaagcggaagaaattgaggaagtattttctactactatcgatacattagaattttaaacttatctattgacaaataacaataactaatgtatactaggtttataagataaagaggggaagttattaatatgacaatcgaacaagtgaaagactacggtaaagcatacggaaaagagctatcaactgaggaagtgaaagaattttttgagaaacacaacggaatgccatcactaatggacttagcaaagttttgcggggcaatgaacgaggacggaacaaaaaattaaaaaaaactattgacaaataacaaaacatagtgtaatataataagtgtaagagggaaagggttaaacctaaaaaggaaaatgagtacttctttcttacacaatacataccagataaagaaagggagtttttaccatgacaccagaactagcagaaatgaacctaactaaaaacgatagtccattttcatttattaatgaagcaggggttttcattgaaccaatcaaaaacccgtttacttgtgagggtacttatttacaagcttgtaaagacgtagtgggagagctagaagcaaataaagaggtagggaacgacatcgacaactatttaaaaatgcaaagttggctagataagttagcatatgaatatatttaaaaagtttcatcttatctattgacaaaaaacaataactagtgtagtataataagtgtaaggaggaataaacctctttacaccacacaacagaaaggaagtttttactatgacagaatttgctaatatgaataaagaagaggtactagaattacttaatgattggtttggtgttagtgactatgatacagtgatggaagagttaggggagatgaaacaagttacctttacaggtagcacggacatacctctattaggtggtaacggtaacttaatcggtttacctcaattctttaaagacaatgaagcagaatcagagttgccaacttatgaagaattactagaggaactagagaaagatacatggaatcttgaagcagaggacaacacttacaattatagcggcttcctagaaagtgaatcagattttaaagttattcaggcagaaaattcagacacgactattgcattttttgcaatccatacgggtatagacataagagcgggctactcaaaagcaatcccagttatttttgaaacttactacgattttgatgagtttttaggtaactacttttacagtcaaggttactatgcttttaaacacgacaacaaggaatacacaatcagtttagacgtttcggctgtttcggagtatgtacgaatttatatagctgatgaaaacaacgaggaactacagcaagtctatgaacaagaaacttgtatggacttagacatagaaagcgtagaagcatacttgaaagaagagggtatttcatttactgacttaaaacccgcattgtaaccatataaggcactagggagcttatacaatagagctacaagcttcctagtgtaattataaggtaaataactagaaacgcttatacgaaagaatagggggctttaaaatgaagtttaaacgagcaatcgaggaattgaaagagaaagagggtatcaccttagaaccagaatataacggggtatatacagttaagggctactattctaacttagaggtattacattgtagcgactacgatgaagaatataataacgaggaactagaggactgccatttacatgaacttgtattcaggtatgagggagagcaagcatataagagcggatattatgaactgctaaaattattgaactaatctattgacaaataacaataactttggtattctatatttgtaagataaatgaagggaattgataagaatgaaaaatctaatgaatgaaactatctatagtaatattacattaaacctaactaagaaggcacaaggcattatatcccaatatgtagaccataacgagggctacccaagacaaaagaacagaggtcactactaccgctatacagttacatttatcaatggtgaaacgttcgaaagtaatagcaaggcggacattgctcactatatttcatctatgacagcggacgaactagaaaccatcatcaaggaagcacaagccgagtagtaagaaataggtatataccagttaaccctgccccgtggttttttgaccgtactttgggggtggggctatttgcctaccccgaccctcgggtacctattttttctaggaaaattattttttatatatttagaaagtcaacaccctacatataaagtcaacataagggctcaccctagggctcctatataggctcacctaaatactcccatataaaaagacccctccccgttgataaggataggtcttttttatttagccgtctgccctatgattggttattaggttcaggttccgccaagcacgacctcggttacattagtgtaatgggtatacccttgtaagtcaccatcctctacaatgtagtagtcagtgaaccctttacacgtactattatcgtaacctatcactctccctatagggctcctacctctaccattacactcgaaggtgaaattattaagtataacttgtgtgttgatagggagcaaaggctcccctcttgaatttagcatgttaatgtggctccttttatttagtattcttaggttaatagcacaaccaagccactaatgacgactaagactacgtaaatttccactaaaaatgagtatatagtaaaaacaatagacaatacagtagtgaatatcgcagaggctatcacccatttagtatttaaactattcgacaaaacaacaataccaacgataaattcaactagagcatagcccaagacataaatacctacaatacttagagtaatgagtacagggtgacttataaatccaccccatacattataaaaaatatttagcatatggttactcccctctaagactaatttgttccgccatagtctttgcgaagtatgtccagtatttatggaagtctgcaaagtccaagaaatactctccgacaatctctcctgcttgattgcggtaaagtggtactttcgttttctcgtctttatggactccatactctgtgccatttaatgaaatccatacaatatccggagtcacactagttactgttgccttatcacaacctagtatcgggtcacgaagttctggttttgtgaaaactgtatccccaaccttcaatttgtctagtcgctctttgtctattaatgttgtctccatttgttctccctccttatacattataaaaaatatttaacgtctaattactaccctcaaataaagaacattactacccatccaataagcataaataacaatgttgatacagtagcttttgcaaataccatcaaaatagaatcttctgtatttgtagtagtcttagctgtaataaagctgataaatacatctagcccaattgcctgcgcccatgtaagtgttaacaccccaaaggttggagcaattaacccgttccacaagaacatcgtaacataccctccaatagcaagggtcaacaccattagaactaaagtcccaaaaaatttacctaatgcatttgccaattcgtcttttgtattcttatccatcgttttcctccttaaatgttataaaaaatatttaataactagttacctcttctaagactaatcgctacctcggttaccctagggtaatgggtacaccttcttaagtcaccattctctacaattatatatctcattatgcatggctatgttaaggctaatttgttctaagatacggacagccatcgctcctccaccaaagccattatcgatgttaacaactgcaataccagaagtacagcttgtcagtgctgtatttaatgctgtatacccatcagcagagatgccatatcctgtagatgtaggtactgccacaataggcaagttagtgagccctgcaagaacgacaggtagtgctccctcaaagccttggacagacacaatagcattacaatcattaatgccttctacggaatacatcaatttatttaagtcgttcacaccaatgtcatggaaagccacaggaacgtaaccactcaataacacagttaggtacacctcatccacaatatctctgtcggctacacctgcagaagtaattgccactgctaggtcactgtttacaggtcgttcttgataaaatgcagtttcactagaccgacagtacacgaaagaggcttccgtaccccattctttctcccaatcatactcctctgccgaatcaataataactttacgagccaacatagggatgtcattagttggtatttcattgaatacttccagtggtagttttgttatcataatatttgtattgtaaggaacctctgcaatttgttctgccagtccttgtgctgttttatttcctccaaataatgtacgtaggttcattcgttaagctctcctttagtttttcccatagccatatagactatttatttttttgttaattctatttagacttgcaaataagttgtttctcttcttctcttgttaattggtcgtgatagcactgtgccattacgtatgtctccctccgtttttatatttgttatagtttagtgttaaaccagtcctatgttctctcctagcgtttacaatagttgtttagggtgataataggtagaatacctatcaccgccctaagaggcatatagggtacttataggactggtgcttagttaactgttttaagtagcatggaaaatatgcgcttaagtcaacattagttcttttactttaaatgatgtcaattgcaatataatgatttgttttggatacattagcaatttcatagtctaagtatggttctagtacttcgttgttacttcctatctttgttctaactgttatggtagcccctgccgtgggataagtaatgagtacgtccactccgggcttcattaacttaactaaatcttttaacttcatctttatctcctctttaatagtatatgcttaaatctattttatgtcaactgcttaatttttgtgctcgtagttgcacagatattgtatcaacaatgactgcccactgttcctccgtaaaattgaagtcaaactgtgtatagtcttcctcacctgttgtagtaattgcaatgtctccataatcatcaactgagaaataaatagttgcagtcgcattttcctccggattttgaggaagtgtaatagctaactccccttgctctactttcatctattgctcccccttagctctcttccttctcaatcatcttgatacctgtaaaggcatatacagggagttcttctgtgtaagtgtactgattagctttaaaggtcacttcgtagtaactctccatgccacaatgtaacatatctacatatctgatttccttacaaggcatagcatttctctcacttccgcagaagagccagtcttctgtacagtattctactctacctgtgtggatacttctaactgctacgtatggtttttccatctattcttcctcctgtataatctcttctaggtcacaagggtgtacatctagtgttcctgtccttagactataacgagagtttcctcgtactgcgtaaacaccatctgggtatactttttcaagatatacagtatccccaattgtaaacccatggtcataacgggtatctccaataaccttaaactgtctttttatcgtatacccatacatgtaagcccgtgctaatacgtattggttaccctcatcaatccattcaactagcttgtcatctggtgtttcatcgtatgggtcaaaatactcattagctaataagctcaacattgaatcgtactttttgattgctcgtgcaacaaaactaggtatctctacttttttagccatctattcttcctcctatttaatcataatacatacaaaatacataaaaaagctaacataagcaaaacacgacccatccagctgttacatatgacatatagaaaacgtatataagatatactactgctgtaattgttaataatacaagtatcgtgttaatgagcttttctaaatcaatcattatacaaccccctaatcctcacaagtataagtctggctctccttatactcttcctcctatttatatccaaagaactttaagtaattttctgcttctttttctaagtgctcgtttagctcatctagggtaacaactttcccacagtcctcacaggtgtaggtctgggtcttcttatcttctagcagttcaccatcacatacaaggttgatacagtgggttgtccaactccctacatcatcaggtaagtgccccattatacgtcacctgcttttacggcaaatgccatgaatcgttcatccattttgcggatttctttctctgtaaaggtagggatgtcttttaggtagtttgtaaagtcaggagaccctaatcggtcgatataggtatatccccaatcatctttatcttttgcgataatgatgtagtactcatcttcttcctcctctaccgtatgacccagtacccatgctttggcaagtttctcgtggttttctggtttattagctaaccaaaagtacatcttactagggtagtttcctttgtcaatcatgttaataaacgttaagttctcttctttgcacttatcaatatattcccctactttataaggaataacaactttttcgggttcatctaaatcatatgctaaggagactgcttttgctatccctgtattgtaacctagagcatgagggttcaatgtaccgtcacattctacttgagtaaaaacctcgtcacttagtctactaataaattcattcttgttcatttagttccctccttaaaactcaagactatttttaataagttctgatagtcggagcataacctcatgaggtagctctaattttgtactcccctgtgagtctgcagagctaacttccaaagtatattttacttctgttttctcatttacacgggaagccttcttctcaacacatactttataaagtgctgagttattgactgcgataattttttcttgtgtatttacactagtattgctatcataagtaatttcatagcccattataaaaccctcctatttgtatactggtacagcaaaagcccagtatctttcatcaatttcttttatttcttgttcggtaaatttgctcttacccatgttgggtttatttgagaagtatacttcattattgtgggatgtttcaacccaaagataacaatcttctcctttattaccaagaccagtaaacacgacatggtaaagtggttctttctccacctcatatccatctttcattcgtattaaagtttcaatagggttattcttactttggtctaaccaacaatcgaactctgattcatgtgggtagctgttgccatctaacattctaatttgtgtaaaaatagctaggtctaatgcatgtttattttcctcgtaccattccgcaacaaactttggcacaattacttttttaggttcgtctagtttctttgctagcaaaatagccgccttgattgcctcattgtagcactggctccgatttgataactcttgaacgactgggacgtgactgaactttaactcttctaatgcattaatcaattcttgtttattcatttaatttctttcctttctttcaatagcccactggctgaatgcttgtaagacttgcaactgcgcaatttctgtcatgtatctataatttttataaacagggctattccgataatctgtttttgtagaattggctctcagtctccaaaatagttctataggttcaatatctgttaacgtatatttttcttttaaccaacccaaaacaatttgttgattctcacttagttctacttggtctaccttatcatcagtgtatttgaaccagattttctcatatgttgtttgtccgcctgtgattgttacaaattcggcgcttggttgctcattttgaaatttttctagttgttcttcgtaatgtatatccccaacatcaaattctttaaatttaatcattgtactccccctgttctaaaaaccaatcagtaaatgcttgaataacttgcgcttcctccgttactgataaagagtaatatgcctcttctgttttagggtcttcataggtctccccaatacgttcgagcgcctcaaataaggaatctttactacagtaacctacttttaaccattcaaatacctgtttctgatttttattcagttctggctttttaactgcctctactgcagtcttatacgctatttttaaaaggtctccttcaaaactattagggaaattaatttcaccatttgctttcatatacatatagcaaactctatcaattatttcgttatatctttgtctactcattcactatccctcctcgtcctctacgtcaccccaaccaaagctacagtaggaagagaacacgtctctagctacttcgtctatctcctcattcgtagcattatcgggtacttctactgtctctatttcaccaatacctgcccaagttgtctcaatatatacatttatttttcgcatagtttaatacctaagactacatagtcgtctttctgtgcataatcggtgatataagtaacttctactttaataaactctcctgtatacgaaccacatgtgtactcatttaataaaagaatatctcccacatggtaatttctatcatttttacgtatttcaaatagttttctaccttccttaactgctttaaagtacgtaggtgcaatcttgaggttgtgtgtaacttctaattcactaactaaatcaaaaatgatagtgtttaattcgttaccttctacctgatgtaccaatttttgtaaatttattgatattacagtaggtctgccctctctgctcccataaaggttattctgttggtctctatattcctctgaatcaataactaagtttcttctgttatgcttagtaagtctttccttctctgtcattgcctgtgctagtccatttattgttttagatacttttacttctccatttacaataacttgtactaggtatctgtcatcctttactgtaagcttattatccataactaatatctctccttcataatcttaattgaccagttatcgttgttactaaatacccgttctaggtcattcaatgagttggtatttacaatttctttttttagttttctatctgaactaatattaccaaatcgtttaaggaatctttgtttaaagtgagcataacccatatagtcaaaggtaatagtgtgttctttaccaaatataccccgctctcttaacttaatttctacactctcataagagctatctactttcataaaattatttagaagagtgtgcaaatcaatacaaatagatgcccctgcactaccgcctataagtatatatttagggcactttatatcgggatagcaaggggcatctaattcataccacatatcatttacatctaacccataccatacactatttacgtctactgaggctctttctgtcttaaatggttttatgcaagtatatttcattgtctctcctccaaatcgtctagtacatcttctaatgttgtgacaaccccctgcaaataacccagttcatgccacccataccctctcttatcttgctctaacaaattattaattctttttgttttacgtttaatttgacggttatatcttctttttagttcctcttcaaaggtcactaccttttttgcttcctctatatcttttaataattggtctagcccatctccattagtaccatagttatacactagagctttctcgtccttataggtagttacccctaatgattggtatacatacttcttactgggaacaaatacaagggtaatgggtctgtacagttgaggtagaagacaatcttctgataacccaccaaaatctgttttagaagataccttctctatcacatacctcatatcttccatacttaacgacttaatacataaaatatatctgttaataaccattttctttagctcctcctacacccaaaatagttttcgattctctgttggtttaaaagactccaattctcgtgaagtattactttttccatctgttaaaccttctttcactttatatataaaaaaaaataaatctagctatctacaaatacattgtatcatcagctagattaaaagtcaacactttgtatttaattattttatattacaatagttctttatctaaaattgttttagggatagcccatattgtagtgtaacgaacccctttatggttacctgtaactaatacgaacgttgccgtatctagcttagtaccgagtacagatgctagtagttcatggtcttctttgctaattggtacttcaaaatcaagttccccaagctctaccacaaagttatttgctctaattcccataaagtattcatcattacgttttacatggaatacattcttcgtatgaaaactttgcataacaatatttgcaaaataactgttatccctagctacaataaatctatctacataaatagcctctttgctgagtagttcttcaatgcagttactcataacatattctcctcctcttctaatagtagctctgatacgtaaatttccatcattagactttgttcaatcccgtctagtaataaatgcatagcacacaagccacacaacatttcatctaaataaaaatcaccacatgtctcctctaatggtacttgacacattaaacactctatcaccacacagtcctccttaatttaaattttccctaacttctgttagcacctctcctaatagattaagaccttcccatttactcgtatcacgagagcgtgggtctgattcacgtagtcctattccccatactttatcaaaaggtgatgcctctgcaaacttagttcctttagggatggacagcataaagttacgtaaactatcattttgtgtgaatttataagtattcccatctaaaacgattgatttacgatgctttacccacaagttttcttcaaaatttttaacttgtctccctaaacgtttagcatcttttgggctctctgtttttaaaattctttttgctgtatcaacatcattaaaaagcattgctttacgtaccatcatatagtgctctgctgtcgggaaaataatatcttcccctaaacccttaataggtgcgctgaactgtgatgggtaccactgtgacaagcactcttttcctagtttattacgtttgcttggggtatgtccccaaaataaaatatatttactcatttaatgttcctcccaatattcatagtagtttcccttatgttcaaaaacaaaatgagggctaggtaactcaccactaacttctatatgctctgggttattataagaatagtagggttcgttaccagtagggctaattgctattactggtttatctttaaaaaatggtggtattttatctccatcatttaagtttaatttataggtacttaatggcacgcttctacgatgccctttcagtgttgtatactcctcaagactaattattctaaggaaacccttagcttttaggggtgataagctatctattgaaacgtcttcttgtttgtaagtgacaagggcactataaatggttgaggggtttttgtatgctactgaataattaattgaagtgtgcttatgatttgctaaaaatttctctaattccttagataaaccataaggtgtatcgctatcaataacttttactttaatcatgtgttatcctccttatatgcttctctaactgttcctatactttggtctgggtctggtttactaaatggtaagcttgcacgctcttccttactcaatgtttttaagcaagccacatgtactgcgatgacccctgacttagcttccatgctgtccggaagtgtaagtatatagtcattatacagtttacttaacactagccaataacgattgctactacttttcatattagctaaaatcatcattttttcatagtaatcttgccccatacaagaaagtcctcctattgcagattggaggagctcatcttcaagagctaagatagtatttgcgtaccctttcagctccttgttaagcccctctatagcaatctcaaaacattctctttctggggagcctacagttaaatcagtaagtggctcatatgctcttacttctctcccactaggggcttgtcttcgtttacctgtacgataggagtctttaaacccatactcggaacgtaaaatttcttcttgactatttctcccagacctacctccaaaatagtaatcttctactactccaccaagaccattataatgtcctctaattgtccctatgactttagaatgtctaacatgggttaactgacagagttcccctccgttatcagggtcacctacaatagctgtctcacatacaggacagatatatccaaaacaaatcactttatctacctccaattaacatacattaagaaaaagttacaattttttcacaaggaatatcatagtatgcctttaaaccgtaatgttccaacatgtattctttagcgtaatctacaaggtgctcagggcatttaaatgtgtaagagccactcatcattacaccactattacttaattcttctctcgaagctttagcaaaccacttagcacactctacaggatacccaataacacaccctaaaacttcctcgtcattaaaccctagcttaattgattcattgtatacttctatacttatcttatctgatgctactaggtatatactaccatataacctcgttttataataagagcgttccaaacaacttagttcttcttcttttaatacgattgctacacgatgacctaaattaaaatctaaaataggctttggaaaaacgtattctgctttctcaacaaactccacttagccaccccttttctcattccctaaattatcttaaatgctgtattagttagaacggtaagtcactattgtgttcttcctcttctgggatatataggatattttcaatcttttcttcaatatgtccaataatctctgtgtgctctttaattttctctttatgattttctctttgcttatttaactttgcatattgctgtaattcatctggtgttagctcttctagtctttttttcatttgttttctccttctaatgtaattttctctacgtcatcaacactaccaaaagatgggggctgtgtcaagttcttatctaaccagttagcgataatgtcatgtaactcgtctatagcctcatcgcttacttcatccgttgggtagtagtcctccgctacttctccatattcactgtatagctgttcccctacagattctaacatacctactacatctacactaataactgctttattgaactgccctactgcaaagcttgttgttcccttttcaggtaatgctccaaatatatcctctacgtgcatccctaagctatctacatatgtttttccttcattaatggacaataatgcaagttttcctgcagacactgcctcttctctcgtagagaacgtctcacacgctttccatacatctgtgtcttcctctagtgctaatacatagtctttatcttccattgtcttgccctctctatccttttaatataatcatatttgtattactgtctagttgccatccatcaaattctaagtagtggtcataagggttaattggttcacctacaactatatagtcctctagtgcgtgaccttggtcttttagctctagtaattcgtctaataaatcttgtgctgtcaaattattcatgccatgtagtatcctttctctttagcttcttcccactcatctacagaaaacaaacgctcattccgagacagagagccataacagtttactgttaagtctaccattaggaaatctttaccgtattgttttgtccaactctctgtatatggtgttactgagtgcacttctttaatatattgctctaacatattcattttatattaccctttcttggtttaaattttcgtgtttgtgttcggttgtttctacttacttctttgtatctacgaatacctaatgccttagactctggacgagaacgtcctagctgtcttacaaataagtctagtgcttcttttactttgtcatcatacatcttccctaattctcggaaatactcaaactcacctaaatacacatggggcatacgccattcatctagggtagcctctactattgttcgtttgtttaaatcataattatagatatataaaaatactatataagtacctgtcatcaaagaatagcttgtcctaatttcacttagagttacgctgtctcttaatgatgttaaataatcattccactttttcttacgttcttctgctaaagtttctatgaagtggtttacctctttctcatccctcactacttgctacctccttgttgtttatgtctatatattatcacctactttgttatatgtcaacacctttgtaataaaaaagagcctagtaatttacttacttaggctctcttgtttatatgtgtttaataagttacctttactatcgacaacaacagcattaagttgaccaccgtatgcacaggcaccatcaataaaaatacttctgtcacctaagaatacatcgtggctgttctcttttctcaaattaattgtgggtgtgtgacccattacaattgtcttatctgttttattcttctgtaacgctagcggtctcgtccaaatcatttcatctttagtagctgttttccaatcaggctgacaataaacaggtatccctgcgtgtacacataaggtgtctccatgctcatagtatggttctaataaactaattacttcaactacatttgttttttctaataacatctttctagtggtaaacgcatcctgtaggtgttcatctaaatccaatagactacttatcgttttatcaccgccattgtacatccacattgcgtacatctcacttaggtcttcgtagtctgattctcttatcaaggggaatgttaaaaaatctaataacatttggtcatgatttcctaacaaagcagtccctccattaaggatatgcttataaacgaaacccagtacttctgcggatttgctacctctatctacgtagtcacctaaaagtattaagtcctcttcatcactattccaatgtttattaagaagcattattagctcatcataacaaccatgtatatctgatacaataaacactttatctttcaatgaataatagcccccttaactacttctgctagccagtaagtagccagtactagtacagtattgacaataaatgttattactataaccagtgctaacttataatttttactcctagcattatagtttatataattttttatgttaactctaatatttttaaacaaagtgctcccatatacgttcagtgctatatatagtaaaagcaatacgataaagtagccttgagctagaaacacgtcagactccctgaacgagttattaaaccatatgctgaatatcgcaatcacttggtgaattataagcattgcgccattaccaaagaggagtatactaaacgctataccgactacgtctctgtactctgaaagtagcctatcatatactattatccctttagtaaaaataaaagcaacaactgccatctgtataagtaaaaatattgttacggctaataagttttctgcatgcatagtcataatttagccactcctttcaaaatgggtaaagaaagaataataggtatatcgttctttttctaccctaccctcatgtataaaggtaaactcaacaaacttaactcttagcggtatatctggtgctttctcccaaggaagtactcttgggaaatcatagaatactcttgtggggtaaacttctttcataagtatcctagggtatcttttctttggtataaatcttttacctattatacttgagtagtcaggtttcattgtaagtcaccatgaatagagtatgttctacttgctttgttaaagtttacttgttttctgtttactaatcttattgcatcctctaataaaagaggttttcctaatttatggttatctaattctggtgagtctacccctacattcagtaagtttggttcgggtctggctaactcatgaatgtgcccatgtagattaattaggttacctctatctcctaaaattagtgggtaatgcgtcatatgcacaaccttatgcattcgttttaaaataataccaacgtcttcccagtgtactctactgtttagtgacgtattatttttaaccgcttttcttaaaggactattatcatgattaccctttattagccatatagtcccgtttagacgctctaacacgttaacaacactatttaccttagcacccattgaaaagtcacctaggtggtacacaacgtcttctggacgtactgtagtgttccactcattgattagatactcgttcatctcttccacatctttaaattgtttccgtgtgtccataaaagaggcttccccacaaatgtttctatggaagaagtgcatatcagatattacatattcttttaccatggtgtcctccttactgtgtaaccaaccatacaaatacgaatagttgtaggtagtgtactgactggtcgataataaactgttttcgtgtatccggtactactttaaggaaccatgcactttgtgcttttaaatagtccatgataaaatgtggaacccataagcatactacaattagcccaagttttactggtgttcctaagaacaatgtagttgcgacaataatatacgtccagatgttcacatggataaatagtacgtataggctcgtttgtttccctttggcgatgtaatcactttgtaaagcgtagtcccctaatgcatgtgccatcacgaggatagctaaataataaaagtaactcaacaacaatagtcctcctctttgtatgggtatgtgcaagtgtcacaggcatatccgtattcatttggcgcaccttctaataaatctccaccacattctgggcaagttgctagtttaatcattatagtttccctccatcaattagtaatagtagaataccacctaataccagtagccagaatactgctaaaattagtaccatttagttgtcctccttgaacagctcaccgtataattcttgaattgcctcacgctttacttttaacattgcaacctgtgctgatatgatgttacttggttttcctactaaacgtccttcaaagtgctcaatatccatattgtattgaataagggtttgttgaaatttttttctaatctctgtgtgttcatcaattgattgcttcggctcaatctcaatcagttcaaactgtgaataccagtattcatttaatctattcagtagctcaccacgtgtttttccagattctgcggttgtgccatcatcatctttaataaagtaccctagaataccattatgcgcaatctggtattctttgccttctgtgaaaaagggttcttgtgggtttgtaattacacatttcattctgtatgtcattattcttcctcctttaaagtgtcttattcaaactcttcgatagttttaatcacttcttcaagtctgcgtgcctttgcgtttaagttaattgcttgctcatttacacggtcacgttttgtaaataaacgttctgcctctgttgtaagtgcatcaattttagcttggatagcttctttcacatctactttaacatctgcaactggatattctttttctaccaattcatattgcgcttctgagatgaaataccttgggtgtttatcatttaaataaatatagcagtcatcagtgagagcatcatcatatgatacaattttataggttttaccaatttctaaaccctttctttcatccattccatcgaatccagcatattttgtaattttagcatatttttgcatttcttttctctcctccacaacctcaaacctaattattgcatttttatcgtttaaaccacgtaaaatactaacttctgtatcgtgtttatcaccttcatcgtctaaaatataatatgctcccatcttgtcaacgtctagtttacttactgattttacaatctcgtactcttttcctatagtccaccacttaacaacggagttgtctacacaacgtaatttcataccttcgtacaaatcttcttctgtgtatttaatttgttcttcctccttaataatttcaaatgctacggggttatgttttccttctaagcagaatagcatgtagtcggcatatgcgtagtcaccattatcgtctatgattattaacataccatcttcacttaatgatacatcatacactttccctacagtccaccatactggttttgctttggtgcaacgtagtttagtccctacttttaaatcattttctgtgtacctcattttgtgtcctccgttaatttttactgtcattctaattttcctccacttctttgattgacttgattatatatctatggttctctctaaatgcgtggtaagcatcatcaagtgaatatgcgtataatcttgttgtttgcgttacagttctatacccgttacgtccaaccattggttcgtcaacttctacttctaactcaaataatagttgttttctaatcattgtttttcctccccttttctatgatataatattaccctactattgtgattctgtcaacaggcaaaaaaaagaagtagaaaaatctactcctattcatcatttgacaacttttccataccatataataactgtaatagtacgtatagtacaatatcaaatccgttaagtggctttccaagcactataactttaacgattgtcaataccgctaaaaaaataaatagccacccggttattttatttgcatgttttctaattctcataactttctagcacctcttcaatattctttctggtttcgttagctaacttaccaatccgttcaagccaacctctaaccatttctttttctactttgctatgtttgttcgcatcctccttgataccttctacattttccatcaagttatctaagtcctccataacatctaataaagtgtttcttagttttaacacatcaatagacaactcatagttccgtttagacaatttttcgttacggataaagttcttttttagttcttgttgtgtttctagtagctgagtagttttaataatatcagcttctaatttgctaattccgtctagttcattcatcacatgtcctcctcatattttactacttcctcaataattttcagtacttcttctgctgtgcctactgtgctatagtccgcaaagttttctgcaggtttcataatagccactgaatagggtttgttctctacataaatatgcccgaatccttttagagaatgaattacctctactacccacccattagcatactcttctcggtatactacttgctcactggttgcccttactggtttattcattgttaaaaccatagaagtttcttacaatattttctacacgttccataccctctaagtaactacgtttctcaggggagatatagttcttgtaatgagggacttccttctcttcctctacctcttttgtagtacgttcaatcagtttaataagttttacttcgttctctaattgagactggataatgtctaacagttcgtttttcgagcagttgtttagtgatttacttccatagtcaataattgtcatagtagtcctcctattttaatgtgtactctttatatcctttatgtgtatcgtgcccacactttgtacaaaagcctgcttgttcaatatctattgaagagtagcctgtttgtactgcatatgtctcataagtatggtcacacgtatcttcttctgattcgtctattttctctaaacgaatagcctctccttctgccacttcgtggttaaagagcaatacagcagttttaataggttcatatatatctgggtggatggcttcattcatcttaaagctatttccgctaaattcattgaaagcgtctagccattcctgactgttctcataggatacaattctgtaattcatacgaaagctcctctcgtgttttcttaaataaccagtaccgttcatcaatttcttttatatctgcctctgttaaatagaagtcttttaatttggatagggtatcttgactcaatggctcgtttctaggtaatatgcttactgctaactgcccatccttgcgtaatagtaacccgtagtctctctcctgtttctgaggcattactacatagtacttaggtaaactagcagtgtttgctactgcttgaataaaagtatctgtatttgcgaaaaaccatttactaaactctgacttagggtactcacatgcgtgtctctgtccttccaagataatttgtaacttatccataagttgttctcgttgtttatcaataaactcttttacctgttttggtacttctacctcattattcatagtttgtctcctttgttatactttgtcaatagtgatatacacatacttgccctctaactgcttaacaatatctagtatgtcatgaccacctagcttaaatgtgtttgtctcaaacccttcgatagtaaaggaagaccaaccataccagccttcttcaaagtccaatacccccatagatttaagtactatatttttagtgatgtcttctaatgaagtatgttctacagcagagaaaatctgaatctctgcgttatcaagatgatacttagaagaaatgcctccatcgtactcctcttctgttgtagtgtcctcagcacctagtgcatgtagtgcattacaaataagcatgtttaaggctctgtgctcatggtgatactcgatttcttttactttttcttttattaagtaaggttcgtttgtttctgcaccgggactgttatcaatagttacccaaccttttaatatctgtttacgcatagtgactacctccatttataattgagaatgatacattaactggtataacatctagtataccatactcacctttttgtgctagtattctagctaaagtacctttcctgtttactgcagagtccttcatagtgaaggcttgtgcatgagaaacgttcgatgaccacgagttaagttttagtcctgctgtgtaatacaaatatacttcatattcctcgtcatactttcggataacaaactgagtgttcatttaaactaacctctcttccttagtataacctagcgctatcaccagtaaatctaagttttcctttgtcagatttactgtttgttcatgtacacagtccggtctaaatacttctactgtacggtttactacagcaatatctacagtatcttccgctgtctggcttaccttaataaactgtttggaaaactccccacggctaacgccttctatcataagctccatttgtttatagcctcctctcgggaaagtccatgaaagttgtggaaagtacgctcttgtagttcgtctaccgtatctggtagtgtacgaaattcgtctacccctagtgaaatacttgaagctacctcttcataataacatttaacgtctactcccattttaccataaggtaacaccattacttctatatgaaactcatatggaatattctgtgttaatggttcatctaataaccatgtctcatggtagtttactggaatatctaatagtaaactgtttaaatcatctaactgttcagataagtacgcaattcttatcattttacggtcttctgccataggcattgaacaacctaacgtatcttttatttctttaatttctttggtaagcttagccattaattcaattattttataaaacatttctattcctcctcttctatatattccccttcttctaagaaaaagcaagcaggttctccctctccattttcagtgtcacctaggtattcatcatctaagaatgcacagtagccagtagggtatagggttacgcagtccttatggtacattgaaccgtccataatccgtacaacctcgtccatctcactgaacacttcgctacatgcctcacatgatactaactccttactcataggctacctcctttatctattacgtagtctaccagttctctaataacctctgcctcctctacactagagaggtctgcccaaacttgaagtgtcacatcgttagccttaccagatattaaattgtgtaagctatctgcaatagttagctttgtacgttcaagatagtgaattagccagactaatacaattagctgtttctcagttagctcagtagtaggctctgtgtaaacctcgtagttatctaaggataatgggaaggtttcttcattattaattacttgaaacccgttatttggttcgttttcatattctccataagttactctcttacctatttttagttttacatagatgtctttaccttctttgtgtagtttatctaggtcactattataccactctcttgtttcaattttagtaagttccataaagctagccctccttagtaagctccttaattcgattcttaatctctcgttctgtcttttcaagttgtcgcatcttgttcttgtgacgagatacttcgtgttcctttgctgtttctgccatcgctaataggttctctaaacttgctttatttgccattaaattgccacctcatagttgaattttccattgtgtttataatttactacagaaatatctgataatggggaatcaaagaagttcgtccccttcttccatggtaggacaagctttggaacctcttctggaattggtgcgtttacctgtttctctactagctctaaatgtcgttcgtaaatgtgtgcgttgtcactcgtccaatataagttacctaactctaaccctacagtgtttgccataaccaattgtaatgcatgatactgaataatattaaatggaagtcctaagcaaacatccgaagaccgagccttcacatgtaagtttaatttaccctctacaactgtccagtgggttgtccatacacatggttctaatgacatatcgtataaatcttccacgttccataaagtagtcataattctacgagagtttggtgtgctcttaagttgttcaataactgcctctacctgatttagctttaggtatggttgatgtgttcccgctatccaaccgccttttttaggtacgacttttgggatgagtgagcctgctttacgatttttagctaactcaacatctccgctataaacaggaataaaacgttctttgttaaataatgcgtaaccatacgcttttccaatagtgccatcttcttgctcccattcgtcccaaatagttacacctcgttcttttaaccacgaaacctcgttagacatttcttgccaaatccactgcaattctgttagcgcccatttaatacctacatgcttactacgtaataaaggagcccccatatctggtgtaattgtaaagtttacaccttcaataaacttagctgttgcaggagtcccatcagcatatactgcacgagtcccttctggtaattctgttactccgttcttaataatatccattactaaatctttataaattttatcaaaatccatgttgtatcctcccgtatctctttcctatttagcttatttttttttgtgtagctatacccaccagttaggttctttgataacttgccatgctctttttagctgttcttcattgaaatgtccaaaatgacaatcttctttagggatgccaagtttataagctagtacactgtatagctcccctctgctaatctgcttagactgccatatggggtcaaataagccatgacataattttttccatcgtttcataggtggtgtagctaaaatacctaatggttcttttggtctatttttgtgagtccctacactagcaccacaacctgtacagtaatagcatttaccattaccgtatgtcctaccatataacacttcattacttgtccaaataactttttctccacagtaagtgcagttatcgggtactaacaatttattcattagttattcacctctcataagtatagtatattacaatagtgtcactaagtcaacacctctttacaaaaaaagaacagcaaagatgctgtcctaaaaatctaaccaattatctttatcgtaagagtcacttggagacttttctttgtttttcttaacgaagtagtatccgatagagaaagctatccctacaagcaatactacaataagagcaataaagaatttagcaatgaaaaaacccattacttatcattccccacaattgcctgtccgtcacctacgattgtagttacccaaccatgctctttacgaccttgtgcttcttcgtaacgaactagttcatccgtaactgattctgcaatttttttattagcctctgcttgtgcactagctttatctttaatagcttgcgcttctgcttgggctttcaaagtaacttttgtagcctcagtttctgcttgtgttttttgagttttagcatctaactcagcttttttagcttcttgtcctgagcggatgattgcatcaattgatttttgtgtttctgggtcaacgtcaggcacacctaccgttacattctcaaccaagaaacctttactttcaactgagtcagagaaacgggctaacacttccccttcgacctcggaagacttacctgatagaacatctaataaactatacttagaataaacctctctggcagtcttttgtaaacgagatttcaaccaacctttttcaatgtcctcagatgttacattaccaaactctttatacattttagttgcttgcttagggtctactttgtagtcgtacttaatgttaactacagttttcttaccatcggaagtagacactgcaacatctttagcttgaatagtctgtaaacggattggatactgggttactttatcaatacctacccacttaacaccgggttgtaatgcttcagatttaacacccccgttaggtgagaatcgtacaccgacatacccattgtcaattcgctctaagaaacgaaatgcacagattgttcccccaatcaataggataactgcaataactcctgctacaaccaaccgtttaaacgtatttgtgctcataaaaaaaataattcctccaatatttattttttatttgtaagtttcatcctacaaaaatagtataccagtttttgtatgtgtagtcaacaatatatttaaatttttttattaaaaatatgaggcacaaaagaatgtaaaatatgcctcatatctattaaattctatactaaactaactagcttgtctaaagcgttatagtccgcatcggttactggctctactttacctaataagtacccattacctacctgagagaagaagtcatggttactagttgttgtagagataccattcattacaatagggtttacgtctgcgcttgttgttgggaataaagggttcatccctaagttagctagagctttatttaaattgtactccaaaaatgtattaacatcttttgtccaaccaatctcactatatagttcagcagtatactgtgtttcgttctcccataatttaaatgctaaatcaattacccattgttccatttctttttgttcagtttctggtaactggttaaaccctaactggtatttatagccgatataggttccatgtacagactcgtcacgaataattaatttaataatttcggctgtattaaccatttttgcctcacctaaatagcgtagtggtgtgtaaaaacctgagtaaaataggaagctctccaacagtacggatgcactctttttctgtagtggtgtaccgtttcgataaatatcgttaataatgttagctttatattgtaaacgtttatttgtagctacccattcaaagatattgtcaatctctgtaggtgtattaaatgtgctaaaaattgttgaataacttttagcgtgtactgcttccataaaggctatgttatttaaaacagcttcttcatgctgtgttcttacatcatcaagtagtgagtgtacccccgattctgattgaactgtgtctagtaatgttaacccaccaaacactttatttattaaatcttgctccatatcagagagctttgcccaatcattaacatcgttacctacagggacacgtgtgtccaaccaaaattgctcagtaagctttttccatgtggcttggtcaattacatcatcaatcttattccagttaattccttcatacttttcttgcaaattcatgtgctcatctccttaaattgtacaactttcacactctgaaacccctacttcgcttccgtcttcttcggtgaatgttctaacataataaattgtttttataccttttttccatgcataatgtcttaataaagttaagtcccgagtggatgtagcgccaccctccaccttccaaggataaagattttctggtaactctgagcgcataaacaatgttaaactcataccttggtcaatatgtttttgtgccgttgcatacacgttgattacgtcaatcatacttgtattgtacgctgacttgtaatatggaattgtcttctcagataaccggggtgcagggtaataagttttacctgttttcccatcttgacgttcttcaatcatagagataattgggtgcaatgaagaagtagtctcattaacataagcaatactaccatttggagccacagctaaacggttttgatggtatagaccaccttcaataatactgtttcttaatgtttcccaatcttttacattaggtagtaccactccgtcaaatacttccgccaccttactagagatttcctgatttagtgttggtacatattcatcaaagtaagaaccgtctgcgtatttagagttctcaaagttatggaatgttgtgccacgctctttagcaatcttattacttgccaccaaagtagcatagttaattaattcaaacagggcatccgtcatttcaatagattcaggtgagccgtattctacttggttacttgcgaaccacgcatgtagccccattgcccctaaaccaattgtatgggctttatcattaccattcttgatagtaggtactgcttttatatttgacacatctgtaatatatgttagagctcttaccatcgtctcaattgatgccactacatcattagatgtttctaacaagttaaggatgttaattgaacctaaattacagctaatgtctgtacctaactcttcgtatacttgttcatcatttagcttagatggtgtctgcacttgaaggatttctgaacataggttgctcataatgattttgccatcaataggattggctttatttacagtgctaacattcataatatatggataacctgactcttgttgtaaacggctaatttcttcttccaaatctcgtgcattcattaccttacttctgatatttgggttctgaatcatgttattgtattctgaatcaatgtctacaaatgagaatggtacaccgtattctttagcaacatcatatggagaaaataagtgaattacatccccagactcagctagctcataaaatttatcgggaacaactactcccaaagaaagtgttttgatttgctttgcggattctgcattttcttttctagttgataaaaagtccataatatccgcatggaaaacatttaggtagactgcgcctgcaccttgtctttgccctaattgatttgcgtaccggaaaccatcttctagcattttcataacagggacgacaccactacctgccccctcaattcccttaatagggtcaccaagagctcttaagtttgaaagatttatgccgactcctccgccaagtttagatagttgaagtgcactgttcaacgttctaccaattgaagacattgaatcttctacttgaattagaaaacagctgacatactcccctcttctaagtctacctgcatttaaaaaagtaggggttgcaggttggtatctacggttaattaactcatctgcaatcttcatagcaagctcttcgtcaccatcgcccattgtgagtgcattaaacgctactctatcttcataacgctctaggtatcgttccccatcatctgttttcattgcgtattgtttatagaaacggtaagcccccataaatgtgtcgaatctgaactttttactatagatgaacttaaataatttttttataaattccattgagtatttgtcagtaattactgtcttatcaatgtatccttctcgaattagataactaattttctcctctaaagagtagaaaaatactgtgttcttgtttacatgttctaaaaagaatgccctaacagcttctttatctttgtctaactgaatctttccgttgttagggatattcaattggttattaagctcaatatatgtgttcatatctacaccccaagttctttttctatttttttaaccaaatcaggtctaacaccattaaatgagaaaccatcatcagcttctacatatggtaatgaagacactcctttttcttttagataagctaaagcttgctcatcatgagagatatttttctcatcaaatgggatattctttccatttaacatattttttaaaaataaacattgaccacacatatcttttgaatatactgttacttttgtcatttactacattctcctaacttttttatcgtatttattaattatacactattttgcattaattgtcaaaaataaagaaccaaaaaaaactagagactacaaaagttaattgtagccccttttaataagttacaccttgtcacctaagaaatcttctgattcctgcgatttattcgtaaccttatcaagtagtaaaacagaatagtttttaccagtttcaacctgcaacctattaaacactttctgatattcttctggtgtgtctgtaacttctaataagtattttgcatttaaaaatgatagtaattcgagtagttctgccttattacctgccatttgaattacaactttaccttcatcgttcgtgtatgttccaattttatttttagtagccaaacaaatcagtccttcgtaaatttaatgtatttgttaactgtatacacatttttaactttttcccaaacgttagggtatacgttcatcaataacttagtatctgcgctagcacgtttgtactctggcattaatttaaatgtgcccccatcaatactaattttagattcccctaactcttccatatcagaaatgatactactttttagctcatcaatctcttctttaatacttttttgcaattcatataatttcttgtaagagcgtaagtccttctctagtagctcctttttttgtaaatctgttgttttcatacatatctctccctctcttatttctataataagattatagcatgtcagatatgtaaagtcaacacttttttatacaaaaagaattgcccattataaaggcaattcttgtccgttgtcttcttcaaaaagaatctctacaggtatattatataatttagcaagttttactctgtttttaacacgaggttcttttataccgttttcccaataagagattgatgaccagtgaactcctatcatattggctaactctcgtagagattcacccctagcctctctcactcttttaaggactttaggttttggtaccttttcttccatgactatccctccttattcttaaaaaataagagcctatcaggtagacaggctcagcatcaggaactatgcgaaagccccgatataagttactttaccattacatttacgtgcaaaagcctctaactctttacgagcacctaaacggtcaggttgcgaagattgaacagtaatttctttaggtaccgttacttcacgtgtacgaatttcagttttaccattttttaatttcttctcatactcctctgtacgagttgttgtcgtagaaatgattccataaaattttttcatattaatagctccttactgttttttttaataaattataatagtggtaatagggttttatgagactcctactcattgtaagccttccttaatagatacttacaactatacgtttatagacctctctcaaaaagtctccgtaacgtcttaccttcatcatttaaaacgtacacagaatttacaaaagttttatacccttctttttcagttctgtactcgtaaggtgaatttggcacacgagtgatttcttcatgtttagagttaagtaatgaaaaagcattctcttgctctaaaagaaactgtaccccatcaattaattcaataaaaggcttaacaataggttcctctgtactgtctttacaatcgtattttcgttctcctaaaatttgtagtatcatgtctatctatctcctctgtttaagtattaatttgtgagtttccactcacaatgctgattgagagagtcgaactcccattcccgggttacaaaaccggagtaatagccattatactaaatcagcgtaacccctcaatagagggagtttcttatactaagttaccatatccgtaggtatttgtttgagcttgtttaaacatgatactcttgatagctaagtcgttgtacttttcatcgtcttcgtcagcaagatacacatcaacattaacgttcttacctaaaatatcgattgtttcatctttacctacttgtaagtctttactgttaaatgatacatcaaaatagatgtcatcttcttttgtacgactaccgaatgatactctatcttcaaataaatttaaacttcgagaattaattacaacaggctgtagcccaccttcaaattgaatagtaactgtataatactgattttcaatattaaggatgttcaaatcagcaatagcctcaccaaatgagatgccaaagcttaattccaacgcaattgctcgtaagcaatcatagtttaacttaattctacggctaaacttaacaacactgtcaatttccccatagtaagctttgtctaacttatcttgcaagtaagtacgaatttcgtctgcatcaggatagtcgaaacgtaaatggtaatggaaacgtcccggacgattaagcataaagtcatttacacgatttaagttattaacagtaagtgcataaatacgcttgcgttgtgataacccatcgaataagctcaatagtttttcttgtgattcacgaccgtcacggtcattgaatactttttcaaattcatcgaacaagataagagactcttggtcaatactgtcaataaattctgcgatacccggataagcttctgtaattaagataacaggcataccctcttcaatagctcgttgagataagatttgagtaaatagtgatttaccaatccctttatccccacttaaaataacccctaaactacgattgattgttttaaatgtgtgtagtactttctcaattttaactaaacggtcaccataaactttagattctttcaatttaaaatcgtctgtaaccgccaatgagaacccagacattgggttgaatctaactttataagtttgcgcaggcaacttatcataagttttcaggtcgtttgcataaatctcataattactaccataattgattactttcatatattctctccttaatactttttaataaattataaatggagagtgatggagtcgcaccacccgagcctaagcaacagatttacagtctgctccgctactacttacggaataactctccttgaataaccaaccagataggcacctgacacacggacagcacctgtgtctctctgttactggctgattaattgtgtatttcgcaccacctaatatagttatcaaggtaggacttgactatactagatttagctaggttctaactataatttcctatggtagctcctactactaatcatatttatatacagttaaactacacataactaaaccgattaccttggttcgcatgttaagaggcgcaggcaaccaatggacattgcaggtctcgaacctgcgaccgttcggttatgagccgaatgctctgaccaactgagctaaacgtccttaatataattagttgggtacacggcactccgtatatttaatctgcgaataggcgcagacccaacttagtttttatagctacccacttaatttggtctttttacattatcatgcgtgggtaaagttaaaactcttgtaataagagaaagactgtcatcgttagatggatgataatgttttacacacaaaacctagagtgtgtgtagtgttgggggtactaaaaccaacgtttttccctataatagatacctattaattatcggatagggcttgagtcctacctcttagagtagctcattaattaaatctaaaattttatattagccgttttaatgtcttgccatctttattaagcaaccatgcttgttcaatagtatagtcttcatattcatcaatatagttatataaagtcatgttgcctgctgaatcattttctaaggttattacagtcctatctgataggtgtagttttacaccaccatctacatatgttgtacgaacaacgtcttcaataaaatctgattgaagtactgtaggggctcctttcggtacgttctttgtcttgtctacttgtaaaatcatgtatatctacctttctgtctaatatttatctatatagatatgcagaaggcgggagtcgaacccacacgtccattacagaccgatgtttctaagacaccgttgtctaccagttccatcacttctgcagttgtactaccaaggttacctctaaaaggctaaactcgtgtctggtagggagccataccttacatggattgcaaggaacctaacctcagtagtaccttttactattaagggtttctgatttaacgttttccctagaccgtaccttaacagtaatactggtgagaggatttgaacccctcttacctacaagcctccgacaagcgctatgggagacaagcggtgcattaaccataactatgctaaccagtacactactgttttacgcaatccgcatattagtagttctcacggactcaacccaattcagggaagtggtagccttgttctagtggggaactggttgactccttgtacgccaaacattcccagactactcacttacattgttatttaatgtcacatgtattatgacagccgtactcaaaagtttcaaggacttttacctcactatccaccgacagtatgctcggcaacgtctttctaataagaagtacttgttaaacttagggaaggttaacaagtatgtgcacataacccgaatgctactccctaaagtagcctatacgggctaacgtagatggagggaatcgaacccccaaccgtccgcttagaaggcggatgctctatccgattgagctacacctacatattatgggggtaatgagtccccataataactcatattagtctcatctatcaaagcttaaaatgaatatctaacttaaataaagtatatcactatttatagaacatgtcaacacttttattaaaataaatttctatacctcactaatgccaatgactatacgtaaggagagaacgttaatggcactggctacttatttggagataagctaatgaggtatagaaagagaactacattgaccgaatcgtattatacagccttagtagttgttaacaatgtttggaacttagtagcttactgcaataagctaccccgaagcatcggggtgggactgtggtaagccccaaacccttgggtatgctccatcctgcctccggagaagtcaggctcaattctcattagttaaaggtttactagttaacggttagctgattagtcatactacgtaaactactagtaaaatatagttttccaattaaggctaacaatcggtagaggatttgaacccctattattggttttggagaccaacgtgttaccagttacactaaccgactatgtaaagaaggaaagcgaagaccttctttacagtaattaaaaaaacaaacaaaaaaaaatacaatcatggaatcaaggtgggatgtaaggagatgccatgtgtccacttatagtggaataggtaactacagatttgcactgttttacatcttgtttaatggttacctgttaatagctaacttccagtgataagtcctcttatatgttagttcatgtagtatttgtttgacaatatggtagctagctaaaatgacgacagtaacgggactcgaacccgtgtgttccacgctgacaacgtgttgggacaacctctgccctatactgccattatttatgccgtagttttttttagaaaggaaagtttttttataatatacaacaacagttgaagcgctgatgttactacggcataaatatattttttatgagcttttaataactcaaaacttaatataatagggcttttactcctgcttcatgcacactcactgcattcggtcacatacccagagaggctgaactatcgggagctacccaatagcttctctctgcttttaatgtatggtctagcacggtggctaacaacgttcacctaaacattcagcattaaatagtattgttataagtttttcttatatcatatattttgtaagctgacttaggctacttgtaccaaggtctcttgttaaacccatatttctatggttctcgtcaaagggagtcttttgaggtttctaaggtctgccaagtttctcacgcttccacctaacttagattcttttccacaggaacgtctattatatcccccaagagtcttgtaccttacaatccatgtacactccacataacgcttaactgatactagcttttactagtagttactcttatgtcaccataagacatacctgctacctaaaacactatcgcactctaggctacattcagaattacgccacgttatcctagcaagctgttcaaactttctgcactaggttctattatattaagttttcaattatcaaaagcagtttttctttactatttaataatagcacaacctctattattttgtcaactactttttttagagagggtgcccctaattttatataggggcaagagatattaaacatagctgagctatgtaaaagctaatgtaggaattgaacctacttaatgtaagcaacaccagtttagcttataagcattgtctgcaaagtccatgctatgcactcagtaggaatcgaacctattgccgaaggtaccaaactaccctgagtgctcctatagatatattataccacataacgagatattaattttttttggtgatatagtatatctatattttttttaattttagaaatacctttgatacattccggttatccaacgtatgagtatataatatcatgttattctgattgtgtcaataacttttttacatttttttttataattgtgtaatgtcctatttctttttttctgattccttatatttattatatatactatatagtactatatatatatattaattactattagatattatatatagataataagaatctaggaaatttaaaataggacattctaatgctgactcagttacaatcaacttatggtgactacctttttacaaaaatgtcctatttcattttttctctctccttatattattatttataatatatagtattaagtattatatattattatatatatagtatataatataagaaagacaaaaatttaaaataggacattttacttaaacaaaaaaaagagagcagttaagccctcttatcttaatgagaaatagttattaattgtccccaatctgacttcctcatcttctggtgtttcatcctctggtttcgtatacaagctagtgattctttctggaacatctactatgttctcagtagctgtctgtatctggaatgcatttacataagttaactcagaatctctatcagctagcttatgaactggaatcattggcaactctgcacctttcattgcaccatttacaattgacattgcatcattagttgttacaccgagtcttctagggaacacagaatatgtcccagatgctaatgaaacctctacaactttctctcttttagggtaattcatgatacttctcctttcaaaatatgttataataatactatatactataataacacattttacaaattacttaaacatgctgaacacgtaagtgacaactgctcctaagacaatcataaaaacgttctctacaatatccctcttgtggtcgaactctttatcgtttactccttccagtgcatctatagtttcttccaacttagtaatctggtaggttaggtgtgcatatttttcttcgtgaacagcaagtcgcttatcaagactattgacaatatctctcaactcactaaccgcactattcagttctacattgtcctgtttaatgcccttctcggttagctctccatgttggagtttatcttccagtcgttgtagtcttaaaacaaggtcatttacttggtaatcgttcatatatgctcctccctgtaagttatgttgctctatattagtcagatgtaaaccacagacagactagtatacctataatagcgcctaccgtgtagactataggtatatagtacccattaaaaaacaagtatagactacctatactaattccatagagcacgaaagtgaacttgtatgatgccaaagggctcagttctgcttgtagtttcttatactgtagtgcacgagtgacatagtaggacatcaagtaaacaaacatttgtagtaacagtacacagaccagtgctgtttcatgcattatgttgtcccctcctctcccttagagctactattaatatagtacactgagctgttagcaaacaggtgtgctaaattaatatagagactaaaaatgtagacgggagagtggtaaaaatggataatagcaaacgaattattaaaaaaattatttttattaccatctctgcattagtcatggttactttaagtaagctgttctctaaatatgttattgtagaacaaaatgccccatttcaggcattaattggcggtgcctcatgtgcattgctatctagtattctgtttgattggtatactaataaaaagaaaaaagagaacgtagagaatcaacttaaagaagcaatcagtgacttgcagaagattaaagctatcataaaaagatagcctattaggaggtgggatgggtgtctattaccaataaagacattaaggataaacgtagatatatttttagtcaatctagtaaaacaacaactataaaaagaggggacaaacgcataagtagtgcaacaagaatatgtgcagtttgtggaagaccgctatctaagctcgtattaagaacaggcgtaccaacagtagtagtagaccatattagctgtaagatttcagacattgttagactaaatgtctgtgaggatataaggtcttgttatgcgtattctagtaagaaaggggaaagctagctaatgggtatggcagatagacttaaagataatgcaaaacaaaaaaagttagaaagaacaccagagcaacaactaagagacacatttaatcaagcttcaataaagcttatcaatcaatttatggctaacgttacatcaggtgctatagaagttgatgatattgcagatttaacaaggctatttcagatttatttacaggttaataatataaatgatggaatgcaagaaggtacaggaactctacctgcacttacatctgagcataaagacatcatatctgaaaaggttagcacagaaaagattattaaggacggtgaagaagaggagctaatttctcttgatgagttagccagtcttccagatgaccaacttgaggaagtcttagttaacagagagctacagatgaacagagagaacgaggcgaccttctaatgacgacaaaagcacaacatatagctaaaatggctaaagaaatgtatggtacagataaaattacaacggagcagttagcttatataacagatatgctgaccccatcaacgtacctattgagaaatcactctgtgcgtaatcacccaataacttttattatatcaggaagggatgcaacaaaagcacaggcacatagaccataaaccaacaaagctgtggtctttaaactctactaaacgggcatagtaaaataatatacacgtaaacaatatactggtaagagagactaaatcctagtgtataaggacagggttgacctaccgtgctaaatcagttatactataactgtaaaagcctaacgactaaatttctaggtagctaactaaaaggagttagtgagaactagataaaaggcaaataaactatgcaagctcaataaaaataataatatacaggaggtgattaagatagctaaggacagaaaagaaattttcatagaaaagctcaaaagtgtaaaaggaactgactttgaacttataggtgagtttacaaagcaacgagaaaagactctatttcgtcataacgtgtgtgggcatgtttgggaaaccactccggttgtcctacttaactcaaaaaaaggcggagggtgccctcattgtcaatacagaaacaaagcaacttcaccaaaagaatacgaaaaaagagtaacagacacatttaaaggagaatacgttgtactcaatattaatgagtataaaaacaatagtactaaacttaaattcttgcattctaaatgtggtacagaattttatctgcgaccagcaagtttattcattaatgcaactagttgtcctaactgctctaaaaataaccgttgctcaactaagagaactactaatgagtttagagagttgcttcttaaaacaaaaggatattcctatgagctaacggaagatgcagagtacaccggagctaacaaaaaaattaaggttaggcatacaacttgtggttatgtttgggaggctagagctaaccacctgctacagggttcgggttgtcctagatgtaacgaatcaaaaggagagctattagtagctactgttcttaagctaagcaacacctcttttttgagagagtacacctttgacgattgtagaagtactagacccttaccttttgactttgcattaattagtaataacaaagtgcgtggtctaattgaatatgatggagaacagcacacgaaaccagtaagttgtttcggaggggaacagaagtttaaaagtacagtaagaaatgataacactaagaatgattattgtactcataaaaaaatacccttactcagagtatcatacactaatagtcctgaacagattgagcacctagttcagcagtttttaaaaagcatagatttgttgtaacgtagagcaacctaaaccaaaaattaggttgatgatatagtctagtcccctaataaatatcgggaaaccgagggtaaccaaatgggcaaccaaagataataaatgaccaacatagagacaaggcaataattaaatcaagacagctaggactatcagagatgggtgtcggttctatgctacagtttgcagacacacatagttatgatgctgttaaatgcttgtatacattccctaaactaattggggcttaggtcagtaatgacctttgaaaaactctgttaaacgggcataggagaataaccaatatcctgataagagaccctaaaccctaaaatagggcagtgggaatcccgtgctaaatcaagcgtaagcttgtaaatgcctaacgactaaatttctaggtagctaaccaaatggggttagtgagaactagataagaaaacctcttaagaggaagtaaagcagagcaacctaaaccaaaaagtaggttgatgatatagtctagtccgactgccaagcgcagtgttaaagtatagtgaaaactacggtagattcgacaaatgagcaaatgactaaatttgtacagacaaggttagaccctgttttacagaatgggtactacagcacaattgtagaccaagaagttaactcattaaaagctaaaaaaataagaaatagttttttgtattttcgttcaagctcaaaaccgggcgctgtagagggtgtcgatattgactatctatctatggacgagtatgaccgtgtacctgcattagcagaggcttctgcgttggagtctatgtcttcttcaccttataagatagttaacagatggagcactccgagtgccccagatatgggaatacatgggctctttaaagggtcagaccaacattggtatttacataaatgtgagaaatgtaattattacaacgaaatgagttatgatgcatatacaccagaggctcctgtagagagtagaggtaacattctttgtgttaacccaaaaggggtcgatgttgttgctaaaacagtagttgacggctcattccagtttgtttgtcaaaagtgtggagaaccgttagacagatggtacaacggtgtatgggtacctaaataccctgatagaacaaaaaatggtctaggtactagaggatatatgatttcccaaatgaacgcagtatgggtaactgccgaccagttaaagactaaagaacttcaatcattatctaaacaagccttttataactacactcttgggtacccttatgcagacttgaaattaactgttaacgactctgacgttgatagccataagagaaactatttagtagaacctgctaaagacagaggtgattataaatttatatctgttggtattgactggggttatagattagcccccataagtagtaatacttaatgtaaaaccttgttaaacgggcatagtaaaataatatacacataaacaatatactggtaagagagactaaatcctagtgtataaggacagagttaacctaccgtgctaaatcagttataatgtaactgtaaaagcctaacgactaaatttctaggtagctaaccaaatggggttagtgagaactagataaaaactatgatattgaattgcgaacatgagaaaatgaggtaaaaaaataatggcgttaagaaaaactaatgaagaatgggtgaacgaagtacaccatgctgtaggtaacgattatgtattcctagagcactatataaacagtactacaaaaatactagtaaaacacaacaagtgtaatcatatctataaagtacagccaaaagctttcttatcaggtaaccggtgccctaactgcaatactgctagaaaactaacaaccgcagagttcactgttaagatgagtaacctcccaaaaggaggagagtacacattagtatccgagtatgagggacaccacaaaaagattacagttacacataaaatatgtggtaacacctattcagttcttgcaagtgactttataaaaggcagacgttgtgttagttgtctaagagctagtaacacaaaaacgcaagaacaatgggaaaaacaagtaagcacattatcaggtgatgagtataccttcttggaaccatacgttatggataacgtaaagattagctaccgtcacaatacttgtggtacagaacataaggtaacccctaacaactttataaatgggacgagatgtcctaggtgtaaagatagtaaaggggaagctcatatacgtaaattcttaactgataacaacattgagtttatacaacaaaaaaaattcaatgatttgcaggtagttaaacagctatcctacgacttctacttacctgaactaaacatactcatagagtttcaaggagagcaacactacaaacctattgacttctttggtggcagtgacaaatttaaatgtcagcaagaacacgataagctaaagagagactatgctaagcataacggaataggttttatagaagttaaatatacaaaagatacttacaaaaaagtagcaaactacttagctcccgcaatggctaaagcagttaataaatcatagtgtaaagcaagggttccttaacctaaattaaggaacatgatatagtctagtcccctaataaatatcgggaaaccgagggtataaacgaatcgtcactgggtatctatacatggtgttaaaacaaatggtacggtagatttgataaaacttttttctgtgggtaagtccaacccgctagaccctaatgcaatagatgtagacatacagtctataaaattacagctagctccttacaatccagatataatcgtagctgacgtaggagactcaggggataaagttgctaaacttatgcaaatttatggaaaagaacgagtttttgggtgtgtctacccatcaacccctaaatctactggtaacctagtacctacttggagcccacaagcaaataaagtatccgctgacaagttaatgcagaataaacgttacattaacaagatgaaagaaggagaaataggttactactcaaaaccagatacagagcttaatttatataaagagcactggaagaatgttgttatacgtgatattgaggacgaaaagacatcgacaggttttagacaaattatcggcagaaaaggtgatgaccactactcacaagcaagcgtttattccatgttagggtatgagtacctaatgaatgtatttacaggagtaaaagagtatggatttgactctgattgggtgtccactcagttagcacctacgaaacctgatatatttacagaatttgtataggtagattgccttatttagttttagtcaagctgaataaggcattttgtattgtttgatatatatttttgtggtataataaatatagttagggggatagttatggtagacaacaatgtaaaaataagtaaaagtacaattgaaggactaataaataagtctttaagttatgagtacgttataaaaaataacgaattgctgacaaacgaatatcaacatattgtcaaagcgtatgggtttgacaatttttatgacatgtacctatacgcagatagctgtgattctaaagatatgtatttagtaaaaggtggtcaaaaagacttgtctaagctgaaacctgttaaaagaaaagttgttagaaatggtaaaactatgactacaactatttatgaagatacaggcagttcagacagtaataacagtaaccctttagacaaagagagtaaaaagaaaaaagagttagaaccagttaatgctaaggaactacataaggttagcttaggtagcgatgaagaagagaagttagaccctaaaaagatagcaaaattgctagcagacactaaaaaatttggaaataattttgatacgcagtgtactgattatcttattcttgaacaagactctgttacacgaggagtggtagggtttactagagaaggttcttatttaaaaatgtctttctcaatgtcagatgaggctgtcaaaggtataaagatgttggctttttcacagctcacattaaaggcttggaaattgggtctaggggctaagataagcacagataatgcacctgatgtagaggaactaattagcttatatggatacaaaagaaataagacagagtatattgtgtcaatgagctcactgcgtagtcttctaggggagccttagtcataagtgctacgttagtcattatactagtattttcaactattatttttttaatttttaatttactagtaagtaaggtagttaaagaattttataacttaaagatgctagcaaaagagggactaacagataaagtaacagacataacaaatgatataatgcatatattgaaaggagaaataaataatatggaattaattatgaataataagaaactagatgaattaactaataaggtagctactgatgaggactacgatatttttgtagagaagatgggtaagctagttaaagatttgtatgagaactaccagtttttacaacaaaacccaccagaaggagattacacatcaggttattttttagggttccaagtaatcagagcagaatacccagttgagtatgagaatttatttagattagctgtagataaaaatttaaatgaactagagataaataagagatttgtcgaagctgttaaggatggcaaggttttaccattaggtgaggctgtcattgatgaattacaaacagggtgtagctctatactacaggcacaagaagtacgtgtaaatatcgtatttggtacaaaagaatacatggctaagcaagaagaagagcgtaaagaacgtcaagctaagttagaagaagaacgggaaagagctatggaagtactaaaaacaaaagatgacgtacttaacacattaagagtaaccgaggctttggctaatgaattaacagacgaagtcgcagaaaaatatgatttaatggagctagttaacagtatgagagaggggctgaaagctcataatggaaattaaaaattctttgggaaaagctcttataaaaaatttaagattgcttaaagaaaaaagagatgcaatgcccgataacttagagtatacaagtcaagttatgattcctgtgccctactatctaataaaaaaaggggataacgcagtagaatcattccttatgtgtgcggggatgataaataggaataaagatttaggtttacctatttctttagagaaaggtaagcaacaagttaagcttaacaatggagaactaacaaccattgtagaatgtattgctacctattcagataaaactgacattgacggtgtagaaagattcttagttgaacatctataaaaattaaattaacgagttctattttgtgctatattagatatgcatgaaatagaactcgtttcttttttatgactaactaaggaggtttaatagatggcaggagaagtatttagtagcttgattacaagtgtaaatcctaacccaatgaacgcaggtagccgtaatggtatcactatcgacaccattatcctacatcacaatgcaacaacaaataaagatgttgctatgaacacatggctattaggtggtggcgcaggtacgtctgctcattatgaatgtacaccaacagaaattattgggtgtgtcggtgagcagtattcagcattccatgccggaggtacaggtggtatagatgttcctaagattgctaaccctaaccaacgttcaataggtattgaaaatgtaaactcgtcaggagcacctaattgggctgtagaccctagaacaattacaaattgtgctcgtttagtggcagatatttgtacacgttatggtattccatgtgaccgacaacacgtgttaggacataacgaagtaactgcaacagcatgccccggaggtatggacgtagacgaagttgtacgtcaagctcaacagttcatggcaggaggctctaacaatgcagttaagccggagccaagtaagcctacaccaagcaaaccaagtaataataaaaataaagaaggagtggcaactatgtattgtttatacgaaagacctattaactcaaaaacaggagtactagagtggaatggtgatgcatggacagttatgttctgtaatggggtaaactgtcgcagagtatctcatccagatgaaatgaaagtaattgaggatatttacagaaaaaataacggaaaagacattccattttacagtcaaaaagaatggaataaaaatgcaccatggtataacagattagagacagtatgtccagtagtaggtattactaaaaaatcttaatactagatttaagaccatcttatggtggtcttttttctttctttgtaatattcgtaataatatgtaatggctatgtaaccgcttgttcttttggctgtaacaattacatgttatattagttcttgtaagcaacacaaataaagaaaatattgaggagaattttattttgaagaaaactagtattttaggtttaagtttattaagtttaggtttagtagtaggtttaggaaccgaagctaaggcagaagaagtaacagagaatgggaagacatattggaaggtagagtcaggagacacactatcagaaattggagctaagtacaacttagacttcactaacattcataaagttaacaaaggtgttgtagctgaccctaatatcattttcgtaggtgacaaattgttattacctttagatgaaaatggcaagctagcggaacaagtaaatgccactgaaccagatattgaagtacagtataacgcaccagtagcacctgaacaacctgtagttgtagaacaagaagttgtagagcaacctgtagttgtagcagaagcccctgcccctgtagtagaggtacctgctgatagtagctcagcaaaagagtggattgcacaacgtgaatctagtggttcttatgatgcaacaaatggtcagtatattggtcgttaccaattatctgcctcttatttaaatggtgactattcacctgccaaccaagaacgagtagctgatgagtatgtagcaggtcgctatggctcatgggagaacgcaaaatctttctggttagcaaatggttggtactaaaattaaatagcaataatgacccctttattaaggggtctttttttatgctatattaatatatagtaaatagtaatataaaatggttatgattagttatgtggtataatagacttatgttaaaaccatttaaggaggggaaacatgggttatattcaagatgagacatggcagatggttaaaaaagttgctaaaaagaatgggtttgttggtgattggattttaattatccactcatactatgagtatggtggaaatcacgtccagatacatacaacaataaacggagaaagctatagaattttaaggttgttagatagcagagagatacttttattagatagaaaaggtaaccctgtaatttatgattacgaaatagttaatgatggtcaaaaaagcttcttttataatgatatggaagagaaagaaatcgaaatacctaatggaagatgcttaaacgataagacaagggtaaaaatttatgtataaggtaggtgtaacagttgccaaaatggttagataaagcactaggtatagaaaaatcgtccatagaagaaactcggaatatggaaaattataagatgcatttaagggaaatagacaccaatgtggtcaataacgagccgtacagcatggaaagtattgaaaaaggtatgaatggcaagaccactgcatatatgcaaccaattattggagagatgtcagtaaaccccgggtataaaactaaaccgtctatacgtaactctcaagacttacataagacacttaaaaagtttggtaataacataatattaaatgctattattaatacacggtcaaaccaagtatcgatgtattgtaagcccgcaagaaactctgaaacaggtgttggttacgagatacgtttaaaagatattgaagcagaacctacttcacatgacattgctaatattaagcgtattgaaagttttttagagaacacagcacaatttagagaccctaatagagataactttacaactttttgtaaaaaacttgttcgggcaacatatatgtatgaccaagtaaactttgaaaaggtatttgataaagatggtaactttatcaaatttgatactgtagacccaactacaattttcttagcaacaaatggtgaagggaaactaattaaaaatggtgaaagatttgttcaggttatcgataacagaattgttgccaaattcaatgagagagagctagcttttgctgttagaaaccctagagcagacatagaagttgggcaatatggttatccagagcttgaaatagcactaaaacaatttattgctcatgaaaatacagaagtatttaatgacagatttttctcacatggaggtactacaagaggtatcttgcatgtaaaaactgggcaacagcagagccaacaagcgttagacatatttagaagagaatggagaagctcactagcaggaataaatggttcatggcaaatacctgtagtatctgcagaagatgtaaaatttgttaacatgacaccctctgctaatgatatgcaatttgaaaaatggcttaactacttaattaatgttatatctgctttgtatggaattgaccccgcagagataaacttccctaacaatggtggtgcaacaggctctaaaggaggctccctaaatgagggaaactcaaaagagaaaatgcaagcttctcagaataaagggctacaaccccttttaagatttatagaggacaccgttaacacatatattgttgcggaatttggagaaaaataccaattccaatttagagggggagacctaagtgctcaactagataagcttaaaataattgagcaagaaggtaaagtattcagaacagttaacgagataagacatgataaaggtttagaaccgattaaaggcggagacgttatactaaatggtgttcacatacaagctattgggcaagcgttacaagaagaacagctagaataccaaagaagccaagaccgcttaaacagactattagagctatctggtggagatgtagaacaaccagaaccagaagagcctaaagacagtcaaaatgatacagatgtttcatttcaggatgaacaacaaggtttaaacggtaagtctaagaaagttaatggtaaggtagacgacaatgttggcaaggacggtcagttaaaatcggaagaaaacaccaactcaactaagcatggtactgatggtataaaaaaagaataaaagataatggagggggctagctccccctcaactttgtttgtgttactagatagtacatgttctgctatattaaatacagtaaaacgtttaggtggtgaaaagcaaattgtcagaagttagagaaaaatatagtattttcgtaccactggatattgaaaattctatacagaagtctgaatctgtgaatgatggtgaatggtatgtgcaaggatatgcaactaccccagatttagatttacaaggagatattattttaccacagggcattgatatttcttattttattgaaaatggttggataaactatgagcataaaaatgatgctgagtttattataggtgccccaactagcaattgttatgttgatgtggacaaagggttattcgtagaggctaagctattaaaggataacaagtacgcacagtctatgtggaagctagctaatacaatccagaaatcaggaatatctcgtcagttaggtttttctattgagggtgcagtagttagtagaaatgcacaagataacagaatcatagaaggtgttaaaatacataatgttgcattaacgacacatccggctaacccaagagctacatgggagacactagttaaatcttggactacaggatatggtacagcaccagatgcacaagtagatgcaggagcacttagaagagagatgtttaaagaggacatttctaatttgacgtatgcagtaagaactattgcaggactatataataaaaaacctgcagagaaagagtttattttacgtgaagtggctaaggatatagaagtagacacttccgaaaatgaattatctaaatttatgttacaattaagtagagggatttccttgaaagaagcaacaaactttattgaaaaaagaaaggggtaagaaatagtggctaaaacattaaacgatattatagaagattttgatgcacagttaaatgaaaaagtaaaacctactacagatgaggaaattacaaagtctgtagaagaacctactgaaccagaaaaagttgaagaaggtgctgaggttgagccggaagaaaagcctaatgaatctgaggagactgcaggcaatgacggagaagaatccggagttactgaaacagttgaagcagaacaggaagaaccagaaactgttgaagaagtagcagttgaggaacctgttgaagaatcagcagaaactgttgaaaaatctgataaaactaaagaaaataaagatgaagaagaggaggaagacgaagaagacgaagacaaaaagaaagaaaaagacaaaaaagacaaagacaaagaagataaagaagacatcgagaagtctaccgaagtcgaacaagttatcaaatcttctgaaatcttaggagctatggaagctatctttaaaaatatgttaggtctaagtgaaaagttagacgaaattcatagagagtttaaagaagctaaagaagctaaagaaaaagacgaagcagagtctgttgagaaatctttacttgataaccctgaaattaaaacaggaaaagaggactcagaaggcaaggctgttgggtttgttaataagtctgtagcagttgaggaagaggtagctaccgaagaaccaactgtagaagtagttgttgatggtgaacaagacacagcagaacctgaaaaagaagtaccgttccgtgatagagtacaagctattagaacagactttatggaaacatataaacgtgtatctgttagcggagtagctcaacgtggtgaattagaatcagttagacacacttggggaactgctagaacaaatgatgacctagctaaaattgaagggtttatcaataaatataaataaatttactatatagtgaacgatttttaattgtactgctatattaatagcagaaacaataaaagacctgacattccctcctgaacctccacagggtcaggtcttatattgagaggaatataaagaaaggtgatttatacataatgacagagaaaaagaatacagaacgacaattaacttccgtacaggaagaagtaattaaaggcttcactacaggatatggtattacaccagaatctcaaactgatgccgccgctttaagacgagagttcttagacgaccaaatcacaatgctaacttgggcagacggagacttatcattttaccgtgacatcactaaacgtccggcaacttcaactgtagctaaatatgacgtatatctagcacatggacgtgtaggtcatactcgttttactcgtgaaatcggtgtagcaccaatttcagaccctaacttacgtcaaaaaacagttaacatgaaatatgtttctgatactaaaaatatgagtatcgcaacaggtttagttaacaacattgaagacccaatgcgtatcttgacagacgatgctatctcagttgttgctaaaacaattgaatgggcttctttctacggtgactctgacttatcagaaaacccagatgcaggttcaggtttagagttcgatggtttagctaaactaattgacaaacacaatgtactagatgctaaaggtgctagcttaacagaggctttacttaaccaagcatcagtattagttggtaaaggctatggtacaccaacagatgcttatatgcctatcggtgttcaagcagacttcgttaaccaacaattagaccgccaagttcaagtaattagcgacaacggtcaaaacgctacaatgggattcaacgttaaaggtttcaactctgcacgtggtttcattcgtttacatggttcaactgtaatggaattagaacaaattttagatgaaaatagaatgcaacttcctaatgctcctcaaaaagcaactgttaaagctactttagaagcaggaacaaaaggtaaattccgtgatgaagatttaacaatcgacacagaatacaaagttgtagtagtatctgacgatgcagaatctgcaccatctgatgttgcatctgtagtaattgacgacaagaaaaaacaagttaaattagaaatcactattaataacatgtatcaagctcgtccacaatatgttgcaatttaccgtaaaggtttggaaacaggattgttctaccaaatcgctagagtacctgcaagtaaagcagttgaaggagttatcacatttatcgatgtgaacgatgaaattcctgaaacagcggacgtattcgttggagaattaactccatcagtagttcacttgttcgagttactaccaatgatgcgcttaccattagcacaagttaatgcttctgtgacattcgcagtattatggtatggagctttagcattacgtgcacctaagaaatgggctcgcattaagaacgttaaatatatcgcaacaggtaacgtgtttaactaacgctaatctttagaagactaagcaaaattgaataaaaatggaataggggacggttaatactgttccctatttttattataaaatacatgtatggaggaaataatatgttaaaatcagaaatcttaataaataaaacagtaacaacagcttttggtgaggtaacatttgaccataatggagaaaccacagacctaacggtagaacagcaagaacatttagggactaaagttccatatatacaatatataccagatgcacctaaagctaaagaaaaagaagctactgcagaaaaagcagacgaggcacctaaaaaggctaagaaagcacctgctaagaaaactacaaaatctaaaaaagaggaagactaaggaggtatttatatgtacccagactacggatacgaggaacaaggggacaatacataccaataccaaccatatgcacatgggaatcctaagcatatagatttagacaaaattgatgatatacagcctgctgattacggttggacacctgctacactgaaacaatacatgtttggtgtagaagttgttaaccctgaaacaggagaacctttaggagatactttctatgaacatatcatagactcagcaatagctaaagcagagaaacgactagatattgctattatgcctagacttataagaggagaacatcatgattaccaccaatcagatttcaactcttacatgtatactcatgtgtttaaaagacctattattcaagcagaaaagcttcaattagaagttaatggtagagggctatacagatacccgtctaattggtggaaagtgtatgcactagcaggtcatatacaaatgtacccaacatccctcatgcagactggaacacagtttggttatgaaatgacgttctcagggtacccacaattagcgggaatgcctccatcaggagggcaagttgatgctcctcaaatgattcatattgactatgtggcaggaatgctacctaggaaaaatagaggatacaatgaagactgggagtgccctgcggacttagaacagcttgtaataaaatatgcgttaaaagagatattccaacaatggggtagacttataattggtgcaggtattgctagtaagtcactaacagtagacggtattagtgaaagtatacaaacaacacagtctgctatgtatggtggtgcttctgcagatattcgacaaattgacgaagatatacaagaattagaaaaatcattggtatcttactttggaatgaacttaggtattatttaaaaaaggggggttaacaaatgggtgaaaaaccaattagatttggtggagcaggtgaaacaggtaaccctaacaagcaattaaatactagtagggttgaatttgaaacaaaaggtatggctagcttcattgaaaatagaggtattgacgttttgtgggaaagagcatggctatgtacatgtcgtaacccaatgaccctctcacctaagtcggattgccccatctgtaggggaagagggattgcttaccaacctgcagtaaaattaagaatggctatacagagtcaagagaaaggtatttctaatcaagatttaggactactagatacaggaaccgctattggaacgactgagctagattctaagataacctttagggatagaataacagtccccgaagttaaaatatatcaaagctttatttttaatgtaaataaaagaagagtagctaatggactatttttaagctatgatgtgaacagcatagaagatatttatggtaaagacggacgtatcttagttgatggagtcgattttagaatggactatgacacaaatactatttacccaaatgaatctttaatagatactaatatatccataaatatgtctgttacacttagatatattgttatagacctgttaaaagaaagtagataccagtataccacatttggtgttaaacaaacacaatttgaatcattacctaaaaagctcttattgaagcgtgaggacgtgtttattgatagtgaaccattttcattagacatagatacggcaagccgtatggaagagctagagggcaaaaaagatacaagtgaagctatggtagaccctaagcgtaaagctacaaaatcaggaggcttctttggaggtaagttaaatggctagaaaaggacagagacccgtattatttactgattcaaaagcaatacttggcaatctgactcgtgcagtggttgatgaagtactaagtgatgcgcaagatgttgctctacgtaatgggtcttctgtacagagaatgcctagctatttgatagtaacagagtctaggatggcaaaaaatggggttatagatttgaaacctttctttgcacgttctaataaaaagaaatataataaaaaaggggaatggtacctatacatccctattagtatgaaaacacgcaacatgtcaagacgattatacgatgagctgagagcagttccagtaggcacaaaacctgtaactgttaaaatggactatctgtatgatagacggaagcagagcccatcagtgtctagtattaactataaacctaaatctactaatgtaactgttataccacagagttggggtaaaggtacacgtaacacctatgtagctttccgtacagttaacgctaattctcctgcgaatagttgggtaataaatcgtagaaacgttaatgatgatgatatgagtaagacaatgctaagaaacatagacaggctcatgaagtggaaactaaagaatttaggaggatagcgtatgataccaagtttagacacttacttgtataaagaatttgaagaaaggctaaggataattctatcagagtgctatattatagacgaagctttaaagggaatggacaaagaagctttagaatcttttaaaaacacttattgctctatagatggtaagccacctaagagagaagtagagatgtcctattcattcccacaagaacatctggattcatttgctcgatttgtagtaactctcggtagtagcgaagaagatagcaagtctattggaggaatccaaggaggctacgagtatcgagaaggtaatgtaattagtgaggaagccactattattagagaaggtgacaagctgattataaatacatcaaaaccagtagcggactatctaaacagctcagacataagttttgcagaaagcgaccattttaggattgaagataataaacctgtatttgatttttcatacaatgaggagttagaaggtatatccattaatgtgtcgtatataagcaaaatatcagatgatgatgttgcaggtgtatacaaaggataccaatctaacgataatgttagtataattggaataagttctaatatagatactgctaggtgtttagatgcaattgctagaattatactaatcaccatgagagacagtttagatgaaaaaacagggtatatgttgcaaacactacattttggtgatatgcaagttgtcatagaatcaggtgaaacacttgtgtttggtagaccttgtactgtaaactacagagttactaattctattggatttgatttacaacaaagaatcacagagattattacaaaaaggaggatgaaatcctagtggctaaagaaacagagaaagtagtaaaaaaagaagttaaaaaggagcaacctaaaaaacctaaaggttatgtccatgtcgatacatttttagattatgcaaaagtattatatggactaaataaatatcaggtagcgggtttcagagcactaatggcaggtagagaataccaacacgaggatgctgattttgttccatttttagaaaagtatataggaaaggaagttaaataataaatggctgtagaacaattcccaagaaaaaaagtatcacgtccacatactgagattaccgtagacacaagcggtattggtggggcatcaagtagctctgacaaaacattaatgttagtcggctctgctaaaggtggtaaaccagatactgtttatcgtttccgtaattatcaacaagctaaacaagtactacgtagtggagatttgctagatgctatcgagttagcatggaatgcatctgacgttaataccgcatcagcaggagacatcttagcagttcgtgttgaagatgctaaaaacgcaactcttgtaaaaggtggtttaacatttgcttcaacaatttatggggtagatgcaaatgaaattcaagtagcattagaagacaacaatttaacacacacaaaaagattgactgttgcattttctaaagatggttataagaaagttttcgataacttaggtaaaatcttctctatccaatataaaggtagtgaagctcaagccaactttacaattgcacaagataaaattagtaagaaagcaacaacactaactttaaatgtgggctctgaaccagaaagtacaacagaagtaatgaagtatgagctaggtcaaggtgtttactctgagacaaatgtcttagttagtgcaatcaatagtttaccagattgggaggctaaattcttccctataggtgataaaaacttacctactgatgctttagaggcagtagacaaagtagatgttaagacagaggctgtattcgtaggggctttagcaggagatattgctaaacagctagaatacaatgactatgtaactgtagctgtagatgctacaaaacctgtagaagactttgaattaacaaacttaacaggtggttctgacggaactgctcctgagtcttgggctaataaatttccattactagctaatgaaggtggttactacttagtaccattaacagataaacaagcagttcattctgaggctttagcttttgttaaagaccgtacagacaatggtgacccaatgcgtattatcgttggtggaggcactaatgaaacagtagaggaaagcattactcgtgcaacaaacttacgtgaccctagagcttctttagtaggcttctctggaactcgtaaaatggatgacggacgtttgcttaaattaccgggttacatgatggcttcacaaattgcaggtattgcaagtggtttagaagttggtgaagcaatcactttcaaacacttcaacgtaacatctgtagaccgtgtatttgaaagtagccagttagacatgttaaacgaaagtggagtaatctctatcgagtttgtacgtaaccgtactttaactgcattccgtgtagtacaagacgtaactacttacaatgacaaatctgacccagttaaaaatgaaatgtctgttggtgaagcaaacgacttcttagtttcagaattgaaaattgaactagataacaacttcatcggaactaaagtgattgacacaagcgcaagcttaatcaaaaactttatccaatcattcttagataacaaaaaacgtgctcgtgaaatccaagattacacaccggaagaagtacaagttgtcttagaaggtgacgtggcatcaatcagtatgactgtaatgcctatccgtagcttgaataagattaccgttcagttagtatacaaacaacaaatcttaacagcatagagtgtaggggcagttgcccctcctcttacataatgaataaaatataggagtgatatatacatggctagtgttggaaatcaaacagtccacacaggtaacacagtttacctaatgattggtaataaaattatcggtcgtgcgcaatctgcatcaggtgagcgccaatatggtacacaaggtatctatgaaatcggtagtattatgccacaagaacacgtatacttgaaatatgaaggtacaattactttagaacgtatgcgtatgaaaaaagaagacttagcaagtttaggaattacagcgttaggtgaagacatcttacaacgtgatattatcgacatcgtaatgatggacaatttaactaaagaaatcgtagtagcttatcgtggttgctctgcaatttcttactcagagtcattcacagctaacgaagttacatcagaatcaacccaattcacatacctaacctcagctaaagtaaaataacaataatgttaaccacccacattgtatgggtggttattttatgttatactatttgtattgaataaaaaaaagcttaaggagggcattagtagtgaaatatccatatttagtagaattgtatgcaaaacatgtaattagagactcagggtacatagaaaatgtaccaccagttatttatgaagacgttgtgaaacgtgttgaagaaataaaaagagaagaaaacttaacaatagattagctagtataagtttaaatagacctaattcagatataggtctatttttttttgccactataaatacaaataactagatagtgtggtataataaagatagtgctatattaatttatgagtaaactaggaggaactaataatgacagaaaataatgaaaaggtatttacacctacaccaaatttatcacgtgagcaattaatagaaaagttgcaacgtggagaaaacttgacggatgaagaagtaaatattttgaaatattacaatgatgcagaagaacgtaaacagttagatagaattatcccgggtgttaatgatgtattttctaagcattataacttaaaagaatatggtttagaatttgatattaaaataaaagcacctaatattatcgaaaacggtaaaattcaggcaagaagagaagcctatttagaaggcatgggaatggcagttagcaactttattttccaaagttaccaaatgttagcaactattcgagtatgtggggtagaggtgccagaagtactagctgatgatgaaaaaatttataatttatatgtattaggagtaattgcaaaggactatggtgaatggctaaactcctttcgatactagagttaaagaactaggtggaataaaagccctcgttagaaatagctatagtagaaatctatgggcaataatgaaaaaatttaaggtactaccgagtgaccctgcgtggcaaaatctcacaagtgaccaagtagagtggattttatataatatggaaagagacatagaagaacaagaacgactagctaagggaatgcagttagaaagtgagttccaagactatgacgattcatggtatgataagccacatgatgagttctccccaattcgtgaaggtgacaatgaggaagaaattgctcgtaaacttagtgaaatcacaagtgaggaagacatggctaaacttaaagctcgttgggaggcaagccaagaagttgatgctatccgtgcagaaggtggaacaacaattgaagaagatacgattaacgaacttattgctaacaatgttaaaaaagcaatggaagaggctagacgtattgagaaacatggtggaaataaatggcaagagaaatcatcaattgagttagaagaggaacgtaagaacctagagtttaactcacagttgaaacaaggagatattcaggaggctatcgatttgtttaacaaagatgtcgagccaacatcattagatgacgaatttcaaatttaagggtagggtattccctgccctttacttgtaaaggaagtgtggaaatgagcaacaattatcgtttttatgttgaggcaatgactggggatgctgttgcaaaacttaatgaaatagacaagttaatggataaaattgattcaaagagtgcaaagggcacccagaattttttccatacaagtcagaaagacattgataaagctgttgaggaaatgcaaaagcttatcaaggcaaaaaaagaactagacagagcttttgataatcagaagataaatgcagaaagcatgggagacatgacagcatataaacgtgctgtatcagatgcagaagaactaactagaagatttaacaagacacaaaaagaatttcaaaatcatgctagaatgcaagctaaccctaattacataaatgctagcacactaaggcaacaaaaggcatttcgtgatgagttaacagagcaggagagagcaataagaaatatctctagggcacaacaggagctaaacagggtgaactctagggttaaccatcgtgcaaaccaagcaagcgcaacaggaagaatgacttacaatcagtcagagagtatgaagcgtgaccttagacgtactggtgtatttgaaagcttaggctcagaaaataaaagcagacagcaagagctacgagaacgatataaacagagacaagaagaactagcagagactagaagtaatacaaatttagatagacaagttcgtaagaatagggaaactagtattcaggctgagataaaagaaattgagaaagaaattgaagctagaaaacgcctagcagactctattaaagagactgtagagaacttaaaatctaaggaagcttcactaaatgcatctgacataaaagtagatgctgatagaaagagtattcgtggtgttctagctgagcgtgcaccttctatatcaatagctatgctaggaggtaccgccgccgccataggtggtttatacgcaaaaggagcaactgctaacgcaggtatgcgtgatgcatctatatctttaggacaacgtacaggtactagtgatttccgtgcattaagaaaaagtatgcaagagatgggtattgagaaacagttaggctataaaggtgccgacatgttacaattccaagaagatgcattgagtaatattggttttacaagtaaagaggacttagcaggaagtacaagagccttagcagagggttcaagagcagttccagtagacaatgagactttaagtgacttcatgaatagtcaaatgaagagtggggctattagtggtagagaccaaattaagaatattcaagaaggtttcttaggtgctatccaacgctcagggatggcaggtcgtgagaaagaacaactagaagctcttaaaacattaagtgagcaaagctttactggtcgtaacggaagtaatcaagagcttaaagaacaaatggcaatgttaacaatgctaaaccaaacaggtaagcgtgctgttcaaggtgagcaaggggcacagttaatgtcaagtttatccgcaggtattcaaggtagtgtatggaacaataaggcatctttacttttaggtaaagggacacaattccaagggctagcaggaatgtacgacttaaaagctatgcaagaacaaggagcaacaccagagaacttacagaaaattattggcagtgtccaacagtcagttcccggtgacgaaaagtctcagaagtatgcttttggtagtgccttacatgaactattcggtacagatgctaagaatgaccaaatagatgcaatttgggaagcttatgccaacggtggtttaagtcaggataacgtagatagaataatgaatgaaagccaatctacaggaaaaggcaaatatgataagaacatagaagactacgcaaactctaaagaaggaaccgctaaccgttcagaagctgttacagagaaacaagcttcacagattaatgatatgggggacatcttaagagagactaactctaaattaggggcactcccccctgcgttatatgcattaggagcaggcttaggagctatggctgtttcactagccacatcaggctctattggtggtttatctagtttaatcaaaaaaggaactcgttctacttttagtacaggtggaggagtaactgcagggggcggatttttaaaatcagctaaagaggcattttctgcaggtaaaggctctggcggatggttccaaggtattaaaaatgtaggtagtgtagctaaggactcagcaatgggtgtcggagctaaggctgttgaaggagcaaaaggactgaaaggtgccggaatgggcagtaaggttcttggtgggttaggtaaagcaggtaagtttttaggtaaggtagcaacccctctagcgataggttcttctctattagatattgcaaccgcagatgataaaacaaaggcggtaggggagagtgttggagctattggtggaggtatcggaggagctaagctaggggcaatgattggtacatttattgcacccggtcttggtacaggtatcggaggagctattggtggaggtatcggcgccataggaggtactctagcaggttctaaactaggcggtaaattcgttgatggtgttcgtaaattcttcggaggagaagaagctcatgcggaagaagcagacttaactgcaggacaagatgttgcttctggacaagagagtaaatcaggagtacaggaagccagagacacagctaacaaacgagtcctatctgagaaaacacgtgcagaaaataacgcagaagagtctgctaatctatctatctattctaagttattagatagagctcaacgtatactaaaccaagcaaaaaatcaaaatggtatattcggtaactcaggaagctctaaagattcttctgacggaatgggttcagatgcttctagtaaagactttggaggggactgggagaaagccattagacaagcatctaagaaaatgggagtagatgtttctgacgatgaaatagatacaatacttagattgattcaggcggagtctagtggtgacgagagtgctgttcaacagataattgatgagaacaacttcaatggcaatggcggagctaaaggtttactacagtacgttcaaagtacttttgatgcatacaaagtagacggtcatgacaacataatgagcgggtatgaccaattgctagctttctttaataatagtaattggaaaaatgacctaaattcttgggatagccgttatcaaaacggtagcacaggatggggtcctactggtaataaaactagagcgatgggtggacacataactagcccagagtatgcactactgggagaggtagcaggtcaagatgagtatgttataaaccctagtcagccaacagccccacgtttattagcagaggctactagaaaaacagctcaaaacttccgattaacaggtaacggttctggtcatgattggggagcttcaatttctaaaataaatgcaacaggaggagctcaatcaggttcagctcctacgatgacaaatacaaatgaggtatcagttaatgtcaccatacaaggtggaggaacaagtgatagtatagctagagaaattggtgataagtcagcaggaattatcagcaaaacactcgatgctaccttcacagatttcttcgcaaaagaatacaggagagtgtagaggcttaatgtctctacttttctctaaggaggtttataatgtcagtagagttaagatacccgagatttgatttaactttttttacagagacagacaactaccatatagtttatgatgcaaaagatgggcttactggacgtaataacaataacggtgaagcagagaaggtaagcaataattttatggcagagtctgtaattagtttaactacaaaaaatgctttagaggatgatagtgcagtcttctcattcgtattagcaggggacgtgtattgggatagagtactaaatgctaatgatgcagttattcttaaaatagacccagatacttcctccactaaaaaatcagataaccctgtactactagttggtttaatatctgaggttagactagagggtgactatggggaaaactctaagatgtacaggataacaggacagtctttcgcaaaggcgctaatgcagtttgacttaggtgttattcaggaagtaagtgtagttttgacagacttaggttggttgcctgacgatgcacaagaaggtataaaaatgtctggtagtagtgcaagtcaaatagcagaaagcttaatgaaaagatttttacaatatatgaaatttaattttaatggtcaaggtatagataagttcttagagtgggagctagatagttggacagaggcggagagactaatagatagtaccccttacataaactatgaaggttctttaaagcaacttatagatgatgtaaccgctaaaccgtttaacgaattgtatttcgatgcaacaccagagggtaaatgcagaatgattatgcgtagaaccccatttgataaatctgattgggaaaaattacaaacctacacagtaacatctgcagaagtaatatccgaatctgtagcagttaacgatacagaagcttactctatatttaacatatctattaataacttgtatgggactgactctatgatgttaggttctaaaccacaagtattccctgatttagtttctaaatatggttataagaaactagaagtacctaataggtatttagaaggagcaattatagataagagtaacagtggggataaagctaacacaaaacccgaaagtgataatgatagtaaagagagcgatggctcaacagctaaacagatgtttgacagagagtacgctcttgtcttaaactatctaaaagggtatcctgttgatgtgctaagagttaaaaaaagtaatgtaagaacatcaataacacaggtagacagacgtataacagataacgaagcagataaaattatagaccactatattacaaatcaagcattaagtaaagaagacttttctaaatttacaggaataaccgaagaaaatatagaagagggtaacggtaaagtagaacctacatataccgcagttagggacttccttaatgggttagataaaaacctaggagtatctagcataaaagagaaattaatgaactacttcaatttgatacctaaccaagccacatctattgctagtgaatacaaagcacaaggtaacttaggtaaactaaaatatgaagaaataatggagaataacccaagtgatagctcaaccgtcactggttcagataaacgatttgtttctgagttcactaaaagactagcaaattggtactgtgaaaacgctaatttctacagtggggatattgtagttaaaggtgaccctaaatatagattaggtaaccgtttgtttgttcaagatgagcaaaatggtgagttgtgggaatactacatagaatcagtagaacactcgttctcctatacacaagggtatatcactactcttggggtcactagaggtttacaaaatggaggaaacgatagattcactcatttatggggtaaatctgaggacttctccggaggtatgctaggtgagaaaaccttgcaagcactactagacgaacaagcagaagcaaacagtaaaaatgatggaggctctgatagtggaggtagctcaggtaaagagtatacagcaggtgcaggaactcagctagcggtattcccattagacgtaattaatgtaactcagggagaaaatggtggatacagccacatgggagcacttgcaatagacttttcagatggtactcctcataaaccttactatgcaccatttgactgtgaatgtgtgtacactgactcttactcaggggttgcatggcaatctcaaaagcctgttaaatgtgtagatggtagcgttacttacgtaacactattatgcgtacacgacaacaactgggcaagcaacaaagtaggagataagaaagctaaaggggaagtaatagggcactcagggactgcaggacaagcatcaggagaccatgcccacttcgaggtgtctaaaggtaaatggcaaggttggagcacaagcagtgcgggagtttactttataaaaaatccttctcatttatacgatgtattttctataaaaaataatgtaactggaaaaacaactaaaattatgaatggtggagggtacaattggcgtagtatagactgggacgataaatctggttcaggttctggaaagaaaaagacagttggtgctagagccatggctaccccattcggatttcgtatgatgcgtagcgctccggtagaaccagaagagccgaaagttgttaaacaaatagaaactaaaacagttgttgaaaagaaaccaaaagtagaagttaaaaagctcccaatactttatgggaataacattgccacagaagcaactaaatggggaagagctcactcgaaaacagagtccacattccaatctgccttcaattttggagctaatacagataaagacccatttgaggaagacattatagcaacagacagcgcaggatttgtttggtggtgtttctaccatgcgggcatatcgctatctggtggagctagaatggtcaccactcgttcactactttatgataaccaattacaaactatctctactagaggtcagaagtctttagagttatttgataagatgaaggtaggagatttagtttggtttaacagagctagccatgtagggatatattgtggtgagggtaagatggtctcttgtaatggaaaaggtaacatggatgagtccccaaaaacaggtattgttattgtcgacatgtctaaaggttattggtggaacgcttttgacggaaatgtacgtagatataaataaccctattttttagggttattttttttgtataggtagtttttagttattgtgctataatagataatgaggacacatgatgagaggatgaataaattggtaaaacgtagatttcaggcaggtctaggctcagaaattaaaagagtatataaagaaggacaacaaattaacacgctactattagcacaagtaattcaagtaaactataaatataatacagtagacctactagctttacagcataaagaagtatttcaaaattcctatgcaaatgagggacgtttctcggcaagacttcctatggaatttggcggtagaaatatcgttggacagccttatggacaggttaacccgatagcagtaggaacagtagtactagttggttttattaattccgataaagacatgcctattgtaattagtgtttataataacaacgatgtaagcaagcaactttcaagaacacaattttcaaattcagaccctaaagatttagagttaattggggatatgcaccaaaaatttagtttatacccttcattgacatatgatagcgttgatggagaaggagggcgtgtcgttactttttctggtaaatcatttattgcttttgatacaaaagaagtagctaactcctctacagctgatgcaggttacggtactaaatacgaggacttagagacatcatactataataatggtgacctaatagagcctatgaaaggtagagcaccaaatgtactgtttaagcatcaaggggtacttgatgatgacggcaaaccagatttacacgatttgctaattcatattaacccagatggtacttatagaacttctatgatgaacaaagaagaggattggcgcacactatttgaaatgacaccagatggcagagttaaattaagaaaacaagactctattaatattgatggtggcatagaaataagtgagctaggaatcaacaatgaggggttcgtttatttacgtaatggggatatggatttagaagtacgaaaagacggtatctattcacaagggaaactgtttacagccgatatagacctatccgatgtatatgacaaactaaatgggttgtctatacagattaaggaaacaaatggtcaattagagattatagctaatggtgtagaagaacaaaatggaaaaatatcagaactttctacagaaataacaattgtagcaggtaaagttgaatcaaaagtaacaaagacagaagttcaggatatgattgacagttcttttgtagatatgtctgatgcaattaaaaaagcacaagaagatgctgacaaagcaaataaagtgattgcagatatgtctagtgataacagactgactccaagtgaaaaaatagatttattaaaagaatgggatattataaaaaatgaatatccaagctatctcgaacaagcagaaacctacgaggttgatagtaaagactacactgctaagtataattcattagagctatttgttacccctatattggctgacatggaatcaactagctcggtagacggagcaacactccgcaaaacgtttaattcttactatacagcaagaatagctttactaaattctattagtaaaaaactaaaagacggtatcacagaggctatgaaaaaagcatcccaagcatcactagatgcaacacaagcaatggcagatgcctcacaagctaagattgatgcagataatgctaacaaacttatatctgatatagcaagtgataacaagctaacaccttctgaaaaataccaacttaaaaaggaatgggatgtaattgttaaggaataccctacaacaattgcacaagcagagaagtacgcagtagacacagcagagtatacagctaaatataaagccctagagctgtttgtagagcctttgtttaaagacatggatgaaactagtatagtagacggagaacgccttagagcgacattctcggactattacgcaagtaagattgctttactaaaagaagtaacggactcagctaaaacagagctagatgcctatggtaataaaatatctgtaatggaaacaaacattactcaaacatcagaagctattactttactagctactagagtacaaactgtagaagacggtgtacaatcgaataaggcacaaatcgaaatacaagctgaacaaattagtcaaaaagtaactgctagtgaggttaaaggaattgtagacgattctattaacaatctaacattaggtggaactaacttatttgttataaagactcagacagcaggtttactaaacgagaatgatggaactgtaggtactgcagtagacaactcagtagtgtcagactacattaaagttaatcaaaaaacaccatatattgctacactttatggtaacactggcacaaacatgattataacagactggtacgataaaaatagaacatttatttctggggaagctgtggcagactctggggattttagtaaaaagtatgtgtcacctgagaatgcagtctatgctagggtaagttataagaaagcaaactctgtgaatatcaaattcgaggcaggtacaaaggctactgattacagcccttcatgggaagacataaaaggtgaccaaactgctttagaggaatacattaaaaaagtagaagaacaagccaagaaagctcaacaagatgctgaaaatgctaaaaatgatgctgaaaatgcaaataacgcaatagctgatatgtcaaatgacaatatgttagcaccgaatgagaaaaaacaaatactcttacaatgggaacagattaaaacagagtatccaataaacttagaccaagcaactaaatttggggtgtcttctcaacagtatacaacagcgtataacgcactagacgagtacttaaaaccaatactagctgacatgacaacaacttctgtagtagttggttctactttaagaaatacgtttaacaattactatgacaaaagaactactttactaaacagaatatctgacgtagcaaaaaatgtagcagacaaggcacaagaaactgcagatactatcaatgataatttacaaaatattggtgggtacaactatgtagggttctcttccggagacaatatgttgcctagactgatgattaaaaacgttggttactacacattaggttcgtcaaccacagagttcattgacagcatggtagctgtaaaaggtgatgcaacgacccaacctttcgattatactgtaggtacttctgataaagaaattgctggtggcggtttagctgattatcgtatgaaagaaataaaagaaggtcagtggctaacagcttctgcgaatgtgcaggtaataggtggtggctccgctaggttagctatctacactttagaaggggataactgggtaggttctaacagtacacctatacaagtaagtgatggtttgaaacgtgttgtggctcaaagaaaagtaacaggcttaacaaaaggtgtgttaatacgtattgagtcagctgacactaatgttaaagagtttcgatttggtaatgttcaactagaagtgggtatcatcccaactccttggaaaaagtctgatatagatattcaagaggacataaacaatgttgttcagaatatcaaaacatacactgcttgggctaacgatttacagggtcttgattttacaagagaaaaggttgaaggaaaaacttacatgtatgtaggtacctctatgaaagatagtgataactatccagattatacatggaggctaactgatgaacatatagaaggtcagattaatggtaaggaaggcgcatggatttactctccaacagcccctgctaacccatcgcaaggacttatatgggtagacttgtcaaaagtccccaaccaacctaagcgttgggtagattcagaaactgggtgggttgcattaacaccagaagaggttaaagatttgccttggggtgaagatggcacaagcttagccgactgggtgtcacaggcagagcaaagaatatcttctgatagcattataaatactgtactaggttctgaggatttcactagtgtgttcgatacaaaagctaacacttctgacctaggtaacttggctacctatgaagacttagactcaataaaagaggactataaccggctaatcaaagaaggcataaatggtattgattttactccttatgtgactaactccgaactacaacagcttaaagacagctttaacttctctgttcaacaagccggaggggttaacatgcttaaaaactctttaggattctctgggttagacttctggaatggtacagtagggaagaacttactacctaactctacttggaatttaggttttggtagatggggtggtacttcaatcactagttttgaaatattaccaccagaagatgacaagcctacgagtaacatattagcctcaatgccacttcgctcttctactaaagaaataggtaacagacctcacccattaaaagttaactcgggtgaaacgtacacagtaagcttcgactataaagaagaagcattatcttacaacaaggacagacctatccttgttgtaagaaactaccctgataaggacacagaccaatggatggagtactcaatagaaggttgggcagtaatggctaacggaagcactactgacttgactgtttggagacgttttacaaaaacatttacaataggtactagtggctacttagatattttaccgaaaaccataatagaatcgtgggaacacaggtctttttggagagagctaaaaatagagaaagggacacaagccactacttgggtacctaacaaggaagacggggcgtttactggtgatattgttgagactattcaaacagaagaattagccaacctcgggtttggttccggatttattagttctaaaagacctagctcttcattaacacaatctgtagaactacctgaaataggtgctaaccttgagtattcactatctttttatatgaaggtaactacagataaccctgtagctgactttaaatgcggtattcgggtttatgatggaggtactctaacttatacattaggcatagaagatgcaacacagccaataccactagggttccaacaatacaagcttgtgttcactcccacaagtacctctactaaaatagaaatgtttgtagaaaatgggcaagaggcatctgttattatatcaggtattatgtataatatcgggagtatacctcttaaatggcaaccatatccaagtgagatatacaatacgaatgttaagattgatattaatggggtaaccgttaaaaacaatcaaacagatgggtatacaatgattactccccaagagttttcaggatactctcgtattgatggtaacatagaacgtattttcactttaaatggacaggtaacagaagttaaaatgctaaaggctgaaaaacgtataactatggaaccagtatctgtattcgctatgaacacagtaacggatacaaaaagaattagaggttgggcatttgtgccatcatttgaataaataattcaactttttaaccgaaaacctcacttacaagttgttttcggttaatttgtgttatactatatagagaaggacacgaaaggacgatgagactatatggctttaaacggaacaaagtatacagcctttgcccgacatagattagttttagagtggcgcgcaaatcaaaacattgcagggaactactcaacaatcagcgtatggctatatctacaatctatggataaatgggggagacttgatgctcccgctattggtgatgccaaagttaccgtagaaggaactacacagacagaaaaagcttcctctatgttaaatgctttccaaaagaaactattactagctaaagagtggagagttaaccataataatgacggttctaaaagaataactattgggggtagctactttgtaaacgttacttttactgataatggtgtaccaacatattacggtacgataactatacctaacttttcagtagacctgaatagaatacctagaagaagttcattaaaccctgtccctacattaaatttaccggggaacttaccaataacaataaataggcagagctccacattcaaacataatctaactgcttgggtggctaacagagataaccccacattaagtaatgatgcccattggacgtacttgacaaatcttaataatgtagacactagtgggtcatttagttttacagtagcaaataacaaaactatttttactgcattaaacaataggactagttggcaaggcaaggttaaactatggactatagggttagatgatgtagttagtcaggagagaacatacaagattgtccccccaatgaatgcacaagcatcgggaggtaagataaccttaaatgtgggagagaaaatcaatgtatcactaagtaactatcggtctgatgcaaactttacttatgatggggtatttaacattagtgggctcaacatacccattgctacaaattcagcagggaacacaatgtcgtatacactaactcaaacagatgtagacaatatattgaagaaaataccaaatgcggattcctcgtggggtcaagtaactgtaacaagtaagtatagtggagtacaatacaggactccatggacaggacagagaatagatataactatacctaaaaacaagtatgtaccaagcattaatggtacccctacttatgaagatacaagtagtgtctctgtcgggcttacaggtgacaatcaggtagctcttcaaggtaagtctaatatcaaagtgactatccctgctaactttgcaacggctaatggttactctactttgaaaacaattgatgtgtctttaggcggtacatcaaaaacagtcaactattcaaatgcagaaacagttgtagagttaggggctcccgctaaccatacatcagacaccttaatagtcacagtaactgacagtcgtgggtttaagtctaactggacaaagcatgtagacatttacccctacgaaaacccaaatatgtactttacagtcacacgtagaaataactttgagacaactacagatattaatgttaatagtacatggtctcctatcactataggtggtgtaaataaaaatgctatccaatcagtgacctatgcaacaaaagtagtgggtgttggcacttatggagaagaaactgctttaaattttacagctaatggtagtatggtaacagtaaaaaatacaccaatagaattagataatactaatacatacgacgtcaggttaagcataactgataagtttagcacgtttactagaacagctactgttaaaccgggtaaccctatcatgtttattgatgccgataatcgaagtctgtttttaggtaatgcctttgttgacaataacaataatgagctaagaggcttattagagatagaaagagataagtggcaggataggggtttagtaggaatatcattaaacaatagtgatatatcagcagtcaacggtatatggttctcaacagatacctcagataacgcaggtgaagggctccactttattaaatcaggtaaatcaagaaattcattgacttgggatgattatgattacttttatatgagagataacgggttctatgttaaccacaatactaaccctatatttgaagtttctgacactggtacgatgacgtttcctactaaacagctttggaaaggggttgtctatttaaacgatatacaagaaattttacctagtaaaaaactgacagaatgccgaaacggatgggttttgaccttctcagcatatgagtcaggtgcggggcaaccttgggatataaatcaggtatacatccacaagtctgtgttaaaccagtttaatacaagaggatttagggctatgataggtcatggaactacgataacatttaaatatatgtatataactgacggtcgtataggtggacatgctggcaatggtataggagttaacagtggcattgccataagggaggtatcagaatggtagaagaaactcaaaaaatatttgtaacactagatgataaaaattttgttagtaactgggggacgtcactcaatagtggcgaacctgtctatgaagtagatttgccaatggactctccattcttttcacattactattcaaattgttttcagtatgtagatggagagctagtcctaagtgaagaaaaattattaaaagtagaactactagcagtagccagtaagtttaaaagggactgtgaagagacctacatattgcaaaaagttccttatacaatatcagacactacctacttatttgatgttattagcgatgatgagtttattaataaaataactctactagacaataaaattcaagattatgttggggtaactgcttatgctacatcagatggttctcaatcgatacttaaatttgataagtcccagtatactactatgactagatatattaaaaaagtagttgagtctagggaaaataaattaaataataaattgcttcctatgattgaaaatgctaagtctgtagaagaagccaaagaaatttcttggagttctgtacctgatgagttgctaccagagcaagagaataatgaacctcaagacgagcaaagtatagatagtttaattaaagaaaataaagaattaagacaaaaagtagaatttaatgagttagccttaatggatgcaataaacatgttctcggaaatgaacaagcaagctactattaattaaaggaggtgatatttctatgtacccatacttatcaatgttatatgcatcttatgtaatcaaagaccctgagaactaccctttagagaaggttccggcactaattagagaagatgtggagaaaatcgtggaagagatggcaaaaaagaacgaaaaacaaggatagttaatttagtaattggagtaacaataggtttaatcatagggctactaatttaaactaatataaatgagggtagtttgtgctaccctcttttttatactatattgtactatattaaggaacgaaaggagcggttactatgggacaatcagatggtatgggcggtacattaaaacgtattgcgattcaagtaggtaacgacccgaataaaggttggtatcgatttcaagtaaacccaactcaatataaatataataagccacatcgtgttactatatttaaaactaaatcaaacattataacagaagactttggtaaagatatagaaactatccaattttctggtacaacaggattcagggtagacagcagaggtaaaaatggggcggacagactgaaagaattagaagaaataatagataattacgcaaaacagggcggtaatgggaataggtctagtgtagagatgaaattttataatttcacagacgataaatattttgttgttcatttagcaccagaagggctatccattgaacgttctgcagaacagccattattatttaattacaccttaagtttagtcgtgttaagagaggcaggacagccctctgagagagctcaagtaagccctcaaataggtaatgtaagtcctagcattggacgggcatacaatgcacaacaggacactagaacacctgctcaaatacttcatgatgaatataggagaagtgttatgccaaatacagcagtaaatcctgcagtaacatcaggagcctataactatggtgtaaatgagttaaaaaaaataatcggatatggagggtaactcatggaaaaagtagaacaatccgcagatttgctaagattttttagatatttaaatgtcgatattagcggtgaagttgtagccaatgttattgacgaccaaccaaactttatatcaaggttctacacccctcatacacgagttaataaaatatctagtacactattagatattgttagagataacgatataggggaaacaaataaggctttgtctaaagactccttaacctacaaatttttaaaaagtgggcttaagctttcatctccacgtatttacgaactagcacaaattgtagtattagaatcctttgctcttatatatgctatcgaagaagaaccagagatgtttaaaatgattaacgaatcagatgtaaagcagactagagaaaatgttaaatacttaattgactgtttagggggagcaaaagactatacagacatagtgatggacttacaatctatggatgtcgctctaggatatattcaggagcaagttcctttaatacaaggaggtttaccagtaaatggcacgatataaaaagcatttaattgtatatggagatacaatgcaatccatagcacaaaaagaaacaggctctgtagataattgggtaaaaattgcggagtacaacgacttagtttacccttacatagtagacactatgcaagaaaaaatgagcaacttagaacatctagctacactcggagacaccctttttatacctgatgaaggtaacttattggacattaacacaagctcattaaaccaaagagatatggatttcttattaggcttagctttaggtaaagatttggatatgacaagtgatacagattactatgagaatcatggaacaagtgacgaagtgtttgcaataacacataatgggcatggggacttgaagatagctagtggggcagacaatattaagcaagccactatatcaagattgatgacagctaaggggtctcttatgttgcatccagaatatggtagtgacttgcacttaatgttcggtaaaacaacaattgaacaaatgaaaataattagtatagaagtatgtgatacagtacttaaagatacacgagtagcggaatgtgttctagtgaaccattatattgaagaagaccgatatgttggtaactatagagcaaccttacagtctactagagagcaatttgagtttgttgttcaaaacgataactcgggggctctaattattgtatagaaaggataaggtttaatgagactaaagaaaatttcagagatactaggtagactaattgatgtaactatgataaacacacatgagataaacgatttttcagttggttctactattcgttctatttatgaagctgtttctatggagttagaacagtactatattttaggtagagagaatattttatggggaattgaacaaggagtcctaaatgcttttgactttaggaaaagagaagcaaaaagagcatatggtatggtaacactagagttccatacggttactcagacccctgtgtatgtgccaacaggaacaacattcgactctagcttgtctggtgcccctagcacgttaacatttcaaacgatgcaagactacataattccagaaggtgttataacagcaaaggtagaagtttattgtacaactgtaggtacaaaagggaacattcctaaaggaagaatcaatcgggtaattaacaatatatcaaacttaaaaaccgtgtataatgagtttgattttttaacaggtactgatgaggaaagtatagagtccgttaaaaaaagatttcatgcatttgtggaatcacgtggtagagcaacgataaaagccttagattacggtacacgtcaagtagaggaagttgcaggagtttatatcaaagaagaagtaggctacgttagaatatacgcacatgatttaaacggtgatttaaaacaagaaacactagataaaataaaagtagctattgaggactacagacctgcaggcattaaactggatgtattccctgtaataaaatccaatgtacaagtgagtgcaactgttactataagtgacaaatcgagaataaatagtaaattagaagaaagagtggagcttacaataagaaactacttaaatagtcaagttgtttcacaaccattaattttagctgacctaattcaggttataatgaatattgacgatgttttaatctatgactgtaaaattaacaacatggaagggaacatggctgttagggacgaagagataattcgtgcaggagaggttattgtagaactgatataaattaggaggaatataatgtgagtaacttttataaaaatatccaccctctgttgagacgtggtaagaaacctaacaagtatgatgatactaactttgcagtgcttaatgcgttgaactatgaattaactcaggcagagcaggagaccattgctagtaagattcattcttcattagaaacagctacaggtgagtacctagatacttggggggactggtttggcgtatatcgtaaagatgattggaatgacgaatactatagaaaaagaattataagagaacttttactaaaaagagccacaattcctgctatcattgatgcgttattggatttccttaacgacaatgatgcagtcatccaaatatatgaaccttggagaaacattttctacacaaataagtcaaaattaaacggtgacgaccatttaatgggtcactactaccgttttgcaattatcgatatttcaattgatagaccgtttccacctgaaattgtagagattattaaagctttcaaacctgcgggagttctattctatctaaggctagacacaagcttaaataagaataaaacaactgtagaaagcccatatgtatacctagacgtgacgaataagacagacttagagttccttaacggtttatactatgacctacgaggtaatattaacctgtctgaccaacgtatacaagttgtggagaacaacatcttccatacaaataactctatgctaaacggggaagatgtgcttgcaggagcatttgaccacggaagaggctacattcacttagcaagtacaacattgcttgattacacaccaaaacctactgactctatgagtgacttaaaaacagctctaggagaatcaggtgctgatatgtataatcaaacaaaagaaaaggatggaagaacagcttctattcaagtacctgcaacaaagaatgtccatacgttatactccaacagcactgacttcgggggctatgattattcggggaatcctaaccttagcgctaaacttaatgcttcaagtttttcttcaggtactggcgcaacagtcgcggatgataatgatgagattgtgtttaccttagatggcacaaatcaactatcgaagtacgctaccggagcgcaaacgccggtaatagaagggaaacagtatactattagttgtgaaatcatgttagaaacgggcttcactggcgacccgtctggaatacgcctacaacacgcatacttgcctggtggtaacgttgttttgcagacaacgacagtacctaaaaatgaattgaataaatggcagaagcttatcggaacacaaacggtaaaatacacctctgacgcaccaaatgaatggtatccattatttagagacattcgaattcttaaaccgaccggtaaagttagactaagaaaaattaagattgaagaaggctcaacagccaccccataccagccaaatttactcgatgcaccatattatctgggtaaggtggctttgggtgagaatattgctaataagtccgttgagtttccaataaaatctagcaattatctcttatataacgctagaatggtagagccttttgttgtaggagaaacttatacaattaccataaaagcaataaaaccagccagtcaaacgtttatggtatataacattggggaaggaactacttattacggaaaactaaatccagttgagggattgacagacgtatggtcactaacattcacaccaagaaatgtttcgtcaactaatcctagcgatttacgtatttttcagtacccatcatcaacattaggcacatgtcaaattgattggttaaaaattgaaaaaggtgataccagaactcctaacattgattcctacgactacgtaggttctctgatagaagatacagaaacacctacgttagacccgactaagtatacatggacagtaaatggggatataacaaataaaaaggcatatatggtgtttgatattaagacatttatcgaagaaaattatgctatagaatttgaaaaacttattactgacctaggagaagaccaagcattaaataccgtgtttgaaaactttaacatctctacaacacttaaggctctagtaagtccaagttcaccaatcagtttctcggttgaactatacgatttttctacgagtgcatggcacaagttaaacacggatagcttagacttacgtatgcgtacattcaacttagtagcaaaccgtatcacagactatctaaatgattacaagctattatttgttcgttacgtgtttgataacgaaacagataaagatgtaacagttgaactagacatgctaaatgtactattcaagtatcgtttaggtgaggggtacagtataggactacaatcatctgtggaatcactaacggaactcgttcctatagaagggtaatagcaaataatatgttataatataaatagggcacctagttgtgtcctattttttaataaacatgctatattaacaattgaatgataaagaaaagaggtaaataatgtggctattgcaactaacaattcacgagtgtatgcctcactccaattaaaaaataaacaagacagtatgtacctagcaattggtaaaaccactccttggactaatgaagatgcccctcctgcaccagaccctactacgactactctaacagaggttatcggttataaaaaggtagcaagagtatctttatgtagagaatatctaccaagtgacgattctaaataccctgtggtgtcctatggttcaagaaaatttacgctaattccagatgaggacggctataaagagcaagcgtggatggtgtatgtagaagcagaaattacaggagatgagttaccaacgggaacatttaggcaagtaggtattcatactgacttagtgtctaaggcaagttcagaaaagaaagctttgttacctacagatgtaacagatgcaggcattttgcaattttttgaaaatagacagcaacaaaatagaacaagtgatgtaattttaaaagagaagtttattattacaatggaaaataagaagtcagttaaacaataggaagggtgacatagatggctaaaaatattacaaatgatgatttaggtaaagagccttataacaatagatattaccaaggcaaacgattttcaggtttactatttaaaccagataagccgttactacaagccgagttaaacgagttacagtcaattattcaaggagatttaggcaatgtggctgaatccatatttagtgacggtgacatccagactggtatggaatatgtactacaagataagaagcttactattaaaaaaggtaaagtattcttaggcggtaaaatgcgtaactttgacgaacagagtattgatattaccggagagggtacagagtatgtaggtgttaaacttgtacaaaaagttattacagcagaagacgacccatcactgttagaccaaacaagtggtgttcctagccatttctcagaaggggctgaccgattagacgaagatgtagtactagcagttaatgatgattctgcatcaaacatttaccactttgttaacggtgagttatacattaacccagatactcctgagatggataagattaataaaatactagcagaaagaacttatgatgaatctggttcatatcgtgttcgtggttttgatatgtacacagaagttcacccaacagaccctaacaataaaattcagttagttgtcgattcaggtcgggcatatgttttaggttttaaagtagataaacctacaactactcgtattgatattgaaaaatcaagagagttagaaacaatcaataacgaaggtttctactatagcaatgcaactcgtaaaaataaattaggtaattccccagtatcttctgtagaccgtgtaactgcacaagttgaagttgctaaagaacaggtttctcgtggggtcgtaggtggaggtaccgattaccttaaaaacacctctgtaacaaaagttatccgtgtatggactgagggttcaggagcacaggagtacaagcaaggtgaagacttccaattagtaaatggtcaagcaatatcttgggcacctacagggcaagagcctcctgcagggggaacctacttcgttcaatatgtttataataaaacaatgattgaaaatacagattacaaagttgtaattactggtgaaggtgatgctagggaatggtatatcgactttaacgagatgacaggttctaaaccagtcgatgaatcacttgttaacgtagactataaatacttcttagctcgtaaagacctaattgtattagaccataacgggaatttcactgtccataaaggacaacctaacgctttaagactagtagaggcacctaaccatgtagacccattagttttagctattggtacagtagtagtttacccagactcaaatactgctgatgctaaacaatggacaatcacacgtctaacaatggaagagctacagaagttatctgttcgtgtggaaaacatggagtataaccaagcagtgttctacttagaccaaccggcaatggcaggagaaaaccctatctatctacgtggagtattctcggatgcgtttatctcactagataaatatgacgttagtcacccagatgccacaattgcttttgactttgacacagcagaaataacattaccttatgcagaaattaataaaacagtcccaacaattatcgaaggttctagtgaagcacatgtgtggggtagactagtaacagcaccgtttaccgaagaagtaggtattagacaaccatttgctacggaagcaatgaacgttaacccatacaacacctttaacaaacagggtgccttaaaactaaacccttctgcagataactggattgaagatgagcgtatcacagttaccaaagaagaaacatctactatgactgttcgtcaatggtggagacatggtggagcatcttggacaaacgatgagatgaacatggtatctaacatcacgcttgaccccggacaatcgtggggaggtgcctctggcacagaagaccaacgtaaacaagggttatccggttctactttaaccagtggtggtcagcaaaccaaagaatcaatgattgagtttatgagacaaattgatgttgaggtttacgcagagaacttgcaacctaatgctaacaacttatacgtaacattcgatggcttgagagtacctgttaccccatcttctggttaccgtaaaggtgccacagaaggtactggtatggcaaatgcagacggaacatttaaaggggtatttaaaatacctgcaggagttcgttgtggtactcgtgaagtatctgttagaaacgatacaaacttagcaagtactacgttcactgcgcaaggtacgttaaaaacaactgaggatattatcataaaaacccatgttactattaacttggttgaccctttggcacaatcattcagttttaacactaacagagttgcaacaagttttgacgtattctttgcatctaaggataacagtacaaatattatttgtcaagtacgtggaatatcagaaggtggacaacctaataaaactgtgtatgctgaaagagtgttaaaaccttctgaaatcaaagtatcagatgatgcaagtgttcctacgaaaatcagctttgatgacccactaatgtgtaaagcaggtcaagaatactgtttagtattcattactgactctgataaatacacaatgtggattgcaactatgggtcaaaatagggtagacgaaccaacacaaacagtaacatcaaacccttacttggaaggtgtcctatatagttcatctaacgcaagtgcatggtctattcaccaactgtctgatttgaagttcactgtatacacggctaaatttaacgaagaagctgtacttgagttcgatgttatgaagaacgttaatgtagaccgcatagtgttaatgtctacctacctaacacccgcaaacactggttgtagatgggatatgaaattagtccttgataatgagcctgcaggtacaacagtagatgacaaaccttggttacctattgctaactacgtagacttagatgttaaccagttagctcgtgaggctaagcttagagcaacatttaaagctaaccaatacatctcaccaatgctatccttggacgacattatgttcgcaggatttttaacagcattgaaaggtagctatgtatctcgtacaatagatttaacagaagctccatacaacacggttaaaatgtcatatgaacaatttacacctgccggaactgttgtgactgctaaatatagtacagacgaaggtaaaacatggaagacatttacagtacagcctacaacaacacaacgtacacaagactttgttcgtgtagactatgttgaaaagattaatacgggtgggacatttaagtccattaaattccgtcttgatatgacgacccaaaactccttcctccgtccaagagtaagaagacttatgactaacatgacagataaatagcaacagaataagtataatagactctatatgttataatagacatatagagtctatttttttgttaggaggaataaaattgccagaaacacacagacaaacaagctcaggtgcgcttatatttaaaccaactatagctgaacaagaacataaaaatgctatggaatctataaaacaagagagaacagagctagagaaagaactagctaatgttaaagctatcaaagatgagttgtcaaaagagctagcagacattaaacaactgaaagatgaattatcaaaatagtttctaaattgtcctatttaggatttatggttcccttatattattaactaatatatttaaataactatatataaaatatataatatataaataatatataataagactacgaaaaatataaaataggacaaatcaactctgtatacaccttgacaattacctactatgtgatataatgaatatatcgattatactaaggtgggtatgtcaaggagcatgtatactaatctaacttaaaggagatttattaataaatgagactagtagtagatattatgcatactcagataaggtatgaagattcggaaaattatcttagaccagaaatccataaggtaatgcattcagaattaggtgttaaagcagatggataccaattcagtcctgcatataaagcgggttactgggatggtattattgatttttttgataaagaaaatgatacgttccccacagggttactacctcatgtagaaacaatactagggaaccttcaatcaactttatcaaagtcaggttacatttttcagtttgagataattgatgatagacctgatgagttcatgtcagtagaggacatggataaggagatagtgcttaatggggacaacaatgacaagataacattaagagactatcaatatgaatctgtggagcaagttattaagaacagaataggtattgtaaacgttagtactggtgggggtaaaacagaaatagcatcaggacttatacaacaaataaccccttacttagagtcaggagaacgtattgcattttttacgaatagttcttctattttttcacagtcaattgaccgtattgagaaaagacttggaataaaagtaggtgcttttggtgcaggtaagaaggacattcagcaagttacttttgtaatgatacctacgattgtatctgcaatatctgcagacccagaggctaagcttaagctgactgctaaagaaagaatgtacaagaaaattgctaaggatatagctccaaaatttgaaagagggtttaaccaaaggagtttgcttgaaggatacctaaataattttcaagttaagacaaaagcagacttgcagttgaaacatgaactagaagaaattttttattcatgtggtacgaacaaacaggttattatgaaaatgaaagggtatcaagcagaataccaaaagattgttgaaaagaaaaatggtaaagttcttaagaagtacaatgaggctatggagtttttagattccatatctgtaatgattgtggatgaggctcaccatacaagttcagatacttggtataaagtgctaacatcttgtaataacgctcagtatagaatggcacttactggttcgattgaccgtacaaatcatgtgctttggcaaagacttcaagctatattcggggagattacgactaaggtgtctaacaacacactcattgagttaggtcactcagccaaacctaaaataacaatattccctattatagctcctgtagatattcaaaccactacgtatatggatgcctatcaaaaaggcatagtagacaatgaatatagaaattctcttattgcaaagttgactaagaagatgtatgataaaggtaatgggattttaattataattaatcgtatagagcacggagaggctattagtaacttactaaaagaagagggagtagctcactactttatcaatggacaacttgaaaatgatttaagggatgaaaaaattcaggacatgcgtgatggagcacttaaagtaatgatttcttctacgattatagatgaaggtgtcgacatttcagggattgacacattaattcttggtgcaggaggtaagtccttaagacagacactgcaacgtgttggtcggggtttacgtaagaaaaaaacaggagaaaacaaagtagaggtattcgatttttatgatttaacaaacaagcatttgaaaaaacattcagagcagagaagaaaaatatatgaagatgaacaatttgaaattgttgatattcctattcctaaataactataacaaagaaagggtggcatacaattgcaaagtccatgcctaaatattgaattaaaagaaaaatttaaattaaacaaaggtataacagactttttagagagagttgcagataaatctcagagatggggtgaaacagtagcctcccctatccgtaaaacggatatggcaaaagagactggaaaaaaccctagaacaattacaagatatattaatcaattagaggaactaggtttaataaaaaccgaaacaaaaagaggaatgaatggagggactttagtcgtgtttaacacagatatgcttaattttgagccaaaggaaaaccctatcacgtctgatactaaacaagcaaaggagattagagaacaagtcttccctaaagccccaaccaaagtaccaaaacgcagatatagaacaaaagcggagatagctgaggcacgtatacttagtgaaaaacttaaaaaacgtgaagacattttaaatgataaaattgagtttaacgttgttactagaagttttttcgatagttttgacgaaccagaggcgtactttaaaggctatttaatttcaagaatgtataatgcttatgtgacaatcattccttatgaaaaatataatagactgaaaaatttagatgagaaaaaggctaagcaacaactacgagcatacgaaagctcttacaattacgatgtgttgcctagaagatttgttgggacacctcagtacaaaaaatttgtagagctagctaaatattgtgaagagaacaatatcaatcctttagtttatttaacagtgcaatttgatagaacagagtttttgatttctgtaggtaaagcacgagtaggggctacaccctatgtaaatactttgttatgtgcagaggctaaagaggcttacacaaatagaaaactgttctacagaatgttacagaaccaatatggattgtatacgtctatcaattcagaagctacttattatggtgcaacttatccaattatttcaggactattgaatgcttacaatatgccacagaaagacttgtcacagttagatactgcgatttgtgacttagaatataaaaaagatattgacaaaaaagcaggtacgttgtactcttattatacagcaacacttaaatcgttaggtgaatctgacgtatctagtgaagctaaagaaagtattgctaacttcttgaaagaacaagttgcaaacttttctagtaaacgtgggctaacttcaacacagtatgccttagcgttccctattcagataaactctgcacgcagtttattaatgaatgaggaagacgaagaactactttatttattgttaggtaaccagtcacgattatctaatgtaacaaatgatgaagctgaaatgtttattaaacaaggtagaaaattaagtatgtcttggtggggctcacagaacttctcaagaacaatgttcatgttagcggactactatgggtttaaaacaaatatatctaagttaggtatgtacattaaagaatttggtgaggagaaaattcctttagattctgtaggtatgttagatgtaaatagaatctatgatgtcttaatgactgaacaagagattttagagattgacaaaagtaactgggagaaccaaaaagatatgcgtgacgacaaataagaaatataaatgggagagggacggatagtaagatgagtcaaattcaaaaacaagttatttatagagcattaagtgagccattctttgcaaaggaaattctaagtaaaattccaatggatgagtttaaagattctggatatgagatgattgtttctacaatcaacttatattacagaacacatgatgagagcttagaggaacagagtttattaacactagtagaagataagatgttaaaacaaaacaaaagtttggaagctcaaaacaaagtctttgaggtagttagcgacctatacgaactagaaaacgaagatgtagactcagaagttatcagtgagaacattcagaactacgttcgtaaggtactaacacgagaagcaatcatgaaatctgtaacaaatgaaggcacactaggctctgatagtaatattcaacagctaatggatgacttgagagacattcttactatcgagacagcaggcaataattcagaattgctagacttcttcgatgacgtagataaaaagatggagttacttgcaaacttgcaacaaaacaaatacccaactggtttcacagctatcgatgctatttccgatggtgggctagctcgtggagaagtcgggatggttgttgcacctactggtggtggtaaaactacttgggcagttaaccaagctagaaactatgttgtacgtggtttaaatgttctttatgttcctttagaggaaaaagtagaccgtatgattgttcgttttgaacaattattatcacaacaaagtaagaagaacatcttagttgatggtgaattgaataaagacttgtatacccaaatccaacaagcgtatggagcgggcaaagaacagatgaattgggggaacctttggattcgtaaatataaaccacaagaactaacacctagcggtttatctcagttaatctccgatgtaatgattcgtaaaggacagcaaattgatgttgtcattattgactacccagatttaatgaaaaatcctcatgcaagtggaagtaatggtgaatcagatgcaggaggtaaactatatgaagatattcgtgcgattgcacaagagtatgattttgtttgttggacgttatctcagttgaaccgagcaagctatggtcaagatattaaaaatgcaggagctatcgaaggttctaaacgtaaaatgaacgcagtagaacttatttttacattaaaccaaacatcagaggagtttagtaacggatatttgagagcttacgtagataagttacgtaataatagtgggattgcctatgataaaatgttgtattttaaagtactcccagaaactatgactattagagatgaaacaccagaagaacgagcagaacatgaagcactgttagcagataatgcgatgaatagagcaagtagccattccgatgagaataactacacagcaaacgatgttaataaaaagataagtaatttgaataataccctttcaggaggttggaattaatgaaacatattattaatttttcagattttcatatgcacttttttaaagatttttcaaaaccagacccggagtatggaactgatagggcaaaagagcaaattactatattagacaacttgatgaactatgcacgaaacaaaaatggggatgttttatttaatggagatatgttccataaacgagtatctattgatgttagaatatttaatatgttatttcaagtaattagtagctaccctgatgttgatgttatcatggtcagtggtaaccatgataaagtaactaattctctatattcagatagcgctttagcaccatttagtgctttaccaaatgttacagtttgttctacgttaaataagattgttaaagatgattatacgttgtatgctgttagttatggggaagaggtcgaagagatgaaagcttggataaaagagcaagctgacaatttagaccatgaaacagttaatattctaagtgcgcacattggtgtagatggctcatctactggaaagtattctcatacacttggtggtgcttttaaagtagctgatttatgccctgacaagtttgacatagttacattgggtcactatcataaacgacaatttttaggaaacctatctaacgtgttttatgtgggcaataccctgcagacttcttttgcggacgaaggtcaagaaaaaggtttttatgatattactatagagggtaagaagtgggaacaaaaatttattaaaactgactatactccgtttgaaacagtaacagcggatagtccatcaacaacaggttctatggagaaatcttatatccagtttattggtaatgtggacgaggttgaggctgtaaagagaataaaagaagagaacaacttaagtaatattagaataaaagtccagaaggactaccatgttgagccacgtataaacataacagcaggttcgacacctagcgaagttgtcaatgcatttattagtaaaaagtaccctaacgcaaaaataattaaagacaaagccctcgactgtttgagagaggcaatggaagtgtaataattgttcagcctagatatatctaggcttttttgcgttgtactattgactttattagtattttgaagtataatatgtttattgaaataattaattaatattttgggaggaaaagatgttaaagtttaaacgagttagcgcagagaactatatgtccattggctctgtatctattgatttagacaaccaaggactcgtacttatcgaaggtataaatgatacaaacgaaacatttcagagtaatggctcaggtaaaagtactttactgtctaccgttacttatgcattgtatggagctacccctagtggtttgaaagctgatgctgtaattaataaacaagcaaagaaaaatatgtcagtgattttagaatttgaaaaagatggggtaccatatcgtatcgaacgttatcgtaaacactctaaacataaaaatactactagatttttccaaggaacaaatgatataactcagaaatctgtagcagacactgataaaaagattcaagatgtgttcggtattgattacctgacttatgctaatagtatcatgtatggtcagggtaacgtagaaatatttgctacagcaactgataagggtaaaaaacaaatcttagagaatttagccgatattggtgtttaccgatatgcacaggatgttgctaaagaaagagcacaaaaagcactagctcttgcagaagagctgaacagacagtacattgctaagacatatgaaaaagatgggttaactcaatcttacaatagtgctttacaacaatatgagaatacagaaaagctgattcaacaaaaagagagtgagttagctaatgcagaattagctattaaacaaagtgagaagaatctatcagagggaagagcgctacgtgaacccgaattagaaaagctaagagagcagatggcacaattaacttcccctgcagacgttcgtgaaattgatgtagaagttgaaacacagtattctaatgttagtagattgtctagtgcgaaaacacagaatgatactgctattgagaaattaaaaaaagaactagaagatgttaaaacaaatacaaattgctacctatgtggtgctttattgagcccacagcatagagaacaagaaattcaacgcattcaaagagaaatagtggataaagaagtgtttatcgaaaagcttaatagtgcattagcagtgtattcccctctgttagagcaagcaagggctaaacaagaggaagcaagaaaagctattcaagaacatactaatatttatcataaacttaatggtgaaatgaatgcgttgtatcatgagatagatacattagaaaacacattgaacacatctattaataatagggacagcattaaggatatgttaacaagactacaagaaatacctaaacctcaatatgactatgacaaagaccgggaaatagaagatgagctaaataacattaatcaattaaagttagatgccgaagaagaagctagtcaatataaaactattgcacaagaaattttctccaataaaggcatacgctctgaggtacttgacctagttacacctttcttaaatgaaagagcaaaccattacttatctaccctttcaggttcagatattgaaataaactttagtacccaaacagagaaagcagatggtagtttagctgataagtttgacttagaggtagtgaatggctctggtggaaacacatatcaagcaaactccgagggagagaagaagagaattgatttagcaatatcttttgctattcaagatttagttcagtcaaaagcaaacattgctgttaatttaggtttgtatgatgagtgttttgatggtttagatgcaattggttgtgagaacgttattaaaatattgaaagaacgccaaaagaacattagtagtattttcgtaataactcactcggagaacttgaagccattgtttgagaacgtaatcactatgaaaaaagttcaaggtcgttcttaccttgaagaaagtaaataggtgattacatgaagatttatacattaagtagagagttaaacgaaggtactatatttgtacctacaagtagtagtaatgaagggagactatttagcttcccgctagagacactattcgattggtacccttgttgcccaagatatgagtatcagtacagcacttctcgaaaaaaactatatttgagattgttagactcagacaaaacattagtagctagatacggtgttggggataataaaaagagagttatttcaaaaatagcttgctttaatgagaatgaatggtataatgaagaagtggcaaatgagaatgcagagttgtttaactttgctgaacaatatgacattgttacacctctaaaagacgattatacattaaaagaagtagatgatagtatttctaaagtactggatattcttgatttactttatacaaaccaaaaagttaaagtagaggaagagctcataaataaagtggatactctacaattgagcaagccagactcagatgaacttaaacaagcgtataaggacatatctgagtatatgcggttagacagagaggaaaaggctacttatgtgttaagcaggtcactagactctttaaatagtgtttatgaaaaatttggtaatgtgtatacaatgttaaatattatgaggaaagtagtggcttaaatgtttacagacttgttatctaacgaattaggttcacctaaatatgcagtaagggactacaggtataattgccctttttgcgactacgatactaagtataagttttatgttagggtagaagaaggacacccaaagaataacttatggcattgttttaagtgtggtagttcaggtaacccagtatcttttgtaatgaagtattacaacgtgtcttttaaagaagcgctagagatattagaagaatatggctataggtttaataataaaaattatgtgcctaaatcagataagttaactgatgaggaatacttattacttcttctaggttcgttaggtaagccaaaagaagaaactaaacaagctaaaaaagagttagtagcacccccattgccagatggttttaaactactaagccagaacctgagagagccagaagcgtaccctttcttattatactgtaataagagggggtttacattgaatgatatttatatgcacaatattggatatgtaaaagattcttgggtacccttagaaaacggtaagtcagttagactcaaagaccacttagtttttttaactcatggaaaagatggtaagtatcaatattggaatactagggctattggggaaagctttattaagtctttgaacgccccaagtaaagagggtgagcattcaaaaaaggatactatatttaatattaatagagctagtcaaacccctcagatagttataacagaaggtgttccagatgcattaacagttggtgagtctggtgtaggtacttttggtaagcaagtgactgacgagcaagttgaacttattttagatagtgtgaatgaagagcaaaaaatatttatctatttagataaagatgctaaaaaagaaattaagaagttagcagagaaactatataagagacataacgagacttatgtagttataagcccaacaactcaagatgcaaatagtctagggagagaagaggcttggaatattataaataactactctgtaaaagcagatggagtagggttaataaaattaatgttatagataggagagggaaaaaaatgaggtatacactagaagatttacatgcaggtatgaaactacgctgtacagacgataaagagtttagccattggactactggtaaaatctatgagctaaaggaagaccctgcaggtcacatgtatattagggacgacaatgaagagaaagtctgtggtataagcatcttagggcgtttaaatggtaacacagggagcgcagaatttgaggttgttgctaaggtgataaaggatacagattacacagaagaagaccttgaagaagggatgttatttcactgcaaagatgatatgagcttcccatggtgggctacagggcaaacctatgaaatctataagggtgaaaaaggaattttatttaccaagtcaggagacggtaaccaatactgtgctaaagaaatagtagctcgattaaatggtagtgcaagcgggtcttttgaattactagaaagaccgcataaagcagagttggaaaagaaagtagaagcaagaattaaggaactagaaaaagagaaacggcgtttacttgacaagcaacagcaacttaaggtggaagaaaacgaagtatctgtcgaaatcagtaaactacgtgaagcattaaaagcaattgacatactaagagaatttgaataagaatcactataggaggctaacaaggatgggaagagtatcagtatattttctaaatagcagaaatattatggatgaggatgagacaaagcgtacttatcatggaacatttaactcgatgaaagaagcagagcagtcagttcgtgactggtggaaagcaaatgactttaagtgtagtacccttagaattattgaaggtactgaggatgggattgttcgttgggactacggaaaccacactggattctatctatttgtaccagaaggtgctgtagtaaaatacacaatccgtgaaggcgctaagaaaccaaaacgaggaagagaaaatgatgtagcacatgacttattcacagcagacgatggagtagttattcccgggagattaggttcaaatgttatttctactggaataaaaacatcgtttgaccccaaacaatatggtttattcattaaccctcggggtggtatgatgaaatacccaatcactttaggaaacacacaaggtgtagtggaaggggaatatcgtggagaagttggtttaccacttaaaaatacattctctttacaattagatgcaagagctgtctctaagaacgttttaacaatcaatgaagaaggtaaacttattaacatcccagtgacagtagctcggtcaatgtatccaagctttaatgctctttatgaaaaacagctagagaagctaagtgaggagttacagctagtctatggaggagaagttagaatatctaatgctgatgagtatgtggttgcgggaacactatttatcccaaaaggcactcgtttatgtcaagctttcttactaccacgatacgacacacagtttgttgaagtatcagcgttaggcacaactgaacgaggagaaggcgcatatggttcatctggggtggtatagccatgctcatcccagaatttaaaccaccattattgtacgttatgggtagcttttctgttatgttagaaaaacatcagtgcagtgtaacttttgatttaagagagccttatttaggtacctcttacgataaaatagtgaagcttattaaaatgacttacccaaactatagcttaacttatatagggatggcagacaataaatataagttcacattaaagaataaggaggactagcatgtacaccaagaaagaagaagtagtaacagttaaacacctagttgataaagagcaaattagagtaggggacatagtaggttacaaaaagactattagagggttcgatgcaaaaagagtaaaagacacactctcagtaacaaatcaaataggtgtagtttctcgtgtatgtgacgagtatattactgttcatgacttttttgataagtgctcacgagaaatatgggcaaagacatttgaaaatttagaagttcgcaaaatagaggatggtaacagtctactacggaggtatgaaaatgaatttcgttgattattttaatcagatgcagaatttagtgattgaagaacaaacagatgagtatgttttattagagaaagaacatggtcaaaagtatgtgacatatcaagagctagaaggagcgttaagtacagtagcacgcaacactgcatttatggtagaaaattataaccttatgcaagatattaatttaaaaattgtattgaaaaaattaaaagacagtggtacaattacagaagagcttgaaaaagagattcttaaagagttcaaaaatattgaaagtttaatggaggacgaaacatatgagtaaagagagtaaacgcaacaaacgtatcggggaattatcggaagcagacatgagagtgtgggctgagtggttagctacggggcaggttcatgataaaaaccaccaaaaacagttagaacgcttaagtaaacgttcagtatcattatccgatgtaactacgattgtcgaatttatgggcaaacggaatgatggttatatttcttctttaattgaacagcaggctgtatttgacaatttattaacaaaactaggtgtaacggaagagaaccgcttagaggctaaagcagagtacgaaaaagagttaagccttattcaagaaaaaattcaaaaagagctagagtcaattaaagaaaataaagaaaaataatatgaaagaggttagctgaatgacagactacagcgcagtaggcaagaaaagtcgtaacaaaggcggtcgttttgaacgtcaaatggctaaagagcttacagattggtggggatatgaatttaatcgagtacctgcctctggcggacttcactgggcttctagtaataacgttgccggggacattgtagtccctagcgatgctaacttcccatttgttatagaatgtaaaaatcgtgaagactggacgattgaaaacttattcttaaataacaaagaaattaagaactggtgggcacaagttgtgggggatgcaaaagaaacgaaaaatatcccactacttatatttactagaaacagagcaaagaattttgttactatggcatataatgaaaaacttgttaatgagattgaaaaaagaggttaccctttgatggtctctaacataacatatgtagacgactacaaggatactcattgttacaagacatttactacagttttagatgcaataactagctttaagccttatggtagcaaagacaaagactactttttattttattttcctagtgactataactgggaagatagtctagtttatgaaacaaccataatggatgatgctaaacaaatggatgcagaggattcactagatgcactagttaattcttatttaggaggagaatagcatggctaaaacatatcaggaagctctagcaacagtacagtcgtatctcgaatcagatagtgtaatgaaagaaacctcaagtatatctgttagtttctcagctaactggacaggtgagcgggaagactacgttattgatacattgacatacgacattgatttacgagtattcagcttagaaactgcacatgttgtagctatagggaagaaactacctcaagatagcaatgagcatgctgagctccttaaaagacttaaaaaagaatttaaacaagcttctaaaaaactacgggaggactagagtagatgatagacaacgttaatagccctagtcattatacgcaaggagaaatagaggtaatagaggttatagaatatattactgctaagtaccctgcggaaattagataccatttagggaatgttattaagtatatttgtcgagcacctttcaagggaaagctacaagaagaccttaacaaaagttcttggtacttgaaacgagcacaactggttttaacacagtcaccaagtagctatataggtaaatgtgagaaatttttagagaatttcctatttaagaatagccatatcagagatgctcagctaatagaaataccagaagaacctattattgagaaatttttatttcaaacagcacagagttacaataaagaacagcaaaactatattatttcagccttaatggaactaaacagtagttcaggtgatgtgaaaactgttcttgaaaatacagaaaactatttaaaatttattacaacttagtacaatataatgctctttctacgaaaagctatcttttatatttatggtataatagagatagggaaattaaaaagtagaaggagcattttataatgacaaaagcacccagagtgaaaagacttaatatttataacactgaccggtactttaatattaatttaatgaaaaaagaagatatagcaaaaaaaattaaagttaaccgattgaatgaagaagagatagagagggaaatggacgaactagcaagcaacccattgaagacccctataggctacatggatagaacaaatgaaaaatcttatatactttatcaagaaaagtatacgaacgacagacttattcagaaattatttaaacatgcagggtctgtgtcttattacacagatacaattgtaccatactacataattgagcaaatatctaagaatttaacaagtgaagtaatctatcctacaaaaaatagctatgagaacagagagattgaaaatgttcaactagctttcactgcttgccctgtaacgattgactgcccagtagtgctacctgatgttagcccgtatgatgtgttatttgcactacatcctctaaaaacaaatgtagacaagattcaaatatcgtttccttgtcttacagaagaagagttcgatactagacatgaagaatactaccacaaagtaggtagccactatgaggttaaatcagagtacaaatataaattctttaagtatgtacaaacttccttatctatttgggctatgaacatttggctagtgtgtgatagcgatgaggactacaataaaatagatagatatattcagaaagaaaaaattaaacgtaatgctaacagagagcgtgcattaaagagaaagggtaatcaggatgagtaaagataaaacgattaaccgaacagacatagctcgaacaatctctcaccatactggttatcgaatgaaagatatattgaagattttagaagtagaagatgaagtagtagctcaagcagtatcacaaggcatctctgtaaagaatcacaaattatggaaactaaacattaaaaaaaagccagagaaggtagcatgggatggtataaactctaaaagttttatacaacctgaaaaatatgtagttaaatttgtaccattatccaagttgaaagagtcaatagacacttataataaagagagcaaataaagctctcttttttgctctttctatattgacaaataacataatacaaagtatactagtgtcatagagcaacagagaggagaaaagtgtaattgaaaattctattcttacaagagtacattagagaaaatcatgtccataatggaaagaacggacaaacagttgattttaaaagaacagaaatgggtaaaaaactcacaggtctgttaaatacaatagggctgacaggtagggactatgctgtagactatgtgtacgacatgattccggaagtacagaaagttaaccctagaacaggtaaaccaattaagtataagacacctacgttaagacagcgtaaagaaccagaggaacggttactcagacgtttaatgaaatacaaaccagatattatcatcccaatgggggagatgggttgtaaaaatttattaggaagtacttctatcacaaagaatagaggagtaccaactaagaaaacaattacgaatgagaacatactgagaacggctgatgagcaagggcttgaggtagacgaagtagtagactcttttgaaacttgggtactacctatgtttagtatggaatactggacagctaacccaaacattgagaactttatcatggcggatattgatacattgggtaagtttgttcaagaaggggaacaagcatttatccctaaaaaagtagattacgaatttgttgataacattgaacgtgtaagacaaatttttggttttttagacaaaacgaagccagtaactgcatgggacttggagacaaatagtttacgtggtgacttattaggggctaagcctcttgtaatgtctatgagttggctagaggggcaaggtgtgactataccgttagaacaccatgaagctaaatggagcccagaagagcttaacgaaatatatgacttatttgagaagtttttagcggacagacagcaaccaaaagtaggacaaaatatccagttcgatattagattcttaatgaacacaaaaggatttacagagtttgaagacaacagagatacaaaaattgcttactatctaattgtttctcagaaagtagacacttcaaaacggctatccgatattgcatatgaattaacagatatgggaggctatgacgaaccgttagagcagtataaaaaacaatataaagaggactatattgctcgtaagaaagcagagattgatgctttcaaagaagctgaaaaagaacgagtagagtgtgagttcaagatagcaatggatagatataagcaagaagttaaagaagctaagcttattggtaaacctactaagtccattattaaaccagtaaaagaaaaagttgtagtacctaagaaatcagatattaaacttattaatgagattgacggtggtaactttaactacgactggattccattagagattatgcacccatatgctagtggagatgtcgattgctgtctacgtatctataacgttctatataaacgaattgaggaacatcctaagatgttggaattatggttgggcttttatcctagactaacagcaacattagcacacatcgaggcttctggtgttctattagactctgaatacgcagaattagtagagaatatctatacagaagaagaacatcggttgcttaatgaaattagaaagctaccagaagtaaaagaagtagaggatgaacatatgatgttctacaaagaggggcttaaagaaatgacaaagcctgtcaaagatagagatgcctctgttgccagactacgagataaatataaaattactgatagtgagaacaaagtacattttaaaccaagttcgtctgaacataaaggtagactgctatttaaagtaatgggacttacactaccttatgataaagaatctattaaaaaagaaacttttgacaatggggtaccagaacatcagctaacatggagagattataaaacagataaacatgcactagcttatattgcagaaaattatccagaggctaaacatgttgctgaacttttgctagagtattctaaagtaaacacactaaaaaataactttgctcaaaagttacctaaattagcatctaacaaggatggcatgattcatggttcatataacagtacaggtacagaatctacacgattgagtgcaaacaacccgaatatgcaacaattaagtagtaaggttggagaccctagacgttttgactataaatatccaattaaacgtttatttagaacaagatttgagggaggtggaatgctacaactagactactccgccctagaaatgcgtattttaggtttgattgcaaaagataaagcaatgacacaagcatttattaatggtgaagatatgcatgaggcaactgcatctcttgtatggaagttacctaaagataatgtaccaaaagatatgcgacaacgtgcgaagagtgtcaacttcggtattgcatatggtgaaagtcctttttcaattgcacctaagttaggtgtaactgtagaagaagcagagcgtatttttgaagagtactttgcaagtaagccaagtattaaacaatttattgaagagacacatcaatttgctcaacgttatggctacgtagaaacactacaaggacatcgtagactattgcgtgactcattctctaaagataagaaagtatttaatggagcaatgcgtaagtcagtaaacacaatcattcaaggaactggtgcctacttaactaacctatcaatggtatacattgacgagtatattagaaaacacaataagcgttcacgtattgttatcactgtacatgactcacttgttatcgattgcccacgtgatgaagtggacgagatggcaaaagtggctaaatatattatggagaacttgccaattgatttcttaatgattgagtgggaaggtaaagagatgcgttaccctattgtggcagatgtagagattggggagaattacaatgatatggttgattatgatgcagaaattatcaacgagtttgcttcttaccgaggatacgtaaaatacttcaaagaccaagctaagattaaagattactatgataacaaacttatctcagaagagcaaaaagaccaaggtatcaaagttatccaagatgcaatagagtcatataaacaaatgacaatttaataaaaaaatgttaaaaatagcttgactattgacgtatagtatgttattataatatggcagacaagttttctgtattaatacaagaagggggagttagagagtgtgcttaatttacgagtagatgatttagagttcaaaacaattaaaattattgatgataacggagaagttgtgacacatgatttacaaacagagttacaagtcaatgaatttaatgtgagaacagcatttttagagcaacctgctaagtatacttattggacttcgatactagagcgcttgcgcatgtatcaagaaaactatgagttaaaagcagagaagaagaaagcagaactatatgagccttctcgggttgccttaatcaatcaaggagtagctaaacctacaaaagaccagattgaggcacagattatgttagatgaagactactacaaacttagacagagtatagttaacctatcatttaacgtgagacaactgcaatatatagttaaagcttttgaacaaagaaaagatatgcttattcagtatggagcagaccttcgtagggaatatgagtatagccaaaaggttagtatgccagacccaatgaaaaataaggtaaataatggtttctcagactttcaatggaatttagaacagtaaaattagtgataaaaaaaataatataatataaaaaaaatagaaatggagaaatttaaatgaattttcaagaacaattacaacaacaattaaaacaacaaaatattggagaacgtgaagcggtagactacccatcaaatcatttaaaacataaagaattatacttccctaaagcagaaaatggtcaaccatcaactctatatgtacgtgtgttacctcctgcagtaccgggagaaaactataatgttagcgctcgtgaggcattcttaacaactcgtaatcgtaatggtaaagatttaaagagcaactttatcttttcagaacaccctaatgcagaagacatcttagaacaagctatgattcgttggaacgcagaaaatcgtgtaccaaacccttatagccgtaatacaaaacctcgtcaacgttactacgttaatgtagtgcagttaattattaaccaacaaacaggggaagttagctacgaaacagattctaatggtcagctaatggttcgtttacttaagttaccacaaacagcttgcatggctattaatgagagcttaagtaacccaatgttacgcccacagttttcacctgatgtaccagaagaagtagcacaatacagctttatttcttctgcagatgcattccctatttcaattacaaaaccaccacgtagtaacaaacctacttcgtacaatgtacaagtgattagtaatcgtagtttaggtgcattaccacaaggttgggaaaacttattagaagacttgaaataccaagctacaccttctgtagagtacaatcgtgagtttattgagtactttattgatgtagtagacgggaaagaacctgtgcatcaaggagcacaaagtcaaggaacccaagctccacaatttaatcagcaaccagtacagcctcaatttaatcaacagcctgtacaaccacaatttaaccaacaacctgtacaaccaatgcaacaaaatactggttggtcgccacagcaaagccaaccacaacagcctgctacaggttttaatgcaacgaacatgggaacacctccaaacattaatggagggtttaaccaacaacctgtacaacaacctgtacaacagcaacaaccgatgggctctttcaatgaacaaccagtgacagacccttctaccatttctgatgcggatatgcctttcaatatgcagtccatgcctgatgtatcaccacaacaaaatgctgtacctgagcaacctgtgactaatacaccagagccagtaagccaaccagttgttaaccaacagccaaataacacacctagtgtagacgacctattagcaggtatggtaggcaacgtttaacaataatgaaacagttaggaggggcatactgccccttttaattctgtgttaactatccaaaatatttagtaggagtgaataatattggcaagaaaaagaaaaagtgaagaaatagattttggaactattgatttgacaaaagaggtaggactaactacatttacggacacaaagttctctaatgtatcagacagactaccaacaatgattccacagcttgattacattttaggtggaggattgccatttggacgtatggtcgaggtctttggtaagaactctagtggtaagtccactttagcagtccatctgacaaaggtagcgcaaatgctagatgtaccaactgtatggattgacgttgaaggtaccgcagacccagaacgcttagcagaactaggggtagactttagtgcaggcggagtattcatggtagagcctaaacagaacaaagacggtagtaaagatacaatcacggtagaacgtgtagcagaagagttacaacgactcttaccagtatttagtaagcttggaaaacctgtattaattatttgggactctgtagcgcaaacagcttctgagaaagagttagaaaaaggtgtaggtaaccaacagcccggaattaaggcaaaagcaatggcacagtttgctcaaatcattgcacctttaatgacaaattcaaaagcattatttattgctattaaccaagcacgtgatgagctaggtagtatgtttggtggagtagactctcccggaggacacgctttacaccactgggctagcttacgattagaggtagttaaagcaagtcagattaagaataaagagctaaatgcattcggtgcagaggaagagacctatgtaggacatatcctgcgtgttaaaacagcaaaatcaaaagtgtcccgccctaaccaaaaagcagaaatgtatttaatgtcagacacaggtctgaacttagaggaaaacatttatcgttcatgttttgcaactaataagcagtatgccttgattagcggaggcacatggaagtcttatacaacggatgcaggacaagagattaaattcaattcagataaagcttgggtagcttatttacgttcagaagagggaagacctgttcgagacgaactatttgctaaaatgatggtacgctcattcccacatcgttatgctccgtttaataatgaggatgtggacgtatgcaaaattccattatatgaatttactaaagaatatatggaaaatcataaggaacaacctaaccaagctactaaagaagaagtccccgaaacaggaacagatgtttcagatttacttaaacaggtagactaacagataagggggcgtttagcccctttattagctataaaggagaggaacttattttgcgcaaaggactagcacctaacccattttttgaaatattagaaaaacatcaagactcctctaaacgtaatatgactatgaacaacagtggtacacctagttcactacaaccaataagagacatgttcttaaaggcaatgagagaaggtaagagagtcctaatagagaactctgacttaagtagtgcgaactctgttgttatagagatagaatatgtaggtaatcgttggtgtttaggttatcaacgagtcttattttatggtatggagttaaaaataccacacactatccacttctgtgatgtatatggagcctatgggcacgatgctcagaaggttaagagacaagttaaggtagtgtttgagggggacaacccttttgagtagagatgttcaaaaagaggaaaaagaaatacgtaatggtaatcggtttattacagagactcatggcaaaggagtgtttcctagagatgtagaccgattgtaccataagtatagtaaccttagatataaagtctataacacccataaagactcgttcaatagtgaggcttcacgtaaggaactcaaaagctatattgatgagcaatttataaagttaacaaaagagtatgatataaatggagaggtagactttccgggatatattaaaaaagctctgaatttacgagtaaggcacagctatgtaaaaggacggttccgagataccgctagagaacgtctaggcacccaagataatgaagtagagttattgttagggattgacgatagctctcaggcagatattgaggatgcagaacttattgaatcgttattatcaaaagccaatttttcagaaattgaattagcagtatttcagcagttaattcaaggaacagtaagggatgctcgtattattactgaattgtcagaaaactacggggtgtctaagaaagccgtaaaagatgctataaaaaatgttcgtgagtttgtcttaataaatttaacagattagagtaatacctcctttatgtctgctatattagcagtagaaacataaaggaggtattttttagtggaacaaaacaacactggcaagtatgcaccatttattcgtttaattgtgatgggaatctcatttgtggcaactggtttgactacaatatttggttgggaacctttaccattcacagacgaacaaatgaaccaaggtttaatgttagtactatctgtaggtcttgccatctacaactggtacaaaaacaacgctgtaacttcttatggtaaagcaaaagaagaagcaggaaaagaagtagtcggaactagacaagacttcaaaaacagagactaagtacgaggggtgtaacccctccttagttaatacaaaggagcttcgacatgaagatagacgagataagcaaattagaattgcctaatctatttggaaaattccttgtggtagagactatttcggatgggtacactggaaccgtatctggtcactataactacgagatagaccaaaaatctgaggaaacttatatttaccctgtattttggaatgataagcttaacaaatttattaggtcagatgaattagttgtatacactaataaaaataaagtatactatgtttgtaaaacaactatagacccatataatcatgcagtagtagatgagctcacagtagaggaaggcatggacaaagataagcgtacccttcaagcgtttaaactttttgtaaatgacctattctcatttggtagctacagtatctttctaacaggtaacctatcattagaaaacaaccctgatattgttctagttagcagtgtatcattagacaaacagaccgctaaaatgtacacagaacggacattagaactaactgctacggttttaccagagggtgctacaaacaaaaaagttagtttctcagtagataaaccagaacttttagggttaacagtctctgacaataaagcaactgtcacaagtaaagataaggcaggtactgcaattgttaccgtaactactgaggatggagaacacactgataagtgtacagttcagatagaagaatacataaaagttacaggaattaatgttagtggagaatctgctttagaaaaaggtaagacatacaaatttactgctagtattgtacctgataatgcaacgaatcctaaatttacttggtcttctagtagtgataccatagctagtgttaatgcaagcggtgacgttgtagctttagcattaggtgaagcagacattatagcaacaactgaggaaggtagtcatgtaggtaaagtacatgttaccgtatcagacccagaaccagtagaaccaactgaataacatagaaaagaggaaagaaaatggctaaagaaatattaaatattgaagacctattaaaaccagagacattagaagtagcgatagatggtaaataccttattgtaccaacattgtcagacgggtttacaggtacagtagcaggcggatatgcatatgctgttacaaaaaaaggaacagactatacagttaatgaattaatctataatcaaaaggataacacatttaaaccttccgatgaaccaattattataactgatgataatgaaatattcttcattactcgtacattggaagacccatataactatcctgtagttgctacagagaaacttaaaactaaagatgtaaaagaaaaacaagttttacaagcgttcttagcatttgctgatgacagatttaagctaggtgtttacaatgtgttcttagcagatgaaccatttgtgtatggggacaaaacagaatagtttcattataagagacccgttaaggtctctttttttttgtatatagtgttgacaatgttgtagtttagttatatactatacttatactgtataaggaggtaaccacatgaacgacaataaggaaaaatgtattaaacaaggaatacgagatacttataaagggtacgatattttattagatgaagagaatggatttttctatgttagcgtacttgacccagatggcaaagaaatcattagtgggtttgtagaggcggataaacctattgaagaatactataaagagctcctaggtaaatgcgaccaagacatattattcaaagaccttttaagacccttaaatggacgtagagacactgaacgtacagacatacgtttttttaaaaagagatggtggggtataagtaaatgacaaagataaaacttattacaaaaaagaacacccaaggctatgtcatgaacaccctgctacgtagattttataaaaataatgtagacgtagaatttcttaataaattcaatctacctgatattcataaccacataggggggcatgatgctgttatcatagtagggtttcccttctttgaaagccaacgaggtgcgttagacacggcactgtcatctatggacaacccatttagcaaggtgtaccatttagcaacatttggggacacatatcgtaatgaaggtagctttcgttcatttgtagatgaggttataagccccgttggtcattttgtagaactaataattgacctaactaagttcactaacacaggtacaaaagaggatgaacaaaatgtagtggctcttgctaaagaggcattagtgtttgccaaagatattatagaagagacagacagctacaatcgttatgaagttacagacagaactatctcttgggttcttcttgtagatttgcttggagagaacctatataaggtgacagagcctagtaaagagctagacaccattctgaaagaacaagaagtacttgtagatgccttgaacataaatatgcaggaatatgtgctaagaacaatagggaagatgtctgcaaacgtcattaatggaacagtggtttgtttcggttatgcagaacagcatgttaacgaagtagcccataaattaattaatttttataagtcacataactatcaaaaagttattgtctttattggtaggcacacaaaaggtgacgacatgtttagtgtaaggagctatggtgtgaatgccggagaggttgtctataaagttcataatggtaaagggaaagacacaacggcaaccgtcttcttaggtaaacctagtgaagctgtaaataacacactgctaagtgtgctttctgaaattttatagtttaactttgtggtataataaagatagataaattttgataggagtgtaaatttttgaaaaacaacgaacctttagagaagttattagacaaattagatgaaccaagaattttacaaacaattattataggtagtttacaacggagctttaatcgagtacacgttggtaaatttaataaactagcacaggagtttgacttagataaggagaacctgtatagcttaaaggcactagttaaggaaattgaagaagataaagagctacatgaactttacgaagctagcatggcaggcaaaattacgctagaggctgttcgtaaagtattgttacaagatgataaatcatcgtttgatgtactgtcttcttacgtagtagaaaatcaagcagtgctagcccgtaaccgagagttcggtaagttacaacgtgagggagcttatcttgaccatctaattagtggtttgaagacatacttattaactgaattaaaagacatgtctagtttaaaatatatcaataaaaatttaaaggcaccgaaagtatcctcagaccgagagcttattctgtgcctatctgattggcatattggtgcttttgttaataacattgacacaggtggatacaactttgaaatctttaaagaacgacttgaaaaattattagaagaagtattccaagtggctatggagcaggatattaagaaaattcatgtttaccatattggggatattattgaacacattaatatgcgtaatgttaaccaagcatttgaagcagagtttcctgctacggagcagattgctaaaggaattagagttcttgctgatacactcaatttactagctaaggcagaatttgaagtgtctttcggtatggttggtggtaaccacgaccgtttccaaggtaataagaatgataaaattcataatgataatgtggcttaccttgtagtagaccaactccatttcttgcaagaattaggggcattaaacaaagatattaaacttgtagataacagaagtgacgtttatagttttaaagacacagttgcaggtaaacgtattaaagtaactcatggtgacactgagggcaaaaaagtagatgttaaaatccctaaacatatcaaagatgaagttattgactatttaatcatggggcatatccatacaacacgtattattcaagaagacttttcaagattccatgtgtatgtcggctctcctatgggggcaaacaactactcagcagagaataacttgccaacaacaagtcctgcacagctaattatggtattagaccctgagcgtgatacgccacagttcatgccagtattcttataggaggtactcgtatggaaggtaatttagtttatattttattggctattgcatatgtaggtgaaggtataactgcctttactaatactaagcgtaaagaaagatacatgattgaagaaggagaggctcctttgccacgtagttcttatgtatttttaggcattaactaccttttaagaatagctatagcaatttctcttatcttcatcataccaactagcttgcaacttaacgtaacaggtattgctttgttcacactaatggtatttgcagttccttttatagcaagaattatcgaagttgttattagaactgcaattgttcgttatgttcaaaaacaatatattaagcagttagaagaacgaaaaggtaaaagagagactaactagtctcttttttgctatattgaataatgaggtgactaagtatgaattttacagaagtaataagccctaatggggagacatcattaatagatgtaaacaacccaccaacattaatcagacgtggagtcttatctattaaaacaaaggttaataatgaagtgaaggaaactcctgtttatattgttgaactagccgaggagctaacaggtactgatgtagtatctgtctataaagtaaaagagattggagattctatccaaaaagactacattgaagaaaaagtgacccctaggttcaaaagcacaacatatctgggtgagttagcacaaaagattaaaggacggtcgataaaggaacaacgaagagttgaaacaaaaccaccattattcctagccccagtagttaatggaatcgatacattcacaggaatcgaaggtaaaggtttctacgaacgtgaagaggacagacatattctattacctgatggaaaaccgggcatagcctatggagataatacaggggtttttatcggtttgagctcaattaagtgggacaaggcatatgtagacgtggagtctataacaaagggttatttgtcacagaaacagatatggtttaacctagatggtcaaagaccacaatttagaagtgagacactataatgacagataaacaattttatgaggcagatattcaagaattaattttaaataagcaaagaatattcggggatataggaaagagcgctattgtttttgagaaagcaatcatgcaaggtaatacaatatgtgattgcctagtcttcacagaaaaacgagggctcattggcatagaaataaaaactgaacgtgactcgacaaaaagattaaataaacaattatctgattatgaaaaggtgtgcgactacgtatatgtattatgccacgataaccatgtacctaaagtagagcaaatacttgctagacataatcataaacacgtaggtatcctagcttacacagagttcatgggagaggcaatgttaggtgagtataaacaacctagccgttcccctaaaaaatcagcttatcatatgttaaatatcttgtggaaagaagatttaataagaatgcttggtacattcagacggtatggtgacaggctagaagctaatggagctaaggttatgaagacaaacagccgttctggtggggtatctgggctttatgttaaatctacaactgctagaagaatgactaaacctgaactcattaataatttaataaatagagtaggcggtaccgaagaggctacgagagtgttctgtgatgtgtttatccataataggaatcacccagagaaagcaattaagttaagacattttaaagcaaaagagaatagaggtgacctagatggggtttaaaggtgcaaaatatggttcttggaatactgtagttggtaagaactatgtaggtactgggggcagaacaagtggtagtaacacaaaacggctgtccacaaaaggctactaccaagtaggatttgttaaagaatatcagaacttgacagaaaaagatattatgttaaaattagagtatggaaaagacttagtgtctagctacactggtgtacctgcagatatgattaagctacgtaaaaagaaagaggagcaaactctagcatctttagacacagtttattatgttagcattggtaaggaacctgtaggtaagctgtctatacgagctcagagacgttttagagaggtagggttaacatttatatacctagagaagaactacgtacagaggaagcttagaggcggtaatgtacgtagtgtaggatatactaacgcaaccaaatctcagaaacgtaaagcagatagacggaagagcaataagtctaaaacaactagatagatggaggactactatgagtatttatgatgagctaattggtaaaagtgttatagccctagaatatgcggaggatgggtgtgaggcaagccttgtactaaatgatgggtcatgcttaaaagtatcttgtaacccagagccagactgttgtgggtataacgattttgaagttattctcccagatggttttgatttcacagataacattattacaaaagttgaagataatagtgaagaatgttatggcggttcaacagttaggataggcatttttaccaatgatgcacggatagctattgaaggggactacggaagtggttctgggtggaactatggagagtatgtagacgtagaaattgtaaaataatttactattagacggaggtaagctatgcttaaaaaagggaaagaagtaacactaagaaaattttataatattatcacagataaggaatcagtattgctaggggcacgttacacaagctcaataatgactacagatattcctattacaacaacttttgaagatgttgaggtagaccttaagaaagaaacagttgcaggaactatttcattcagaccagtaggagaaagacagattaacgcattaagtttgctaaaagaagcaagcacagcttatggaggttacggagagttcctagatgatactattgagaaacctttaatcaatagtgattttgcagtagatgttagcctagcagactctgctttctctaacctacaagagattccatttaacctatatatgtcctctccaaaagttttttatacagaagtatcaattagagggcgtaaacatatgcaatatgtattagaggatgagtcttctagcggtgtaacaagtacattagcactggtattccgtaaaaaattgtatgatggggaaacattgctaggtgtgcatacttacaccgaagcactagcaagagtagagggaattaaagtattacagtttatcgcaaacggttctgtactagaacaagtaatcggtgcagtgtctgttttaggaggtagcacaggaacaaccctgttccctatgtttgacgatatggtgatgcaatttgtgatgattgagtctgttccttgggtgcacatttcttgtgatacaggagcaattgcttttaaggaagaagacatccgcaatgtaaccattaagtcagctagaccgggagaatataaggtgactatctacttgctagacgaaaaagttacattattaattggataaaattttaaaggaagttgacaatatatgttgacttccttttttgtatgtgctaagatagctacagttggataaaaaataaatgatatagagggagagatttaacatggactacaaaacaaaggtatatgtaggagcgcttagttggttaggtactttaggagagaaacgatatttagggcaaatgcgtgatgtattagctgtgtacgagctaggtagtcgatacgcaggttactatacagaagatagcgactatgactatatggttgtatacatgccggctccttatgatttaatgcatcctacaacaatctataaacaggaaacagagattgatggaaataaggtagaggtaaaatatatgtctatcgtggaatatgtttaccgtattgaaaatggagatttagaggctttacaaatgttaaatgccacaagctcccaaagtttcttcggagagcctattgaaggtattgacaataagcgtactcgtttagttacgtatatgaaagaactagaatacagaagagaaacatttacttatttagcaccagagaagttgtttagaggaattaatggtcgtattaaagccacaaaagcacgtatggataaggctattgagaatgacgatatggaagtagcggtaaaatgtgctatccttattcgttattttatggacttattagttgtgctagcggacggggaatctattcgtgaaggtctcacgttctctcctattattgcaaatatcattagagagtttagggaagactgtgagtcagcacaggctaagacacttatcaatactgcacaagtgctattagaggcggatagagaagagattttaaatagtattaaagaccatgggttatcagatggttacgctcaagcagttagacaatatagtttaacaggtcgattgatagatgtactattagggggatactatgattaaattagagcaaaacacagaaattttaaatatttttaatggtaaagggagagtggcagataaagtagaagaatctgccaaaattatgctagaattagaccacgattatgactttggtacaggagacattgcttatacagagcgtggcacagataagaaaggacgtttctacctagaaagtcacttgtttatccataagattatgccttatgggttcattttaaacgctgtagtaagtaaagtgcactatggagaccaagaggagaccttagaacgagaggtatctcgtgtatatgagctagaatataacctacgagataaggtagctgtgctgattaagaatggtaaaccactaaatttctcagaaaataacattagtaacatgttttctgggcacgtagcacagaatattcttgaacaattagatacaatgtctggttcagatatgtatgtatctgtgtatgaacgagttagtcgagttaaggatgaacaaattggtaaagtatctcggttctttgatagattgatgaagtacaacaagattgaattaatttataagagtggcgttcctgaaggctttgctttggcttatgcttataaagtagtcgttgtgggtgaagggaaaaatagggataaatggtaccaacctgtacagaatgtggactatttagacaaggaacaaacaaatccggctaaacacctaggtattccaaagtctatcttcaagattatttgtgaaggcggactagagtggacattctaccaaaaaatggctagtcgtttccataagtcagtggataacagaagtattgtaaaaacccctgaatctcctactgctatgaagaaggcagagaaagaggttgctaagctacgacaagcctatgctcgttttggcggggtactgtataacctttatcagtttactaaagagctagatgaacaatatggcatctcccatagtaaggatattatcgaaacagagtgggaatctatatacgaccatattattgcaggagtagttaacgataactataatagtacaactgcttatgatatttctatggttgcaaacctagacttctacagaactgttcgttatctatactatcgagtatatgttgagcaagggcttacttcacaagcagaggctaggtacacgtacagagactacttgagagcacatcattatatgaatgcggtacctgttaagtaccctaaagctcttaagactgctcatgacattattaatatgaattaccgaacaatgaaggataaggtattgaatagtaactttgaaagtagtgtagctgaatataaatatttagaagaagtatcagttagaggtggctatattgttaaggtacctgatagtgtggaagacttaactaaagaaggttctagtttacaccactgtgttgctacttatgctcatcaagtagcagaaggttctactcgtatcctgtttatgcgagataagacagagccagatactagcttagttacttttgaggtaaaaaataataaactagtacaagcaagagggctagttaaccgtgacttgacaaataaggagcaggagttcctagacaaatggttagctaaagcagaaattggtaagtattagtttgcctaaaaaggcggaatatggagtgtgtatagaccaattcacaatgaaaaaagtacactaaacagttgttatttcattattaggtaaaacaacttattttagatagctgatattgaccgacaaatatttcataatgatttagctattaaagttacaaaaaccctactaatatgtggggtttttgcttgtttcaattacatatgaaattttacagatgttctgctatattagttacagaagctaataaaacaaagggtttcgacccttaaaaatgatatttcatataattaaaagttgaaggagtttataactaaaatggctaaaaaagaagtaaacaacagttcagtattactgaatctatacaataataaattgcttgtatctaaggtagacgaggcgttagacgagggtaaaccatacgattttattattgctttttgtaaagagaagttcgattttgaaattaataaacctgcattatctaggtacaaagagaaacgtagagagtctttagaaacaggagtagacttggaatcactacttgacaaacgtagaaaatcaggtaaaattattgatattaagtctaaagaggtaacaccacttcctaatgaaacatacgataacacatttggacaagtggaacagatatacaatgatgtagaggtgttagacacaatcatacaaaaaggttttaactctctaaaagaggtagactatgttgaagccccacttgctatgaaagcaattgaagtaaaagctaagataacagctaaccagttccaaggtctaagtctaacaggtctaagagagctaagattaagacagtctgctaaagaacaagcaatgactgagattattctacaatttattccagaagaacagcatgaagaagtattcaatgcaattgaatcagcagagaaagagttctatgaaaacttagacttaacagaggaagaccaaagaatcactaaagcgttgcaagcatcaggtatggatataatttaggagggctataatggttgaaaatttgagagaggtaaattataaaacgttaaccttagaagaaagtttacatgcgttacttgaagggaagacccttattgtaaaagggctagaacaaagacgtaagttagatgtgctagtaaggattttctcagaaggtgttgtacctgttacgcagataagttatgatacaacccctgcggatggttattggagaacagggtattggcagatatatgatttaccgattaacgcccttagcacatacccatgttttatctatgatgatttaaatacggatgaactccctaaatttatgataggggacactgtttactacacaagtaaagaggactctatcaaagattccgccattgtaatcagtgtgtacaaagacgatgttaacaataagtggtactacaagttaagtagagacaacgaaatatatgcagagagtgagattagaagagacaggttataagcctgcctctttttttaaatttattgttgacaaatatctataactttgttatagtataaatatcaaggatattgataataaattttggaggtgccatgatggcaaatattttagacacattaaagtggttagataaaggggacaaagttactattgaatttgataaagagcggtcgaagtatgcaaaactgactttgcacgattcatcagctaagacaaatattgtccgtaacattgttttttatgatttagacaaaggagtgtatgcttacactggtgaatacgcccctgtatgggataacttattagatgatatgcgtaagacacaaggcgcagaacctgaaattaagactacaaaagtaaatcagttagctacctatgaagatgccttaaagtttattgaaacaaatggtacgttttatgtaattggtgaggaagtaattgttaaggttaaagatgcaaaagagctagcattgatgttaatgtactttagagatgctattgaagaactaagaggagaatacgaccctaaaaaacatcatgtagatatgacaattgaattatcaaaagatgttttgaaaaaaatggcagtacctagacatgaattagacttatctttaggtggcttaatgagagctgtatcacataatgttggagaagacctttttgaaactcttgggtttgactacatgaagcaagcttgggaagtcttagttaattgcttgtcactagacactatccatgaggtacctttccgtgtgctagacgaattagagaaagtaacagatagcatgtctactacagaccatattgttaccttgtatgccggtagagaacttaaaaaattctattcagaggaagagtactttgatgtaatcacacacaatttagctgatgtgattatggattggtcaactatttttactacagctgttcttaataatacagaagatgatgaacaccttaaagagttacagcttaactttgagagattcaagctagatgtagcagaggttctattaggtaacgttgcaagacatttactatatgcaggagttaccgatagctttaacgaagttaaccattatgttgtcggagcaggtaacctgattagaacagaaggtttgattagaatagaagagttaactggaacattagacgaccctaaagatgaagatagttctgaaacagataataaagagttactagatgacatctttacaaatacaggtgatgatggtgttgaagactttacaaaagaagtagacgagcttgccatgtttgagaacgaacatgctgaggaacttttggaagtcaataacaaattaaaagacaatcaagagttattacgtaaaactgtggaggcgatgggtatggcttcttactctactattgattcatcatttgaacaagaggaagaaacagatagcaaccacacagagacttgtgaggataccagagctttccaaatgttaagctatgcagtacgttcaggagacaaattacttgaacgtgtagagaaccacccacaaactgaaaaagggttgaaaatggctaaagattctctaaaagagtttgaccatacagttaaaccagaattagaagcttatttaaaagcagaagagctagcggaagacgttgatagacatgcaattgtaactacaattatccgtatcaaggacactatggaagcagatatggacggctgtaccgacccaatagagcaaggtttattctacgcaggaagcctaaatatgttagaagaaatggaatcattgttacgggctagcaatagaggttatggttgggacgtaacagctatctatcttatcttgtccattgaactagcttatgggacttacgggttgtctgatttagactttacaattaaaaataaagaggaaactcgtaaagaagaacaagatgcaattaatggtattgtaaacttcttagccaatcttttaaatacagtactagaagaagaatcagaatcagaagaaacacctgtagtagtcgaagaggaagaggaagaagaggataaggatgacttctcactatctacagaagacactgctaagctattggcagactggtcaaatggtctacctacgtatgtcgttactcgtaaatacggaattagtatgggagctttatactctatcttatatgcaaatggggcagacgttaaatcttctaaagtagcagaacgagtggctcatgtggaaaatgacaaagatatgttaaacgcagtcattcgagactacaagaatggtactcgtttagtagatatttatactaaatacaaattatataaaaatggtttgttctatcttttagacaaatatcgagtgccacgtagaggtcgtacaaagaaataacaatgattaaggtagtggtgttaaactactacctaatcttttattagggaggaacttaattgattctttttatatttagcattattacaatgctatccatgttcttactatatttgtttggaatggcttctgtagccctaattgaattagggcttcttataggtagtcaaaatgatataactaaaggtatacactctttacttttcacaggtattgcacttactgtagtcaccgaaataactagacagtgtttactattattttaaaaaattggaggatttagtatgttgttttttattgttttagcagtatttataggcgggcttgctttatggggaatgtatgatagttatgggtacctagactggacttcatggttatttagtattgtagtaggctctgctgtagctacttttttcacactggcggcggttggaattactttcgatgttgcaccaagtcatggggtaacgaaaagtcatgaattacaccctatctatgagaacagtaaagtagttgtagaggctaaaaaagaccagtttgagattaacgtagatggcggtttggtagctattgatgcagaaggtactactatcctatctactaaaggagaggttaagcctaaaattgtgtttacagagaactatataaacaacaactggtggactcgtttcctaggtatagcaggtaaggtaaaagacacttcatcagttctttatttagactcagataccttagtgtacaataagccggaaaagaacagtagcgcaccagatttaaaaattaaataaataatttagaaaaaggggcttgcataaagtctctttttatgttaatgtgtatttatagataggagggctactatggaaggtgaagtagtatacttagatgagtttatgaattttttaactgatagtggaataaatactgatgatattaacgtagtagatgatagacaggagggctagtatgtcttatacaaaagaaacattagaagctagaggctatcagtttgacaatatgtctatgatagataagatggatgccttgttagaactgctagaggaaccagaatttcgagaaaagatgaaagaagagtacgcagagttctctaagaggcacaataacgatgaagaatgaaaagttagagtcttaccttatgaaagagattgcacctattttagatgaaatagagatgagtgaagaattgataaagggaatggagaacaacaacccatccgatacggttacagtctctttttccaaagaagaagtagacatactcttagcaatgttagaccttgagttacctagattagacagtggttctgaattgtttagtcctaatctggtaagggaagctaaactacaattaattaagaacaagctaacttcaaaatgatttctcactagaattaattagtgagaaaaaaattaaataaaactattgacaaataacaaaacatgatgtaaactaagtttataagataaagaagaggagagatacacatgaatgaacaaaagaaagtatatgcaaaattaacagaggacgaggcagtatttgcttgtgaactaggagataaaattaaacaaattagagagagccaagagctatctcgattagaacttgcaaaacgagcaaaagtagaccactcaacattaatcttaattgaacaaggtaaacgactaccaacattacgtattatgatgaagttgagcaaagcactacaccgagaattagctattagctttacagattaaggaggaggtatcatgagagggagcctagagtattacaactatttgtaccaaacaatcgaaaatcgacctacggaggagctagatgtcctttatgatggtctttataaaaaagccggtgacctgtttgcaatagataacttccaaggagttaaagaaggcagactaattttaaaaatattaaaggctattagagaagaaattaatagtagaattgatgaagaaattgacctttacttgtataatatctatgacagtatatcagatgaagacaaacgagtaaattggctatatgaggtgtaagtatgtttagtgttgttaaggctgacagctatgttaaatcagacatagaaatgtatgaatttaagaatgtaaaaagtttggcattaactaagaaagagaaagaactatggtcagacaaaattgctaaatatgctaatgagtttattgtaaatgcttatggagactaccatgggaataaaatacctgaaataaatgttataatcaatggtaagctaaaaaaagtcctagggtcttttgtccaattcactaatacaaacatacactatatagagataagtggtaggtttgttaaagaagttattctattacaagaaacaccactagctcaaagagcgcttgatattttaatggatgtagtcagacacgaggctatccactatacactttgttatttaaatagtttaagtgggggagagcgaccaacgttcaactaccatgacggtggggaggactttgaaaaagatttatgtttgacaggaacttcacctagtggcgctacgaaagaagagtatatttatagttcttttacactaggagctatcagatgccgacaccatagtacttgccctgaatgtgggttggaaacatacatgtatacacgaggacggtactattgctataatgggtgtgtaggggataacggtagaaaaattatatttagaccacaaggtgatattgcaatttatattgatgaaccaaaatctaaagctaaaccaaaagtagaggaagctctgaaagactataaaggagcgttaaagctaccttatacaggtacggaagaaataaaataaaaaaatagttaaatgtattgacaaataacaaaacatgatgtaaactaagtttataagataaagaagaggagagatttattatggcatacgtaacaaatattgatgtagtagcagatgggttagatttatataatgggaactatgtggtagaacgaggtcaagtagttacttttaaactacatgtagcgacatggaacaatgagcctacacccgaaaatgcttatgcgattattcgtaataatggggtagattataagagtaaagttgatgaatttggcaatgcagaggttaccttcccagttaatggtcgtcctgaccaagtaacaactagtatctttgcattaacttctggatatgagggggatatgcctcgtgtaatgtcagctatctttagtgacgaaacagaaattaaacaaactgtaatgaacgttacagcatctattaatggggagccagttagtcgtgaaggtgtatatttagaacgagaccaagtagtcactgtagatgttaaagcaacattatctacaggtaagttctgggaaggtgcacaggtaggtgtgtacaataacagcacggaatacttaggtgacctagatgcagatggttatggttcagtaactttccaagtaaaaggaaaacaagggatggacacatcagctatctacgtattcgttaaagaccatgaacgagaagctacactaacagttcctgtgaagttcactaatactaccgttactacagaaacttctacggaagaatcgtctactgttgatacgactacaggaacagaggagtcttctactacggatacaacaactgtagaaagctctacaacggattctacgtcttctacagtagattctacagaaagtacagtagaaagctctacagagcaaacagtgaccaatgaagacacttctactgattcaggagtagttgaaactacagaaacttcccaagtagcaggctacacagagtctacttctagttctacagagtctaaagaagttaaggaaactcatactagcactacagaacctgctaaagagttaccatctacaggcacagaggtagactatgggcttgttgggttcggtggtgtaacattgacagttgtagtggcactagtagttaaaaaattgttgaataagtaagaaacacaatactgcgaccctcattaattgggggttgcaaattatattatttttttaaataaacctattgacatttaacaaactataaggtaaactaagttcataagataaaggagggaaacaaatgagtcaagtaagcaaacatggagaaaaacgtgtacgtgaacgtgtaggagtgaataagagttcagtagaccgtcagttttacttagcgttagaacgaggataccgacaacatgagctaacaggtcgtctaaagaaatgggtagtatcaagagtatttaactctaagtaccctcaaacatgtattttatataatggtaaatgcttcattgttagcagtgaaggtacactagttactgtgttaaatattccaagtaatttactaaaagattttgcaaaattatctaagaaaagaggaaaataaaatggatattatggatattgagtttttagaagaacataaacaattagtaaaagaacatgtagaacaagaattaaaattaatgcaccctcttaagaagttacaagtaatgacagaatggttagaagatacagaagacaagctttctcaaggagacttagattacttcaatgacttaacagaaacagagctaatagaggcaatggatgctagtgaaattgtagagtcttattcagatgtcttgttagattttattgactactacaatattgatttaactggtttagaggaacagctaggtgtgtaaccatggataaggctgaaaaggtagataacattgtaagacaggtcacaggggctgttattaagacaactgcaaaagtagcttttattgtttttgttttaacctttgcaggtgttttagtaggttattatagttattcatttttaacaagtgcagggtggtttgccttacctatgatattatcagtagatttgctatatgttgcggtactgttaggcgggctagcctttatatttgcagaggtctacaaagtagttttagaagtgaaaaagatagttaaacgaggagggcaattatgagtaataaaacattagaacaaagagtaatagatgctaacaaagagataaacgataagcttaacgaatcatcaattattcgtaaacaaattgaagaattagaggaacaggaagccatcttactatcagacgtagaggacttacttgattacttagaaaatattggagtagacctataggagggacttattatgaataaaaaagtagaggaaatgacaatggaagaaaaagacaaagcactaattgcaatgggacttattgatgtaacgcaggaggcagactgggtagtattagctacttgtgaagaatgtagagaagagtacgaaggagaggagtacgaagaaggggactgtgcagagtgtgaacattgtggaggagaatactttatgattgagacagctctcgagggtactcgttgtggacgttgtgatgactactttgatatgtgggacgactactttgagtttgagaatgaaggtaacccatataaagataaacatatctgtgaacattgttatgaagaactagtatctatgggaatggaagagaaattttaagatactactaagaaggagacttaatcatgaacgaattagaagaacttaaaaatacactgattagacaaaaactttcaatgttggagagttacgaaatgagagaagcatcattttggataatgtttaatgggatattacgtgttatcatgtcagtagctgtcatagcctttgtaaactatgctaagcatgtaagaccagataatgtagccacatggtttctagctctgatttgggttattttcctagcagaaggtattaaaggtgcttatgatgctgttgcatttggtattcaccgcaaaaaatttgctaaacatattaaaaatatgcgtgggataatcgcaatcacacaattacttattgaagaagacgaagagaaattaaaaggaggcaagctagatgagtaaagatgagaaattagttgagtggtttggagttgccttaatggtgattatgtggttgcttatcacgttcagtatcttatacacaatagtttccttacctttcatggtacacgaaggggactggctagggattgtgcgaaacgtactactggacattgtagtattggcaattggggttgtcgctacatggttgcaattaagatttaaaaaaggaatggaggaataattaatgggatatttagagagtgcaattgaagaaattgaacgagtactactaggaaataaaagccgtgacaccgaagaagtttatttaaacaatgcaattcggtatatcaaaaaagagttaaagaaaaaagaggtagagccaacgtggttaaatgaaccacagacattgttccttaattggtttaacgaattatacgcagtaggtggtttaactcatgtaacagaaacagtaggttttttagaatccacaggaggtagaatgaagtatccagaagcatattctgctttcagtaatttaagtgagaatgaattgcttgaggtttacagtagcttttatacagggttattcttaaaagcacaagggggtgaattagaatgaactttcatggtaaagtatttcatgataaagtatttgacattctttctcgtgattaccctgattggcagaggtatcagacagagaaacgaccacatcccaatgagcttagaaaagactttgccattgatagtacagatagtagatatgaagagtatgttatgggagagtttaatgtagaaacggctagcggagatgttaaagtatacgcagtaggaattagaagagtagttcatagttaggaggtagttatagttaaatgaccaattactcagaagaactaaaaagtttagagaaggagctagcttgttctttgaatgatatgtggcacgctagtgtcgctatgaacgagagttactatgcggattgtataaaacgttatgaaaaaatttcagtacgaatggctgaggtaatacagaaggaagctgaggaggcaggggcacagtgaagaagtatacagagaaggatattaaagaaggtatgaaactacgttgcacagatgataatgctaatgtaagctattgggaggctgatagagtttataaggtaacacgtgataaagaccttggcttagttatcgcaggggaaggcgtaagaagccacagaaccgtagagttcattgtaagagcattaaatggtgacgatgcagttaaatttgaagttgtagaggaaaaacctgtacgatttgctaaggtaacttgtgtataccctcctgatagaggtcttgtagaggttgggcattgttacaaagtacttaaagagttccctgagggacgtgtgcgtatttaccttaatagtaaactagggaaccatgtgttactcccagaccagtttgtttttgtagatgagccatcaaatgatggagaaaaagacgtagaagagctagatgtagaagctaagattctagccgatattgaacagctaaaaacagaggcagagtgcctgtttgctaagcgtgaccgtgtaaatgagcaagctcttaacttaaatgcaaaagctcgtaagttagaagaatctttagaggtactaagggagtacatgtagctaagtgtttttatgattacatctattaaggagatgagagaatgatttattatattaattttttagaagattttgcatcaagttggagtgcagataagcgttatcgtgttcgtagaattatgacaactggtagctacgctattattgacaactatggtcatgtcagattcagcagtgacacagcacgaggtgtccttaagaatatagagcaggagtacgccactcataaggttgagcttacactagcagaagaagaaacaacgaataagcctaggtttaaagtaggggaacgagtgaaagtgtctaatgatttacaggcattcgggatagaatataagactcacataacctctaagatgataggctgtgcaggtaatgaggctactattacaagtgtttgggggagtaacgtacgctactttgttaacatcgatggtatacaccaagattggtgttggacggaggacatgttagataagattgaagaagaacctgcactatctgttagatgtgttgaggcagttcaccccttttggacaaaaggtaaagactacgagataaaccttacatctgatggtcgctatcgggtctgggatgatgagggagatggtagcagtggcaaatccataaaagaactactggacgttatcaatagtggcggtaacaaatttgagctactagatgaaacaccctcagaaacagagcctaaggtaaaccatgacatgtctgacttagagaagatagaggctaagattactgccttatcagaagagtcttttcagctattcgagaagagtgaagagttgagtgctagggcaattgaactgcaagatgagtcagtagctttagaagaagcattgtatactattaaacaatatttataggaggagatttaaatgcgtaaaaatgtaatttgtagattagaatgtgtttcaaaagacaaagataacttaggtgaatggactgtagataatatttaccctgtgtttgaatcagaactagggaaagtatacatcctcgatgatgaaggtactacttgttccagagatagcgtgtctcttattatctctagtatggctagctttggggtaacatttagagtagcaaaagataaagcagaagaccctatccctagcaatccacaaagcagtacatcattagaaatagttaaagggtatgaacacttagaggagtttatagactcattaagtagcaaccaacgtgtagtgtctcactcggtagaccctaactctcaatggcactatatcatttatgaaactaagtcagctgaattgggtggagtaaccctagaggaactactagacacactagtgtataatgtacatgttgtagtgatggaacgtagtaagagaacatatgagccactagaaagtcacacgtttaagtgggaatatggaacaaagaataccgaggattgggaaaaaattgtacctttattaggttgcaaggtgtttaatactagccccaatagcaacagagggttccatattactattttgaaataaactgttgacaactactatagtccatgctatagtaggtacatagataaaaaacaaacaatattaggaggtagcatagagtgaaagaaacagaaaagaagtatagcagagagtataaggggctagaatttgaaattattatcacttattaccctgaacaagacatgtattttgtaattgtagttaacggacagcatcgtacactaattaaggttacagggaaagagtgggaaatgagccctcatgcaaaggaagctgttattgagaaggcacttgatacatgctacacctatattgaaaatcaagaagcgcaaaagaactagattaaggagggactatgatggtaggggacttcattttatgggttaaacaagcatggaaagaaacgttctgcattcatgactacacggttaaaggtgtctataaaacactagatagtcatgggtacttaaagtgtaacaagtgtggaagaattaaataggaggacaacaacatgaaccagagacagaagattaaacaagaaaagaaatggtttgaggagcatgggtatgatacacagcaaccacgagaatgtattgagtgcggggctcctttaagttggaaagatgatatgcagaaaagtcatggagtgtgtagtgagtattgttatatgagaagtgttgggctgtcgttatcagattttatttaaggagatgaaacactaatgtttaaaaaagataaaaaagaagaaaagacttatagagaaggagacctccttaaggctgtagggggttactacccagatgctaggttgagtttaggaatcacatatccattgtataaaaccaccaatgagggttggtatattatcaataacgaaggtagtcgggtaaccttaatcgaaatggatgctttaggcattgactacgctgtgatggaagaacccttattagaattaaaagaaggagacccattgctagtcgtgtccgaccttaagagaggtgttcgaggcttagcaggtgcagaagtacagtgtttaaacattaccggtgaaatggcttcactagcaggtacagttgttcattatgataaagatatgagccatattgcaaaggggctgttcactgttaaagagaacgattcttattggtgtgtagcgattgctattccactaaataaagtagctgaccctgattttgcattagagcatttgcttgctacgttaaataaaaaagcctctactaaacgtctggtattgaatgagataaaatatgatttaaaccttcttcacaaagagctagacgaggtgtccaacgaagtagaaaaactaacaaaaaacattgaaacaatctataataacagataggaggtatttgaggtgatttggtggcaggacaatgagaattacaaagtcacacgttttagatgtgcagaagacttaaaggaattcctaaatgagcttgacagggaagacgaagtcattactatttttaatgatggtcactacaattttattattataaagagaggtgaacacgttgaatgatttagagttattattaaagaaacctattgcaataaataagaatcctgatttttatgaattagcgataggtggtaaagttatctacaacgaagaagtaatggagattgctaaagctttctctaataagaagactaagtattacctgttaaacacacggagtaacaaacaagtatgtgtgccagtgtaccatgtaagaccttatgatacccaagtagaaggtcttaaactaggtgatgtcctagatgctatgcacattatgacaactgtaacgttaagggcagtgaacaagcacgggtttgttgtcgatgaggatattattgaatgtttagtagatgacatcccagaaaatgcgcttattgttaccagtgaggtatcccgtattcagccagaggattttggtaaggtaattattgactattttgtttagatatgttaggatagacttaagataggataggataaacttaggatgcgcttttgtttaggataggtttaggataagataggatataggataggataaacttaggatgcactttggtttaggataggatgggggatataattatgagttacgttaatgagtttgaaacaattggagattggttagatagagaaatttatgatgtgctactaagagatggacatgatattgacgaattagataattggggaatggctttgtttgccttaagtgaaggttacgttctaacagatggacctgataaaccattcctagagttaactagagaagacctagtagcaggctacaaccactttaaagaagaactgaatgggtggttacgaggaggtaaactgctagaagtctctgataatttatctacaattcaagggttcagttttgaccatgacgatgttttattcttagaggataacaacaaagcttatgcaatgctagtaggtattatcattgaagcaagagacacatataaaaatggtttctgtaagggttactatgtaataccttaccaagattgagttagccttatggctagctttttcttttaccttaagtagcaggttgcaaaatctcctaagtagcaggttgcaaaatctcctaagtagcaggttgcaaaatctcctaagtagcaggttgcaaaatccggatgcgcccggctagcggtggagagtcaagccatgcgggttcaccttaatagctaccgtccctagattgtcggtttttcttcctttatatagtaagaaaaattatttaaaaaaaatgaacttttctattgacaaaaaacaaaagctagtataatatagagcgtgtaaggaacaacaacaataaaaaaaataaagataaaggggcgctattaaaatgaacaagtataaatttacctatgcagacattaagaatttaccagaggaagaaaaagaaaaagaattaaaaaatcggtgtggtgttttagcggtagagtgcttaagcactaaacagctacaaaagaaaaaacctcgatttatggttttcttaaataccgttatttttgatagtaccgcagaaacaggcggacaatacgcaacggcaaccgttaaaaccgaaccgataggggacggacgttttcgagtgtgtgacggttggggacagctttctaatggaattattgaactgttaaaataatttataaaaaaaactattgacaaataacaaaagataaactacaataaacttgtcagataaagaaaggaagttataaacatgtttaaaagattagaagagtaccgcaaaaatttatccaatgaagaacttaatattttcaacaatacaatgaagctattaaatgataataagacagactttcaagaaagaacaaaaaattttgataaagaactacaatatacagagctagaaaatgaaaaagaaaaaattttgatagtattagacactattcgagtgagtaacgaaaattacaagaatgttatatctgaaaacttatatcattctattaaagaaatgacaattatttttgagttactttataacttatagaaaaaaaattaatcaaaactattgacaaaagacaagaactagcatataataaacttatcagataaagaaaggaagttataaacatgactatcgcagaaaatgagttatacttagttaaaaatccattaaatgagtgggatatgaactaccatttaattgaaaaagaaacaggaaaagactttttgattgatatagatgacgtggcagacaatgaagaagaaagagaaaaatatgaactaggtttactattttcagacttgaaaaatcttatagttgatttgcttattgagtatagttggaaaactatttaaaaaaaattaatcaaaactattgacaaaaaacaacaattgatgtatacttaattcataagataaagaaaggaagttatagacatgacagaacaacaatttaaaaaagagcatttattagaaccaacagaatggcgtagtggaggctacttagacactagtttaatcgaccaatcacaatcttattatattgaatcacgtcctagaatttacggcgggtgttacgtttatcaatatgttacaatgaaagatggcacggtctacgagctttacagcatgacggcaagcactaggggaattgttgcacataactgccacgctagaaacgtattgcaaagagatgtaaaacaatacagagaaagcgccattcattactagggagggttaacctcctttacatagaaaaaaaaattgttttatctattgacaaataacaataactattgtatacttaattcataagataaagagaggaagttttaaatatgaaattatctgatattattctaatggggttgcttgttagcgttgtactactttggggctaccttagcattatgatttgcttacaagtttttagagcgttaggcggttgggatattagaacgttaacggtttgcagtggtttgctatttgcctatgttttcggtttaaaaggaatctgggaacaagggacagggaaaaataaataaaaaaaattaatcaaaactattgacaaaaaacaacaattgatgtatacttaattcataagataaagaaaggaagttatgaacatgaaattaaacgttatccatttactattttgcttatttcaagaacaagaaaactattctattttaagttacgaatcaatagacgaattttattctaggttaggttacgacttagaaagtgagtggttacttagggacttgagtattaatggaacaagtgacttagcagagttgctaacagattacaataatttattagagaatgagataacaaaagcagtcttttctgataaatggttatagtttaaataaaactattgacaaaaaacaataactagtgtattatagacttatagagataagaaaggaagttgtttactatgttaaaaatcacaaaagaaattactttgggagagtttgaagcttgggagggtggaaaagaccgactagaagaaattaaagaattggatattttagaggaagcacagcaagaaatcgagttgatgattgagggggcggaagaggtaacagaaatgactataaacgatatcttatggtttgagatggacgagttcattgcacaatttgaggaagaagaggaagaagaatagaaaaacagggaggggcttcctccctttacatagactaggggggcttgagatgacgacagaggagaaagcgctaaacattgcagagaatagaggtataacagattataaggttaaggggaacgtattaagctattatactagctacccgatggaaaaatgcacatacattgtaactattgacattgaaacgctagaagaagaaagaaaagaactcaaaaaatattataaaaaaggcttgcaaaatgcttgcttgtagtctataatataaagaggggcggcggttactattaataataaaataaaaaaattgcaatatgaaagaatcagaaagctagaaaagagaaaaagaggtgaaccacctgaatttattttcaacggtaactattcattggaagaaatagaattattcttacattttagaaaagagggtaaaaatatgttaaaaacagaagaaataaataattcagagtatacaggataccttatattagatggggataacttaattgtacgagtagaaaaaaatagaaaaacaaaagaaacttactgctatatttatgcagatagcaacgatgatgattttacggcaaagtttaaaattaattaatcaaaactattgacaaataacaaaagataaactataataggtttataagataaagagagggagttataaaaatgacaaaaacagaactacaatataaaaaagcaatcggggttgcaatctttgcaacaagtgaagaggataaaaagcaactaggaaacgtggcaccgtttagtatttatgagattttagaaattgacttaaataaaaatcgggtatactatgccttaaactgtggggaacgacatgcagtgtgctttactaaactacgtaaagaagaagaaacagggaataattttattttaatcaataaacaaccttttttcttaaaagatatgcacaagggtttgacttggtcaaaaagtttataaaaaaactattgacaaaaaaaaacaagctatagtgtattatagacttataaacgggggaggggttaacctcctttacatagaaaaatataaaataagggaagtgctaaacatggaaacattagagaaattcggttatacatggcaaggaatgaaagaagttacaaaagaggaagcagagagaaacgttaaaaacggggttgacacttttctattatatccagataataccgagagtttagttgtatccttggacgaactagaaacagagggggtacgctttggggttgaaaagcccgtgcaattactatttaaggtgtctaacagttaccacaatacagaagaagtttactacggggaaaacgtggaagagatagaaaagaagttgcatgactcactagaaataacagccgaaaacctaacggggcaagaaatagaagaagattgggaaaactactataaaacatatgaggaagcttgggaagcaacttacaatggtattctatcacagttaaaaagtgaatgcactattgaaaaaatgtaaaaaagtttcatcttatctattgacaaaaaacaaactatagtgtattataggtttataagataaagagaggaagttttaaatatgacaatgaacgcaaacgatactttattactagcaatcgcagaggaacacggaacaaaaatagacaaccttaagagctaccttatcagtgatacagacagagcaagcattcttgcttttgtaaaaaaagaactaaacagcgcagtaaactttagtataaaaattggttgcacaacaattgaagcagtgaaagaaagcaatacagtattagataaagtattaagagcttgcaacttgctaccaactgaaaagaacgtgcaaacagctaaaaactttttagaagaacaaggctttaaagtagaagctagtaaagagttagtgggaaaataccttgtaaaattttcaatttaataataataaagaatgaagaggggaagttataaaaatgaaactaacagaaaaggaactaaacacaattttaagagatgatgaaacagggaacgggggaacggctttcttaggtgaaacactagccgacttcctagaagaatcaggtattgactttacaaacctaactattttagaagtaaatgaattactagaaaataatggaattagaccaattgaggtagtgccatgttaaaaaatatcaaaaaatcagataaactcactagaaaagatatacaaggtttttggggagatgaaacaaaaacattagaagaatggtataagtcaatttcaaaagaaccagacaccgaaaaagtagaaactgctaaaatgattaatacattgaaagaatatgcaaataacaacgaatttcatttcgtaaaaggagagcaaggacaatgacaaacacaaacaaccaacaatggaatcagaagtttaatgatggtactatgaaccagaacaaccaacaaaaggaagttatcactttacaagtcgcagaaagtttcgttagccagattttaacaaaagaatattcggtgattggtttagttactctaatgttttattttatgtttggtatgtttggcattttcgggggtgcaatctatattgctttcactcacacaaaccgtaaaatcggggcatggcaacaagtaagaagcgtatacactattaaggaggaacaagaagaatggaacaattaaaagggctaacaattagagaattgattaagaaactagaagaagtgccagaagaaaataaggacttacctatttatacttttgaaaatgaaaactctttgcctattaaaggtatttcattatatgatgaaaatgctaaacactcacaagaaaacccgttaagttttgatgtaatcagataaggaggggtaaagatgaatagttttatgaataaacaagctaaacaggtaaaaagaagtaaagaaataaaactagtggaagaagtaagaagagaaaacgtaaagaaacgcttttcggaggaagttagaaagtacctagaaaaagggtatacaatcaggttagaaaataaagtttttccatttgctttaatatctattgacctagaaaaaggggaaaaggttataagtcttattcttgtgaacgaatatgatggaatagcaaactatacagcaatgaaaaaagttaccttaagagaaaaagggaatagagctatcctgaaaaggacgttaacggataaagatataaaggtgataagtttatggaaaggaatataaaagaatgagcgtatacagtttaaaattactagcaatcttagggatagtattattcttttcagttatcgggatagtgtacgataataaacaggacgaaaagaaataaatatataaagaggtaaagaggtaaagagataaggaactatagaactataaagctataaaactatatacacttataggggaagagatggaggaggggaggaggtaaaggaaagagagggagagagagggagaaagaaagcaataaggaataaggcaataaagaaaaaaagttgtttacctatatacttatacacctatgtacctatatacagatagcccctaacaagcaatagtaagggaataaaggaataagagagagaggggaagaggaaaaaggaaagaggaaaaaggaaagaggaaaaaaagttgtttacctgagattttaaagaaccgacttcaccagaaacccgacaagggggcgcataagtctatttcaaaataatctgaaataagtatagttattactgtttatatatgattatccttaaatatctgaaaataggggcgtttattcgattatatagttaattatcttatactattaatcatctagcctattactgcattcattgctattatattctatatagggggcgcttatatcgtttatagtgtaattatgaggggaacgactaggaacgcttatacgggcttatagggggctttaaatgcataggctatttatacatatattagaatacacaaggaggaggggcggaaagttagtagattagactgttaaaaacaaacgggggatagatgcacattgttattttactgattggactaggttaactagctaaaataagaatgctattgtagcaacgtttataagaaataggggtgtacttttcccctagggtgattttaaaagggtataaaattcttttgttatttaattaaaaagttcatcttagctattgacttttaacaataactagtatagcataagggtataagataaagagaggggacaaggcacatgcaaaacgatatttattacacaatacaggacagggacggctttgcagaatcttataatgaatcaggtttcttacatgaactaggtaacatgaaaattattgattatacttatatagcactagagggggcagaaaaaggggacactgtggaactagtgaaagtgcctattttagagggctaccaatatccttatgattttttagtaattgatgaaataggtaaagaacactatatctattatgaagaagaagaataatttaaaaaaaatttcatcttagctattgacaaataacaataactattgtatagttaacttgtaagataaagagaggggagaacttaaaaaatgaaattaaaagaatttatcaaactagcagaatcaaaaggcgctacattagaagcatacaacgagctaggaggttatgaactaactagaggagatatagtagaccagaaccctattttaattgcttatatgcaaggggcttacagcgtagaaatatcaaacgaggaactagaaaacaaggagttaacagaactagcatttgtctataaaaatgcaagttttatttatccaaacgaaaaagaattattgtcaggactaggactatagcacaacggcatataaagcccgtaactaagttttaacgggctttatatataaatacattatgagggcttataagggcttatgagggcttatgagggggactaataagaatgctagaggtaggcaaatttgtacgaggtaacaacggctcaagaacttgggtgggtcagattgtaggtatagacagactagcgggggaatatcgtgtgcgtgacgttgtaacaggtatatacttttatgtatcttgtagcgctgtttatgaatgcgctagaccataaaacatttaataaaaaaaaattcatcttttctattgacaaataacaaactatattgtatactaggtttatcagatagagagaggaagttatagacatggaatatacattagacgaattactagaagaggaatacagcacactagacgaattgctagatagtaaagaatttaaaaaacaaatggacaattttaatcacatcccacaaatgcaaccgcaaagtcataacagcaatacgctagcagacacgggacgttatcctgaaaaataaaattgaaaaaaggggttgacaagttcagcccctagtggtatactaggtttatcagagagagggaggggcttcataatgaaagcaatcatcatcaaacaaatcaaggacggttaccaaggtatcttagaacgtaaggagaacggttggacgaaagaggaactaagcgactatacagcgggggtaagcaatacactagttgaccttttcaacgagttacaaggcagtagctttattacagaaaaagaagcggaagaaattgaggaagtattttctactactatcgatacattagaattttaaacttatctattgacaaataacaataactaatgtatactaggtttataagataaagaggggaagttattaatatgacaatcgaacaagtgaaagactacggtaaagcatacggaaaagagctatcaactgaggaagtgaaagaattttttgagaaacacaacggaatgccatcactaatggacttagcaaagttttgcggggcaatgaacgaggacggaacaaaaaattaaaaaaaactattgacaaataacaaaacatagtgtaatataataagtgtaagagggaaagggttaaacctaaaaaggaaaatgagtacttctttcttacacaatacataccagataaagaaagggagtttttaccatgacaccagaactagcagaaatgaacctaactaaaaacgatagtccattttcatttattaatgaagcaggggttttcattgaaccaatcaaaaacccgtttacttgtgagggtacttatttacaagcttgtaaagacgtagtgggagagctagaagcaaataaagaggtagggaacgacatcgacaactatttaaaaatgcaaagttggctagataagttagcatatgaatatatttaaaaagtttcatcttatctattgacaaaaaacaataactagtgtagtataataagtgtaaggaggaataaacctctttacaccacacaacagaaaggaagtttttactatgacagaatttgctaatatgaataaagaagaggtactagaattacttaatgattggtttggtgttagtgactatgatacagtgatggaagagttaggggagatgaaacaagttacctttacaggtagcacggacatacctctattaggtggtaacggtaacttaatcggtttacctcaattctttaaagacaatgaagcagaatcagagttgccaacttatgaagaattactagaggaactagagaaagatacatggaatcttgaagcagaggacaacacttacaattatagcggcttcctagaaagtgaatcagattttaaagttattcaggcagaaaattcagacacgactattgcattttttgcaatccatacgggtatagacataagagcgggctactcaaaagcaatcccagttatttttgaaacttactacgattttgatgagtttttaggtaactacttttacagtcaaggttactatgcttttaaacacgacaacaaggaatacacaatcagtttagacgtttcggctgtttcggagtatgtacgaatttatatagctgatgaaaacaacgaggaactacagcaagtctatgaacaagaaacttgtatggacttagacatagaaagcgtagaagcatacttgaaagaagagggtatttcatttactgacttaaaacccgcattgtaaccatataaggcactagggagcttatacaatagagctacaagcttcctagtgtaattataaggtaaataactagaaacgcttatacgaaagaatagggggctttaaaatgaagtttaaacgagcaatcgaggaattgaaagagaaagagggtatcaccttagaaccagaatataacggggtatatacagttaagggctactattctaacttagaggtattacattgtagcgactacgatgaagaatataataacgaggaactagaggactgccatttacatgaacttgtattcaggtatgagggagagcaagcatataagagcggatattatgaactgctaaaattattgaactaatctattgacaaataacaataactttggtattctatatttgtaagataaatgaagggaattgataagaatgaaaaatctaatgaatgaaactatctatagtaatattacattaaacctaactaagaaggcacaaggcattatatcccaatatgtagaccataacgagggctacccaagacaaaagaacagaggtcactactaccgctatacagttacatttatcaatggtgaaacgttcgaaagtaatagcaaggcggacattgctcactatatttcatctatgacagcggacgaactagaaaccatcatcaaggaagcacaagccgagtagtaagaaataggtatataccagttaaccctgccccgtggttttttgaccgtactttgggggtggggctatttgcc

SEQ ID NO: 2 - DNA, 143049 bp, synthetic construct
  source 1..143049: mol_type = other DNA
gcctaacgactaaatttctaggtagctaaccaaatggggttagtgagaactagataaaaactatgatattgaattgcgaacatgagaaaatgaggtaaaaaaataatggcgttaagaaaaactaatgaagaatgggtgaacgaagtacaccatgctgtaggtaacgattatgtattcctagagcactatataaacagtactacaaaaatactagtaaaacacaacaagtgtaatcatatctataaagtacagccaaaagctttcttatcaggtaaccggtgccctaactgcaatactgctagaaaactaacaaccgcagagttcactgttaagatgagtaacctcccaaaaggaggagagtacacattagtatccgagtatgagggacaccacaaaaagattacagttacacataaaatatgtggtaacacctattcagttcttgcaagtgactttataaaaggcagacgttgtgttagttgtctaagagctagtaacacaaaaacgcaagaacaatgggaaaaacaagtaagcacattatcaggtgatgagtataccttcttggaaccatacgttatggataacgtaaagattagctaccgtcacaatacttgtggtacagaacataaggtaacccctaacaactttataaatgggacgagatgtcctaggtgtaaagatagtaaaggggaagctcatatacgtaaattcttaactgataacaacattgagtttatacaacaaaaaaaattcaatgatttgcaggtagttaaacagctatcctacgacttctacttacctgaactaaacatactcatagagtttcaaggagagcaacactacaaacctattgacttctttggtggcagtgacaaatttaaatgtcagcaagaacacgataagctaaagagagactatgctaagcataacggaataggttttatagaagttaaatatacaaaagatacttacaaaaaagtagcaaactacttagctcccgcaatggctaaagcagttaataaatcatagtgtaaagcaagggttccttaacctaaattaaggaacatgatatagtctagtcccctaataaatatcgggaaaccgagggtataaacgaatcgtcactgggtatctatacatggtgttaaaacaaatggtacggtagatttgataaaacttttttctgtgggtaagtccaacccgctagaccctaatgcaatagatgtagacatacagtctataaaattacagctagctccttacaatccagatataatcgtagctgacgtaggagactcaggggataaagttgctaaacttatgcaaatttatggaaaagaacgagtttttgggtgtgtctacccatcaacccctaaatctactggtaacctagtacctacttggagcccacaagcaaataaagtatccgctgacaagttaatgcagaataaacgttacattaacaagatgaaagaaggagaaataggttactactcaaaaccagatacagagcttaatttatataaagagcactggaagaatgttgttatacgtgatattgaggacgaaaagacatcgacaggttttagacaaattatcggcagaaaaggtgatgaccactactcacaagcaagcgtttattccatgttagggtatgagtacctaatgaatgtatttacaggagtaaaagagtatggatttgactctgattgggtgtccactcagttagcacctacgaaacctgatatatttacagaatttgtataggtagattgccttatttagttttagtcaagctgaataaggcattttgtattgtttgatatatatttttgtggtataataaatatagttagggggatagttatggtagacaacaatgtaaaaataagtaaaagtacaattgaaggactaataaataagtctttaagttatgagtacgttataaaaaataacgaattgctgacaaacgaatatcaacatattgtcaaagcgtatgggtttgacaatttttatgacatgtacctatacgcagatagctgtgattctaaagatatgtatttagtaaaaggtggtcaaaaagacttgtctaagctgaaacctgttaaaagaaaagttgttagaaatggtaaaactatgactacaactatttatgaagatacaggcagttcagacagtaataacagtaaccctttagacaaagagagtaaaaagaaaaaagagttagaaccagttaatgctaaggaactacataaggttagcttaggtagcgatgaagaagagaagttagaccctaaaaagatagcaaaattgctagcagacactaaaaaatttggaaataattttgatacgcagtgtactgattatcttattcttgaacaagactctgttacacgaggagtggtagggtttactagagaaggttcttatttaaaaatgtctttctcaatgtcagatgaggctgtcaaaggtataaagatgttggctttttcacagctcacattaaaggcttggaaattgggtctaggggctaagataagcacagataatgcacctgatgtagaggaactaattagcttatatggatacaaaagaaataagacagagtatattgtgtcaatgagctcactgcgtagtcttctaggggagccttagtcataagtgctacgttagtcattatactagtattttcaactattatttttttaatttttaatttactagtaagtaaggtagttaaagaattttataacttaaagatgctagcaaaagagggactaacagataaagtaacagacataacaaatgatataatgcatatattgaaaggagaaataaataatatggaattaattatgaataataagaaactagatgaattaactaataaggtagctactgatgaggactacgatatttttgtagagaagatgggtaagctagttaaagatttgtatgagaactaccagtttttacaacaaaacccaccagaaggagattacacatcaggttattttttagggttccaagtaatcagagcagaatacccagttgagtatgagaatttatttagattagctgtagataaaaatttaaatgaactagagataaataagagatttgtcgaagctgttaaggatggcaaggttttaccattaggtgaggctgtcattgatgaattacaaacagggtgtagctctatactacaggcacaagaagtacgtgtaaatatcgtatttggtacaaaagaatacatggctaagcaagaagaagagcgtaaagaacgtcaagctaagttagaagaagaacgggaaagagctatggaagtactaaaaacaaaagatgacgtacttaacacattaagagtaaccgaggctttggctaatgaattaacagacgaagtcgcagaaaaatatgatttaatggagctagttaacagtatgagagaggggctgaaagctcataatggaaattaaaaattctttgggaaaagctcttataaaaaatttaagattgcttaaagaaaaaagagatgcaatgcccgataacttagagtatacaagtcaagttatgattcctgtgccctactatctaataaaaaaaggggataacgcagtagaatcattccttatgtgtgcggggatgataaataggaataaagatttaggtttacctatttctttagagaaaggtaagcaacaagttaagcttaacaatggagaactaacaaccattgtagaatgtattgctacctattcagataaaactgacattgacggtgtagaaagattcttagttgaacatctataaaaattaaattaacgagttctattttgtgctatattagatatgcatgaaatagaactcgtttcttttttatgactaactaaggaggtttaatagatggcaggagaagtatttagtagcttgattacaagtgtaaatcctaacccaatgaacgcaggtagccgtaatggtatcactatcgacaccattatcctacatcacaatgcaacaacaaataaagatgttgctatgaacacatggctattaggtggtggcgcaggtacgtctgctcattatgaatgtacaccaacagaaattattgggtgtgtcggtgagcagtattcagcattccatgccggaggtacaggtggtatagatgttcctaagattgctaaccctaaccaacgttcaataggtattgaaaatgtaaactcgtcaggagcacctaattgggctgtagaccctagaacaattacaaattgtgctcgtttagtggcagatatttgtacacgttatggtattccatgtgaccgacaacacgtgttaggacataacgaagtaactgcaacagcatgccccggaggtatggacgtagacgaagttgtacgtcaagctcaacagttcatggcaggaggctctaacaatgcagttaagccggagccaagtaagcctacaccaagcaaaccaagtaataataaaaataaagaaggagtggcaactatgtattgtttatacgaaagacctattaactcaaaaacaggagtactagagtggaatggtgatgcatggacagttatgttctgtaatggggtaaactgtcgcagagtatctcatccagatgaaatgaaagtaattgaggatatttacagaaaaaataacggaaaagacattccattttacagtcaaaaagaatggaataaaaatgcaccatggtataacagattagagacagtatgtccagtagtaggtattactaaaaaatcttaatactagatttaagaccatcttatggtggtcttttttctttctttgtaatattcgtaataatatgtaatggctatgtaaccgcttgttcttttggctgtaacaattacatgttatattagttcttgtaagcaacacaaataaagaaaatattgaggagaattttattttgaagaaaactagtattttaggtttaagtttattaagtttaggtttagtagtaggtttaggaaccgaagctaaggcagaagaagtaacagagaatgggaagacatattggaaggtagagtcaggagacacactatcagaaattggagctaagtacaacttagacttcactaacattcataaagttaacaaaggtgttgtagctgaccctaatatcattttcgtaggtgacaaattgttattacctttagatgaaaatggcaagctagcggaacaagtaaatgccactgaaccagatattgaagtacagtataacgcaccagtagcacctgaacaacctgtagttgtagaacaagaagttgtagagcaacctgtagttgtagcagaagcccctgcccctgtagtagaggtacctgctgatagtagctcagcaaaagagtggattgcacaacgtgaatctagtggttcttatgatgcaacaaatggtcagtatattggtcgttaccaattatctgcctcttatttaaatggtgactattcacctgccaaccaagaacgagtagctgatgagtatgtagcaggtcgctatggctcatgggagaacgcaaaatctttctggttagcaaatggttggtactaaaattaaatagcaataatgacccctttattaaggggtctttttttatgctatattaatatatagtaaatagtaatataaaatggttatgattagttatgtggtataatagacttatgttaaaaccatttaaggaggggaaacatgggttatattcaagatgagacatggcagatggttaaaaaagttgctaaaaagaatgggtttgttggtgattggattttaattatccactcatactatgagtatggtggaaatcacgtccagatacatacaacaataaacggagaaagctatagaattttaaggttgttagatagcagagagatacttttattagatagaaaaggtaaccctgtaatttatgattacgaaatagttaatgatggtcaaaaaagcttcttttataatgatatggaagagaaagaaatcgaaatacctaatggaagatgcttaaacgataagacaagggtaaaaatttatgtataaggtaggtgtaacagttgccaaaatggttagataaagcactaggtatagaaaaatcgtccatagaagaaactcggaatatggaaaattataagatgcatttaagggaaatagacaccaatgtggtcaataacgagccgtacagcatggaaagtattgaaaaaggtatgaatggcaagaccactgcatatatgcaaccaattattggagagatgtcagtaaaccccgggtataaaactaaaccgtctatacgtaactctcaagacttacataagacacttaaaaagtttggtaataacataatattaaatgctattattaatacacggtcaaaccaagtatcgatgtattgtaagcccgcaagaaactctgaaacaggtgttggttacgagatacgtttaaaagatattgaagcagaacctacttcacatgacattgctaatattaagcgtattgaaagttttttagagaacacagcacaatttagagaccctaatagagataactttacaactttttgtaaaaaacttgttcgggcaacatatatgtatgaccaagtaaactttgaaaaggtatttgataaagatggtaactttatcaaatttgatactgtagacccaactacaattttcttagcaacaaatggtgaagggaaactaattaaaaatggtgaaagatttgttcaggttatcgataacagaattgttgccaaattcaatgagagagagctagcttttgctgttagaaaccctagagcagacatagaagttgggcaatatggttatccagagcttgaaatagcactaaaacaatttattgctcatgaaaatacagaagtatttaatgacagatttttctcacatggaggtactacaagaggtatcttgcatgtaaaaactgggcaacagcagagccaacaagcgttagacatatttagaagagaatggagaagctcactagcaggaataaatggttcatggcaaatacctgtagtatctgcagaagatgtaaaatttgttaacatgacaccctctgctaatgatatgcaatttgaaaaatggcttaactacttaattaatgttatatctgctttgtatggaattgaccccgcagagataaacttccctaacaatggtggtgcaacaggctctaaaggaggctccctaaatgagggaaactcaaaagagaaaatgcaagcttctcagaataaagggctacaaccccttttaagatttatagaggacaccgttaacacatatattgttgcggaatttggagaaaaataccaattccaatttagagggggagacctaagtgctcaactagataagcttaaaataattgagcaagaaggtaaagtattcagaacagttaacgagataagacatgataaaggtttagaaccgattaaaggcggagacgttatactaaatggtgttcacatacaagctattgggcaagcgttacaagaagaacagctagaataccaaagaagccaagaccgcttaaacagactattagagctatctggtggagatgtagaacaaccagaaccagaagagcctaaagacagtcaaaatgatacagatgtttcatttcaggatgaacaacaaggtttaaacggtaagtctaagaaagttaatggtaaggtagacgacaatgttggcaaggacggtcagttaaaatcggaagaaaacaccaactcaactaagcatggtactgatggtataaaaaaagaataaaagataatggagggggctagctccccctcaactttgtttgtgttactagatagtacatgttctgctatattaaatacagtaaaacgtttaggtggtgaaaagcaaattgtcagaagttagagaaaaatatagtattttcgtaccactggatattgaaaattctatacagaagtctgaatctgtgaatgatggtgaatggtatgtgcaaggatatgcaactaccccagatttagatttacaaggagatattattttaccacagggcattgatatttcttattttattgaaaatggttggataaactatgagcataaaaatgatgctgagtttattataggtgccccaactagcaattgttatgttgatgtggacaaagggttattcgtagaggctaagctattaaaggataacaagtacgcacagtctatgtggaagctagctaatacaatccagaaatcaggaatatctcgtcagttaggtttttctattgagggtgcagtagttagtagaaatgcacaagataacagaatcatagaaggtgttaaaatacataatgttgcattaacgacacatccggctaacccaagagctacatgggagacactagttaaatcttggactacaggatatggtacagcaccagatgcacaagtagatgcaggagcacttagaagagagatgtttaaagaggacatttctaatttgacgtatgcagtaagaactattgcaggactatataataaaaaacctgcagagaaagagtttattttacgtgaagtggctaaggatatagaagtagacacttccgaaaatgaattatctaaatttatgttacaattaagtagagggatttccttgaaagaagcaacaaactttattgaaaaaagaaaggggtaagaaatagtggctaaaacattaaacgatattatagaagattttgatgcacagttaaatgaaaaagtaaaacctactacagatgaggaaattacaaagtctgtagaagaacctactgaaccagaaaaagttgaagaaggtgctgaggttgagccggaagaaaagcctaatgaatctgaggagactgcaggcaatgacggagaagaatccggagttactgaaacagttgaagcagaacaggaagaaccagaaactgttgaagaagtagcagttgaggaacctgttgaagaatcagcagaaactgttgaaaaatctgataaaactaaagaaaataaagatgaagaagaggaggaagacgaagaagacgaagacaaaaagaaagaaaaagacaaaaaagacaaagacaaagaagataaagaagacatcgagaagtctaccgaagtcgaacaagttatcaaatcttctgaaatcttaggagctatggaagctatctttaaaaatatgttaggtctaagtgaaaagttagacgaaattcatagagagtttaaagaagctaaagaagctaaagaaaaagacgaagcagagtctgttgagaaatctttacttgataaccctgaaattaaaacaggaaaagaggactcagaaggcaaggctgttgggtttgttaataagtctgtagcagttgaggaagaggtagctaccgaagaaccaactgtagaagtagttgttgatggtgaacaagacacagcagaacctgaaaaagaagtaccgttccgtgatagagtacaagctattagaacagactttatggaaacatataaacgtgtatctgttagcggagtagctcaacgtggtgaattagaatcagttagacacacttggggaactgctagaacaaatgatgacctagctaaaattgaagggtttatcaataaatataaataaatttactatatagtgaacgatttttaattgtactgctatattaatagcagaaacaataaaagacctgacattccctcctgaacctccacagggtcaggtcttatattgagaggaatataaagaaaggtgatttatacataatgacagagaaaaagaatacagaacgacaattaacttccgtacaggaagaagtaattaaaggcttcactacaggatatggtattacaccagaatctcaaactgatgccgccgctttaagacgagagttcttagacgaccaaatcacaatgctaacttgggcagacggagacttatcattttaccgtgacatcactaaacgtccggcaacttcaactgtagctaaatatgacgtatatctagcacatggacgtgtaggtcatactcgttttactcgtgaaatcggtgtagcaccaatttcagaccctaacttacgtcaaaaaacagttaacatgaaatatgtttctgatactaaaaatatgagtatcgcaacaggtttagttaacaacattgaagacccaatgcgtatcttgacagacgatgctatctcagttgttgctaaaacaattgaatgggcttctttctacggtgactctgacttatcagaaaacccagatgcaggttcaggtttagagttcgatggtttagctaaactaattgacaaacacaatgtactagatgctaaaggtgctagcttaacagaggctttacttaaccaagcatcagtattagttggtaaaggctatggtacaccaacagatgcttatatgcctatcggtgttcaagcagacttcgttaaccaacaattagaccgccaagttcaagtaattagcgacaacggtcaaaacgctacaatgggattcaacgttaaaggtttcaactctgcacgtggtttcattcgtttacatggttcaactgtaatggaattagaacaaattttagatgaaaatagaatgcaacttcctaatgctcctcaaaaagcaactgttaaagctactttagaagcaggaacaaaaggtaaattccgtgatgaagatttaacaatcgacacagaatacaaagttgtagtagtatctgacgatgcagaatctgcaccatctgatgttgcatctgtagtaattgacgacaagaaaaaacaagttaaattagaaatcactattaataacatgtatcaagctcgtccacaatatgttgcaatttaccgtaaaggtttggaaacaggattgttctaccaaatcgctagagtacctgcaagtaaagcagttgaaggagttatcacatttatcgatgtgaacgatgaaattcctgaaacagcggacgtattcgttggagaattaactccatcagtagttcacttgttcgagttactaccaatgatgcgcttaccattagcacaagttaatgcttctgtgacattcgcagtattatggtatggagctttagcattacgtgcacctaagaaatgggctcgcattaagaacgttaaatatatcgcaacaggtaacgtgtttaactaacgctaatctttagaagactaagcaaaattgaataaaaatggaataggggacggttaatactgttccctatttttattataaaatacatgtatggaggaaataatatgttaaaatcagaaatcttaataaataaaacagtaacaacagcttttggtgaggtaacatttgaccataatggagaaaccacagacctaacggtagaacagcaagaacatttagggactaaagttccatatatacaatatataccagatgcacctaaagctaaagaaaaagaagctactgcagaaaaagcagacgaggcacctaaaaaggctaagaaagcacctgctaagaaaactacaaaatctaaaaaagaggaagactaaggaggtatttatatgtacccagactacggatacgaggaacaaggggacaatacataccaataccaaccatatgcacatgggaatcctaagcatatagatttagacaaaattgatgatatacagcctgctgattacggttggacacctgctacactgaaacaatacatgtttggtgtagaagttgttaaccctgaaacaggagaacctttaggagatactttctatgaacatatcatagactcagcaatagctaaagcagagaaacgactagatattgctattatgcctagacttataagaggagaacatcatgattaccaccaatcagatttcaactcttacatgtatactcatgtgtttaaaagacctattattcaagcagaaaagcttcaattagaagttaatggtagagggctatacagatacccgtctaattggtggaaagtgtatgcactagcaggtcatatacaaatgtacccaacatccctcatgcagactggaacacagtttggttatgaaatgacgttctcagggtacccacaattagcgggaatgcctccatcaggagggcaagttgatgctcctcaaatgattcatattgactatgtggcaggaatgctacctaggaaaaatagaggatacaatgaagactgggagtgccctgcggacttagaacagcttgtaataaaatatgcgttaaaagagatattccaacaatggggtagacttataattggtgcaggtattgctagtaagtcactaacagtagacggtattagtgaaagtatacaaacaacacagtctgctatgtatggtggtgcttctgcagatattcgacaaattgacgaagatatacaagaattagaaaaatcattggtatcttactttggaatgaacttaggtattatttaaaaaaggggggttaacaaatgggtgaaaaaccaattagatttggtggagcaggtgaaacaggtaaccctaacaagcaattaaatactagtagggttgaatttgaaacaaaaggtatggctagcttcattgaaaatagaggtattgacgttttgtgggaaagagcatggctatgtacatgtcgtaacccaatgaccctctcacctaagtcggattgccccatctgtaggggaagagggattgcttaccaacctgcagtaaaattaagaatggctatacagagtcaagagaaaggtatttctaatcaagatttaggactactagatacaggaaccgctattggaacgactgagctagattctaagataacctttagggatagaataacagtccccgaagttaaaatatatcaaagctttatttttaatgtaaataaaagaagagtagctaatggactatttttaagctatgatgtgaacagcatagaagatatttatggtaaagacggacgtatcttagttgatggagtcgattttagaatggactatgacacaaatactatttacccaaatgaatctttaatagatactaatatatccataaatatgtctgttacacttagatatattgttatagacctgttaaaagaaagtagataccagtataccacatttggtgttaaacaaacacaatttgaatcattacctaaaaagctcttattgaagcgtgaggacgtgtttattgatagtgaaccattttcattagacatagatacggcaagccgtatggaagagctagagggcaaaaaagatacaagtgaagctatggtagaccctaagcgtaaagctacaaaatcaggaggcttctttggaggtaagttaaatggctagaaaaggacagagacccgtattatttactgattcaaaagcaatacttggcaatctgactcgtgcagtggttgatgaagtactaagtgatgcgcaagatgttgctctacgtaatgggtcttctgtacagagaatgcctagctatttgatagtaacagagtctaggatggcaaaaaatggggttatagatttgaaacctttctttgcacgttctaataaaaagaaatataataaaaaaggggaatggtacctatacatccctattagtatgaaaacacgcaacatgtcaagacgattatacgatgagctgagagcagttccagtaggcacaaaacctgtaactgttaaaatggactatctgtatgatagacggaagcagagcccatcagtgtctagtattaactataaacctaaatctactaatgtaactgttataccacagagttggggtaaaggtacacgtaacacctatgtagctttccgtacagttaacgctaattctcctgcgaatagttgggtaataaatcgtagaaacgttaatgatgatgatatgagtaagacaatgctaagaaacatagacaggctcatgaagtggaaactaaagaatttaggaggatagcgtatgataccaagtttagacacttacttgtataaagaatttgaagaaaggctaaggataattctatcagagtgctatattatagacgaagctttaaagggaatggacaaagaagctttagaatcttttaaaaacacttattgctctatagatggtaagccacctaagagagaagtagagatgtcctattcattcccacaagaacatctggattcatttgctcgatttgtagtaactctcggtagtagcgaagaagatagcaagtctattggaggaatccaaggaggctacgagtatcgagaaggtaatgtaattagtgaggaagccactattattagagaaggtgacaagctgattataaatacatcaaaaccagtagcggactatctaaacagctcagacataagttttgcagaaagcgaccattttaggattgaagataataaacctgtatttgatttttcatacaatgaggagttagaaggtatatccattaatgtgtcgtatataagcaaaatatcagatgatgatgttgcaggtgtatacaaaggataccaatctaacgataatgttagtataattggaataagttctaatatagatactgctaggtgtttagatgcaattgctagaattatactaatcaccatgagagacagtttagatgaaaaaacagggtatatgttgcaaacactacattttggtgatatgcaagttgtcatagaatcaggtgaaacacttgtgtttggtagaccttgtactgtaaactacagagttactaattctattggatttgatttacaacaaagaatcacagagattattacaaaaaggaggatgaaatcctagtggctaaagaaacagagaaagtagtaaaaaaagaagttaaaaaggagcaacctaaaaaacctaaaggttatgtccatgtcgatacatttttagattatgcaaaagtattatatggactaaataaatatcaggtagcgggtttcagagcactaatggcaggtagagaataccaacacgaggatgctgattttgttccatttttagaaaagtatataggaaaggaagttaaataataaatggctgtagaacaattcccaagaaaaaaagtatcacgtccacatactgagattaccgtagacacaagcggtattggtggggcatcaagtagctctgacaaaacattaatgttagtcggctctgctaaaggtggtaaaccagatactgtttatcgtttccgtaattatcaacaagctaaacaagtactacgtagtggagatttgctagatgctatcgagttagcatggaatgcatctgacgttaataccgcatcagcaggagacatcttagcagttcgtgttgaagatgctaaaaacgcaactcttgtaaaaggtggtttaacatttgcttcaacaatttatggggtagatgcaaatgaaattcaagtagcattagaagacaacaatttaacacacacaaaaagattgactgttgcattttctaaagatggttataagaaagttttcgataacttaggtaaaatcttctctatccaatataaaggtagtgaagctcaagccaactttacaattgcacaagataaaattagtaagaaagcaacaacactaactttaaatgtgggctctgaaccagaaagtacaacagaagtaatgaagtatgagctaggtcaaggtgtttactctgagacaaatgtcttagttagtgcaatcaatagtttaccagattgggaggctaaattcttccctataggtgataaaaacttacctactgatgctttagaggcagtagacaaagtagatgttaagacagaggctgtattcgtaggggctttagcaggagatattgctaaacagctagaatacaatgactatgtaactgtagctgtagatgctacaaaacctgtagaagactttgaattaacaaacttaacaggtggttctgacggaactgctcctgagtcttgggctaataaatttccattactagctaatgaaggtggttactacttagtaccattaacagataaacaagcagttcattctgaggctttagcttttgttaaagaccgtacagacaatggtgacccaatgcgtattatcgttggtggaggcactaatgaaacagtagaggaaagcattactcgtgcaacaaacttacgtgaccctagagcttctttagtaggcttctctggaactcgtaaaatggatgacggacgtttgcttaaattaccgggttacatgatggcttcacaaattgcaggtattgcaagtggtttagaagttggtgaagcaatcactttcaaacacttcaacgtaacatctgtagaccgtgtatttgaaagtagccagttagacatgttaaacgaaagtggagtaatctctatcgagtttgtacgtaaccgtactttaactgcattccgtgtagtacaagacgtaactacttacaatgacaaatctgacccagttaaaaatgaaatgtctgttggtgaagcaaacgacttcttagtttcagaattgaaaattgaactagataacaacttcatcggaactaaagtgattgacacaagcgcaagcttaatcaaaaactttatccaatcattcttagataacaaaaaacgtgctcgtgaaatccaagattacacaccggaagaagtacaagttgtcttagaaggtgacgtggcatcaatcagtatgactgtaatgcctatccgtagcttgaataagattaccgttcagttagtatacaaacaacaaatcttaacagcatagagtgtaggggcagttgcccctcctcttacataatgaataaaatataggagtgatatatacatggctagtgttggaaatcaaacagtccacacaggtaacacagtttacctaatgattggtaataaaattatcggtcgtgcgcaatctgcatcaggtgagcgccaatatggtacacaaggtatctatgaaatcggtagtattatgccacaagaacacgtatacttgaaatatgaaggtacaattactttagaacgtatgcgtatgaaaaaagaagacttagcaagtttaggaattacagcgttaggtgaagacatcttacaacgtgatattatcgacatcgtaatgatggacaatttaactaaagaaatcgtagtagcttatcgtggttgctctgcaatttcttactcagagtcattcacagctaacgaagttacatcagaatcaacccaattcacatacctaacctcagctaaagtaaaataacaataatgttaaccacccacattgtatgggtggttattttatgttatactatttgtattgaataaaaaaaagcttaaggagggcattagtagtgaaatatccatatttagtagaattgtatgcaaaacatgtaattagagactcagggtacatagaaaatgtaccaccagttatttatgaagacgttgtgaaacgtgttgaagaaataaaaagagaagaaaacttaacaatagattagctagtataagtttaaatagacctaattcagatataggtctatttttttttgccactataaatacaaataactagatagtgtggtataataaagatagtgctatattaatttatgagtaaactaggaggaactaataatgacagaaaataatgaaaaggtatttacacctacaccaaatttatcacgtgagcaattaatagaaaagttgcaacgtggagaaaacttgacggatgaagaagtaaatattttgaaatattacaatgatgcagaagaacgtaaacagttagatagaattatcccgggtgttaatgatgtattttctaagcattataacttaaaagaatatggtttagaatttgatattaaaataaaagcacctaatattatcgaaaacggtaaaattcaggcaagaagagaagcctatttagaaggcatgggaatggcagttagcaactttattttccaaagttaccaaatgttagcaactattcgagtatgtggggtagaggtgccagaagtactagctgatgatgaaaaaatttataatttatatgtattaggagtaattgcaaaggactatggtgaatggctaaactcctttcgatactagagttaaagaactaggtggaataaaagccctcgttagaaatagctatagtagaaatctatgggcaataatgaaaaaatttaaggtactaccgagtgaccctgcgtggcaaaatctcacaagtgaccaagtagagtggattttatataatatggaaagagacatagaagaacaagaacgactagctaagggaatgcagttagaaagtgagttccaagactatgacgattcatggtatgataagccacatgatgagttctccccaattcgtgaaggtgacaatgaggaagaaattgctcgtaaacttagtgaaatcacaagtgaggaagacatggctaaacttaaagctcgttgggaggcaagccaagaagttgatgctatccgtgcagaaggtggaacaacaattgaagaagatacgattaacgaacttattgctaacaatgttaaaaaagcaatggaagaggctagacgtattgagaaacatggtggaaataaatggcaagagaaatcatcaattgagttagaagaggaacgtaagaacctagagtttaactcacagttgaaacaaggagatattcaggaggctatcgatttgtttaacaaagatgtcgagccaacatcattagatgacgaatttcaaatttaagggtagggtattccctgccctttacttgtaaaggaagtgtggaaatgagcaacaattatcgtttttatgttgaggcaatgactggggatgctgttgcaaaacttaatgaaatagacaagttaatggataaaattgattcaaagagtgcaaagggcacccagaattttttccatacaagtcagaaagacattgataaagctgttgaggaaatgcaaaagcttatcaaggcaaaaaaagaactagacagagcttttgataatcagaagataaatgcagaaagcatgggagacatgacagcatataaacgtgctgtatcagatgcagaagaactaactagaagatttaacaagacacaaaaagaatttcaaaatcatgctagaatgcaagctaaccctaattacataaatgctagcacactaaggcaacaaaaggcatttcgtgatgagttaacagagcaggagagagcaataagaaatatctctagggcacaacaggagctaaacagggtgaactctagggttaaccatcgtgcaaaccaagcaagcgcaacaggaagaatgacttacaatcagtcagagagtatgaagcgtgaccttagacgtactggtgtatttgaaagcttaggctcagaaaataaaagcagacagcaagagctacgagaacgatataaacagagacaagaagaactagcagagactagaagtaatacaaatttagatagacaagttcgtaagaatagggaaactagtattcaggctgagataaaagaaattgagaaagaaattgaagctagaaaacgcctagcagactctattaaagagactgtagagaacttaaaatctaaggaagcttcactaaatgcatctgacataaaagtagatgctgatagaaagagtattcgtggtgttctagctgagcgtgcaccttctatatcaatagctatgctaggaggtaccgccgccgccataggtggtttatacgcaaaaggagcaactgctaacgcaggtatgcgtgatgcatctatatctttaggacaacgtacaggtactagtgatttccgtgcattaagaaaaagtatgcaagagatgggtattgagaaacagttaggctataaaggtgccgacatgttacaattccaagaagatgcattgagtaatattggttttacaagtaaagaggacttagcaggaagtacaagagccttagcagagggttcaagagcagttccagtagacaatgagactttaagtgacttcatgaatagtcaaatgaagagtggggctattagtggtagagaccaaattaagaatattcaagaaggtttcttaggtgctatccaacgctcagggatggcaggtcgtgagaaagaacaactagaagctcttaaaacattaagtgagcaaagctttactggtcgtaacggaagtaatcaagagcttaaagaacaaatggcaatgttaacaatgctaaaccaaacaggtaagcgtgctgttcaaggtgagcaaggggcacagttaatgtcaagtttatccgcaggtattcaaggtagtgtatggaacaataaggcatctttacttttaggtaaagggacacaattccaagggctagcaggaatgtacgacttaaaagctatgcaagaacaaggagcaacaccagagaacttacagaaaattattggcagtgtccaacagtcagttcccggtgacgaaaagtctcagaagtatgcttttggtagtgccttacatgaactattcggtacagatgctaagaatgaccaaatagatgcaatttgggaagcttatgccaacggtggtttaagtcaggataacgtagatagaataatgaatgaaagccaatctacaggaaaaggcaaatatgataagaacatagaagactacgcaaactctaaagaaggaaccgctaaccgttcagaagctgttacagagaaacaagcttcacagattaatgatatgggggacatcttaagagagactaactctaaattaggggcactcccccctgcgttatatgcattaggagcaggcttaggagctatggctgtttcactagccacatcaggctctattggtggtttatctagtttaatcaaaaaaggaactcgttctacttttagtacaggtggaggagtaactgcagggggcggatttttaaaatcagctaaagaggcattttctgcaggtaaaggctctggcggatggttccaaggtattaaaaatgtaggtagtgtagctaaggactcagcaatgggtgtcggagctaaggctgttgaaggagcaaaaggactgaaaggtgccggaatgggcagtaaggttcttggtgggttaggtaaagcaggtaagtttttaggtaaggtagcaacccctctagcgataggttcttctctattagatattgcaaccgcagatgataaaacaaaggcggtaggggagagtgttggagctattggtggaggtatcggaggagctaagctaggggcaatgattggtacatttattgcacccggtcttggtacaggtatcggaggagctattggtggaggtatcggcgccataggaggtactctagcaggttctaaactaggcggtaaattcgttgatggtgttcgtaaattcttcggaggagaagaagctcatgcggaagaagcagacttaactgcaggacaagatgttgcttctggacaagagagtaaatcaggagtacaggaagccagagacacagctaacaaacgagtcctatctgagaaaacacgtgcagaaaataacgcagaagagtctgctaatctatctatctattctaagttattagatagagctcaacgtatactaaaccaagcaaaaaatcaaaatggtatattcggtaactcaggaagctctaaagattcttctgacggaatgggttcagatgcttctagtaaagactttggaggggactgggagaaagccattagacaagcatctaagaaaatgggagtagatgtttctgacgatgaaatagatacaatacttagattgattcaggcggagtctagtggtgacgagagtgctgttcaacagataattgatgagaacaacttcaatggcaatggcggagctaaaggtttactacagtacgttcaaagtacttttgatgcatacaaagtagacggtcatgacaacataatgagcgggtatgaccaattgctagctttctttaataatagtaattggaaaaatgacctaaattcttgggatagccgttatcaaaacggtagcacaggatggggtcctactggtaataaaactagagcgatgggtggacacataactagcccagagtatgcactactgggagaggtagcaggtcaagatgagtatgttataaaccctagtcagccaacagccccacgtttattagcagaggctactagaaaaacagctcaaaacttccgattaacaggtaacggttctggtcatgattggggagcttcaatttctaaaataaatgcaacaggaggagctcaatcaggttcagctcctacgatgacaaatacaaatgaggtatcagttaatgtcaccatacaaggtggaggaacaagtgatagtatagctagagaaattggtgataagtcagcaggaattatcagcaaaacactcgatgctaccttcacagatttcttcgcaaaagaatacaggagagtgtagaggcttaatgtctctacttttctctaaggaggtttataatgtcagtagagttaagatacccgagatttgatttaactttttttacagagacagacaactaccatatagtttatgatgcaaaagatgggcttactggacgtaataacaataacggtgaagcagagaaggtaagcaataattttatggcagagtctgtaattagtttaactacaaaaaatgctttagaggatgatagtgcagtcttctcattcgtattagcaggggacgtgtattgggatagagtactaaatgctaatgatgcagttattcttaaaatagacccagatacttcctccactaaaaaatcagataaccctgtactactagttggtttaatatctgaggttagactagagggtgactatggggaaaactctaagatgtacaggataacaggacagtctttcgcaaaggcgctaatgcagtttgacttaggtgttattcaggaagtaagtgtagttttgacagacttaggttggttgcctgacgatgcacaagaaggtataaaaatgtctggtagtagtgcaagtcaaatagcagaaagcttaatgaaaagatttttacaatatatgaaatttaattttaatggtcaaggtatagataagttcttagagtgggagctagatagttggacagaggcggagagactaatagatagtaccccttacataaactatgaaggttctttaaagcaacttatagatgatgtaaccgctaaaccgtttaacgaattgtatttcgatgcaacaccagagggtaaatgcagaatgattatgcgtagaaccccatttgataaatctgattgggaaaaattacaaacctacacagtaacatctgcagaagtaatatccgaatctgtagcagttaacgatacagaagcttactctatatttaacatatctattaataacttgtatgggactgactctatgatgttaggttctaaaccacaagtattccctgatttagtttctaaatatggttataagaaactagaagtacctaataggtatttagaaggagcaattatagataagagtaacagtggggataaagctaacacaaaacccgaaagtgataatgatagtaaagagagcgatggctcaacagctaaacagatgtttgacagagagtacgctcttgtcttaaactatctaaaagggtatcctgttgatgtgctaagagttaaaaaaagtaatgtaagaacatcaataacacaggtagacagacgtataacagataacgaagcagataaaattatagaccactatattacaaatcaagcattaagtaaagaagacttttctaaatttacaggaataaccgaagaaaatatagaagagggtaacggtaaagtagaacctacatataccgcagttagggacttccttaatgggttagataaaaacctaggagtatctagcataaaagagaaattaatgaactacttcaatttgatacctaaccaagccacatctattgctagtgaatacaaagcacaaggtaacttaggtaaactaaaatatgaagaaataatggagaataacccaagtgatagctcaaccgtcactggttcagataaacgatttgtttctgagttcactaaaagactagcaaattggtactgtgaaaacgctaatttctacagtggggatattgtagttaaaggtgaccctaaatatagattaggtaaccgtttgtttgttcaagatgagcaaaatggtgagttgtgggaatactacatagaatcagtagaacactcgttctcctatacacaagggtatatcactactcttggggtcactagaggtttacaaaatggaggaaacgatagattcactcatttatggggtaaatctgaggacttctccggaggtatgctaggtgagaaaaccttgcaagcactactagacgaacaagcagaagcaaacagtaaaaatgatggaggctctgatagtggaggtagctcaggtaaagagtatacagcaggtgcaggaactcagctagcggtattcccattagacgtaattaatgtaactcagggagaaaatggtggatacagccacatgggagcacttgcaatagacttttcagatggtactcctcataaaccttactatgcaccatttgactgtgaatgtgtgtacactgactcttactcaggggttgcatggcaatctcaaaagcctgttaaatgtgtagatggtagcgttacttacgtaacactattatgcgtacacgacaacaactgggcaagcaacaaagtaggagataagaaagctaaaggggaagtaatagggcactcagggactgcaggacaagcatcaggagaccatgcccacttcgaggtgtctaaaggtaaatggcaaggttggagcacaagcagtgcgggagtttactttataaaaaatccttctcatttatacgatgtattttctataaaaaataatgtaactggaaaaacaactaaaattatgaatggtggagggtacaattggcgtagtatagactgggacgataaatctggttcaggttctggaaagaaaaagacagttggtgctagagccatggctaccccattcggatttcgtatgatgcgtagcgctccggtagaaccagaagagccgaaagttgttaaacaaatagaaactaaaacagttgttgaaaagaaaccaaaagtagaagttaaaaagctcccaatactttatgggaataacattgccacagaagcaactaaatggggaagagctcactcgaaaacagagtccacattccaatctgccttcaattttggagctaatacagataaagacccatttgaggaagacattatagcaacagacagcgcaggatttgtttggtggtgtttctaccatgcgggcatatcgctatctggtggagctagaatggtcaccactcgttcactactttatgataaccaattacaaactatctctactagaggtcagaagtctttagagttatttgataagatgaaggtaggagatttagtttggtttaacagagctagccatgtagggatatattgtggtgagggtaagatggtctcttgtaatggaaaaggtaacatggatgagtccccaaaaacaggtattgttattgtcgacatgtctaaaggttattggtggaacgcttttgacggaaatgtacgtagatataaataaccctattttttagggttattttttttgtataggtagtttttagttattgtgctataatagataatgaggacacatgatgagaggatgaataaattggtaaaacgtagatttcaggcaggtctaggctcagaaattaaaagagtatataaagaaggacaacaaattaacacgctactattagcacaagtaattcaagtaaactataaatataatacagtagacctactagctttacagcataaagaagtatttcaaaattcctatgcaaatgagggacgtttctcggcaagacttcctatggaatttggcggtagaaatatcgttggacagccttatggacaggttaacccgatagcagtaggaacagtagtactagttggttttattaattccgataaagacatgcctattgtaattagtgtttataataacaacgatgtaagcaagcaactttcaagaacacaattttcaaattcagaccctaaagatttagagttaattggggatatgcaccaaaaatttagtttatacccttcattgacatatgatagcgttgatggagaaggagggcgtgtcgttactttttctggtaaatcatttattgcttttgatacaaaagaagtagctaactcctctacagctgatgcaggttacggtactaaatacgaggacttagagacatcatactataataatggtgacctaatagagcctatgaaaggtagagcaccaaatgtactgtttaagcatcaaggggtacttgatgatgacggcaaaccagatttacacgatttgctaattcatattaacccagatggtacttatagaacttctatgatgaacaaagaagaggattggcgcacactatttgaaatgacaccagatggcagagttaaattaagaaaacaagactctattaatattgatggtggcatagaaataagtgagctaggaatcaacaatgaggggttcgtttatttacgtaatggggatatggatttagaagtacgaaaagacggtatctattcacaagggaaactgtttacagccgatatagacctatccgatgtatatgacaaactaaatgggttgtctatacagattaaggaaacaaatggtcaattagagattatagctaatggtgtagaagaacaaaatggaaaaatatcagaactttctacagaaataacaattgtagcaggtaaagttgaatcaaaagtaacaaagacagaagttcaggatatgattgacagttcttttgtagatatgtctgatgcaattaaaaaagcacaagaagatgctgacaaagcaaataaagtgattgcagatatgtctagtgataacagactgactccaagtgaaaaaatagatttattaaaagaatgggatattataaaaaatgaatatccaagctatctcgaacaagcagaaacctacgaggttgatagtaaagactacactgctaagtataattcattagagctatttgttacccctatattggctgacatggaatcaactagctcggtagacggagcaacactccgcaaaacgtttaattcttactatacagcaagaatagctttactaaattctattagtaaaaaactaaaagacggtatcacagaggctatgaaaaaagcatcccaagcatcactagatgcaacacaagcaatggcagatgcctcacaagctaagattgatgcagataatgctaacaaacttatatctgatatagcaagtgataacaagctaacaccttctgaaaaataccaacttaaaaaggaatgggatgtaattgttaaggaataccctacaacaattgcacaagcagagaagtacgcagtagacacagcagagtatacagctaaatataaagccctagagctgtttgtagagcctttgtttaaagacatggatgaaactagtatagtagacggagaacgccttagagcgacattctcggactattacgcaagtaagattgctttactaaaagaagtaacggactcagctaaaacagagctagatgcctatggtaataaaatatctgtaatggaaacaaacattactcaaacatcagaagctattactttactagctactagagtacaaactgtagaagacggtgtacaatcgaataaggcacaaatcgaaatacaagctgaacaaattagtcaaaaagtaactgctagtgaggttaaaggaattgtagacgattctattaacaatctaacattaggtggaactaacttatttgttataaagactcagacagcaggtttactaaacgagaatgatggaactgtaggtactgcagtagacaactcagtagtgtcagactacattaaagttaatcaaaaaacaccatatattgctacactttatggtaacactggcacaaacatgattataacagactggtacgataaaaatagaacatttatttctggggaagctgtggcagactctggggattttagtaaaaagtatgtgtcacctgagaatgcagtctatgctagggtaagttataagaaagcaaactctgtgaatatcaaattcgaggcaggtacaaaggctactgattacagcccttcatgggaagacataaaaggtgaccaaactgctttagaggaatacattaaaaaagtagaagaacaagccaagaaagctcaacaagatgctgaaaatgctaaaaatgatgctgaaaatgcaaataacgcaatagctgatatgtcaaatgacaatatgttagcaccgaatgagaaaaaacaaatactcttacaatgggaacagattaaaacagagtatccaataaacttagaccaagcaactaaatttggggtgtcttctcaacagtatacaacagcgtataacgcactagacgagtacttaaaaccaatactagctgacatgacaacaacttctgtagtagttggttctactttaagaaatacgtttaacaattactatgacaaaagaactactttactaaacagaatatctgacgtagcaaaaaatgtagcagacaaggcacaagaaactgcagatactatcaatgataatttacaaaatattggtgggtacaactatgtagggttctcttccggagacaatatgttgcctagactgatgattaaaaacgttggttactacacattaggttcgtcaaccacagagttcattgacagcatggtagctgtaaaaggtgatgcaacgacccaacctttcgattatactgtaggtacttctgataaagaaattgctggtggcggtttagctgattatcgtatgaaagaaataaaagaaggtcagtggctaacagcttctgcgaatgtgcaggtaataggtggtggctccgctaggttagctatctacactttagaaggggataactgggtaggttctaacagtacacctatacaagtaagtgatggtttgaaacgtgttgtggctcaaagaaaagtaacaggcttaacaaaaggtgtgttaatacgtattgagtcagctgacactaatgttaaagagtttcgatttggtaatgttcaactagaagtgggtatcatcccaactccttggaaaaagtctgatatagatattcaagaggacataaacaatgttgttcagaatatcaaaacatacactgcttgggctaacgatttacagggtcttgattttacaagagaaaaggttgaaggaaaaacttacatgtatgtaggtacctctatgaaagatagtgataactatccagattatacatggaggctaactgatgaacatatagaaggtcagattaatggtaaggaaggcgcatggatttactctccaacagcccctgctaacccatcgcaaggacttatatgggtagacttgtcaaaagtccccaaccaacctaagcgttgggtagattcagaaactgggtgggttgcattaacaccagaagaggttaaagatttgccttggggtgaagatggcacaagcttagccgactgggtgtcacaggcagagcaaagaatatcttctgatagcattataaatactgtactaggttctgaggatttcactagtgtgttcgatacaaaagctaacacttctgacctaggtaacttggctacctatgaagacttagactcaataaaagaggactataaccggctaatcaaagaaggcataaatggtattgattttactccttatgtgactaactccgaactacaacagcttaaagacagctttaacttctctgttcaacaagccggaggggttaacatgcttaaaaactctttaggattctctgggttagacttctggaatggtacagtagggaagaacttactacctaactctacttggaatttaggttttggtagatggggtggtacttcaatcactagttttgaaatattaccaccagaagatgacaagcctacgagtaacatattagcctcaatgccacttcgctcttctactaaagaaataggtaacagacctcacccattaaaagttaactcgggtgaaacgtacacagtaagcttcgactataaagaagaagcattatcttacaacaaggacagacctatccttgttgtaagaaactaccctgataaggacacagaccaatggatggagtactcaatagaaggttgggcagtaatggctaacggaagcactactgacttgactgtttggagacgttttacaaaaacatttacaataggtactagtggctacttagatattttaccgaaaaccataataaaatcgtgggaacacaggtctttttggagagagctaaaaatagagaaagggacacaagccactacttgggtacctaacaaggaagacggggcgtttactggtgatattgttgagactattcaaacagaagaattagccaacctcgggtttggttccggatttattagttctaaaagacctagctcttcattaacacaatctgtagaactacctgaaataggtgctaaccttgagtattcactatctttttatatgaaggtaactacagataaccctgtagctgactttaaatgcggtattcgggtttatgatggaggtactctaacttatacattaggcatagaagatgcaacacagccaataccactagggttccaacaatacaagcttgtgttcactcccacaagtacctctactaaaatagaaatgtttgtagaaaatgggcaagaggcatctgttattatatcaggtattatgtataatatcgggagtatacctcttaaatggcaaccatatccaagtgagatatacaatacgaatgttaagattgatattaatggggtaaccgttaaaaacaatcaaacagatgggtatacaatgattactccccaagagttttcaggatactctcgtattgatggtaacatagaacgtattttcactttaaatggacaggtaacagaagttaaaatgctaaaggctgaaaaacgtataactatggaaccagtatctgtattcgctatgaacacagtaacggatacaaaaagaattagaggttgggcatttgtgccatcatttgaataaataattcaactttttaaccgaaaacctcacttacaagttgttttcggttaatttgtgttatactatatagagaaggacacgaaaggacgatgagactatatggctttaaacggaacaaagtatacagcctttgcccgacatagattagttttagagtggcgcgcaaatcaaaacattgcagggaactactcaacaatcagcgtatggctatatctacaatctatggataaatgggggagacttgatgctcccgctattggtgatgccaaagttaccgtagaaggaactacacagacagaaaaagcttcctctatgttaaatgctttccaaaagaaactattactagctaaagagtggagagttaaccataataatgacggttctaaaagaataactattgggggtagctactttgtaaacgttacttttactgataatggtgtaccaacatattacggtacgataactatacctaacttttcagtagacctgaatagaatacctagaagaagttcattaaaccctgtccctacattaaatttaccggggaacttaccaataacaataaataggcagagctccacattcaaacataatctaactgcttgggtggctaacagagataaccccacattaagtaatgatgcccattggacgtacttgacaaatcttaataatgtagacactagtgggtcatttagttttacagtagcaaataacaaaactatttttactgcattaaacaataggactagttggcaaggcaaggttaaactatggactatagggttagatgatgtagttagtcaggagagaacatacaagattgtccccccaatgaatgcacaagcatcgggaggtaagataaccttaaatgtgggagagaaaatcaatgtatcactaagtaactatcggtctgatgcaaactttacttatgatggggtatttaacattagtgggctcaacatacccattgctacaaattcagcagggaacacaatgtcgtatacactaactcaaacagatgtagacaatatattgaagaaaataccaaatgcggattcctcgtggggtcaagtaactgtaacaagtaagtatagtggagtacaatacaggactccatggacaggacagagaatagatataactatacctaaaaacaagtatgtaccaagcattaatggtacccctacttatgaagatacaagtagtgtctctgtcgggcttacaggtgacaatcaggtagctcttcaaggtaagtctaatatcaaagtgactatccctgctaactttgcaacggctaatggttactctactttgaaaacaattgatgtgtctttaggcggtacatcaaaaacagtcaactattcaaatgcagaaacagttgtagagttaggggctcccgctaaccatacatcagacaccttaatagtcacagtaactgacagtcgtgggtttaagtctaactggacaaagcatgtagacatttacccctacgaaaacccaaatatgtactttacagtcacacgtagaaataactttgagacaactacagatattaatgttaatagtacatggtctcctatcactataggtggtgtaaataaaaatgctatccaatcagtgacctatgcaacaaaagtagtgggtgttggcacttatggagaagaaactgctttaaattttacagctaatggtagtatggtaacagtaaaaaatacaccaatagaattagataatactaatacatacgacgtcaggttaagcataactgataagtttagcacgtttactagaacagctactgttaaaccgggtaaccctatcatgtttattgatgccgataatcgaagtctgtttttaggtaatgcctttgttgacaataacaataatgagctaagaggcttattagagatagaaagagataagtggcaggataggggtttagtaggaatatcattaaacaatagtgatatatcagcagtcaacggtatatggttctcaacagatacctcagataacgcaggtgaagggctccactttattaaatcaggtaaatcaagaaattcattgacttgggatgattatgattacttttatatgagagataacgggttctatgttaaccacaatactaaccctatatttgaagtttctgacactggtacgatgacgtttcctactaaacagctttggaaaggggttgtctatttaaacgatatacaagaaattttacctagtaaaaaactgacagaatgccgaaacggatgggttttgaccttctcagcatatgagtcaggtgcggggcaaccttgggatataaatcaggtatacatccacaagtctgtgttaaaccagtttaatacaagaggatttagggctatgataggtcatggaactacgataacatttaaatatatgtatataactgacggtcgtataggtggacatgctggcaatggtataggagttaacagtggcattgccataagggaggtatcagaatggtagaagaaactcaaaaaatatttgtaacactagatgataaaaattttgttagtaactgggggacgtcactcaatagtggcgaacctgtctatgaagtagatttgccaatggactctccattcttttcacattactattcaaattgttttcagtatgtagatggagagctagtcctaagtgaagaaaaattattaaaagtagaactactagcagtagccagtaagtttaaaagggactgtgaagagacctacatattgcaaaaagttccttatacaatatcagacactacctacttatttgatgttattagcgatgatgagtttattaataaaataactctactagacaataaaattcaagattatgttggggtaactgcttatgctacatcagatggttctcaatcgatacttaaatttgataagtcccagtatactactatgactagatatattaaaaaagtagttgagtctagggaaaataaattaaataataaattgcttcctatgattgaaaatgctaagtctgtagaagaagccaaagaaatttcttggagttctgtacctgatgagttgctaccagagcaagagaataatgaacctcaagacgagcaaagtatagatagtttaattaaagaaaataaagaattaagacaaaaagtagaatttaatgagttagccttaatggatgcaataaacatgttctcggaaatgaacaagcaagctactattaattaaaggaggtgatatttctatgtacccatacttatcaatgttatatgcatcttatgtaatcaaagaccctgagaactaccctttagagaaggttccggcactaattagagaagatgtggagaaaatcgtggaagagatggcaaaaaagaacgaaaaacaaggatagttaatttagtaattggagtaacaataggtttaatcatagggctactaatttaaactaatataaatgagggtagtttgtgctaccctcttttttatactatattgtactatattaaggaacgaaaggagcggttactatgggacaatcagatggtatgggcggtacattaaaacgtattgcgattcaagtaggtaacgacccgaataaaggttggtatcgatttcaagtaaacccaactcaatataaatataataagccacatcgtgttactatatttaaaactaaatcaaacattataacagaagactttggtaaagatatagaaactatccaattttctggtacaacaggattcagggtagacagcagaggtaaaaatggggcggacagactgaaagaattagaagaaataatagataattacgcaaaacagggcggtaatgggaataggtctagtgtagagatgaaattttataatttcacagacgataaatattttgttgttcatttagcaccagaagggctatccattgaacgttctgcagaacagccattattatttaattacaccttaagtttagtcgtgttaagagaggcaggacagccctctgagagagctcaagtaagccctcaaataggtaatgtaagtcctagcattggacgggcatacaatgcacaacaggacactagaacacctgctcaaatacttcatgatgaatataggagaagtgttatgccaaatacagcagtaaatcctgcagtaacatcaggagcctataactatggtgtaaatgagttaaaaaaaataatcggatatggagggtaactcatggaaaaagtagaacaatccgcagatttgctaagattttttagatatttaaatgtcgatattagcggtgaagttgtagccaatgttattgacgaccaaccaaactttatatcaaggttctacacccctcatacacgagttaataaaatatctagtacactattagatattgttagagataacgatataggggaaacaaataaggctttgtctaaagactccttaacctacaaatttttaaaaagtgggcttaagctttcatctccacgtatttacgaactagcacaaattgtagtattagaatcctttgctcttatatatgctatcgaagaagaaccagagatgtttaaaatgattaacgaatcagatgtaaagcagactagagaaaatgttaaatacttaattgactgtttagggggagcaaaagactatacagacatagtgatggacttacaatctatggatgtcgctctaggatatattcaggagcaagttcctttaatacaaggaggtttaccagtaaatggcacgatataaaaagcatttaattgtatatggagatacaatgcaatccatagcacaaaaagaaacaggctctgtagataattgggtaaaaattgcggagtacaacgacttagtttacccttacatagtagacactatgcaagaaaaaatgagcaacttagaacatctagctacactcggagacaccctttttatacctgatgaaggtaacttattggacattaacacaagctcattaaaccaaagagatatggatttcttattaggcttagctttaggtaaagatttggatatgacaagtgatacagattactatgagaatcatggaacaagtgacgaagtgtttgcaataacacataatgggcatggggacttgaagatagctagtggggcagacaatattaagcaagccactatatcaagattgatgacagctaaggggtctcttatgttgcatccagaatatggtagtgacttgcacttaatgttcggtaaaacaacaattgaacaaatgaaaataattagtatagaagtatgtgatacagtacttaaagatacacgagtagcggaatgtgttctagtgaaccattatattgaagaagaccgatatgttggtaactatagagcaaccttacagtctactagagagcaatttgagtttgttgttcaaaacgataactcgggggctctaattattgtatagaaaggataaggtttaatgagactaaagaaaatttcagagatactaggtagactaattgatgtaactatgataaacacacatgagataaacgatttttcagttggttctactattcgttctatttatgaagctgtttctatggagttagaacagtactatattttaggtagagagaatattttatggggaattgaacaaggagtcctaaatgcttttgactttaggaaaagagaagcaaaaagagcatatggtatggtaacactagagttccatacggttactcagacccctgtgtatgtgccaacaggaacaacattcgactctagcttgtctggtgcccctagcacgttaacatttcaaacgatgcaagactacataattccagaaggtgttataacagcaaaggtagaagtttattgtacaactgtaggtacaaaagggaacattcctaaaggaagaatcaatcgggtaattaacaatatatcaaacttaaaaaccgtgtataatgagtttgattttttaacaggtactgatgaggaaagtatagagtccgttaaaaaaagatttcatgcatttgtggaatcacgtggtagagcaacgataaaagccttagattacggtacacgtcaagtagaggaagttgcaggagtttatatcaaagaagaagtaggctacgttagaatatacgcacatgatttaaacggtgatttaaaacaagaaacactagataaaataaaagtagctattgaggactacagacctgcaggcattaaactggatgtattccctgtaataaaatccaatgtacaagtgagtgcaactgttactataagtgacaaatcgagaataaatagtaaattagaagaaagagtggagcttacaataagaaactacttaaatagtcaagttgtttcacaaccattaattttagctgacctaattcaggttataatgaatattgacgatgttttaatctatgactgtaaaattaacaacatggaagggaacatggctgttagggacgaagagataattcgtgcaggagaggttattgtagaactgatataaattaggaggaatataatgtgagtaacttttataaaaatatccaccctctgttgagacgtggtaagaaacctaacaagtatgatgatactaactttgcagtgcttaatgcgttgaactatgaattaactcaggcagagcaggagaccattgctagtaagattcattcttcattagaaacagctacaggtgagtacctagatacttggggggactggtttggcgtatatcgtaaagatgattggaatgacgaatactatagaaaaagaattataagagaacttttactaaaaagagccacaattcctgctatcattgatgcgttattggatttccttaacgacaatgatgcagtcatccaaatatatgaaccttggagaaacattttctacacaaataagtcaaaattaaacggtgacgaccatttaatgggtcactactaccgttttgcaattatcgatatttcaattgatagaccgtttccacctgaaattgtagagattattaaagctttcaaacctgcgggagttctattctatctaaggctagacacaagcttaaataagaataaaacaactgtagaaagcccatatgtatacctagacgtgacgaataagacagacttagagttccttaacggtttatactatgacctacgaggtaatattaacctgtctgaccaacgtatacaagttgtggagaacaacatcttccatacaaataactctatgctaaacggggaagatgtgcttgcaggagcatttgaccacggaagaggctacattcacttagcaagtacaacattgcttgattacacaccaaaacctactgactctatgagtgacttaaaaacagctctaggagaatcaggtgctgatatgtataatcaaacaaaagaaaaggatggaagaacagcttctattcaagtacctgcaacaaagaatgtccatacgttatactccaacagcactgacttcgggggctatgattattcggggaatcctaaccttagcgctaaacttaatgcttcaagtttttcttcaggtactggcgcaacagtcgcggatgataatgatgagattgtgtttaccttagatggcacaaatcaactatcgaagtacgctaccggagcgcaaacgccggtaatagaagggaaacagtatactattagttgtgaaatcatgttagaaacgggcttcactggcgacccgtctggaatacgcctacaacacgcatacttgcctggtggtaacgttgttttgcagacaacgacagtacctaaaaatgaattgaataaatggcagaagcttatcggaacacaaacggtaaaatacacctctgacgcaccaaatgaatggtatccattatttagagacattcgaattcttaaaccgaccggtaaagttagactaagaaaaattaagattgaagaaggctcaacagccaccccataccagccaaatttactcgatgcaccatattatctgggtaaggtggctttgggtgagaatattgctaataagtccgttgagtttccaataaaatctagcaattatctcttatataacgctagaatggtagagccttttgttgtaggagaaacttatacaattaccataaaagcaataaaaccagccagtcaaacgtttatggtatataacattggggaaggaactacttattacggaaaactaaatccagttgagggattgacagacgtatggtcactaacattcacaccaagaaatgtttcgtcaactaatcctagcgatttacgtatttttcagtacccatcatcaacattaggcacatgtcaaattgattggttaaaaattgaaaaaggtgataccagaactcctaacattgattcctacgactacgtaggttctctgatagaagatacagaaacacctacgttagacccgactaagtatacatggacagtaaatggggatataacaaataaaaaggcatatatggtgtttgatattaagacatttatcgaagaaaattatgctatagaatttgaaaaacttattactgacctaggagaagaccaagcattaaataccgtgtttgaaaactttaacatctctacaacacttaaggctctagtaagtccaagttcaccaatcagtttctcggttgaactatacgatttttctacgagtgcatggcacaagttaaacacggatagcttagacttacgtatgcgtacattcaacttagtagcaaaccgtatcacagactatctaaatgattacaagctattatttgttcgttacgtgtttgataacgaaacagataaagatgtaacagttgaactagacatgctaaatgtactattcaagtatcgtttaggtgaggggtacagtataggactacaatcatctgtggaatcactaacggaactcgttcctatagaagggtaatagcaaataatatgttataatataaatagggcacctagttgtgtcctattttttaataaacatgctatattaacaattgaatgataaagaaaagaggtaaataatgtggctattgcaactaacaattcacgagtgtatgcctcactccaattaaaaaataaacaagacagtatgtacctagcaattggtaaaaccactccttggactaatgaagatgcccctcctgcaccagaccctactacgactactctaacagaggttatcggttataaaaaggtagcaagagtatctttatgtagagaatatctaccaagtgacgattctaaataccctgtggtgtcctatggttcaagaaaatttacgctaattccagatgaggacggctataaagagcaagcgtggatggtgtatgtagaagcagaaattacaggagatgagttaccaacgggaacatttaggcaagtaggtattcatactgacttagtgtctaaggcaagttcagaaaagaaagctttgttacctacagatgtaacagatgcaggcattttgcaattttttgaaaatagacagcaacaaaatagaacaagtgatgtaattttaaaagagaagtttattattacaatggaaaataagaagtcagttaaacaataggaagggtgacatagatggctaaaaatattacaaatgatgatttaggtaaagagccttataacaatagatattaccaaggcaaacgattttcaggtttactatttaaaccagataagccgttactacaagccgagttaaacgagttacagtcaattattcaaggagatttaggcaatgtggctgaatccatatttagtgacggtgacatccagactggtatggaatatgtactacaagataagaagcttactattaaaaaaggtaaagtattcttaggcggtaaaatgcgtaactttgacgaacagagtattgatattaccggagagggtacagagtatgtaggtgttaaacttgtacaaaaagttattacagcagaagacgacccatcactgttagaccaaacaagtggtgttcctagccatttctcagaaggggctgaccgattagacgaagatgtagtactagcagttaatgatgattctgcatcaaacatttaccactttgttaacggtgagttatacattaacccagatactcctgagatggataagattaataaaatactagcagaaagaacttatgatgaatctggttcatatcgtgttcgtggttttgatatgtacacagaagttcacccaacagaccctaacaataaaattcagttagttgtcgattcaggtcgggcatatgttttaggttttaaagtagataaacctacaactactcgtattgatattgaaaaatcaagagagttagaaacaatcaataacgaaggtttctactatagcaatgcaactcgtaaaaataaattaggtaattccccagtatcttctgtagaccgtgtaactgcacaagttgaagttgctaaagaacaggtttctcgtggggtcgtaggtggaggtaccgattaccttaaaaacacctctgtaacaaaagttatccgtgtatggactgagggttcaggagcacaggagtacaagcaaggtgaagacttccaattagtaaatggtcaagcaatatcttgggcacctacagggcaagagcctcctgcagggggaacctacttcgttcaatatgtttataataaaacaatgattgaaaatacagattacaaagttgtaattactggtgaaggtgatgctagggaatggtatatcgactttaacgagatgacaggttctaaaccagtcgatgaatcacttgttaacgtagactataaatacttcttagctcgtaaagacctaattgtattagaccataacgggaatttcactgtccataaaggacaacctaacgctttaagactagtagaggcacctaaccatgtagacccattagttttagctattggtacagtagtagtttacccagactcaaatactgctgatgctaaacaatggacaatcacacgtctaacaatggaagagctacagaagttatctgttcgtgtggaaaacatggagtataaccaagcagtgttctacttagaccaaccggcaatggcaggagaaaaccctatctatctacgtggagtattctcggatgcgtttatctcactagataaatatgacgttagtcacccagatgccacaattgcttttgactttgacacagcagaaataacattaccttatgcagaaattaataaaacagtcccaacaattatcgaaggttctagtgaagcacatgtgtggggtagactagtaacagcaccgtttaccgaagaagtaggtattagacaaccatttgctacggaagcaatgaacgttaacccatacaacacctttaacaaacagggtgccttaaaactaaacccttctgcagataactggattgaagatgagcgtatcacagttaccaaagaagaaacatctactatgactgttcgtcaatggtggagacatggtggagcatcttggacaaacgatgagatgaacatggtatctaacatcacgcttgaccccggacaatcgtggggaggtgcctctggcacagaagaccaacgtaaacaagggttatccggttctactttaaccagtggtggtcagcaaaccaaagaatcaatgattgagtttatgagacaaattgatgttgaggtttacgcagagaacttgcaacctaatgctaacaacttatacgtaacattcgatggcttgagagtacctgttaccccatcttctggttaccgtaaaggtgccacagaaggtactggtatggcaaatgcagacggaacatttaaaggggtatttaaaatacctgcaggagttcgttgtggtactcgtgaagtatctgttagaaacgatacaaacttagcaagtactacgttcactgcgcaaggtacgttaaaaacaactgaggatattatcataaaaacccatgttactattaacttggttgaccctttggcacaatcattcagttttaacactaacagagttgcaacaagttttgacgtattctttgcatctaaggataacagtacaaatattatttgtcaagtacgtggaatatcagaaggtggacaacctaataaaactgtgtatgctgaaagagtgttaaaaccttctgaaatcaaagtatcagatgatgcaagtgttcctacgaaaatcagctttgatgacccactaatgtgtaaagcaggtcaagaatactgtttagtattcattactgactctgataaatacacaatgtggattgcaactatgggtcaaaatagggtagacgaaccaacacaaacagtaacatcaaacccttacttggaaggtgtcctatatagttcatctaacgcaagtgcatggtctattcaccaactgtctgatttgaagttcactgtatacacggctaaatttaacgaagaagctgtacttgagttcgatgttatgaagaacgttaatgtagaccgcatagtgttaatgtctacctacctaacacccgcaaacactggttgtagatgggatatgaaattagtccttgataatgagcctgcaggtacaacagtagatgacaaaccttggttacctattgctaactacgtagacttagatgttaaccagttagctcgtgaggctaagcttagagcaacatttaaagctaaccaatacatctcaccaatgctatccttggacgacattatgttcgcaggatttttaacagcattgaaaggtagctatgtatctcgtacaatagatttaacagaagctccatacaacacggttaaaatgtcatatgaacaatttacacctgccggaactgttgtgactgctaaatatagtacagacgaaggtaaaacatggaagacatttacagtacagcctacaacaacacaacgtacacaagactttgttcgtgtagactatgttgaaaagattaatacgggtgggacatttaagtccattaaattccgtcttgatatgacgacccaaaactccttcctccgtccaagagtaagaagacttatgactaacatgacagataaatagcaacagaataagtataatagactctatatgttataatagacatatagagtctatttttttgttaggaggaataaaattgccagaaacacacagacaaacaagctcaggtgcgcttatatttaaaccaactatagctgaacaagaacataaaaatgctatggaatctataaaacaagagagaacagagctagagaaagaactagctaatgttaaagctatcaaagatgagttgtcaaaagagctagcagacattaaacaactgaaagatgaattatcaaaatagtttctaaattgtcctatttaggatttatggttcccttatattattaactaatatatttaaataactatatataaaatatataatatataaataatatataataagactacgaaaaatataaaataggacaaatcaactctgtatacaccttgacaattacctactatgtgatataatgaatatatcgattatactaaggtgggtatgtcaaggagcatgtatactaatctaacttaaaggagatttattaataaatgagactagtagtagatattatgcatactcagataaggtatgaagattcggaaaattatcttagaccagaaatccataaggtaatgcattcagaattaggtgttaaagcagatggataccaattcagtcctgcatataaagcgggttactgggatggtattattgatttttttgataaagaaaatgatacgttccccacagggttactacctcatgtagaaacaatactagggaaccttcaatcaactttatcaaagtcaggttacatttttcagtttgagataattgatgatagacctgatgagttcatgtcagtagaggacatggataaggagatagtgcttaatggggacaacaatgacaagataacattaagagactatcaatatgaatctgtggagcaagttattaagaacagaataggtattgtaaacgttagtactggtgggggtaaaacagaaatagcatcaggacttatacaacaaataaccccttacttagagtcaggagaacgtattgcattttttacgaatagttcttctattttttcacagtcaattgaccgtattgagaaaagacttggaataaaagtaggtgcttttggtgcaggtaagaaggacattcagcaagttacttttgtaatgatacctacgattgtatctgcaatatctgcagacccagaggctaagcttaagctgactgctaaagaaagaatgtacaagaaaattgctaaggatatagctccaaaatttgaaagagggtttaaccaaaggagtttgcttgaaggatacctaaataattttcaagttaagacaaaagcagacttgcagttgaaacatgaactagaagaaattttttattcatgtggtacgaacaaacaggttattatgaaaatgaaagggtatcaagcagaataccaaaagattgttgaaaagaaaaatggtaaagttcttaagaagtacaatgaggctatggagtttttagattccatatctgtaatgattgtggatgaggctcaccatacaagttcagatacttggtataaagtgctaacatcttgtaataacgctcagtatagaatggcacttactggttcgattgaccgtacaaatcatgtgctttggcaaagacttcaagctatattcggggagattacgactaaggtgtctaacaacacactcattgagttaggtcactcagccaaacctaaaataacaatattccctattatagctcctgtagatattcaaaccactacgtatatggatgcctatcaaaaaggcatagtagacaatgaatatagaaattctcttattgcaaagttgactaagaagatgtatgataaaggtaatgggattttaattataattaatcgtatagagcacggagaggctattagtaacttactaaaagaagagggagtagctcactactttatcaatggacaacttgaaaatgatttaagggatgaaaaaattcaggacatgcgtgatggagcacttaaagtaatgatttcttctacgattatagatgaaggtgtcgacatttcagggattgacacattaattcttggtgcaggaggtaagtccttaagacagacactgcaacgtgttggtcggggtttacgtaagaaaaaaacaggagaaaacaaagtagaggtattcgatttttatgatttaacaaacaagcatttgaaaaaacattcagagcagagaagaaaaatatatgaagatgaacaatttgaaattgttgatattcctattcctaaataactataacaaagaaagggtggcatacaattgcaaagtccatgcctaaatattgaattaaaagaaaaatttaaattaaacaaaggtataacagactttttagagagagttgcagataaatctcagagatggggtgaaacagtagcctcccctatccgtaaaacggatatggcaaaagagactggaaaaaaccctagaacaattacaagatatattaatcaattagaggaactaggtttaataaaaaccgaaacaaaaagaggaatgaatggagggactttagtcgtgtttaacacagatatgcttaattttgagccaaaggaaaaccctatcacgtctgatactaaacaagcaaaggagattagagaacaagtcttccctaaagccccaaccaaagtaccaaaacgcagatatagaacaaaagcggagatagctgaggcacgtatacttagtgaaaaacttaaaaaacgtgaagacattttaaatgataaaattgagtttaacgttgttactagaagttttttcgatagttttgacgaaccagaggcgtactttaaaggctatttaatttcaagaatgtataatgcttatgtgacaatcattccttatgaaaaatataatagactgaaaaatttagatgagaaaaaggctaagcaacaactacgagcatacgaaagctcttacaattacgatgtgttgcctagaagatttgttgggacacctcagtacaaaaaatttgtagagctagctaaatattgtgaagagaacaatatcaatcctttagtttatttaacagtgcaatttgatagaacagagtttttgatttctgtaggtaaagcacgagtaggggctacaccctatgtaaatactttgttatgtgcagaggctaaagaggcttacacaaatagaaaactgttctacagaatgttacagaaccaatatggattgtatacgtctatcaattcagaagctacttattatggtgcaacttatccaattatttcaggactattgaatgcttacaatatgccacagaaagacttgtcacagttagatactgcgatttgtgacttagaatataaaaaagatattgacaaaaaagcaggtacgttgtactcttattatacagcaacacttaaatcgttaggtgaatctgacgtatctagtgaagctaaagaaagtattgctaacttcttgaaagaacaagttgcaaacttttctagtaaacgtgggctaacttcaacacagtatgccttagcgttccctattcagataaactctgcacgcagtttattaatgaatgaggaagacgaagaactactttatttattgttaggtaaccagtcacgattatctaatgtaacaaatgatgaagctgaaatgtttattaaacaaggtagaaaattaagtatgtcttggtggggctcacagaacttctcaagaacaatgttcatgttagcggactactatgggtttaaaacaaatatatctaagttaggtatgtacattaaagaatttggtgaggagaaaattcctttagattctgtaggtatgttagatgtaaatagaatctatgatgtcttaatgactgaacaagagattttagagattgacaaaagtaactgggagaaccaaaaagatatgcgtgacgacaaataagaaatataaatgggagagggacggatagtaagatgagtcaaattcaaaaacaagttatttatagagcattaagtgagccattctttgcaaaggaaattctaagtaaaattccaatggatgagtttaaagattctggatatgagatgattgtttctacaatcaacttatattacagaacacatgatgagagcttagaggaacagagtttattaacactagtagaagataagatgttaaaacaaaacaaaagtttggaagctcaaaacaaagtctttgaggtagttagcgacctatacgaactagaaaacgaagatgtagactcagaagttatcagtgagaacattcagaactacgttcgtaaggtactaacacgagaagcaatcatgaaatctgtaacaaatgaaggcacactaggctctgatagtaatattcaacagctaatggatgacttgagagacattcttactatcgagacagcaggcaataattcagaattgctagacttcttcgatgacgtagataaaaagatggagttacttgcaaacttgcaacaaaacaaatacccaactggtttcacagctatcgatgctatttccgatggtgggctagctcgtggagaagtcgggatggttgttgcacctactggtggtggtaaaactacttgggcagttaaccaagctagaaactatgttgtacgtggtttaaatgttctttatgttcctttagaggaaaaagtagaccgtatgattgttcgttttgaacaattattatcacaacaaagtaagaagaacatcttagttgatggtgaattgaataaagacttgtatacccaaatccaacaagcgtatggagcgggcaaagaacagatgaattgggggaacctttggattcgtaaatataaaccacaagaactaacacctagcggtttatctcagttaatctccgatgtaatgattcgtaaaggacagcaaattgatgttgtcattattgactacccagatttaatgaaaaatcctcatgcaagtggaagtaatggtgaatcagatgcaggaggtaaactatatgaagatattcgtgcgattgcacaagagtatgattttgtttgttggacgttatctcagttgaaccgagcaagctatggtcaagatattaaaaatgcaggagctatcgaaggttctaaacgtaaaatgaacgcagtagaacttatttttacattaaaccaaacatcagaggagtttagtaacggatatttgagagcttacgtagataagttacgtaataatagtgggattgcctatgataaaatgttgtattttaaagtactcccagaaactatgactattagagatgaaacaccagaagaacgagcagaacatgaagcactgttagcagataatgcgatgaatagagcaagtagccattccgatgagaataactacacagcaaacgatgttaataaaaagataagtaatttgaataataccctttcaggaggttggaattaatgaaacatattattaatttttcagattttcatatgcacttttttaaagatttttcaaaaccagacccggagtatggaactgatagggcaaaagagcaaattactatattagacaacttgatgaactatgcacgaaacaaaaatggggatgttttatttaatggagatatgttccataaacgagtatctattgatgttagaatatttaatatgttatttcaagtaattagtagctaccctgatgttgatgttatcatggtcagtggtaaccatgataaagtaactaattctctatattcagatagcgctttagcaccatttagtgctttaccaaatgttacagtttgttctacgttaaataagattgttaaagatgattatacgttgtatgctgttagttatggggaagaggtcgaagagatgaaagcttggataaaagagcaagctgacaatttagaccatgaaacagttaatattctaagtgcgcacattggtgtagatggctcatctactggaaagtattctcatacacttggtggtgcttttaaagtagctgatttatgccctgacaagtttgacatagttacattgggtcactatcataaacgacaatttttaggaaacctatctaacgtgttttatgtgggcaataccctgcagacttcttttgcggacgaaggtcaagaaaaaggtttttatgatattactatagagggtaagaagtgggaacaaaaatttattaaaactgactatactccgtttgaaacagtaacagcggatagtccatcaacaacaggttctatggagaaatcttatatccagtttattggtaatgtggacgaggttgaggctgtaaagagaataaaagaagagaacaacttaagtaatattagaataaaagtccagaaggactaccatgttgagccacgtataaacataacagcaggttcgacacctagcgaagttgtcaatgcatttattagtaaaaagtaccctaacgcaaaaataattaaagacaaagccctcgactgtttgagagaggcaatggaagtgtaataattgttcagcctagatatatctaggcttttttgcgttgtactattgactttattagtattttgaagtataatatgtttattgaaataattaattaatattttgggaggaaaagatgttaaagtttaaacgagttagcgcagagaactatatgtccattggctctgtatctattgatttagacaaccaaggactcgtacttatcgaaggtataaatgatacaaacgaaacatttcagagtaatggctcaggtaaaagtactttactgtctaccgttacttatgcattgtatggagctacccctagtggtttgaaagctgatgctgtaattaataaacaagcaaagaaaaatatgtcagtgattttagaatttgaaaaagatggggtaccatatcgtatcgaacgttatcgtaaacactctaaacataaaaatactactagatttttccaaggaacaaatgatataactcagaaatctgtagcagacactgataaaaagattcaagatgtgttcggtattgattacctgacttatgctaatagtatcatgtatggtcagggtaacgtagaaatatttgctacagcaactgataagggtaaaaaacaaatcttagagaatttagccgatattggtgtttaccgatatgcacaggatgttgctaaagaaagagcacaaaaagcactagctcttgcagaagagctgaacagacagtacattgctaagacatatgaaaaagatgggttaactcaatcttacaatagtgctttacaacaatatgagaatacagaaaagctgattcaacaaaaagagagtgagttagctaatgcagaattagctattaaacaaagtgagaagaatctatcagagggaagagcgctacgtgaacccgaattagaaaagctaagagagcagatggcacaattaacttcccctgcagacgttcgtgaaattgatgtagaagttgaaacacagtattctaatgttagtagattgtctagtgcgaaaacacagaatgatactgctattgagaaattaaaaaaagaactagaagatgttaaaacaaatacaaattgctacctatgtggtgctttattgagcccacagcatagagaacaagaaattcaacgcattcaaagagaaatagtggataaagaagtgtttatcgaaaagcttaatagtgcattagcagtgtattcccctctgttagagcaagcaagggctaaacaagaggaagcaagaaaagctattcaagaacatactaatatttatcataaacttaatggtgaaatgaatgcgttgtatcatgagatagatacattagaaaacacattgaacacatctattaataatagggacagcattaaggatatgttaacaagactacaagaaatacctaaacctcaatatgactatgacaaagaccgggaaatagaagatgagctaaataacattaatcaattaaagttagatgccgaagaagaagctagtcaatataaaactattgcacaagaaattttctccaataaaggcatacgctctgaggtacttgacctagttacacctttcttaaatgaaagagcaaaccattacttatctaccctttcaggttcagatattgaaataaactttagtacccaaacagagaaagcagatggtagtttagctgataagtttgacttagaggtagtgaatggctctggtggaaacacatatcaagcaaactccgagggagagaagaagagaattgatttagcaatatcttttgctattcaagatttagttcagtcaaaagcaaacattgctgttaatttaggtttgtatgatgagtgttttgatggtttagatgcaattggttgtgagaacgttattaaaatattgaaagaacgccaaaagaacattagtagtattttcgtaataactcactcggagaacttgaagccattgtttgagaacgtaatcactatgaaaaaagttcaaggtcgttcttaccttgaagaaagtaaataggtgattacatgaagatttatacattaagtagagagttaaacgaaggtactatatttgtacctacaagtagtagtaatgaagggagactatttagcttcccgctagagacactattcgattggtacccttgttgcccaagatatgagtatcagtacagcacttctcgaaaaaaactatatttgagattgttagactcagacaaaacattagtagctagatacggtgttggggataataaaaagagagttatttcaaaaatagcttgctttaatgagaatgaatggtataatgaagaagtggcaaatgagaatgcagagttgtttaactttgctgaacaatatgacattgttacacctctaaaagacgattatacattaaaagaagtagatgatagtatttctaaagtactggatattcttgatttactttatacaaaccaaaaagttaaagtagaggaagagctcataaataaagtggatactctacaattgagcaagccagactcagatgaacttaaacaagcgtataaggacatatctgagtatatgcggttagacagagaggaaaaggctacttatgtgttaagcaggtcactagactctttaaatagtgtttatgaaaaatttggtaatgtgtatacaatgttaaatattatgaggaaagtagtggcttaaatgtttacagacttgttatctaacgaattaggttcacctaaatatgcagtaagggactacaggtataattgccctttttgcgactacgatactaagtataagttttatgttagggtagaagaaggacacccaaagaataacttatggcattgttttaagtgtggtagttcaggtaacccagtatcttttgtaatgaagtattacaacgtgtcttttaaagaagcgctagagatattagaagaatatggctataggtttaataataaaaattatgtgcctaaatcagataagttaactgatgaggaatacttattacttcttctaggttcgttaggtaagccaaaagaagaaactaaacaagctaaaaaagagttagtagcacccccattgccagatggttttaaactactaagccagaacctgagagagccagaagcgtaccctttcttattatactgtaataagagggggtttacattgaatgatatttatatgcacaatattggatatgtaaaagattcttgggtacccttagaaaacggtaagtcagttagactcaaagaccacttagtttttttaactcatggaaaagatggtaagtatcaatattggaatactagggctattggggaaagctttattaagtctttgaacgccccaagtaaagagggtgagcattcaaaaaaggatactatatttaatattaatagagctagtcaaacccctcagatagttataacagaaggtgttccagatgcattaacagttggtgagtctggtgtaggtacttttggtaagcaagtgactgacgagcaagttgaacttattttagatagtgtgaatgaagagcaaaaaatatttatctatttagataaagatgctaaaaaagaaattaagaagttagcagagaaactatataagagacataacgagacttatgtagttataagcccaacaactcaagatgcaaatagtctagggagagaagaggcttggaatattataaataactactctgtaaaagcagatggagtagggttaataaaattaatgttatagataggagagggaaaaaaatgaggtatacactagaagatttacatgcaggtatgaaactacgctgtacagacgataaagagtttagccattggactactggtaaaatctatgagctaaaggaagaccctgcaggtcacatgtatattagggacgacaatgaagagaaagtctgtggtataagcatcttagggcgtttaaatggtaacacagggagcgcagaatttgaggttgttgctaaggtgataaaggatacagattacacagaagaagaccttgaagaagggatgttatttcactgcaaagatgatatgagcttcccatggtgggctacagggcaaacctatgaaatctataagggtgaaaaaggaattttatttaccaagtcaggagacggtaaccaatactgtgctaaagaaatagtagctcgattaaatggtagtgcaagcgggtcttttgaattactagaaagaccgcataaagcagagttggaaaagaaagtagaagcaagaattaaggaactagaaaaagagaaacggcgtttacttgacaagcaacagcaacttaaggtggaagaaaacgaagtatctgtcgaaatcagtaaactacgtgaagcattaaaagcaattgacatactaagagaatttgaataagaatcactataggaggctaacaaggatgggaagagtatcagtatattttctaaatagcagaaatattatggatgaggatgagacaaagcgtacttatcatggaacatttaactcgatgaaagaagcagagcagtcagttcgtgactggtggaaagcaaatgactttaagtgtagtacccttagaattattgaaggtactgaggatgggattgttcgttgggactacggaaaccacactggattctatctatttgtaccagaaggtgctgtagtaaaatacacaatccgtgaaggcgctaagaaaccaaaacgaggaagagaaaatgatgtagcacatgacttattcacagcagacgatggagtagttattcccgggagattaggttcaaatgttatttctactggaataaaaacatcgtttgaccccaaacaatatggtttattcattaaccctcggggtggtatgatgaaatacccaatcactttaggaaacacacaaggtgtagtggaaggggaatatcgtggagaagttggtttaccacttaaaaatacattctctttacaattagatgcaagagctgtctctaagaacgttttaacaatcaatgaagaaggtaaacttattaacatcccagtgacagtagctcggtcaatgtatccaagctttaatgctctttatgaaaaacagctagagaagctaagtgaggagttacagctagtctatggaggagaagttagaatatctaatgctgatgagtatgtggttgcgggaacactatttatcccaaaaggcactcgtttatgtcaagctttcttactaccacgatacgacacacagtttgttgaagtatcagcgttaggcacaactgaacgaggagaaggcgcatatggttcatctggggtggtatagccatgctcatcccagaatttaaaccaccattattgtacgttatgggtagcttttctgttatgttagaaaaacatcagtgcagtgtaacttttgatttaagagagccttatttaggtacctcttacgataaaatagtgaagcttattaaaatgacttacccaaactatagcttaacttatatagggatggcagacaataaatataagttcacattaaagaataaggaggactagcatgtacaccaagaaagaagaagtagtaacagttaaacacctagttgataaagagcaaattagagtaggggacatagtaggttacaaaaagactattagagggttcgatgcaaaaagagtaaaagacacactctcagtaacaaatcaaataggtgtagtttctcgtgtatgtgacgagtatattactgttcatgacttttttgataagtgctcacgagaaatatgggcaaagacatttgaaaatttagaagttcgcaaaatagaggatggtaacagtctactacggaggtatgaaaatgaatttcgttgattattttaatcagatgcagaatttagtgattgaagaacaaacagatgagtatgttttattagagaaagaacatggtcaaaagtatgtgacatatcaagagctagaaggagcgttaagtacagtagcacgcaacactgcatttatggtagaaaattataaccttatgcaagatattaatttaaaaattgtattgaaaaaattaaaagacagtggtacaattacagaagagcttgaaaaagagattcttaaagagttcaaaaatattgaaagtttaatggaggacgaaacatatgagtaaagagagtaaacgcaacaaacgtatcggggaattatcggaagcagacatgagagtgtgggctgagtggttagctacggggcaggttcatgataaaaaccaccaaaaacagttagaacgcttaagtaaacgttcagtatcattatccgatgtaactacgattgtcgaatttatgggcaaacggaatgatggttatatttcttctttaattgaacagcaggctgtatttgacaatttattaacaaaactaggtgtaacggaagagaaccgcttagaggctaaagcagagtacgaaaaagagttaagccttattcaagaaaaaattcaaaaagagctagagtcaattaaagaaaataaagaaaaataatatgaaagaggttagctgaatgacagactacagcgcagtaggcaagaaaagtcgtaacaaaggcggtcgttttgaacgtcaaatggctaaagagcttacagattggtggggatatgaatttaatcgagtacctgcctctggcggacttcactgggcttctagtaataacgttgccggggacattgtagtccctagcgatgctaacttcccatttgttatagaatgtaaaaatcgtgaagactggacgattgaaaacttattcttaaataacaaagaaattaagaactggtgggcacaagttgtgggggatgcaaaagaaacgaaaaatatcccactacttatatttactagaaacagagcaaagaattttgttactatggcatataatgaaaaacttgttaatgagattgaaaaaagaggttaccctttgatggtctctaacataacatatgtagacgactacaaggatactcattgttacaagacatttactacagttttagatgcaataactagctttaagccttatggtagcaaagacaaagactactttttattttattttcctagtgactataactgggaagatagtctagtttatgaaacaaccataatggatgatgctaaacaaatggatgcagaggattcactagatgcactagttaattcttatttaggaggagaatagcatggctaaaacatatcaggaagctctagcaacagtacagtcgtatctcgaatcagatagtgtaatgaaagaaacctcaagtatatctgttagtttctcagctaactggacaggtgagcgggaagactacgttattgatacattgacatacgacattgatttacgagtattcagcttagaaactgcacatgttgtagctatagggaagaaactacctcaagatagcaatgagcatgctgagctccttaaaagacttaaaaaagaatttaaacaagcttctaaaaaactacgggaggactagagtagatgatagacaacgttaatagccctagtcattatacgcaaggagaaatagaggtaatagaggttatagaatatattactgctaagtaccctgcggaaattagataccatttagggaatgttattaagtatatttgtcgagcacctttcaagggaaagctacaagaagaccttaacaaaagttcttggtacttgaaacgagcacaactggttttaacacagtcaccaagtagctatataggtaaatgtgagaaatttttagagaatttcctatttaagaatagccatatcagagatgctcagctaatagaaataccagaagaacctattattgagaaatttttatttcaaacagcacagagttacaataaagaacagcaaaactatattatttcagccttaatggaactaaacagtagttcaggtgatgtgaaaactgttcttgaaaatacagaaaactatttaaaatttattacaacttagtacaatataatgctctttctacgaaaagctatcttttatatttatggtataatagagatagggaaattaaaaagtagaaggagcattttataatgacaaaagcacccagagtgaaaagacttaatatttataacactgaccggtactttaatattaatttaatgaaaaaagaagatatagcaaaaaaaattaaagttaaccgattgaatgaagaagagatagagagggaaatggacgaactagcaagcaacccattgaagacccctataggctacatggatagaacaaatgaaaaatcttatatactttatcaagaaaagtatacgaacgacagacttattcagaaattatttaaacatgcagggtctgtgtcttattacacagatacaattgtaccatactacataattgagcaaatatctaagaatttaacaagtgaagtaatctatcctacaaaaaatagctatgagaacagagagattgaaaatgttcaactagctttcactgcttgccctgtaacgattgactgcccagtagtgctacctgatgttagcccgtatgatgtgttatttgcactacatcctctaaaaacaaatgtagacaagattcaaatatcgtttccttgtcttacagaagaagagttcgatactagacatgaagaatactaccacaaagtaggtagccactatgaggttaaatcagagtacaaatataaattctttaagtatgtacaaacttccttatctatttgggctatgaacatttggctagtgtgtgatagcgatgaggactacaataaaatagatagatatattcagaaagaaaaaattaaacgtaatgctaacagagagcgtgcattaaagagaaagggtaatcaggatgagtaaagataaaacgattaaccgaacagacatagctcgaacaatctctcaccatactggttatcgaatgaaagatatattgaagattttagaagtagaagatgaagtagtagctcaagcagtatcacaaggcatctctgtaaagaatcacaaattatggaaactaaacattaaaaaaaagccagagaaggtagcatgggatggtataaactctaaaagttttatacaacctgaaaaatatgtagttaaatttgtaccattatccaagttgaaagagtcaatagacacttataataaagagagcaaataaagctctcttttttgctctttctatattgacaaataacataatacaaagtatactagtgtcatagagcaacagagaggagaaaagtgtaattgaaaattctattcttacaagagtacattagagaaaatcatgtccataatggaaagaacggacaaacagttgattttaaaagaacagaaatgggtaaaaaactcacaggtctgttaaatacaatagggctgacaggtagggactatgctgtagactatgtgtacgacatgattccggaagtacagaaagttaaccctagaacaggtaaaccaattaagtataagacacctacgttaagacagcgtaaagaaccagaggaacggttactcagacgtttaatgaaatacaaaccagatattatcatcccaatgggggagatgggttgtaaaaatttattaggaagtacttctatcacaaagaatagaggagtaccaactaagaaaacaattacgaatgagaacatactgagaacggctgatgagcaagggcttgaggtagacgaagtagtagactcttttgaaacttgggtactacctatgtttagtatggaatactggacagctaacccaaacattgagaactttatcatggcggatattgatacattgggtaagtttgttcaagaaggggaacaagcatttatccctaaaaaagtagattacgaatttgttgataacattgaacgtgtaagacaaatttttggttttttagacaaaacgaagccagtaactgcatgggacttggagacaaatagtttacgtggtgacttattaggggctaagcctcttgtaatgtctatgagttggctagaggggcaaggtgtgactataccgttagaacaccatgaagctaaatggagcccagaagagcttaacgaaatatatgacttatttgagaagtttttagcggacagacagcaaccaaaagtaggacaaaatatccagttcgatattagattcttaatgaacacaaaaggatttacagagtttgaagacaacagagatacaaaaattgcttactatctaattgtttctcagaaagtagacacttcaaaacggctatccgatattgcatatgaattaacagatatgggaggctatgacgaaccgttagagcagtataaaaaacaatataaagaggactatattgctcgtaagaaagcagagattgatgctttcaaagaagctgaaaaagaacgagtagagtgtgagttcaagatagcaatggatagatataagcaagaagttaaagaagctaagcttattggtaaacctactaagtccattattaaaccagtaaaagaaaaagttgtagtacctaagaaatcagatattaaacttattaatgagattgacggtggtaactttaactacgactggattccattagagattatgcacccatatgctagtggagatgtcgattgctgtctacgtatctataacgttctatataaacgaattgaggaacatcctaagatgttggaattatggttgggcttttatcctagactaacagcaacattagcacacatcgaggcttctggtgttctattagactctgaatacgcagaattagtagagaatatctatacagaagaagaacatcggttgcttaatgaaattagaaagctaccagaagtaaaagaagtagaggatgaacatatgatgttctacaaagaggggcttaaagaaatgacaaagcctgtcaaagatagagatgcctctgttgccagactacgagataaatataaaattactgatagtgagaacaaagtacattttaaaccaagttcgtctgaacataaaggtagactgctatttaaagtaatgggacttacactaccttatgataaagaatctattaaaaaagaaacttttgacaatggggtaccagaacatcagctaacatggagagattataaaacagataaacatgcactagcttatattgcagaaaattatccagaggctaaacatgttgctgaacttttgctagagtattctaaagtaaacacactaaaaaataactttgctcaaaagttacctaaattagcatctaacaaggatggcatgattcatggttcatataacagtacaggtacagaatctacacgattgagtgcaaacaacccgaatatgcaacaattaagtagtaaggttggagaccctagacgttttgactataaatatccaattaaacgtttatttagaacaagatttgagggaggtggaatgctacaactagactactccgccctagaaatgcgtattttaggtttgattgcaaaagataaagcaatgacacaagcatttattaatggtgaagatatgcatgaggcaactgcatctcttgtatggaagttacctaaagataatgtaccaaaagatatgcgacaacgtgcgaagagtgtcaacttcggtattgcatatggtgaaagtcctttttcaattgcacctaagttaggtgtaactgtagaagaagcagagcgtatttttgaagagtactttgcaagtaagccaagtattaaacaatttattgaagagacacatcaatttgctcaacgttatggctacgtagaaacactacaaggacatcgtagactattgcgtgactcattctctaaagataagaaagtatttaatggagcaatgcgtaagtcagtaaacacaatcattcaaggaactggtgcctacttaactaacctatcaatggtatacattgacgagtatattagaaaacacaataagcgttcacgtattgttatcactgtacatgactcacttgttatcgattgcccacgtgatgaagtggacgagatggcaaaagtggctaaatatattatggagaacttgccaattgatttcttaatgattgagtgggaaggtaaagagatgcgttaccctattgtggcagatgtagagattggggagaattacaatgatatggttgattatgatgcagaaattatcaacgagtttgcttcttaccgaggatacgtaaaatacttcaaagaccaagctaagattaaagattactatgataacaaacttatctcagaagagcaaaaagaccaaggtatcaaagttatccaagatgcaatagagtcatataaacaaatgacaatttaataaaaaaatgttaaaaatagcttgactattgacgtatagtatgttattataatatggcagacaagttttctgtattaatacaagaagggggagttagagagtgtgcttaatttacgagtagatgatttagagttcaaaacaattaaaattattgatgataacggagaagttgtgacacatgatttacaaacagagttacaagtcaatgaatttaatgtgagaacagcatttttagagcaacctgctaagtatacttattggacttcgatactagagcgcttgcgcatgtatcaagaaaactatgagttaaaagcagagaagaagaaagcagaactatatgagccttctcgggttgccttaatcaatcaaggagtagctaaacctacaaaagaccagattgaggcacagattatgttagatgaagactactacaaacttagacagagtatagttaacctatcatttaacgtgagacaactgcaatatatagttaaagcttttgaacaaagaaaagatatgcttattcagtatggagcagaccttcgtagggaatatgagtatagccaaaaggttagtatgccagacccaatgaaaaataaggtaaataatggtttctcagactttcaatggaatttagaacagtaaaattagtgataaaaaaaataatataatataaaaaaaatagaaatggagaaatttaaatgaattttcaagaacaattacaacaacaattaaaacaacaaaatattggagaacgtgaagcggtagactacccatcaaatcatttaaaacataaagaattatacttccctaaagcagaaaatggtcaaccatcaactctatatgtacgtgtgttacctcctgcagtaccgggagaaaactataatgttagcgctcgtgaggcattcttaacaactcgtaatcgtaatggtaaagatttaaagagcaactttatcttttcagaacaccctaatgcagaagacatcttagaacaagctatgattcgttggaacgcagaaaatcgtgtaccaaacccttatagccgtaatacaaaacctcgtcaacgttactacgttaatgtagtgcagttaattattaaccaacaaacaggggaagttagctacgaaacagattctaatggtcagctaatggttcgtttacttaagttaccacaaacagcttgcatggctattaatgagagcttaagtaacccaatgttacgcccacagttttcacctgatgtaccagaagaagtagcacaatacagctttatttcttctgcagatgcattccctatttcaattacaaaaccaccacgtagtaacaaacctacttcgtacaatgtacaagtgattagtaatcgtagtttaggtgcattaccacaaggttgggaaaacttattagaagacttgaaataccaagctacaccttctgtagagtacaatcgtgagtttattgagtactttattgatgtagtagacgggaaagaacctgtgcatcaaggagcacaaagtcaaggaacccaagctccacaatttaatcagcaaccagtacagcctcaatttaatcaacagcctgtacaaccacaatttaaccaacaacctgtacaaccaatgcaacaaaatactggttggtcgccacagcaaagccaaccacaacagcctgctacaggttttaatgcaacgaacatgggaacacctccaaacattaatggagggtttaaccaacaacctgtacaacaacctgtacaacagcaacaaccgatgggctctttcaatgaacaaccagtgacagacccttctaccatttctgatgcggatatgcctttcaatatgcagtccatgcctgatgtatcaccacaacaaaatgctgtacctgagcaacctgtgactaatacaccagagccagtaagccaaccagttgttaaccaacagccaaataacacacctagtgtagacgacctattagcaggtatggtaggcaacgtttaacaataatgaaacagttaggaggggcatactgccccttttaattctgtgttaactatccaaaatatttagtaggagtgaataatattggcaagaaaaagaaaaagtgaagaaatagattttggaactattgatttgacaaaagaggtaggactaactacatttacggacacaaagttctctaatgtatcagacagactaccaacaatgattccacagcttgattacattttaggtggaggattgccatttggacgtatggtcgaggtctttggtaagaactctagtggtaagtccactttagcagtccatctgacaaaggtagcgcaaatgctagatgtaccaactgtatggattgacgttgaaggtaccgcagacccagaacgcttagcagaactaggggtagactttagtgcaggcggagtattcatggtagagcctaaacagaacaaagacggtagtaaagatacaatcacggtagaacgtgtagcagaagagttacaacgactcttaccagtatttagtaagcttggaaaacctgtattaattatttgggactctgtagcgcaaacagcttctgagaaagagttagaaaaaggtgtaggtaaccaacagcccggaattaaggcaaaagcaatggcacagtttgctcaaatcattgcacctttaatgacaaattcaaaagcattatttattgctattaaccaagcacgtgatgagctaggtagtatgtttggtggagtagactctcccggaggacacgctttacaccactgggctagcttacgattagaggtagttaaagcaagtcagattaagaataaagagctaaatgcattcggtgcagaggaagagacctatgtaggacatatcctgcgtgttaaaacagcaaaatcaaaagtgtcccgccctaaccaaaaagcagaaatgtatttaatgtcagacacaggtctgaacttagaggaaaacatttatcgttcatgttttgcaactaataagcagtatgccttgattagcggaggcacatggaagtcttatacaacggatgcaggacaagagattaaattcaattcagataaagcttgggtagcttatttacgttcagaagagggaagacctgttcgagacgaactatttgctaaaatgatggtacgctcattcccacatcgttatgctccgtttaataatgaggatgtggacgtatgcaaaattccattatatgaatttactaaagaatatatggaaaatcataaggaacaacctaaccaagctactaaagaagaagtccccgaaacaggaacagatgtttcagatttacttaaacaggtagactaacagataagggggcgtttagcccctttattagctataaaggagaggaacttattttgcgcaaaggactagcacctaacccattttttgaaatattagaaaaacatcaagactcctctaaacgtaatatgactatgaacaacagtggtacacctagttcactacaaccaataagagacatgttcttaaaggcaatgagagaaggtaagagagtcctaatagagaactctgacttaagtagtgcgaactctgttgttatagagatagaatatgtaggtaatcgttggtgtttaggttatcaacgagtcttattttatggtatggagttaaaaataccacacactatccacttctgtgatgtatatggagcctatgggcacgatgctcagaaggttaagagacaagttaaggtagtgtttgagggggacaacccttttgagtagagatgttcaaaaagaggaaaaagaaatacgtaatggtaatcggtttattacagagactcatggcaaaggagtgtttcctagagatgtagaccgattgtaccataagtatagtaaccttagatataaagtctataacacccataaagactcgttcaatagtgaggcttcacgtaaggaactcaaaagctatattgatgagcaatttataaagttaacaaaagagtatgatataaatggagaggtagactttccgggatatattaaaaaagctctgaatttacgagtaaggcacagctatgtaaaaggacggttccgagataccgctagagaacgtctaggcacccaagataatgaagtagagttattgttagggattgacgatagctctcaggcagatattgaggatgcagaacttattgaatcgttattatcaaaagccaatttttcagaaattgaattagcagtatttcagcagttaattcaaggaacagtaagggatgctcgtattattactgaattgtcagaaaactacggggtgtctaagaaagccgtaaaagatgctataaaaaatgttcgtgagtttgtcttaataaatttaacagattagagtaatacctcctttatgtctgctatattagcagtagaaacataaaggaggtattttttagtggaacaaaacaacactggcaagtatgcaccatttattcgtttaattgtgatgggaatctcatttgtggcaactggtttgactacaatatttggttgggaacctttaccattcacagacgaacaaatgaaccaaggtttaatgttagtactatctgtaggtcttgccatctacaactggtacaaaaacaacgctgtaacttcttatggtaaagcaaaagaagaagcaggaaaagaagtagtcggaactagacaagacttcaaaaacagagactaagtacgaggggtgtaacccctccttagttaatacaaaggagcttcgacatgaagatagacgagataagcaaattagaattgcctaatctatttggaaaattccttgtggtagagactatttcggatgggtacactggaaccgtatctggtcactataactacgagatagaccaaaaatctgaggaaacttatatttaccctgtattttggaatgataagcttaacaaatttattaggtcagatgaattagttgtatacactaataaaaataaagtatactatgtttgtaaaacaactatagacccatataatcatgcagtagtagatgagctcacagtagaggaaggcatggacaaagataagcgtacccttcaagcgtttaaactttttgtaaatgacctattctcatttggtagctacagtatctttctaacaggtaacctatcattagaaaacaaccctgatattgttctagttagcagtgtatcattagacaaacagaccgctaaaatgtacacagaacggacattagaactaactgctacggttttaccagagggtgctacaaacaaaaaagttagtttctcagtagataaaccagaacttttagggttaacagtctctgacaataaagcaactgtcacaagtaaagataaggcaggtactgcaattgttaccgtaactactgaggatggagaacacactgataagtgtacagttcagatagaagaatacataaaagttacaggaattaatgttagtggagaatctgctttagaaaaaggtaagacatacaaatttactgctagtattgtacctgataatgcaacgaatcctaaatttacttggtcttctagtagtgataccatagctagtgttaatgcaagcggtgacgttgtagctttagcattaggtgaagcagacattatagcaacaactgaggaaggtagtcatgtaggtaaagtacatgttaccgtatcagacccagaaccagtagaaccaactgaataacatagaaaagaggaaagaaaatggctaaagaaatattaaatattgaagacctattaaaaccagagacattagaagtagcgatagatggtaaataccttattgtaccaacattgtcagacgggtttacaggtacagtagcaggcggatatgcatatgctgttacaaaaaaaggaacagactatacagttaatgaattaatctataatcaaaaggataacacatttaaaccttccgatgaaccaattattataactgatgataatgaaatattcttcattactcgtacattggaagacccatataactatcctgtagttgctacagagaaacttaaaactaaagatgtaaaagaaaaacaagttttacaagcgttcttagcatttgctgatgacagatttaagctaggtgtttacaatgtgttcttagcagatgaaccatttgtgtatggggacaaaacagaatagtttcattataagagacccgttaaggtctctttttttttgtatatagtgttgacaatgttgtagtttagttatatactatacttatactgtataaggaggtaaccacatgaacgacaataaggaaaaatgtattaaacaaggaatacgagatacttataaagggtacgatattttattagatgaagagaatggatttttctatgttagcgtacttgacccagatggcaaagaaatcattagtgggtttgtagaggcggataaacctattgaagaatactataaagagctcctaggtaaatgcgaccaagacatattattcaaagaccttttaagacccttaaatggacgtagagacactgaacgtacagacatacgtttttttaaaaagagatggtggggtataagtaaatgacaaagataaaacttattacaaaaaagaacacccaaggctatgtcatgaacaccctgctacgtagattttataaaaataatgtagacgtagaatttcttaataaattcaatctacctgatattcataaccacataggggggcatgatgctgttatcatagtagggtttcccttctttgaaagccaacgaggtgcgttagacacggcactgtcatctatggacaacccatttagcaaggtgtaccatttagcaacatttggggacacatatcgtaatgaaggtagctttcgttcatttgtagatgaggttataagccccgttggtcattttgtagaactaataattgacctaactaagttcactaacacaggtacaaaagaggatgaacaaaatgtagtggctcttgctaaagaggcattagtgtttgccaaagatattatagaagagacagacagctacaatcgttatgaagttacagacagaactatctcttgggttcttcttgtagatttgcttggagagaacctatataaggtgacagagcctagtaaagagctagacaccattctgaaagaacaagaagtacttgtagatgccttgaacataaatatgcaggaatatgtgctaagaacaatagggaagatgtctgcaaacgtcattaatggaacagtggtttgtttcggttatgcagaacagcatgttaacgaagtagcccataaattaattaatttttataagtcacataactatcaaaaagttattgtctttattggtaggcacacaaaaggtgacgacatgtttagtgtaaggagctatggtgtgaatgccggagaggttgtctataaagttcataatggtaaagggaaagacacaacggcaaccgtcttcttaggtaaacctagtgaagctgtaaataacacactgctaagtgtgctttctgaaattttatagtttaactttgtggtataataaagatagataaattttgataggagtgtaaatttttgaaaaacaacgaacctttagagaagttattagacaaattagatgaaccaagaattttacaaacaattattataggtagtttacaacggagctttaatcgagtacacgttggtaaatttaataaactagcacaggagtttgacttagataaggagaacctgtatagcttaaaggcactagttaaggaaattgaagaagataaagagctacatgaactttacgaagctagcatggcaggcaaaattacgctagaggctgttcgtaaagtattgttacaagatgataaatcatcgtttgatgtactgtcttcttacgtagtagaaaatcaagcagtgctagcccgtaaccgagagttcggtaagttacaacgtgagggagcttatcttgaccatctaattagtggtttgaagacatacttattaactgaattaaaagacatgtctagtttaaaatatatcaataaaaatttaaaggcaccgaaagtatcctcagaccgagagcttattctgtgcctatctgattggcatattggtgcttttgttaataacattgacacaggtggatacaactttgaaatctttaaagaacgacttgaaaaattattagaagaagtattccaagtggctatggagcaggatattaagaaaattcatgtttaccatattggggatattattgaacacattaatatgcgtaatgttaaccaagcatttgaagcagagtttcctgctacggagcagattgctaaaggaattagagttcttgctgatacactcaatttactagctaaggcagaatttgaagtgtctttcggtatggttggtggtaaccacgaccgtttccaaggtaataagaatgataaaattcataatgataatgtggcttaccttgtagtagaccaactccatttcttgcaagaattaggggcattaaacaaagatattaaacttgtagataacagaagtgacgtttatagttttaaagacacagttgcaggtaaacgtattaaagtaactcatggtgacactgagggcaaaaaagtagatgttaaaatccctaaacatatcaaagatgaagttattgactatttaatcatggggcatatccatacaacacgtattattcaagaagacttttcaagattccatgtgtatgtcggctctcctatgggggcaaacaactactcagcagagaataacttgccaacaacaagtcctgcacagctaattatggtattagaccctgagcgtgatacgccacagttcatgccagtattcttataggaggtactcgtatggaaggtaatttagtttatattttattggctattgcatatgtaggtgaaggtataactgcctttactaatactaagcgtaaagaaagatacatgattgaagaaggagaggctcctttgccacgtagttcttatgtatttttaggcattaactaccttttaagaatagctatagcaatttctcttatcttcatcataccaactagcttgcaacttaacgtaacaggtattgctttgttcacactaatggtatttgcagttccttttatagcaagaattatcgaagttgttattagaactgcaattgttcgttatgttcaaaaacaatatattaagcagttagaagaacgaaaaggtaaaagagagactaactagtctcttttttgctatattgaataatgaggtgactaagtatgaattttacagaagtaataagccctaatggggagacatcattaatagatgtaaacaacccaccaacattaatcagacgtggagtcttatctattaaaacaaaggttaataatgaagtgaaggaaactcctgtttatattgttgaactagccgaggagctaacaggtactgatgtagtatctgtctataaagtaaaagagattggagattctatccaaaaagactacattgaagaaaaagtgacccctaggttcaaaagcacaacatatctgggtgagttagcacaaaagattaaaggacggtcgataaaggaacaacgaagagttgaaacaaaaccaccattattcctagccccagtagttaatggaatcgatacattcacaggaatcgaaggtaaaggtttctacgaacgtgaagaggacagacatattctattacctgatggaaaaccgggcatagcctatggagataatacaggggtttttatcggtttgagctcaattaagtgggacaaggcatatgtagacgtggagtctataacaaagggttatttgtcacagaaacagatatggtttaacctagatggtcaaagaccacaatttagaagtgagacactataatgacagataaacaattttatgaggcagatattcaagaattaattttaaataagcaaagaatattcggggatataggaaagagcgctattgtttttgagaaagcaatcatgcaaggtaatacaatatgtgattgcctagtcttcacagaaaaacgagggctcattggcatagaaataaaaactgaacgtgactcgacaaaaagattaaataaacaattatctgattatgaaaaggtgtgcgactacgtatatgtattatgccacgataaccatgtacctaaagtagagcaaatacttgctagacataatcataaacacgtaggtatcctagcttacacagagttcatgggagaggcaatgttaggtgagtataaacaacctagccgttcccctaaaaaatcagcttatcatatgttaaatatcttgtggaaagaagatttaataagaatgcttggtacattcagacggtatggtgacaggctagaagctaatggagctaaggttatgaagacaaacagccgttctggtggggtatctgggctttatgttaaatctacaactgctagaagaatgactaaacctgaactcattaataatttaataaatagagtaggcggtaccgaagaggctacgagagtgttctgtgatgtgtttatccataataggaatcacccagagaaagcaattaagttaagacattttaaagcaaaagagaatagaggtgacctagatggggtttaaaggtgcaaaatatggttcttggaatactgtagttggtaagaactatgtaggtactgggggcagaacaagtggtagtaacacaaaacggctgtccacaaaaggctactaccaagtaggatttgttaaagaatatcagaacttgacagaaaaagatattatgttaaaattagagtatggaaaagacttagtgtctagctacactggtgtacctgcagatatgattaagctacgtaaaaagaaagaggagcaaactctagcatctttagacacagtttattatgttagcattggtaaggaacctgtaggtaagctgtctatacgagctcagagacgttttagagaggtagggttaacatttatatacctagagaagaactacgtacagaggaagcttagaggcggtaatgtacgtagtgtaggatatactaacgcaaccaaatctcagaaacgtaaagcagatagacggaagagcaataagtctaaaacaactagatagatggaggactactatgagtatttatgatgagctaattggtaaaagtgttatagccctagaatatgcggaggatgggtgtgaggcaagccttgtactaaatgatgggtcatgcttaaaagtatcttgtaacccagagccagactgttgtgggtataacgattttgaagttattctcccagatggttttgatttcacagataacattattacaaaagttgaagataatagtgaagaatgttatggcggttcaacagttaggataggcatttttaccaatgatgcacggatagctattgaaggggactacggaagtggttctgggtggaactatggagagtatgtagacgtagaaattgtaaaataatttactattagacggaggtaagctatgcttaaaaaagggaaagaagtaacactaagaaaattttataatattatcacagataaggaatcagtattgctaggggcacgttacacaagctcaataatgactacagatattcctattacaacaacttttgaagatgttgaggtagaccttaagaaagaaacagttgcaggaactatttcattcagaccagtaggagaaagacagattaacgcattaagtttgctaaaagaagcaagcacagcttatggaggttacggagagttcctagatgatactattgagaaacctttaatcaatagtgattttgcagtagatgttagcctagcagactctgctttctctaacctacaagagattccatttaacctatatatgtcctctccaaaagttttttatacagaagtatcaattagagggcgtaaacatatgcaatatgtattagaggatgagtcttctagcggtgtaacaagtacattagcactggtattccgtaaaaaattgtatgatggggaaacattgctaggtgtgcatacttacaccgaagcactagcaagagtagagggaattaaagtattacagtttatcgcaaacggttctgtactagaacaagtaatcggtgcagtgtctgttttaggaggtagcacaggaacaaccctgttccctatgtttgacgatatggtgatgcaatttgtgatgattgagtctgttccttgggtgcacatttcttgtgatacaggagcaattgcttttaaggaagaagacatccgcaatgtaaccattaagtcagctagaccgggagaatataaggtgactatctacttgctagacgaaaaagttacattattaattggataaaattttaaaggaagttgacaatatatgttgacttccttttttgtatgtgctaagatagctacagttggataaaaaataaatgatatagagggagagatttaacatggactacaaaacaaaggtatatgtaggagcgcttagttggttaggtactttaggagagaaacgatatttagggcaaatgcgtgatgtattagctgtgtacgagctaggtagtcgatacgcaggttactatacagaagatagcgactatgactatatggttgtatacatgccggctccttatgatttaatgcatcctacaacaatctataaacaggaaacagagattgatggaaataaggtagaggtaaaatatatgtctatcgtggaatatgtttaccgtattgaaaatggagatttagaggctttacaaatgttaaatgccacaagctcccaaagtttcttcggagagcctattgaaggtattgacaataagcgtactcgtttagttacgtatatgaaagaactagaatacagaagagaaacatttacttatttagcaccagagaagttgtttagaggaattaatggtcgtattaaagccacaaaagcacgtatggataaggctattgagaatgacgatatggaagtagcggtaaaatgtgctatccttattcgttattttatggacttattagttgtgctagcggacggggaatctattcgtgaaggtctcacgttctctcctattattgcaaatatcattagagagtttagggaagactgtgagtcagcacaggctaagacacttatcaatactgcacaagtgctattagaggcggatagagaagagattttaaatagtattaaagaccatgggttatcagatggttacgctcaagcagttagacaatatagtttaacaggtcgattgatagatgtactattagggggatactatgattaaattagagcaaaacacagaaattttaaatatttttaatggtaaagggagagtggcagataaagtagaagaatctgccaaaattatgctagaattagaccacgattatgactttggtacaggagacattgcttatacagagcgtggcacagataagaaaggacgtttctacctagaaagtcacttgtttatccataagattatgccttatgggttcattttaaacgctgtagtaagtaaagtgcactatggagaccaagaggagaccttagaacgagaggtatctcgtgtatatgagctagaatataacctacgagataaggtagctgtgctgattaagaatggtaaaccactaaatttctcagaaaataacattagtaacatgttttctgggcacgtagcacagaatattcttgaacaattagatacaatgtctggttcagatatgtatgtatctgtgtatgaacgagttagtcgagttaaggatgaacaaattggtaaagtatctcggttctttgatagattgatgaagtacaacaagattgaattaatttataagagtggcgttcctgaaggctttgctttggcttatgcttataaagtagtcgttgtgggtgaagggaaaaatagggataaatggtaccaacctgtacagaatgtggactatttagacaaggaacaaacaaatccggctaaacacctaggtattccaaagtctatcttcaagattatttgtgaaggcggactagagtggacattctaccaaaaaatggctagtcgtttccataagtcagtggataacagaagtattgtaaaaacccctgaatctcctactgctatgaagaaggcagagaaagaggttgctaagctacgacaagcctatgctcgttttggcggggtactgtataacctttatcagtttactaaagagctagatgaacaatatggcatctcccatagtaaggatattatcgaaacagagtgggaatctatatacgaccatattattgcaggagtagttaacgataactataatagtacaactgcttatgatatttctatggttgcaaacctagacttctacagaactgttcgttatctatactatcgagtatatgttgagcaagggcttacttcacaagcagaggctaggtacacgtacagagactacttgagagcacatcattatatgaatgcggtacctgttaagtaccctaaagctcttaagactgctcatgacattattaatatgaattaccgaacaatgaaggataaggtattgaatagtaactttgaaagtagtgtagctgaatataaatatttagaagaagtatcagttagaggtggctatattgttaaggtacctgatagtgtggaagacttaactaaagaaggttctagtttacaccactgtgttgctacttatgctcatcaagtagcagaaggttctactcgtatcctgtttatgcgagataagacagagccagatactagcttagttacttttgaggtaaaaaataataaactagtacaagcaagagggctagttaaccgtgacttgacaaataaggagcaggagttcctagacaaatggttagctaaagcagaaattggtaagtattagtttgcctaaaaaggcggaatatggagtgtgtatagaccaattcacaatgaaaaaagtacactaaacagttgttatttcattattaggtaaaacaacttattttagatagctgatattgaccgacaaatatttcataatgatttagctattaaagttacaaaaaccctactaatatgtggggtttttgcttgtttcaattacatatgaaattttacagatgttctgctatattagttacagaagctaataaaacaaagggtttcgacccttaaaaatgatatttcatataattaaaagttgaaggagtttataactaaaatggctaaaaaagaagtaaacaacagttcagtattactgaatctatacaataataaattgcttgtatctaaggtagacgaggcgttagacgagggtaaaccatacgattttattattgctttttgtaaagagaagttcgattttgaaattaataaacctgcattatctaggtacaaagagaaacgtagagagtctttagaaacaggagtagacttggaatcactacttgacaaacgtagaaaatcaggtaaaattattgatattaagtctaaagaggtaacaccacttcctaatgaaacatacgataacacatttggacaagtggaacagatatacaatgatgtagaggtgttagacacaatcatacaaaaaggttttaactctctaaaagaggtagactatgttgaagccccacttgctatgaaagcaattgaagtaaaagctaagataacagctaaccagttccaaggtctaagtctaacaggtctaagagagctaagattaagacagtctgctaaagaacaagcaatgactgagattattctacaatttattccagaagaacagcatgaagaagtattcaatgcaattgaatcagcagagaaagagttctatgaaaacttagacttaacagaggaagaccaaagaatcactaaagcgttgcaagcatcaggtatggatataatttaggagggctataatggttgaaaatttgagagaggtaaattataaaacgttaaccttagaagaaagtttacatgcgttacttgaagggaagacccttattgtaaaagggctagaacaaagacgtaagttagatgtgctagtaaggattttctcagaaggtgttgtacctgttacgcagataagttatgatacaacccctgcggatggttattggagaacagggtattggcagatatatgatttaccgattaacgcccttagcacatacccatgttttatctatgatgatttaaatacggatgaactccctaaatttatgataggggacactgtttactacacaagtaaagaggactctatcaaagattccgccattgtaatcagtgtgtacaaagacgatgttaacaataagtggtactacaagttaagtagagacaacgaaatatatgcagagagtgagattagaagagacaggttataagcctgcctctttttttaaatttattgttgacaaatatctataactttgttatagtataaatatcaaggatattgataataaattttggaggtgccatgatggcaaatattttagacacattaaagtggttagataaaggggacaaagttactattgaatttgataaagagcggtcgaagtatgcaaaactgactttgcacgattcatcagctaagacaaatattgtccgtaacattgttttttatgatttagacaaaggagtgtatgcttacactggtgaatacgcccctgtatgggataacttattagatgatatgcgtaagacacaaggcgcagaacctgaaattaagactacaaaagtaaatcagttagctacctatgaagatgccttaaagtttattgaaacaaatggtacgttttatgtaattggtgaggaagtaattgttaaggttaaagatgcaaaagagctagcattgatgttaatgtactttagagatgctattgaagaactaagaggagaatacgaccctaaaaaacatcatgtagatatgacaattgaattatcaaaagatgttttgaaaaaaatggcagtacctagacatgaattagacttatctttaggtggcttaatgagagctgtatcacataatgttggagaagacctttttgaaactcttgggtttgactacatgaagcaagcttgggaagtcttagttaattgcttgtcactagacactatccatgaggtacctttccgtgtgctagacgaattagagaaagtaacagatagcatgtctactacagaccatattgttaccttgtatgccggtagagaacttaaaaaattctattcagaggaagagtactttgatgtaatcacacacaatttagctgatgtgattatggattggtcaactatttttactacagctgttcttaataatacagaagatgatgaacaccttaaagagttacagcttaactttgagagattcaagctagatgtagcagaggttctattaggtaacgttgcaagacatttactatatgcaggagttaccgatagctttaacgaagttaaccattatgttgtcggagcaggtaacctgattagaacagaaggtttgattagaatagaagagttaactggaacattagacgaccctaaagatgaagatagttctgaaacagataataaagagttactagatgacatctttacaaatacaggtgatgatggtgttgaagactttacaaaagaagtagacgagcttgccatgtttgagaacgaacatgctgaggaacttttggaagtcaataacaaattaaaagacaatcaagagttattacgtaaaactgtggaggcgatgggtatggcttcttactctactattgattcatcatttgaacaagaggaagaaacagatagcaaccacacagagacttgtgaggataccagagctttccaaatgttaagctatgcagtacgttcaggagacaaattacttgaacgtgtagagaaccacccacaaactgaaaaagggttgaaaatggctaaagattctctaaaagagtttgaccatacagttaaaccagaattagaagcttatttaaaagcagaagagctagcggaagacgttgatagacatgcaattgtaactacaattatccgtatcaaggacactatggaagcagatatggacggctgtaccgacccaatagagcaaggtttattctacgcaggaagcctaaatatgttagaagaaatggaatcattgttacgggctagcaatagaggttatggttgggacgtaacagctatctatcttatcttgtccattgaactagcttatgggacttacgggttgtctgatttagactttacaattaaaaataaagaggaaactcgtaaagaagaacaagatgcaattaatggtattgtaaacttcttagccaatcttttaaatacagtactagaagaagaatcagaatcagaagaaacacctgtagtagtcgaagaggaagaggaagaagaggataaggatgacttctcactatctacagaagacactgctaagctattggcagactggtcaaatggtctacctacgtatgtcgttactcgtaaatacggaattagtatgggagctttatactctatcttatatgcaaatggggcagacgttaaatcttctaaagtagcagaacgagtggctcatgtggaaaatgacaaagatatgttaaacgcagtcattcgagactacaagaatggtactcgtttagtagatatttatactaaatacaaattatataaaaatggtttgttctatcttttagacaaatatcgagtgccacgtagaggtcgtacaaagaaataacaatgattaaggtagtggtgttaaactactacctaatcttttattagggaggaacttaattgattctttttatatttagcattattacaatgctatccatgttcttactatatttgtttggaatggcttctgtagccctaattgaattagggcttcttataggtagtcaaaatgatataactaaaggtatacactctttacttttcacaggtattgcacttactgtagtcaccgaaataactagacagtgtttactattattttaaaaaattggaggatttagtatgttgttttttattgttttagcagtatttataggcgggcttgctttatggggaatgtatgatagttatgggtacctagactggacttcatggttatttagtattgtagtaggctctgctgtagctacttttttcacactggcggcggttggaattactttcgatgttgcaccaagtcatggggtaacgaaaagtcatgaattacaccctatctatgagaacagtaaagtagttgtagaggctaaaaaagaccagtttgagattaacgtagatggcggtttggtagctattgatgcagaaggtactactatcctatctactaaaggagaggttaagcctaaaattgtgtttacagagaactatataaacaacaactggtggactcgtttcctaggtatagcaggtaaggtaaaagacacttcatcagttctttatttagactcagataccttagtgtacaataagccggaaaagaacagtagcgcaccagatttaaaaattaaataaataatttagaaaaaggggcttgcataaagtctctttttatgttaatgtgtatttatagataggagggctactatggaaggtgaagtagtatacttagatgagtttatgaattttttaactgatagtggaataaatactgatgatattaacgtagtagatgatagacaggagggctagtatgtcttatacaaaagaaacattagaagctagaggctatcagtttgacaatatgtctatgatagataagatggatgccttgttagaactgctagaggaaccagaatttcgagaaaagatgaaagaagagtacgcagagttctctaagaggcacaataacgatgaagaatgaaaagttagagtcttaccttatgaaagagattgcacctattttagatgaaatagagatgagtgaagaattgataaagggaatggagaacaacaacccatccgatacggttacagtctctttttccaaagaagaagtagacatactcttagcaatgttagaccttgagttacctagattagacagtggttctgaattgtttagtcctaatctggtaagggaagctaaactacaattaattaagaacaagctaacttcaaaatgatttctcactagaattaattagtgagaaaaaaattaaataaaactattgacaaataacaaaacatgatgtaaactaagtttataagataaagaagaggagagatacacatgaatgaacaaaagaaagtatatgcaaaattaacagaggacgaggcagtatttgcttgtgaactaggagataaaattaaacaaattagagagagccaagagctatctcgattagaacttgcaaaacgagcaaaagtagaccactcaacattaatcttaattgaacaaggtaaacgactaccaacattacgtattatgatgaagttgagcaaagcactacaccgagaattagctattagctttacagattaaggaggaggtatcatgagagggagcctagagtattacaactatttgtaccaaacaatcgaaaatcgacctacggaggagctagatgtcctttatgatggtctttataaaaaagccggtgacctgtttgcaatagataacttccaaggagttaaagaaggcagactaattttaaaaatattaaaggctattagagaagaaattaatagtagaattgatgaagaaattgacctttacttgtataatatctatgacagtatatcagatgaagacaaacgagtaaattggctatatgaggtgtaagtatgtttagtgttgttaaggctgacagctatgttaaatcagacatagaaatgtatgaatttaagaatgtaaaaagtttggcattaactaagaaagagaaagaactatggtcagacaaaattgctaaatatgctaatgagtttattgtaaatgcttatggagactaccatgggaataaaatacctgaaataaatgttataatcaatggtaagctaaaaaaagtcctagggtcttttgtccaattcactaatacaaacatacactatatagagataagtggtaggtttgttaaagaagttattctattacaagaaacaccactagctcaaagagcgcttgatattttaatggatgtagtcagacacgaggctatccactatacactttgttatttaaatagtttaagtgggggagagcgaccaacgttcaactaccatgacggtggggaggactttgaaaaagatttatgtttgacaggaacttcacctagtggcgctacgaaagaagagtatatttatagttcttttacactaggagctatcagatgccgacaccatagtacttgccctgaatgtgggttggaaacatacatgtatacacgaggacggtactattgctataatgggtgtgtaggggataacggtagaaaaattatatttagaccacaaggtgatattgcaatttatattgatgaaccaaaatctaaagctaaaccaaaagtagaggaagctctgaaagactataaaggagcgttaaagctaccttatacaggtacggaagaaataaaataaaaaaatagttaaatgtattgacaaataacaaaacatgatgtaaactaagtttataagataaagaagaggagagatttattatggcatacgtaacaaatattgatgtagtagcagatgggttagatttatataatgggaactatgtggtagaacgaggtcaagtagttacttttaaactacatgtagcgacatggaacaatgagcctacacccgaaaatgcttatgcgattattcgtaataatggggtagattataagagtaaagttgatgaatttggcaatgcagaggttaccttcccagttaatggtcgtcctgaccaagtaacaactagtatctttgcattaacttctggatatgagggggatatgcctcgtgtaatgtcagctatctttagtgacgaaacagaaattaaacaaactgtaatgaacgttacagcatctattaatggggagccagttagtcgtgaaggtgtatatttagaacgagaccaagtagtcactgtagatgttaaagcaacattatctacaggtaagttctgggaaggtgcacaggtaggtgtgtacaataacagcacggaatacttaggtgacctagatgcagatggttatggttcagtaactttccaagtaaaaggaaaacaagggatggacacatcagctatctacgtattcgttaaagaccatgaacgagaagctacactaacagttcctgtgaagttcactaatactaccgttactacagaaacttctacggaagaatcgtctactgttgatacgactacaggaacagaggagtcttctactacggatacaacaactgtagaaagctctacaacggattctacgtcttctacagtagattctacagaaagtacagtagaaagctctacagagcaaacagtgaccaatgaagacacttctactgattcaggagtagttgaaactacagaaacttcccaagtagcaggctacacagagtctacttctagttctacagagtctaaagaagttaaggaaactcatactagcactacagaacctgctaaagagttaccatctacaggcacagaggtagactatgggcttgttgggttcggtggtgtaacattgacagttgtagtggcactagtagttaaaaaattgttgaataagtaagaaacacaatactgcgaccctcattaattgggggttgcaaattatattatttttttaaataaacctattgacatttaacaaactataaggtaaactaagttcataagataaaggagggaaacaaatgagtcaagtaagcaaacatggagaaaaacgtgtacgtgaacgtgtaggagtgaataagagttcagtagaccgtcagttttacttagcgttagaacgaggataccgacaacatgagctaacaggtcgtctaaagaaatgggtagtatcaagagtatttaactctaagtaccctcaaacatgtattttatataatggtaaatgcttcattgttagcagtgaaggtacactagttactgtgttaaatattccaagtaatttactaaaagattttgcaaaattatctaagaaaagaggaaaataaaatggatattatggatattgagtttttagaagaacataaacaattagtaaaagaacatgtagaacaagaattaaaattaatgcaccctcttaagaagttacaagtaatgacagaatggttagaagatacagaagacaagctttctcaaggagacttagattacttcaatgacttaacagaaacagagctaatagaggcaatggatgctagtgaaattgtagagtcttattcagatgtcttgttagattttattgactactacaatattgatttaactggtttagaggaacagctaggtgtgtaaccatggataaggctgaaaaggtagataacattgtaagacaggtcacaggggctgttattaagacaactgcaaaagtagcttttattgtttttgttttaacctttgcaggtgttttagtaggttattatagttattcatttttaacaagtgcagggtggtttgccttacctatgatattatcagtagatttgctatatgttgcggtactgttaggcgggctagcctttatatttgcagaggtctacaaagtagttttagaagtgaaaaagatagttaaacgaggagggcaattatgagtaataaaacattagaacaaagagtaatagatgctaacaaagagataaacgataagcttaacgaatcatcaattattcgtaaacaaattgaagaattagaggaacaggaagccatcttactatcagacgtagaggacttacttgattacttagaaaatattggagtagacctataggagggacttattatgaataaaaaagtagaggaaatgacaatggaagaaaaagacaaagcactaattgcaatgggacttattgatgtaacgcaggaggcagactgggtagtattagctacttgtgaagaatgtagagaagagtacgaaggagaggagtacgaagaaggggactgtgcagagtgtgaacattgtggaggagaatactttatgattgagacagctctcgagggtactcgttgtggacgttgtgatgactactttgatatgtgggacgactactttgagtttgagaatgaaggtaacccatataaagataaacatatctgtgaacattgttatgaagaactagtatctatgggaatggaagagaaattttaagatactactaagaaggagacttaatcatgaacgaattagaagaacttaaaaatacactgattagacaaaaactttcaatgttggagagttacgaaatgagagaagcatcattttggataatgtttaatgggatattacgtgttatcatgtcagtagctgtcatagcctttgtaaactatgctaagcatgtaagaccagataatgtagccacatggtttctagctctgatttgggttattttcctagcagaaggtattaaaggtgcttatgatgctgttgcatttggtattcaccgcaaaaaatttgctaaacatattaaaaatatgcgtgggataatcgcaatcacacaattacttattgaagaagacgaagagaaattaaaaggaggcaagctagatgagtaaagatgagaaattagttgagtggtttggagttgccttaatggtgattatgtggttgcttatcacgttcagtatcttatacacaatagtttccttacctttcatggtacacgaaggggactggctagggattgtgcgaaacgtactactggacattgtagtattggcaattggggttgtcgctacatggttgcaattaagatttaaaaaaggaatggaggaataattaatgggatatttagagagtgcaattgaagaaattgaacgagtactactaggaaataaaagccgtgacaccgaagaagtttatttaaacaatgcaattcggtatatcaaaaaagagttaaagaaaaaagaggtagagccaacgtggttaaatgaaccacagacattgttccttaattggtttaacgaattatacgcagtaggtggtttaactcatgtaacagaaacagtaggttttttagaatccacaggaggtagaatgaagtatccagaagcatattctgctttcagtaatttaagtgagaatgaattgcttgaggtttacagtagcttttatacagggttattcttaaaagcacaagggggtgaattagaatgaactttcatggtaaagtatttcatgataaagtatttgacattctttctcgtgattaccctgattggcagaggtatcagacagagaaacgaccacatcccaatgagcttagaaaagactttgccattgatagtacagatagtagatatgaagagtatgttatgggagagtttaatgtagaaacggctagcggagatgttaaagtatacgcagtaggaattagaagagtagttcatagttaggaggtagttatagttaaatgaccaattactcagaagaactaaaaagtttagagaaggagctagcttgttctttgaatgatatgtggcacgctagtgtcgctatgaacgagagttactatgcggattgtataaaacgttatgaaaaaatttcagtacgaatggctgaggtaatacagaaggaagctgaggaggcaggggcacagtgaagaagtatacagagaaggatattaaagaaggtatgaaactacgttgcacagatgataatgctaatgtaagctattgggaggctgatagagtttataaggtaacacgtgataaagaccttggcttagttatcgcaggggaaggcgtaagaagccacagaaccgtagagttcattgtaagagcattaaatggtgacgatgcagttaaatttgaagttgtagaggaaaaacctgtacgatttgctaaggtaacttgtgtataccctcctgatagaggtcttgtagaggttgggcattgttacaaagtacttaaagagttccctgagggacgtgtgcgtatttaccttaatagtaaactagggaaccatgtgttactcccagaccagtttgtttttgtagatgagccatcaaatgatggagaaaaagacgtagaagagctagatgtagaagctaagattctagccgatattgaacagctaaaaacagaggcagagtgcctgtttgctaagcgtgaccgtgtaaatgagcaagctcttaacttaaatgcaaaagctcgtaagttagaagaatctttagaggtactaagggagtacatgtagctaagtgtttttatgattacatctattaaggagatgagagaatgatttattatattaattttttagaagattttgcatcaagttggagtgcagataagcgttatcgtgttcgtagaattatgacaactggtagctacgctattattgacaactatggtcatgtcagattcagcagtgacacagcacgaggtgtccttaagaatatagagcaggagtacgccactcataaggttgagcttacactagcagaagaagaaacaacgaataagcctaggtttaaagtaggggaacgagtgaaagtgtctaatgatttacaggcattcgggatagaatataagactcacataacctctaagatgataggctgtgcaggtaatgaggctactattacaagtgtttgggggagtaacgtacgctactttgttaacatcgatggtatacaccaagattggtgttggacggaggacatgttagataagattgaagaagaacctgcactatctgttagatgtgttgaggcagttcaccccttttggacaaaaggtaaagactacgagataaaccttacatctgatggtcgctatcgggtctgggatgatgagggagatggtagcagtggcaaatccataaaagaactactggacgttatcaatagtggcggtaacaaatttgagctactagatgaaacaccctcagaaacagagcctaaggtaaaccatgacatgtctgacttagagaagatagaggctaagattactgccttatcagaagagtcttttcagctattcgagaagagtgaagagttgagtgctagggcaattgaactgcaagatgagtcagtagctttagaagaagcattgtatactattaaacaatatttataggaggagatttaaatgcgtaaaaatgtaatttgtagattagaatgtgtttcaaaagacaaagataacttaggtgaatggactgtagataatatttaccctgtgtttgaatcagaactagggaaagtatacatcctcgatgatgaaggtactacttgttccagagatagcgtgtctcttattatctctagtatggctagctttggggtaacatttagagtagcaaaagataaagcagaagaccctatccctagcaatccacaaagcagtacatcattagaaatagttaaagggtatgaacacttagaggagtttatagactcattaagtagcaaccaacgtgtagtgtctcactcggtagaccctaactctcaatggcactatatcatttatgaaactaagtcagctgaattgggtggagtaaccctagaggaactactagacacactagtgtataatgtacatgttgtagtgatggaacgtagtaagagaacatatgagccactagaaagtcacacgtttaagtgggaatatggaacaaagaataccgaggattgggaaaaaattgtacctttattaggttgcaaggtgtttaatactagccccaatagcaacagagggttccatattactattttgaaataaactgttgacaactactatagtccatgctatagtaggtacatagataaaaaacaaacaatattaggaggtagcatagagtgaaagaaacagaaaagaagtatagcagagagtataaggggctagaatttgaaattattatcacttattaccctgaacaagacatgtattttgtaattgtagttaacggacagcatcgtacactaattaaggttacagggaaagagtgggaaatgagccctcatgcaaaggaagctgttattgagaaggcacttgatacatgctacacctatattgaaaatcaagaagcgcaaaagaactagattaaggagggactatgatggtaggggacttcattttatgggttaaacaagcatggaaagaaacgttctgcattcatgactacacggttaaaggtgtctataaaacactagatagtcatgggtacttaaagtgtaacaagtgtggaagaattaaataggaggacaacaacatgaaccagagacagaagattaaacaagaaaagaaatggtttgaggagcatgggtatgatacacagcaaccacgagaatgtattgagtgcggggctcctttaagttggaaagatgatatgcagaaaagtcatggagtgtgtagtgagtattgttatatgagaagtgttgggctgtcgttatcagattttatttaaggagatgaaacactaatgtttaaaaaagataaaaaagaagaaaagacttatagagaaggagacctccttaaggctgtagggggttactacccagatgctaggttgagtttaggaatcacatatccattgtataaaaccaccaatgagggttggtatattatcaataacgaaggtagtcgggtaaccttaatcgaaatggatgctttaggcattgactacgctgtgatggaagaacccttattagaattaaaagaaggagacccattgctagtcgtgtccgaccttaagagaggtgttcgaggcttagcaggtgcagaagtacagtgtttaaacattaccggtgaaatggcttcactagcaggtacagttgttcattatgataaagatatgagccatattgcaaaggggctgttcactgttaaagagaacgattcttattggtgtgtagcgattgctattccactaaataaagtagctgaccctgattttgcattagagcatttgcttgctacgttaaataaaaaagcctctactaaacgtctggtattgaatgagataaaatatgatttaaaccttcttcacaaagagctagacgaggtgtccaacgaagtagaaaaactaacaaaaaacattgaaacaatctataataacagataggaggtatttgaggtgatttggtggcaggacaatgagaattacaaagtcacacgttttagatgtgcagaagacttaaaggaattcctaaatgagcttgacagggaagacgaagtcattactatttttaatgatggtcactacaattttattattataaagagaggtgaacacgttgaatgatttagagttattattaaagaaacctattgcaataaataagaatcctgatttttatgaattagcgataggtggtaaagttatctacaacgaagaagtaatggagattgctaaagctttctctaataagaagactaagtattacctgttaaacacacggagtaacaaacaagtatgtgtgccagtgtaccatgtaagaccttatgatacccaagtagaaggtcttaaactaggtgatgtcctagatgctatgcacattatgacaactgtaacgttaagggcagtgaacaagcacgggtttgttgtcgatgaggatattattgaatgtttagtagatgacatcccagaaaatgcgcttattgttaccagtgaggtatcccgtattcagccagaggattttggtaaggtaattattgactattttgtttagatatgttaggatagacttaagataggataggataaacttaggatgcgcttttgtttaggataggtttaggataagataggatataggataggataaacttaggatgcactttggtttaggataggatgggggatataattatgagttacgttaatgagtttgaaacaattggagattggttagatagagaaatttatgatgtgctactaagagatggacatgatattgacgaattagataattggggaatggctttgtttgccttaagtgaaggttacgttctaacagatggacctgataaaccattcctagagttaactagagaagacctagtagcaggctacaaccactttaaagaagaactgaatgggtggttacgaggaggtaaactgctagaagtctctgataatttatctacaattcaagggttcagttttgaccatgacgatgttttattcttagaggataacaacaaagcttatgcaatgctagtaggtattatcattgaagcaagagacacatataaaaatggtttctgtaagggttactatgtaataccttaccaagattgagttagccttatggctagctttttcttttaccttaagtagcaggttgcaaaatctcctaagtagcaggttgcaaaatctcctaagtagcaggttgcaaaatccggatgcgcccggctagcggtggagagtcaagccatgcgggttcaccttaatagctaccgtccctagattgtcggtttttcttcctttatatagtaagaaaaattatttaaaaaaaatgaacttttctattgacaaaaaacaaaagctagtataatatagagcgtgtaaggaacaacaacaataaaaaaaataaagataaaggggcgctattaaaatgaacaagtataaatttacctatgcagacattaagaatttaccagaggaagaaaaagaaaaagaattaaaaaatcggtgtggtgttttagcggtagagtgcttaagcactaaacagctacaaaagaaaaaacctcgatttatggttttcttaaataccgttatttttgatagtaccgcagaaacaggcggacaatacgcaacggcaaccgttaaaaccgaaccgataggggacggacgttttcgagtgtgtgacggttggggacagctttctaatggaattattgaactgttaaaataatttataaaaaaaactattgacaaataacaaaagataaactacaataaacttgtcagataaagaaaggaagttataaacatgtttaaaagattagaagagtaccgcaaaaatttatccaatgaagaacttaatattttcaacaatacaatgaagctattaaatgataataagacagactttcaagaaagaacaaaaaattttgataaagaactacaatatacagagctagaaaatgaaaaagaaaaaattttgatagtattagacactattcgagtgagtaacgaaaattacaagaatgttatatctgaaaacttatatcattctattaaagaaatgacaattatttttgagttactttataacttatagaaaaaaaattaatcaaaactattgacaaaagacaagaactagcatataataaacttatcagataaagaaaggaagttataaacatgactatcgcagaaaatgagttatacttagttaaaaatccattaaatgagtgggatatgaactaccatttaattgaaaaagaaacaggaaaagactttttgattgatatagatgacgtggcagacaatgaagaagaaagagaaaaatatgaactaggtttactattttcagacttgaaaaatcttatagttgatttgcttattgagtatagttggaaaactatttaaaaaaaattaatcaaaactattgacaaaaaacaacaattgatgtatacttaattcataagataaagaaaggaagttatagacatgacagaacaacaatttaaaaaagagcatttattagaaccaacagaatggcgtagtggaggctacttagacactagtttaatcgaccaatcacaatcttattatattgaatcacgtcctagaatttacggcgggtgttacgtttatcaatatgttacaatgaaagatggcacggtctacgagctttacagcatgacggcaagcactaggggaattgttgcacataactgccacgctagaaacgtattgcaaagagatgtaaaacaatacagagaaagcgccattcattactagggagggttaacctcctttacatagaaaaaaaaattgttttatctattgacaaataacaataactattgtatacttaattcataagataaagagaggaagttttaaatatgaaattatctgatattattctaatggggttgcttgttagcgttgtactactttggggctaccttagcattatgatttgcttacaagtttttagagcgttaggcggttgggatattagaacgttaacggtttgcagtggtttgctatttgcctatgttttcggtttaaaaggaatctgggaacaagggacagggaaaaataaataaaaaaaattaatcaaaactattgacaaaaaacaacaattgatgtatacttaattcataagataaagaaaggaagttatgaacatgaaattaaacgttatccatttactattttgcttatttcaagaacaagaaaactattctattttaagttacgaatcaatagacgaattttattctaggttaggttacgacttagaaagtgagtggttacttagggacttgagtattaatggaacaagtgacttagcagagttgctaacagattacaataatttattagagaatgagataacaaaagcagtcttttctgataaatggttatagtttaaataaaactattgacaaaaaacaataactagtgtattatagacttatagagataagaaaggaagttgtttactatgttaaaaatcacaaaagaaattactttgggagagtttgaagcttgggagggtggaaaagaccgactagaagaaattaaagaattggatattttagaggaagcacagcaagaaatcgagttgatgattgagggggcggaagaggtaacagaaatgactataaacgatatcttatggtttgagatggacgagttcattgcacaatttgaggaagaagaggaagaagaatagaaaaacagggaggggcttcctccctttacatagactaggggggcttgagatgacgacagaggagaaagcgctaaacattgcagagaatagaggtataacagattataaggttaaggggaacgtattaagctattatactagctacccgatggaaaaatgcacatacattgtaactattgacattgaaacgctagaagaagaaagaaaagaactcaaaaaatattataaaaaaggcttgcaaaatgcttgcttgtagtctataatataaagaggggcggcggttactattaataataaaataaaaaaattgcaatatgaaagaatcagaaagctagaaaagagaaaaagaggtgaaccacctgaatttattttcaacggtaactattcattggaagaaatagaattattcttacattttagaaaagagggtaaaaatatgttaaaaacagaagaaataaataattcagagtatacaggataccttatattagatggggataacttaattgtacgagtagaaaaaaatagaaaaacaaaagaaacttactgctatatttatgcagatagcaacgatgatgattttacggcaaagtttaaaattaattaatcaaaactattgacaaataacaaaagataaactataataggtttataagataaagagagggagttataaaaatgacaaaaacagaactacaatataaaaaagcaatcggggttgcaatctttgcaacaagtgaagaggataaaaagcaactaggaaacgtggcaccgtttagtatttatgagattttagaaattgacttaaataaaaatcgggtatactatgccttaaactgtggggaacgacatgcagtgtgctttactaaactacgtaaagaagaagaaacagggaataattttattttaatcaataaacaaccttttttcttaaaagatatgcacaagggtttgacttggtcaaaaagtttataaaaaaactattgacaaaaaaaaacaagctatagtgtattatagacttataaacgggggaggggttaacctcctttacatagaaaaatataaaataagggaagtgctaaacatggaaacattagagaaattcggttatacatggcaaggaatgaaagaagttacaaaagaggaagcagagagaaacgttaaaaacggggttgacacttttctattatatccagataataccgagagtttagttgtatccttggacgaactagaaacagagggggtacgctttggggttgaaaagcccgtgcaattactatttaaggtgtctaacagttaccacaatacagaagaagtttactacggggaaaacgtggaagagatagaaaagaagttgcatgactcactagaaataacagccgaaaacctaacggggcaagaaatagaagaagattgggaaaactactataaaacatatgaggaagcttgggaagcaacttacaatggtattctatcacagttaaaaagtgaatgcactattgaaaaaatgtaaaaaagtttcatcttatctattgacaaaaaacaaactatagtgtattataggtttataagataaagagaggaagttttaaatatgacaatgaacgcaaacgatactttattactagcaatcgcagaggaacacggaacaaaaatagacaaccttaagagctaccttatcagtgatacagacagagcaagcattcttgcttttgtaaaaaaagaactaaacagcgcagtaaactttagtataaaaattggttgcacaacaattgaagcagtgaaagaaagcaatacagtattagataaagtattaagagcttgcaacttgctaccaactgaaaagaacgtgcaaacagctaaaaactttttagaagaacaaggctttaaagtagaagctagtaaagagttagtgggaaaataccttgtaaaattttcaatttaataataataaagaatgaagaggggaagttataaaaatgaaactaacagaaaaggaactaaacacaattttaagagatgatgaaacagggaacgggggaacggctttcttaggtgaaacactagccgacttcctagaagaatcaggtattgactttacaaacctaactattttagaagtaaatgaattactagaaaataatggaattagaccaattgaggtagtgccatgttaaaaaatatcaaaaaatcagataaactcactagaaaagatatacaaggtttttggggagatgaaacaaaaacattagaagaatggtataagtcaatttcaaaagaaccagacaccgaaaaagtagaaactgctaaaatgattaatacattgaaagaatatgcaaataacaacgaatttcatttcgtaaaaggagagcaaggacaatgacaaacacaaacaaccaacaatggaatcagaagtttaatgatggtactatgaaccagaacaaccaacaaaaggaagttatcactttacaagtcgcagaaagtttcgttagccagattttaacaaaagaatattcggtgattggtttagttactctaatgttttattttatgtttggtatgtttggcattttcgggggtgcaatctatattgctttcactcacacaaaccgtaaaatcggggcatggcaacaagtaagaagcgtatacactattaaggaggaacaagaagaatggaacaattaaaagggctaacaattagagaattgattaagaaactagaagaagtgccagaagaaaataaggacttacctatttatacttttgaaaatgaaaactctttgcctattaaaggtatttcattatatgatgaaaatgctaaacactcacaagaaaacccgttaagttttgatgtaatcagataaggaggggtaaagatgaatagttttatgaataaacaagctaaacaggtaaaaagaagtaaagaaataaaactagtggaagaagtaagaagagaaaacgtaaagaaacgcttttcggaggaagttagaaagtacctagaaaaagggtatacaatcaggttagaaaataaagtttttccatttgctttaatatctattgacctagaaaaaggggaaaaggttataagtcttattcttgtgaacgaatatgatggaatagcaaactatacagcaatgaaaaaagttaccttaagagaaaaagggaatagagctatcctgaaaaggacgttaacggataaagatataaaggtgataagtttatggaaaggaatataaaagaatgagcgtatacagtttaaaattactagcaatcttagggatagtattattcttttcagttatcgggatagtgtacgataataaacaggacgaaaagaaataaatatataaagaggtaaagaggtaaagagataaggaactatagaactataaagctataaaactatatacacttataggggaagagatggaggaggggaggaggtaaaggaaagagagggagagagagggagaaagaaagcaataaggaataaggcaataaagaaaaaaagttgtttacctatatacttatacacctatgtacctatatacagatagcccctaacaagcaatagtaagggaataaaggaataagagagagaggggaagaggaaaaaggaaagaggaaaaaggaaagaggaaaaaaagttgtttacctgagattttaaagaaccgacttcaccagaaacccgacaagggggcgcataagtctatttcaaaataatctgaaataagtatagttattactgtttatatatgattatccttaaatatctgaaaataggggcgtttattcgattatatagttaattatcttatactattaatcatctagcctattactgcattcattgctattatattctatatagggggcgcttatatcgtttatagtgtaattatgaggggaacgactaggaacgcttatacgggcttatagggggctttaaatgcataggctatttatacatatattagaatacacaaggaggaggggcggaaagttagtagattagactgttaaaaacaaacgggggatagatgcacattgttattttactgattggactaggttaactagctaaaataagaatgctattgtagcaacgtttataagaaataggggtgtacttttcccctagggtgattttaaaagggtataaaattcttttgttatttaattaaaaagttcatcttagctattgacttttaacaataactagtatagcataagggtataagataaagagaggggacaaggcacatgcaaaacgatatttattacacaatacaggacagggacggctttgcagaatcttataatgaatcaggtttcttacatgaactaggtaacatgaaaattattgattatacttatatagcactagagggggcagaaaaaggggacactgtggaactagtgaaagtgcctattttagagggctaccaatatccttatgattttttagtaattgatgaaataggtaaagaacactatatctattatgaagaagaagaataatttaaaaaaaatttcatcttagctattgacaaataacaataactattgtatagttaacttgtaagataaagagaggggagaacttaaaaaatgaaattaaaagaatttatcaaactagcagaatcaaaaggcgctacattagaagcatacaacgagctaggaggttatgaactaactagaggagatatagtagaccagaaccctattttaattgcttatatgcaaggggcttacagcgtagaaatatcaaacgaggaactagaaaacaaggagttaacagaactagcatttgtctataaaaatgcaagttttatttatccaaacgaaaaagaattattgtcaggactaggactatagcacaacggcatataaagcccgtaactaagttttaacgggctttatatataaatacattatgagggcttataagggcttatgagggcttatgagggggactaataagaatgctagaggtaggcaaatttgtacgaggtaacaacggctcaagaacttgggtgggtcagattgtaggtatagacagactagcgggggaatatcgtgtgcgtgacgttgtaacaggtatatacttttatgtatcttgtagcgctgtttatgaatgcgctagaccataaaacatttaataaaaaaaaattcatcttttctattgacaaataacaaactatattgtatactaggtttatcagatagagagaggaagttatagacatggaatatacattagacgaattactagaagaggaatacagcacactagacgaattgctagatagtaaagaatttaaaaaacaaatggacaattttaatcacatcccacaaatgcaaccgcaaagtcataacagcaatacgctagcagacacgggacgttatcctgaaaaataaaattgaaaaaaggggttgacaagttcagcccctagtggtatactaggtttatcagagagagggaggggcttcataatgaaagcaatcatcatcaaacaaatcaaggacggttaccaaggtatcttagaacgtaaggagaacggttggacgaaagaggaactaagcgactatacagcgggggtaagcaatacactagttgaccttttcaacgagttacaaggcagtagctttattacagaaaaagaagcggaagaaattgaggaagtattttctactactatcgatacattagaattttaaacttatctattgacaaataacaataactaatgtatactaggtttataagataaagaggggaagttattaatatgacaatcgaacaagtgaaagactacggtaaagcatacggaaaagagctatcaactgaggaagtgaaagaattttttgagaaacacaacggaatgccatcactaatggacttagcaaagttttgcggggcaatgaacgaggacggaacaaaaaattaaaaaaaactattgacaaataacaaaacatagtgtaatataataagtgtaagagggaaagggttaaacctaaaaaggaaaatgagtacttctttcttacacaatacataccagataaagaaagggagtttttaccatgacaccagaactagcagaaatgaacctaactaaaaacgatagtccattttcatttattaatgaagcaggggttttcattgaaccaatcaaaaacccgtttacttgtgagggtacttatttacaagcttgtaaagacgtagtgggagagctagaagcaaataaagaggtagggaacgacatcgacaactatttaaaaatgcaaagttggctagataagttagcatatgaatatatttaaaaagtttcatcttatctattgacaaaaaacaataactagtgtagtataataagtgtaaggaggaataaacctctttacaccacacaacagaaaggaagtttttactatgacagaatttgctaatatgaataaagaagaggtactagaattacttaatgattggtttggtgttagtgactatgatacagtgatggaagagttaggggagatgaaacaagttacctttacaggtagcacggacatacctctattaggtggtaacggtaacttaatcggtttacctcaattctttaaagacaatgaagcagaatcagagttgccaacttatgaagaattactagaggaactagagaaagatacatggaatcttgaagcagaggacaacacttacaattatagcggcttcctagaaagtgaatcagattttaaagttattcaggcagaaaattcagacacgactattgcattttttgcaatccatacgggtatagacataagagcgggctactcaaaagcaatcccagttatttttgaaacttactacgattttgatgagtttttaggtaactacttttacagtcaaggttactatgcttttaaacacgacaacaaggaatacacaatcagtttagacgtttcggctgtttcggagtatgtacgaatttatatagctgatgaaaacaacgaggaactacagcaagtctatgaacaagaaacttgtatggacttagacatagaaagcgtagaagcatacttgaaagaagagggtatttcatttactgacttaaaacccgcattgtaaccatataaggcactagggagcttatacaatagagctacaagcttcctagtgtaattataaggtaaataactagaaacgcttatacgaaagaatagggggctttaaaatgaagtttaaacgagcaatcgaggaattgaaagagaaagagggtatcaccttagaaccagaatataacggggtatatacagttaagggctactattctaacttagaggtattacattgtagcgactacgatgaagaatataataacgaggaactagaggactgccatttacatgaacttgtattcaggtatgagggagagcaagcatataagagcggatattatgaactgctaaaattattgaactaatctattgacaaataacaataactttggtattctatatttgtaagataaatgaagggaattgataagaatgaaaaatctaatgaatgaaactatctatagtaatattacattaaacctaactaagaaggcacaaggcattatatcccaatatgtagaccataacgagggctacccaagacaaaagaacagaggtcactactaccgctatacagttacatttatcaatggtgaaacgttcgaaagtaatagcaaggcggacattgctcactatatttcatctatgacagcggacgaactagaaaccatcatcaaggaagcacaagccgagtagtaagaaataggtatataccagttaaccctgccccgtggttttttgaccgtactttgggggtggggctatttgcctaccccgaccctcgggtacctattttttctaggaaaattattttttatatatttagaaagtcaacaccctacatataaagtcaacataagggctcaccctagggctcctatataggctcacctaaatactcccatataaaaagacccctccccgttgataaggataggtcttttttatttagccgtctgccctatgattggttattaggttcaggttccgccaagcacgacctcggttacattagtgtaatgggtatacccttgtaagtcaccatcctctacaatgtagtagtcagtgaaccctttacacgtactattatcgtaacctatcactctccctatagggctcctacctctaccattacactcgaaggtgaaattattaagtataacttgtgtgttgatagggagcaaaggctcccctcttgaatttagcatgttaatgtggctccttttatttagtattcttaggttaatagcacaaccaagccactaatgacgactaagactacgtaaatttccactaaaaatgagtatatagtaaaaacaatagacaatacagtagtgaatatcgcagaggctatcacccatttagtatttaaactattcgacaaaacaacaataccaacgataaattcaactagagcatagcccaagacataaatacctacaatacttagagtaatgagtacagggtgacttataaatccaccccatacattataaaaaatatttagcatatggttactcccctctaagactaatttgttccgccatagtctttgcgaagtatgtccagtatttatggaagtctgcaaagtccaagaaatactctccgacaatctctcctgcttgattgcggtaaagtggtactttcgttttctcgtctttatggactccatactctgtgccatttaatgaaatccatacaatatccggagtcacactagttactgttgccttatcacaacctagtatcgggtcacgaagttctggttttgtgaaaactgtatccccaaccttcaatttgtctagtcgctctttgtctattaatgttgtctccatttgttctccctccttatacattataaaaaatatttaacgtctaattactaccctcaaataaagaacattactacccatccaataagcataaataacaatgttgatacagtagcttttgcaaataccatcaaaatagaatcttctgtatttgtagtagtcttagctgtaataaagctgataaatacatctagcccaattgcctgcgcccatgtaagtgttaacaccccaaaggttggagcaattaacccgttccacaagaacatcgtaacataccctccaatagcaagggtcaacaccattagaactaaagtcccaaaaaatttacctaatgcatttgccaattcgtcttttgtattcttatccatcgttttcctccttaaatgttataaaaaatatttaataactagttacctcttctaagactaatcgctacctcggttaccctagggtaatgggtacaccttcttaagtcaccattctctacaattatatatctcattatgcatggctatgttaaggctaatttgttctaagatacggacagccatcgctcctccaccaaagccattatcgatgttaacaactgcaataccagaagtacagcttgtcagtgctgtatttaatgctgtatacccatcagcagagatgccatatcctgtagatgtaggtactgccacaataggcaagttagtgagccctgcaagaacgacaggtagtgctccctcaaagccttggacagacacaatagcattacaatcattaatgccttctacggaatacatcaatttatttaagtcgttcacaccaatgtcatggaaagccacaggaacgtaaccactcaataacacagttaggtacacctcatccacaatatctctgtcggctacacctgcagaagtaattgccactgctaggtcactgtttacaggtcgttcttgataaaatgcagtttcactagaccgacagtacacgaaagaggcttccgtaccccattctttctcccaatcatactcctctgccgaatcaataataactttacgagccaacatagggatgtcattagttggtatttcattgaatacttccagtggtagttttgttatcataatatttgtattgtaaggaacctctgcaatttgttctgccagtccttgtgctgttttatttcctccaaataatgtacgtaggttcattcgttaagctctcctttagtttttcccatagccatatagactatttatttttttgttaattctatttagacttgcaaataagttgtttctcttcttctcttgttaattggtcgtgatagcactgtgccattacgtatgtctccctccgtttttatatttgttatagtttagtgttaaaccagtcctatgttctctcctagcgtttacaatagttgtttagggtgataataggtagaatacctatcaccgccctaagaggcatatagggtacttataggactggtgcttagttaactgttttaagtagcatggaaaatatgcgcttaagtcaacattagttcttttactttaaatgatgtcaattgcaatataatgatttgttttggatacattagcaatttcatagtctaagtatggttctagtacttcgttgttacttcctatctttgttctaactgttatggtagcccctgccgtgggataagtaatgagtacgtccactccgggcttcattaacttaactaaatcttttaacttcatctttatctcctctttaatagtatatgcttaaatctattttatgtcaactgcttaatttttgtgctcgtagttgcacagatattgtatcaacaatgactgcccactgttcctccgtaaaattgaagtcaaactgtgtatagtcttcctcacctgttgtagtaattgcaatgtctccataatcatcaactgagaaataaatagttgcagtcgcattttcctccggattttgaggaagtgtaatagctaactccccttgctctactttcatctattgctcccccttagctctcttccttctcaatcatcttgatacctgtaaaggcatatacagggagttcttctgtgtaagtgtactgattagctttaaaggtcacttcgtagtaactctccatgccacaatgtaacatatctacatatctgatttccttacaaggcatagcatttctctcacttccgcagaagagccagtcttctgtacagtattctactctacctgtgtggatacttctaactgctacgtatggtttttccatctattcttcctcctgtataatctcttctaggtcacaagggtgtacatctagtgttcctgtccttagactataacgagagtttcctcgtactgcgtaaacaccatctgggtatactttttcaagatatacagtatccccaattgtaaacccatggtcataacgggtatctccaataaccttaaactgtctttttatcgtatacccatacatgtaagcccgtgctaatacgtattggttaccctcatcaatccattcaactagcttgtcatctggtgtttcatcgtatgggtcaaaatactcattagctaataagctcaacattgaatcgtactttttgattgctcgtgcaacaaaactaggtatctctacttttttagccatctattcttcctcctatttaatcataatacatacaaaatacataaaaaagctaacataagcaaaacacgacccatccagctgttacatatgacatatagaaaacgtatataagatatactactgctgtaattgttaataatacaagtatcgtgttaatgagcttttctaaatcaatcattatacaaccccctaatcctcacaagtataagtctggctctccttatactcttcctcctatttatatccaaagaactttaagtaattttctgcttctttttctaagtgctcgtttagctcatctagggtaacaactttcccacagtcctcacaggtgtaggtctgggtcttcttatcttctagcagttcaccatcacatacaaggttgatacagtgggttgtccaactccctacatcatcaggtaagtgccccattatacgtcacctgcttttacggcaaatgccatgaatcgttcatccattttgcggatttctttctctgtaaaggtagggatgtcttttaggtagtttgtaaagtcaggagaccctaatcggtcgatataggtatatccccaatcatctttatcttttgcgataatgatgtagtactcatcttcttcctcctctaccgtatgacccagtacccatgctttggcaagtttctcgtggttttctggtttattagctaaccaaaagtacatcttactagggtagtttcctttgtcaatcatgttaataaacgttaagttctcttctttgcacttatcaatatattcccctactttataaggaataacaactttttcgggttcatctaaatcatatgctaaggagactgcttttgctatccctgtattgtaacctagagcatgagggttcaatgtaccgtcacattctacttgagtaaaaacctcgtcacttagtctactaataaattcattcttgttcatttagttccctccttaaaactcaagactatttttaataagttctgatagtcggagcataacctcatgaggtagctctaattttgtactcccctgtgagtctgcagagctaacttccaaagtatattttacttctgttttctcatttacacgggaagccttcttctcaacacatactttataaagtgctgagttattgactgcgataattttttcttgtgtatttacactagtattgctatcataagtaatttcatagcccattataaaaccctcctatttgtatactggtacagcaaaagcccagtatctttcatcaatttcttttatttcttgttcggtaaatttgctcttacccatgttgggtttatttgagaagtatacttcattattgtgggatgtttcaacccaaagataacaatcttctcctttattaccaagaccagtaaacacgacatggtaaagtggttctttctccacctcatatccatctttcattcgtattaaagtttcaatagggttattcttactttggtctaaccaacaatcgaactctgattcatgtgggtagctgttgccatctaacattctaatttgtgtaaaaatagctaggtctaatgcatgtttattttcctcgtaccattccgcaacaaactttggcacaattacttttttaggttcgtctagtttctttgctagcaaaatagccgccttgattgcctcattgtagcactggctccgatttgataactcttgaacgactgggacgtgactgaactttaactcttctaatgcattaatcaattcttgtttattcatttaatttctttcctttctttcaatagcccactggctgaatgcttgtaagacttgcaactgcgcaatttctgtcatgtatctataatttttataaacagggctattccgataatctgtttttgtagaattggctctcagtctccaaaatagttctataggttcaatatctgttaacgtatatttttcttttaaccaacccaaaacaatttgttgattctcacttagttctacttggtctaccttatcatcagtgtatttgaaccagattttctcatatgttgtttgtccgcctgtgattgttacaaattcggcgcttggttgctcattttgaaatttttctagttgttcttcgtaatgtatatccccaacatcaaattctttaaatttaatcattgtactccccctgttctaaaaaccaatcagtaaatgcttgaataacttgcgcttcctccgttactgataaagagtaatatgcctcttctgttttagggtcttcataggtctccccaatacgttcgagcgcctcaaataaggaatctttactacagtaacctacttttaaccattcaaatacctgtttctgatttttattcagttctggctttttaactgcctctactgcagtcttatacgctatttttaaaaggtctccttcaaaactattagggaaattaatttcaccatttgctttcatatacatatagcaaactctatcaattatttcgttatatctttgtctactcattcactatccctcctcgtcctctacgtcaccccaaccaaagctacagtaggaagagaacacgtctctagctacttcgtctatctcctcattcgtagcattatcgggtacttctactgtctctatttcaccaatacctgcccaagttgtctcaatatatacatttatttttcgcatagtttaatacctaagactacatagtcgtctttctgtgcataatcggtgatataagtaacttctactttaataaactctcctgtatacgaaccacatgtgtactcatttaataaaagaatatctcccacatggtaatttctatcatttttacgtatttcaaatagttttctaccttccttaactgctttaaagtacgtaggtgcaatcttgaggttgtgtgtaacttctaattcactaactaaatcaaaaatgatagtgtttaattcgttaccttctacctgatgtaccaatttttgtaaatttattgatattacagtaggtctgccctctctgctcccataaaggttattctgttggtctctatattcctctgaatcaataactaagtttcttctgttatgcttagtaagtctttccttctctgtcattgcctgtgctagtccatttattgttttagatacttttacttctccatttacaataacttgtactaggtatctgtcatcctttactgtaagcttattatccataactaatatctctccttcataatcttaattgaccagttatcgttgttactaaatacccgttctaggtcattcaatgagttggtatttacaatttctttttttagttttctatctgaactaatattaccaaatcgtttaaggaatctttgtttaaagtgagcataacccatatagtcaaaggtaatagtgtgttctttaccaaatataccccgctctcttaacttaatttctacactctcataagagctatctactttcataaaattatttagaagagtgtgcaaatcaatacaaatagatgcccctgcactaccgcctataagtatatatttagggcactttatatcgggatagcaaggggcatctaattcataccacatatcatttacatctaacccataccatacactatttacgtctactgaggctctttctgtcttaaatggttttatgcaagtatatttcattgtctctcctccaaatcgtctagtacatcttctaatgttgtgacaaccccctgcaaataacccagttcatgccacccataccctctcttatcttgctctaacaaattattaattctttttgttttacgtttaatttgacggttatatcttctttttagttcctcttcaaaggtcactaccttttttgcttcctctatatcttttaataattggtctagcccatctccattagtaccatagttatacactagagctttctcgtccttataggtagttacccctaatgattggtatacatacttcttactgggaacaaatacaagggtaatgggtctgtacagttgaggtagaagacaatcttctgataacccaccaaaatctgttttagaagataccttctctatcacatacctcatatcttccatacttaacgacttaatacataaaatatatctgttaataaccattttctttagctcctcctacacccaaaatagttttcgattctctgttggtttaaaagactccaattctcgtgaagtattactttttccatctgttaaaccttctttcactttatatataaaaaaaaataaatctagctatctacaaatacattgtatcatcagctagattaaaagtcaacactttgtatttaattattttatattacaatagttctttatctaaaattgttttagggatagcccatattgtagtgtaacgaacccctttatggttacctgtaactaatacgaacgttgccgtatctagcttagtaccgagtacagatgctagtagttcatggtcttctttgctaattggtacttcaaaatcaagttccccaagctctaccacaaagttatttgctctaattcccataaagtattcatcattacgttttacatggaatacattcttcgtatgaaaactttgcataacaatatttgcaaaataactgttatccctagctacaataaatctatctacataaatagcctctttgctgagtagttcttcaatgcagttactcataacatattctcctcctcttctaatagtagctctgatacgtaaatttccatcattagactttgttcaatcccgtctagtaataaatgcatagcacacaagccacacaacatttcatctaaataaaaatcaccacatgtctcctctaatggtacttgacacattaaacactctatcaccacacagtcctccttaatttaaattttccctaacttctgttagcacctctcctaatagattaagaccttcccatttactcgtatcacgagagcgtgggtctgattcacgtagtcctattccccatactttatcaaaaggtgatgcctctgcaaacttagttcctttagggatggacagcataaagttacgtaaactatcattttgtgtgaatttataagtattcccatctaaaacgattgatttacgatgctttacccacaagttttcttcaaaatttttaacttgtctccctaaacgtttagcatcttttgggctctctgtttttaaaattctttttgctgtatcaacatcattaaaaagcattgctttacgtaccatcatatagtgctctgctgtcgggaaaataatatcttcccctaaacccttaataggtgcgctgaactgtgatgggtaccactgtgacaagcactcttttcctagtttattacgtttgcttggggtatgtccccaaaataaaatatatttactcatttaatgttcctcccaatattcatagtagtttcccttatgttcaaaaacaaaatgagggctaggtaactcaccactaacttctatatgctctgggttattataagaatagtagggttcgttaccagtagggctaattgctattactggtttatctttaaaaaatggtggtattttatctccatcatttaagtttaatttataggtacttaatggcacgcttctacgatgccctttcagtgttgtatactcctcaagactaattattctaaggaaacccttagcttttaggggtgataagctatctattgaaacgtcttcttgtttgtaagtgacaagggcactataaatggttgaggggtttttgtatgctactgaataattaattgaagtgtgcttatgatttgctaaaaatttctctaattccttagataaaccataaggtgtatcgctatcaataacttttactttaatcatgtgttatcctccttatatgcttctctaactgttcctatactttggtctgggtctggtttactaaatggtaagcttgcacgctcttccttactcaatgtttttaagcaagccacatgtactgcgatgacccctgacttagcttccatgctgtccggaagtgtaagtatatagtcattatacagtttacttaacactagccaataacgattgctactacttttcatattagctaaaatcatcattttttcatagtaatcttgccccatacaagaaagtcctcctattgcagattggaggagctcatcttcaagagctaagatagtatttgcgtaccctttcagctccttgttaagcccctctatagcaatctcaaaacattctctttctggggagcctacagttaaatcagtaagtggctcatatgctcttacttctctcccactaggggcttgtcttcgtttacctgtacgataggagtctttaaacccatactcggaacgtaaaatttcttcttgactatttctcccagacctacctccaaaatagtaatcttctactactccaccaagaccattataatgtcctctaattgtccctatgactttagaatgtctaacatgggttaactgacagagttcccctccgttatcagggtcacctacaatagctgtctcacatacaggacagatatatccaaaacaaatcactttatctacctccaattaacatacattaagaaaaagttacaattttttcacaaggaatatcatagtatgcctttaaaccgtaatgttccaacatgtattctttagcgtaatctacaaggtgctcagggcatttaaatgtgtaagagccactcatcattacaccactattacttaattcttctctcgaagctttagcaaaccacttagcacactctacaggatacccaataacacaccctaaaacttcctcgtcattaaaccctagcttaattgattcattgtatacttctatacttatcttatctgatgctactaggtatatactaccatataacctcgttttataataagagcgttccaaacaacttagttcttcttcttttaatacgattgctacacgatgacctaaattaaaatctaaaataggctttggaaaaacgtattctgctttctcaacaaactccacttagccaccccttttctcattccctaaattatcttaaatgctgtattagttagaacggtaagtcactattgtgttcttcctcttctgggatatataggatattttcaatcttttcttcaatatgtccaataatctctgtgtgctctttaattttctctttatgattttctctttgcttatttaactttgcatattgctgtaattcatctggtgttagctcttctagtctttttttcatttgttttctccttctaatgtaattttctctacgtcatcaacactaccaaaagatgggggctgtgtcaagttcttatctaaccagttagcgataatgtcatgtaactcgtctatagcctcatcgcttacttcatccgttgggtagtagtcctccgctacttctccatattcactgtatagctgttcccctacagattctaacatacctactacatctacactaataactgctttattgaactgccctactgcaaagcttgttgttcccttttcaggtaatgctccaaatatatcctctacgtgcatccctaagctatctacatatgtttttccttcattaatggacaataatgcaagttttcctgcagacactgcctcttctctcgtagagaacgtctcacacgctttccatacatctgtgtcttcctctagtgctaatacatagtctttatcttccattgtcttgccctctctatccttttaatataatcatatttgtattactgtctagttgccatccatcaaattctaagtagtggtcataagggttaattggttcacctacaactatatagtcctctagtgcgtgaccttggtcttttagctctagtaattcgtctaataaatcttgtgctgtcaaattattcatgccatgtagtatcctttctctttagcttcttcccactcatctacagaaaacaaacgctcattccgagacagagagccataacagtttactgttaagtctaccattaggaaatctttaccgtattgttttgtccaactctctgtatatggtgttactgagtgcacttctttaatatattgctctaacatattcattttatattaccctttcttggtttaaattttcgtgtttgtgttcggttgtttctacttacttctttgtatctacgaatacctaatgccttagactctggacgagaacgtcctagctgtcttacaaataagtctagtgcttcttttactttgtcatcatacatcttccctaattctcggaaatactcaaactcacctaaatacacatggggcatacgccattcatctagggtagcctctactattgttcgtttgtttaaatcataattatagatatataaaaatactatataagtacctgtcatcaaagaatagcttgtcctaatttcacttagagttacgctgtctcttaatgatgttaaataatcattccactttttcttacgttcttctgctaaagtttctatgaagtggtttacctctttctcatccctcactacttgctacctccttgttgtttatgtctatatattatcacctactttgttatatgtcaacacctttgtaataaaaaagagcctagtaatttacttacttaggctctcttgtttatatgtgtttaataagttacctttactatcgacaacaacagcattaagttgaccaccgtatgcacaggcaccatcaataaaaatacttctgtcacctaagaatacatcgtggctgttctcttttctcaaattaattgtgggtgtgtgacccattacaattgtcttatctgttttattcttctgtaacgctagcggtctcgtccaaatcatttcatctttagtagctgttttccaatcaggctgacaataaacaggtatccctgcgtgtacacataaggtgtctccatgctcatagtatggttctaataaactaattacttcaactacatttgttttttctaataacatctttctagtggtaaacgcatcctgtaggtgttcatctaaatccaatagactacttatcgttttatcaccgccattgtacatccacattgcgtacatctcacttaggtcttcgtagtctgattctcttatcaaggggaatgttaaaaaatctaataacatttggtcatgatttcctaacaaagcagtccctccattaaggatatgcttataaacgaaacccagtacttctgcggatttgctacctctatctacgtagtcacctaaaagtattaagtcctcttcatcactattccaatgtttattaagaagcattattagctcatcataacaaccatgtatatctgatacaataaacactttatctttcaatgaataatagcccccttaactacttctgctagccagtaagtagccagtactagtacagtattgacaataaatgttattactataaccagtgctaacttataatttttactcctagcattatagtttatataattttttatgttaactctaatatttttaaacaaagtgctcccatatacgttcagtgctatatatagtaaaagcaatacgataaagtagccttgagctagaaacacgtcagactccctgaacgagttattaaaccatatgctgaatatcgcaatcacttggtgaattataagcattgcgccattaccaaagaggagtatactaaacgctataccgactacgtctctgtactctgaaagtagcctatcatatactattatccctttagtaaaaataaaagcaacaactgccatctgtataagtaaaaatattgttacggctaataagttttctgcatgcatagtcataatttagccactcctttcaaaatgggtaaagaaagaataataggtatatcgttctttttctaccctaccctcatgtataaaggtaaactcaacaaacttaactcttagcggtatatctggtgctttctcccaaggaagtactcttgggaaatcatagaatactcttgtggggtaaacttctttcataagtatcctagggtatcttttctttggtataaatcttttacctattatacttgagtagtcaggtttcattgtaagtcaccatgaatagagtatgttctacttgctttgttaaagtttacttgttttctgtttactaatcttattgcatcctctaataaaagaggttttcctaatttatggttatctaattctggtgagtctacccctacattcagtaagtttggttcgggtctggctaactcatgaatgtgcccatgtagattaattaggttacctctatctcctaaaattagtgggtaatgcgtcatatgcacaaccttatgcattcgttttaaaataataccaacgtcttcccagtgtactctactgtttagtgacgtattatttttaaccgcttttcttaaaggactattatcatgattaccctttattagccatatagtcccgtttagacgctctaacacgttaacaacactatttaccttagcacccattgaaaagtcacctaggtggtacacaacgtcttctggacgtactgtagtgttccactcattgattagatactcgttcatctcttccacatctttaaattgtttccgtgtgtccataaaagaggcttccccacaaatgtttctatggaagaagtgcatatcagatattacatattcttttaccatggtgtcctccttactgtgtaaccaaccatacaaatacgaatagttgtaggtagtgtactgactggtcgataataaactgttttcgtgtatccggtactactttaaggaaccatgcactttgtgcttttaaatagtccatgataaaatgtggaacccataagcatactacaattagcccaagttttactggtgttcctaagaacaatgtagttgcgacaataatatacgtccagatgttcacatggataaatagtacgtataggctcgtttgtttccctttggcgatgtaatcactttgtaaagcgtagtcccctaatgcatgtgccatcacgaggatagctaaataataaaagtaactcaacaacaatagtcctcctctttgtatgggtatgtgcaagtgtcacaggcatatccgtattcatttggcgcaccttctaataaatctccaccacattctgggcaagttgctagtttaatcattatagtttccctccatcaattagtaatagtagaataccacctaataccagtagccagaatactgctaaaattagtaccatttagttgtcctccttgaacagctcaccgtataattcttgaattgcctcacgctttacttttaacattgcaacctgtgctgatatgatgttacttggttttcctactaaacgtccttcaaagtgctcaatatccatattgtattgaataagggtttgttgaaatttttttctaatctctgtgtgttcatcaattgattgcttcggctcaatctcaatcagttcaaactgtgaataccagtattcatttaatctattcagtagctcaccacgtgtttttccagattctgcggttgtgccatcatcatctttaataaagtaccctagaataccattatgcgcaatctggtattctttgccttctgtgaaaaagggttcttgtgggtttgtaattacacatttcattctgtatgtcattattcttcctcctttaaagtgtcttattcaaactcttcgatagttttaatcacttcttcaagtctgcgtgcctttgcgtttaagttaattgcttgctcatttacacggtcacgttttgtaaataaacgttctgcctctgttgtaagtgcatcaattttagcttggatagcttctttcacatctactttaacatctgcaactggatattctttttctaccaattcatattgcgcttctgagatgaaataccttgggtgtttatcatttaaataaatatagcagtcatcagtgagagcatcatcatatgatacaattttataggttttaccaatttctaaaccctttctttcatccattccatcgaatccagcatattttgtaattttagcatatttttgcatttcttttctctcctccacaacctcaaacctaattattgcatttttatcgtttaaaccacgtaaaatactaacttctgtatcgtgtttatcaccttcatcgtctaaaatataatatgctcccatcttgtcaacgtctagtttacttactgattttacaatctcgtactcttttcctatagtccaccacttaacaacggagttgtctacacaacgtaatttcataccttcgtacaaatcttcttctgtgtatttaatttgttcttcctccttaataatttcaaatgctacggggttatgttttccttctaagcagaatagcatgtagtcggcatatgcgtagtcaccattatcgtctatgattattaacataccatcttcacttaatgatacatcatacactttccctacagtccaccatactggttttgctttggtgcaacgtagtttagtccctacttttaaatcattttctgtgtacctcattttgtgtcctccgttaatttttactgtcattctaattttcctccacttctttgattgacttgattatatatctatggttctctctaaatgcgtggtaagcatcatcaagtgaatatgcgtataatcttgttgtttgcgttacagttctatacccgttacgtccaaccattggttcgtcaacttctacttctaactcaaataatagttgttttctaatcattgtttttcctccccttttctatgatataatattaccctactattgtgattctgtcaacaggcaaaaaaaagaagtagaaaaatctactcctattcatcatttgacaacttttccataccatataataactgtaatagtacgtatagtacaatatcaaatccgttaagtggctttccaagcactataactttaacgattgtcaataccgctaaaaaaataaatagccacccggttattttatttgcatgttttctaattctcataactttctagcacctcttcaatattctttctggtttcgttagctaacttaccaatccgttcaagccaacctctaaccatttctttttctactttgctatgtttgttcgcatcctccttgataccttctacattttccatcaagttatctaagtcctccataacatctaataaagtgtttcttagttttaacacatcaatagacaactcatagttccgtttagacaatttttcgttacggataaagttcttttttagttcttgttgtgtttctagtagctgagtagttttaataatatcagcttctaatttgctaattccgtctagttcattcatcacatgtcctcctcatattttactacttcctcaataattttcagtacttcttctgctgtgcctactgtgctatagtccgcaaagttttctgcaggtttcataatagccactgaatagggtttgttctctacataaatatgcccgaatccttttagagaatgaattacctctactacccacccattagcatactcttctcggtatactacttgctcactggttgcccttactggtttattcattgttaaaaccatagaagtttcttacaatattttctacacgttccataccctctaagtaactacgtttctcaggggagatatagttcttgtaatgagggacttccttctcttcctctacctcttttgtagtacgttcaatcagtttaataagttttacttcgttctctaattgagactggataatgtctaacagttcgtttttcgagcagttgtttagtgatttacttccatagtcaataattgtcatagtagtcctcctattttaatgtgtactctttatatcctttatgtgtatcgtgcccacactttgtacaaaagcctgcttgttcaatatctattgaagagtagcctgtttgtactgcatatgtctcataagtatggtcacacgtatcttcttctgattcgtctattttctctaaacgaatagcctctccttctgccacttcgtggttaaagagcaatacagcagttttaataggttcatatatatctgggtggatggcttcattcatcttaaagctatttccgctaaattcattgaaagcgtctagccattcctgactgttctcataggatacaattctgtaattcatacgaaagctcctctcgtgttttcttaaataaccagtaccgttcatcaatttcttttatatctgcctctgttaaatagaagtcttttaatttggatagggtatcttgactcaatggctcgtttctaggtaatatgcttactgctaactgcccatccttgcgtaatagtaacccgtagtctctctcctgtttctgaggcattactacatagtacttaggtaaactagcagtgtttgctactgcttgaataaaagtatctgtatttgcgaaaaaccatttactaaactctgacttagggtactcacatgcgtgtctctgtccttccaagataatttgtaacttatccataagttgttctcgttgtttatcaataaactcttttacctgttttggtacttctacctcattattcatagtttgtctcctttgttatactttgtcaatagtgatatacacatacttgccctctaactgcttaacaatatctagtatgtcatgaccacctagcttaaatgtgtttgtctcaaacccttcgatagtaaaggaagaccaaccataccagccttcttcaaagtccaatacccccatagatttaagtactatatttttagtgatgtcttctaatgaagtatgttctacagcagagaaaatctgaatctctgcgttatcaagatgatacttagaagaaatgcctccatcgtactcctcttctgttgtagtgtcctcagcacctagtgcatgtagtgcattacaaataagcatgtttaaggctctgtgctcatggtgatactcgatttcttttactttttcttttattaagtaaggttcgtttgtttctgcaccgggactgttatcaatagttacccaaccttttaatatctgtttacgcatagtgactacctccatttataattgagaatgatacattaactggtataacatctagtataccatactcacctttttgtgctagtattctagctaaagtacctttcctgtttactgcagagtccttcatagtgaaggcttgtgcatgagaaacgttcgatgaccacgagttaagttttagtcctgctgtgtaatacaaatatacttcatattcctcgtcatactttcggataacaaactgagtgttcatttaaactaacctctcttccttagtataacctagcgctatcaccagtaaatctaagttttcctttgtcagatttactgtttgttcatgtacacagtccggtctaaatacttctactgtacggtttactacagcaatatctacagtatcttccgctgtctggcttaccttaataaactgtttggaaaactccccacggctaacgccttctatcataagctccatttgtttatagcctcctctcgggaaagtccatgaaagttgtggaaagtacgctcttgtagttcgtctaccgtatctggtagtgtacgaaattcgtctacccctagtgaaatacttgaagctacctcttcataataacatttaacgtctactcccattttaccataaggtaacaccattacttctatatgaaactcatatggaatattctgtgttaatggttcatctaataaccatgtctcatggtagtttactggaatatctaatagtaaactgtttaaatcatctaactgttcagataagtacgcaattcttatcattttacggtcttctgccataggcattgaacaacctaacgtatcttttatttctttaatttctttggtaagcttagccattaattcaattattttataaaacatttctattcctcctcttctatatattccccttcttctaagaaaaagcaagcaggttctccctctccattttcagtgtcacctaggtattcatcatctaagaatgcacagtagccagtagggtatagggttacgcagtccttatggtacattgaaccgtccataatccgtacaacctcgtccatctcactgaacacttcgctacatgcctcacatgatactaactccttactcataggctacctcctttatctattacgtagtctaccagttctctaataacctctgcctcctctacactagagaggtctgcccaaacttgaagtgtcacatcgttagccttaccagatattaaattgtgtaagctatctgcaatagttagctttgtacgttcaagatagtgaattagccagactaatacaattagctgtttctcagttagctcagtagtaggctctgtgtaaacctcgtagttatctaaggataatgggaaggtttcttcattattaattacttgaaacccgttatttggttcgttttcatattctccataagttactctcttacctatttttagttttacatagatgtctttaccttctttgtgtagtttatctaggtcactattataccactctcttgtttcaattttagtaagttccataaagctagccctccttagtaagctccttaattcgattcttaatctctcgttctgtcttttcaagttgtcgcatcttgttcttgtgacgagatacttcgtgttcctttgctgtttctgccatcgctaataggttctctaaacttgctttatttgccattaaattgccacctcatagttgaattttccattgtgtttataatttactacagaaatatctgataatggggaatcaaagaagttcgtccccttcttccatggtaggacaagctttggaacctcttctggaattggtgcgtttacctgtttctctactagctctaaatgtcgttcgtaaatgtgtgcgttgtcactcgtccaatataagttacctaactctaaccctacagtgtttgccataaccaattgtaatgcatgatactgaataatattaaatggaagtcctaagcaaacatccgaagaccgagccttcacatgtaagtttaatttaccctctacaactgtccagtgggttgtccatacacatggttctaatgacatatcgtataaatcttccacgttccataaagtagtcataattctacgagagtttggtgtgctcttaagttgttcaataactgcctctacctgatttagctttaggtatggttgatgtgttcccgctatccaaccgccttttttaggtacgacttttgggatgagtgagcctgctttacgatttttagctaactcaacatctccgctataaacaggaataaaacgttctttgttaaataatgcgtaaccatacgcttttccaatagtgccatcttcttgctcccattcgtcccaaatagttacacctcgttcttttaaccacgaaacctcgttagacatttcttgccaaatccactgcaattctgttagcgcccatttaatacctacatgcttactacgtaataaaggagcccccatatctggtgtaattgtaaagtttacaccttcaataaacttagctgttgcaggagtcccatcagcatatactgcacgagtcccttctggtaattctgttactccgttcttaataatatccattactaaatctttataaattttatcaaaatccatgttgtatcctcccgtatctctttcctatttagcttatttttttttgtgtagctatacccaccagttaggttctttgataacttgccatgctctttttagctgttcttcattgaaatgtccaaaatgacaatcttctttagggatgccaagtttataagctagtacactgtatagctcccctctgctaatctgcttagactgccatatggggtcaaataagccatgacataattttttccatcgtttcataggtggtgtagctaaaatacctaatggttcttttggtctatttttgtgagtccctacactagcaccacaacctgtacagtaatagcatttaccattaccgtatgtcctaccatataacacttcattacttgtccaaataactttttctccacagtaagtgcagttatcgggtactaacaatttattcattagttattcacctctcataagtatagtatattacaatagtgtcactaagtcaacacctctttacaaaaaaagaacagcaaagatgctgtcctaaaaatctaaccaattatctttatcgtaagagtcacttggagacttttctttgtttttcttaacgaagtagtatccgatagagaaagctatccctacaagcaatactacaataagagcaataaagaatttagcaatgaaaaaacccattacttatcattccccacaattgcctgtccgtcacctacgattgtagttacccaaccatgctctttacgaccttgtgcttcttcgtaacgaactagttcatccgtaactgattctgcaatttttttattagcctctgcttgtgcactagctttatctttaatagcttgcgcttctgcttgggctttcaaagtaacttttgtagcctcagtttctgcttgtgttttttgagttttagcatctaactcagcttttttagcttcttgtcctgagcggatgattgcatcaattgatttttgtgtttctgggtcaacgtcaggcacacctaccgttacattctcaaccaagaaacctttactttcaactgagtcagagaaacgggctaacacttccccttcgacctcggaagacttacctgatagaacatctaataaactatacttagaataaacctctctggcagtcttttgtaaacgagatttcaaccaacctttttcaatgtcctcagatgttacattaccaaactctttatacattttagttgcttgcttagggtctactttgtagtcgtacttaatgttaactacagttttcttaccatcggaagtagacactgcaacatctttagcttgaatagtctgtaaacggattggatactgggttactttatcaatacctacccacttaacaccgggttgtaatgcttcagatttaacacccccgttaggtgagaatcgtacaccgacatacccattgtcaattcgctctaagaaacgaaatgcacagattgttcccccaatcaataggataactgcaataactcctgctacaaccaaccgtttaaacgtatttgtgctcataaaaaaaataattcctccaatatttattttttatttgtaagtttcatcctacaaaaatagtataccagtttttgtatgtgtagtcaacaatatatttaaatttttttattaaaaatatgaggcacaaaagaatgtaaaatatgcctcatatctattaaattctatactaaactaactagcttgtctaaagcgttatagtccgcatcggttactggctctactttacctaataagtacccattacctacctgagagaagaagtcatggttactagttgttgtagagataccattcattacaatagggtttacgtctgcgcttgttgttgggaataaagggttcatccctaagttagctagagctttatttaaattgtactccaaaaatgtattaacatcttttgtccaaccaatctcactatatagttcagcagtatactgtgtttcgttctcccataatttaaatgctaaatcaattacccattgttccatttctttttgttcagtttctggtaactggttaaaccctaactggtatttatagccgatataggttccatgtacagactcgtcacgaataattaatttaataatttcggctgtattaaccatttttgcctcacctaaatagcgtagtggtgtgtaaaaacctgagtaaaataggaagctctccaacagtacggatgcactctttttctgtagtggtgtaccgtttcgataaatatcgttaataatgttagctttatattgtaaacgtttatttgtagctacccattcaaagatattgtcaatctctgtaggtgtattaaatgtgctaaaaattgttgaataacttttagcgtgtactgcttccataaaggctatgttatttaaaacagcttcttcatgctgtgttcttacatcatcaagtagtgagtgtacccccgattctgattgaactgtgtctagtaatgttaacccaccaaacactttatttattaaatcttgctccatatcagagagctttgcccaatcattaacatcgttacctacagggacacgtgtgtccaaccaaaattgctcagtaagctttttccatgtggcttggtcaattacatcatcaatcttattccagttaattccttcatacttttcttgcaaattcatgtgctcatctccttaaattgtacaactttcacactctgaaacccctacttcgcttccgtcttcttcggtgaatgttctaacataataaattgtttttataccttttttccatgcataatgtcttaataaagttaagtcccgagtggatgtagcgccaccctccaccttccaaggataaagattttctggtaactctgagcgcataaacaatgttaaactcataccttggtcaatatgtttttgtgccgttgcatacacgttgattacgtcaatcatacttgtattgtacgctgacttgtaatatggaattgtcttctcagataaccggggtgcagggtaataagttttacctgttttcccatcttgacgttcttcaatcatagagataattgggtgcaatgaagaagtagtctcattaacataagcaatactaccatttggagccacagctaaacggttttgatggtatagaccaccttcaataatactgtttcttaatgtttcccaatcttttacattaggtagtaccactccgtcaaatacttccgccaccttactagagatttcctgatttagtgttggtacatattcatcaaagtaagaaccgtctgcgtatttagagttctcaaagttatggaatgttgtgccacgctctttagcaatcttattacttgccaccaaagtagcatagttaattaattcaaacagggcatccgtcatttcaatagattcaggtgagccgtattctacttggttacttgcgaaccacgcatgtagccccattgcccctaaaccaattgtatgggctttatcattaccattcttgatagtaggtactgcttttatatttgacacatctgtaatatatgttagagctcttaccatcgtctcaattgatgccactacatcattagatgtttctaacaagttaaggatgttaattgaacctaaattacagctaatgtctgtacctaactcttcgtatacttgttcatcatttagcttagatggtgtctgcacttgaaggatttctgaacataggttgctcataatgattttgccatcaataggattggctttatttacagtgctaacattcataatatatggataacctgactcttgttgtaaacggctaatttcttcttccaaatctcgtgcattcattaccttacttctgatatttgggttctgaatcatgttattgtattctgaatcaatgtctacaaatgagaatggtacaccgtattctttagcaacatcatatggagaaaataagtgaattacatccccagactcagctagctcataaaatttatcgggaacaactactcccaaagaaagtgttttgatttgctttgcggattctgcattttcttttctagttgataaaaagtccataatatccgcatggaaaacatttaggtagactgcgcctgcaccttgtctttgccctaattgatttgcgtaccggaaaccatcttctagcattttcataacagggacgacaccactacctgccccctcaattcccttaatagggtcaccaagagctcttaagtttgaaagatttatgccgactcctccgccaagtttagatagttgaagtgcactgttcaacgttctaccaattgaagacattgaatcttctacttgaattagaaaacagctgacatactcccctcttctaagtctacctgcatttaaaaaagtaggggttgcaggttggtatctacggttaattaactcatctgcaatcttcatagcaagctcttcgtcaccatcgcccattgtgagtgcattaaacgctactctatcttcataacgctctaggtatcgttccccatcatctgttttcattgcgtattgtttatagaaacggtaagcccccataaatgtgtcgaatctgaactttttactatagatgaacttaaataatttttttataaattccattgagtatttgtcagtaattactgtcttatcaatgtatccttctcgaattagataactaattttctcctctaaagagtagaaaaatactgtgttcttgtttacatgttctaaaaagaatgccctaacagcttctttatctttgtctaactgaatctttccgttgttagggatattcaattggttattaagctcaatatatgtgttcatatctacaccccaagttctttttctatttttttaaccaaatcaggtctaacaccattaaatgagaaaccatcatcagcttctacatatggtaatgaagacactcctttttcttttagataagctaaagcttgctcatcatgagagatatttttctcatcaaatgggatattctttccatttaacatattttttaaaaataaacattgaccacacatatcttttgaatatactgttacttttgtcatttactacattctcctaacttttttatcgtatttattaattatacactattttgcattaattgtcaaaaataaagaaccaaaaaaaactagagactacaaaagttaattgtagccccttttaataagttacaccttgtcacctaagaaatcttctgattcctgcgatttattcgtaaccttatcaagtagtaaaacagaatagtttttaccagtttcaacctgcaacctattaaacactttctgatattcttctggtgtgtctgtaacttctaataagtattttgcatttaaaaatgatagtaattcgagtagttctgccttattacctgccatttgaattacaactttaccttcatcgttcgtgtatgttccaattttatttttagtagccaaacaaatcagtccttcgtaaatttaatgtatttgttaactgtatacacatttttaactttttcccaaacgttagggtatacgttcatcaataacttagtatctgcgctagcacgtttgtactctggcattaatttaaatgtgcccccatcaatactaattttagattcccctaactcttccatatcagaaatgatactactttttagctcatcaatctcttctttaatacttttttgcaattcatataatttcttgtaagagcgtaagtccttctctagtagctcctttttttgtaaatctgttgttttcatacatatctctccctctcttatttctataataagattatagcatgtcagatatgtaaagtcaacacttttttatacaaaaagaattgcccattataaaggcaattcttgtccgttgtcttcttcaaaaagaatctctacaggtatattatataatttagcaagttttactctgtttttaacacgaggttcttttataccgttttcccaataagagattgatgaccagtgaactcctatcatattggctaactctcgtagagattcacccctagcctctctcactcttttaaggactttaggttttggtaccttttcttccatgactatccctccttattcttaaaaaataagagcctatcaggtagacaggctcagcatcaggaactatgcgaaagccccgatataagttactttaccattacatttacgtgcaaaagcctctaactctttacgagcacctaaacggtcaggttgcgaagattgaacagtaatttctttaggtaccgttacttcacgtgtacgaatttcagttttaccattttttaatttcttctcatactcctctgtacgagttgttgtcgtagaaatgattccataaaattttttcatattaatagctccttactgttttttttaataaattataatagtggtaatagggttttatgagactcctactcattgtaagccttccttaatagatacttacaactatacgtttatagacctctctcaaaaagtctccgtaacgtcttaccttcatcatttaaaacgtacacagaatttacaaaagttttatacccttctttttcagttctgtactcgtaaggtgaatttggcacacgagtgatttcttcatgtttagagttaagtaatgaaaaagcattctcttgctctaaaagaaactgtaccccatcaattaattcaataaaaggcttaacaataggttcctctgtactgtctttacaatcgtattttcgttctcctaaaatttgtagtatcatgtctatctatctcctctgtttaagtattaatttgtgagtttccactcacaatgctgattgagagagtcgaactcccattcccgggttacaaaaccggagtaatagccattatactaaatcagcgtaacccctcaatagagggagtttcttatactaagttaccatatccgtaggtatttgtttgagcttgtttaaacatgatactcttgatagctaagtcgttgtacttttcatcgtcttcgtcagcaagatacacatcaacattaacgttcttacctaaaatatcgattgtttcatctttacctacttgtaagtctttactgttaaatgatacatcaaaatagatgtcatcttcttttgtacgactaccgaatgatactctatcttcaaataaatttaaacttcgagaattaattacaacaggctgtagcccaccttcaaattgaatagtaactgtataatactgattttcaatattaaggatgttcaaatcagcaatagcctcaccaaatgagatgccaaagcttaattccaacgcaattgctcgtaagcaatcatagtttaacttaattctacggctaaacttaacaacactgtcaatttccccatagtaagctttgtctaacttatcttgcaagtaagtacgaatttcgtctgcatcaggatagtcgaaacgtaaatggtaatggaaacgtcccggacgattaagcataaagtcatttacacgatttaagttattaacagtaagtgcataaatacgcttgcgttgtgataacccatcgaataagctcaatagtttttcttgtgattcacgaccgtcacggtcattgaatactttttcaaattcatcgaacaagataagagactcttggtcaatactgtcaataaattctgcgatacccggataagcttctgtaattaagataacaggcataccctcttcaatagctcgttgagataagatttgagtaaatagtgatttaccaatccctttatccccacttaaaataacccctaaactacgattgattgttttaaatgtgtgtagtactttctcaattttaactaaacggtcaccataaactttagattctttcaatttaaaatcgtctgtaaccgccaatgagaacccagacattgggttgaatctaactttataagtttgcgcaggcaacttatcataagttttcaggtcgtttgcataaatctcataattactaccataattgattactttcatatattctctccttaatactttttaataaattataaatggagagtgatggagtcgcaccacccgagcctaagcaacagatttacagtctgctccgctactacttacggaataactctccttgaataaccaaccagataggcacctgacacacggacagcacctgtgtctctctgttactggctgattaattgtgtatttcgcaccacctaatatagttatcaaggtaggacttgactatactagatttagctaggttctaactataatttcctatggtagctcctactactaatcatatttatatacagttaaactacacataactaaaccgattaccttggttcgcatgttaagaggcgcaggcaaccaatggacattgcaggtctcgaacctgcgaccgttcggttatgagccgaatgctctgaccaactgagctaaacgtccttaatataattagttgggtacacggcactccgtatatttaatctgcgaataggcgcagacccaacttagtttttatagctacccacttaatttggtctttttacattatcatgcgtgggtaaagttaaaactcttgtaataagagaaagactgtcatcgttagatggatgataatgttttacacacaaaacctagagtgtgtgtagtgttgggggtactaaaaccaacgtttttccctataatagatacctattaattatcggatagggcttgagtcctacctcttagagtagctcattaattaaatctaaaattttatattagccgttttaatgtcttgccatctttattaagcaaccatgcttgttcaatagtatagtcttcatattcatcaatatagttatataaagtcatgttgcctgctgaatcattttctaaggttattacagtcctatctgataggtgtagttttacaccaccatctacatatgttgtacgaacaacgtcttcaataaaatctgattgaagtactgtaggggctcctttcggtacgttctttgtcttgtctacttgtaaaatcatgtatatctacctttctgtctaatatttatctatatagatatgcagaaggcgggagtcgaacccacacgtccattacagaccgatgtttctaagacaccgttgtctaccagttccatcacttctgcagttgtactaccaaggttacctctaaaaggctaaactcgtgtctggtagggagccataccttacatggattgcaaggaacctaacctcagtagtaccttttactattaagggtttctgatttaacgttttccctagaccgtaccttaacagtaatactggtgagaggatttgaacccctcttacctacaagcctccgacaagcgctatgggagacaagcggtgcattaaccataactatgctaaccagtacactactgttttacgcaatccgcatattagtagttctcacggactcaacccaattcagggaagtggtagccttgttctagtggggaactggttgactccttgtacgccaaacattcccagactactcacttacattgttatttaatgtcacatgtattatgacagccgtactcaaaagtttcaaggacttttacctcactatccaccgacagtatgctcggcaacgtctttctaataagaagtacttgttaaacttagggaaggttaacaagtatgtgcacataacccgaatgctactccctaaagtagcctatacgggctaacgtagatggagggaatcgaacccccaaccgtccgcttagaaggcggatgctctatccgattgagctacacctacatattatgggggtaatgagtccccataataactcatattagtctcatctatcaaagcttaaaatgaatatctaacttaaataaagtatatcactatttatagaacatgtcaacacttttattaaaataaatttctatacctcactaatgccaatgactatacgtaaggagagaacgttaatggcactggctacttatttggagataagctaatgaggtatagaaagagaactacattgaccgaatcgtattatacagccttagtagttgttaacaatgtttggaacttagtagcttactgcaataagctaccccgaagcatcggggtgggactgtggtaagccccaaacccttgggtatgctccatcctgcctccggagaagtcaggctcaattctcattagttaaaggtttactagttaacggttagctgattagtcatactacgtaaactactagtaaaatatagttttccaattaaggctaacaatcggtagaggatttgaacccctattattggttttggagaccaacgtgttaccagttacactaaccgactatgtaaagaaggaaagcgaagaccttctttacagtaattaaaaaaacaaacaaaaaaaaatacaatcatggaatcaaggtgggatgtaaggagatgccatgtgtccacttatagtggaataggtaactacagatttgcactgttttacatcttgtttaatggttacctgttaatagctaacttccagtgataagtcctcttatatgttagttcatgtagtatttgtttgacaatatggtagctagctaaaatgacgacagtaacgggactcgaacccgtgtgttccacgctgacaacgtgttgggacaacctctgccctatactgccattatttatgccgtagttttttttagaaaggaaagtttttttataatatacaacaacagttgaagcgctgatgttactacggcataaatatattttttatgagcttttaataactcaaaacttaatataatagggcttttactcctgcttcatgcacactcactgcattcggtcacatacccagagaggctgaactatcgggagctacccaatagcttctctctgcttttaatgtatggtctagcacggtggctaacaacgttcacctaaacattcagcattaaatagtattgttataagtttttcttatatcatatattttgtaagctgacttaggctacttgtaccaaggtctcttgttaaacccatatttctatggttctcgtcaaagggagtcttttgaggtttctaaggtctgccaagtttctcacgcttccacctaacttagattcttttccacaggaacgtctattatatcccccaagagtcttgtaccttacaatccatgtacactccacataacgcttaactgatactagcttttactagtagttactcttatgtcaccataagacatacctgctacctaaaacactatcgcactctaggctacattcagaattacgccacgttatcctagcaagctgttcaaactttctgcactaggttctattatattaagttttcaattatcaaaagcagtttttctttactatttaataatagcacaacctctattattttgtcaactactttttttagagagggtgcccctaattttatataggggcaagagatattaaacatagctgagctatgtaaaagctaatgtaggaattgaacctacttaatgtaagcaacaccagtttagcttataagcattgtctgcaaagtccatgctatgcactcagtaggaatcgaacctattgccgaaggtaccaaactaccctgagtgctcctatagatatattataccacataacgagatattaattttttttggtgatatagtatatctatattttttttaattttagaaatacctttgatacattccggttatccaacgtatgagtatataatatcatgttattctgattgtgtcaataacttttttacatttttttttataattgtgtaatgtcctatttctttttttctgattccttatatttattatatatactatatagtactatatatatatatattaattactattagatattatatatagataataagaatctaggaaatttaaaataggacattctaatgctgactcagttacaatcaacttatggtgactacctttttacaaaaatgtcctatttcattttttctctctccttatattattatttataatatatagtattaagtattatatattattatatatatagtatataatataagaaagacaaaaatttaaaataggacattttacttaaacaaaaaaaagagagcagttaagccctcttatcttaatgagaaatagttattaattgtccccaatctgacttcctcatcttctggtgtttcatcctctggtttcgtatacaagctagtgattctttctggaacatctactatgttctcagtagctgtctgtatctggaatgcatttacataagttaactcagaatctctatcagctagcttatgaactggaatcattggcaactctgcacctttcattgcaccatttacaattgacattgcatcattagttgttacaccgagtcttctagggaacacagaatatgtcccagatgctaatgaaacctctacaactttctctcttttagggtaattcatgatacttctcctttcaaaatatgttataataatactatatactataataacacattttacaaattacttaaacatgctgaacacgtaagtgacaactgctcctaagacaatcataaaaacgttctctacaatatccctcttgtggtcgaactctttatcgtttactccttccagtgcatctatagtttcttccaacttagtaatctggtaggttaggtgtgcatatttttcttcgtgaacagcaagtcgcttatcaagactattgacaatatctctcaactcactaaccgcactattcagttctacattgtcctgtttaatgcccttctcggttagctctccatgttggagtttatcttccagtcgttgtagtcttaaaacaaggtcatttacttggtaatcgttcatatatgctcctccctgtaagttatgttgctctatattagtcagatgtaaaccacagacagactagtatacctataatagcgcctaccgtgtagactataggtatatagtacccattaaaaaacaagtatagactacctatactaattccatagagcacgaaagtgaacttgtatgatgccaaagggctcagttctgcttgtagtttcttatactgtagtgcacgagtgacatagtaggacatcaagtaaacaaacatttgtagtaacagtacacagaccagtgctgtttcatgcattatgttgtcccctcctctcccttagagctactattaatatagtacactgagctgttagcaaacaggtgtgctaaattaatatagagactaaaaatgtagacgggagagtggtaaaaatggataatagcaaacgaattattaaaaaaattatttttattaccatctctgcattagtcatggttactttaagtaagctgttctctaaatatgttattgtagaacaaaatgccccatttcaggcattaattggcggtgcctcatgtgcattgctatctagtattctgtttgattggtatactaataaaaagaaaaaagagaacgtagagaatcaacttaaagaagcaatcagtgacttgcagaagattaaagctatcataaaaagatagcctattaggaggtgggatgggtgtctattaccaataaagacattaaggataaacgtagatatatttttagtcaatctagtaaaacaacaactataaaaagaggggacaaacgcataagtagtgcaacaagaatatgtgcagtttgtggaagaccgctatctaagctcgtattaagaacaggcgtaccaacagtagtagtagaccatattagctgtaagatttcagacattgttagactaaatgtctgtgaggatataaggtcttgttatgcgtattctagtaagaaaggggaaagctagctaatgggtatggcagatagacttaaagataatgcaaaacaaaaaaagttagaaagaacaccagagcaacaactaagagacacatttaatcaagcttcaataaagcttatcaatcaatttatggctaacgttacatcaggtgctatagaagttgatgatattgcagatttaacaaggctatttcagatttatttacaggttaataatataaatgatggaatgcaagaaggtacaggaactctacctgcacttacatctgagcataaagacatcatatctgaaaaggttagcacagaaaagattattaaggacggtgaagaagaggagctaatttctcttgatgagttagccagtcttccagatgaccaacttgaggaagtcttagttaacagagagctacagatgaacagagagaacgaggcgaccttctaatgacgacaaaagcacaacatatagctaaaatggctaaagaaatgtatggtacagataaaattacaacggagcagttagcttatataacagatatgctgaccccatcaacgtacctattgagaaatcactctgtgcgtaatcacccaataacttttattatatcaggaagggatgcaacaaaagcacaggcacatagaccataaaccaacaaagctgtggtctttaaactctactaaacgggcatagtaaaataatatacacgtaaacaatatactggtaagagagactaaatcctagtgtataaggacagggttgacctaccgtgctaaatcagttatactataactgtaaaagcctaacgactaaatttctaggtagctaactaaaaggagttagtgagaactagataaaaggcaaataaactatgcaagctcaataaaaataataatatacaggaggtgattaagatagctaaggacagaaaagaaattttcatagaaaagctcaaaagtgtaaaaggaactgactttgaacttataggtgagtttacaaagcaacgagaaaagactctatttcgtcataacgtgtgtgggcatgtttgggaaaccactccggttgtcctacttaactcaaaaaaaggcggagggtgccctcattgtcaatacagaaacaaagcaacttcaccaaaagaatacgaaaaaagagtaacagacacatttaaaggagaatacgttgtactcaatattaatgagtataaaaacaatagtactaaacttaaattcttgcattctaaatgtggtacagaattttatctgcgaccagcaagtttattcattaatgcaactagttgtcctaactgctctaaaaataaccgttgctcaactaagagaactactaatgagtttagagagttgcttcttaaaacaaaaggatattcctatgagctaacggaagatgcagagtacaccggagctaacaaaaaaattaaggttaggcatacaacttgtggttatgtttgggaggctagagctaaccacctgctacagggttcgggttgtcctagatgtaacgaatcaaaaggagagctattagtagctactgttcttaagctaagcaacacctcttttttgagagagtacacctttgacgattgtagaagtactagacccttaccttttgactttgcattaattagtaataacaaagtgcgtggtctaattgaatatgatggagaacagcacacgaaaccagtaagttgtttcggaggggaacagaagtttaaaagtacagtaagaaatgataacactaagaatgattattgtactcataaaaaaatacccttactcagagtatcatacactaatagtcctgaacagattgagcacctagttcagcagtttttaaaaagcatagatttgttgtaacgtagagcaacctaaaccaaaaattaggttgatgatatagtctagtcccctaataaatatcgggaaaccgagggtaaccaaatgggcaaccaaagataataaatgaccaacatagagacaaggcaataattaaatcaagacagctaggactatcagagatgggtgtcggttctatgctacagtttgcagacacacatagttatgatgctgttaaatgcttgtatacattccctaaactaattggggcttaggtcagtaatgacctttgaaaaactctgttaaacgggcataggagaataaccaatatcctgataagagaccctaaaccctaaaatagggcagtgggaatcccgtgctaaatcaagcgtaagcttgtaaatgcctaacgactaaatttctaggtagctaaccaaatggggttagtgagaactagataagaaaacctcttaagaggaagtaaagcagagcaacctaaaccaaaaagtaggttgatgatatagtctagtccgactgccaagcgcagtgttaaagtatagtgaaaactacggtagattcgacaaatgagcaaatgactaaatttgtacagacaaggttagaccctgttttacagaatgggtactacagcacaattgtagaccaagaagttaactcattaaaagctaaaaaaataagaaatagttttttgtattttcgttcaagctcaaaaccgggcgctgtagagggtgtcgatattgactatctatctatggacgagtatgaccgtgtacctgcattagcagaggcttctgcgttggagtctatgtcttcttcaccttataagatagttaacagatggagcactccgagtgccccagatatgggaatacatgggctctttaaagggtcagaccaacattggtatttacataaatgtgagaaatgtaattattacaacgaaatgagttatgatgcatatacaccagaggctcctgtagagagtagaggtaacattctttgtgttaacccaaaaggggtcgatgttgttgctaaaacagtagttgacggctcattccagtttgtttgtcaaaagtgtggagaaccgttagacagatggtacaacggtgtatgggtacctaaataccctgatagaacaaaaaatggtctaggtactagaggatatatgatttcccaaatgaacgcagtatgggtaactgccgaccagttaaagactaaagaacttcaatcattatctaaacaagccttttataactacactcttgggtacccttatgcagacttgaaattaactgttaacgactctgacgttgatagccataagagaaactatttagtagaacctgctaaagacagaggtgattataaatttatatctgttggtattgactggggttatagattagcccccataagtagtaatacttaatgtaaaaccttgttaaacgggcatagtaaaataatatacacataaacaatatactggtaagagagactaaatcctagtgtataaggacagagttaacctaccgtgctaaatcagttataatgtaactgtaaaa

SEQ ID NO: 3-9 - skipped (7 entries)

SEQ ID NO: 10 - DNA, 33 bp, synthetic construct
  source 1..33: mol_type = other DNA
gtaaaataagtaaaatcaagaaaactattactc

SEQ ID NO: 11 - DNA, 27 bp, synthetic construct
  source 1..27: mol_type = other DNA
caaaagaaggaccaacaagttctaatt

SEQ ID NO: 12 - DNA, 17 bp, synthetic construct
  source 1..17: mol_type = other DNA
ctgttgcggcgacagct

SEQ ID NO: 13 - DNA, 18 bp, synthetic construct
  source 1..18: mol_type = other DNA
ccaccaacccagccacaa

SEQ ID NO: 14 - DNA, 14 bp, synthetic construct
  source 1..14: mol_type = other DNA
taaaaaaaaataaa

SEQ ID NO: 15 - DNA, 13 bp, synthetic construct
  source 1..13: mol_type = other DNA
taaaaaaaataaa

SEQ ID NO: 16 - DNA, 11 bp, synthetic construct
  source 1..11: mol_type = other DNA
taaaaaaattg

SEQ ID NO: 17 - DNA, 12 bp, synthetic construct
  source 1..12: mol_type = other DNA
taaaaaaaattg

SEQ ID NO: 18 - protein, 21 aa, synthetic construct
ETGWVALTPEKVKDLPWGEDG

SEQ ID NO: 19 - protein, 21 aa, synthetic construct
ETGWVALTPEEVKDLPWGEDG

SEQ ID NO: 20 - protein, 21 aa, synthetic construct
ETGWVALTPGEVKDLPWGEDG

SEQ ID NO: 21 - protein, 21 aa, synthetic construct
YLDILPKTIIKSWEHRSFWRE

SEQ ID NO: 22 - protein, 21 aa, synthetic construct
YLDILPKTPVESWTHRSFWRE

SEQ ID NO: 23 - protein, 21 aa, synthetic construct
YLDILPKTPIESWEHRSFWRE

SEQ ID NO: 24 - protein, 21 aa, synthetic construct
VLEWNGDAWTAMFCNGVNCRR

SEQ ID NO: 25 - protein, 21 aa, synthetic construct
VLEWNGDAWTVMFCNGVNCRR

SEQ ID NO: 26 - protein, 21 aa, synthetic construct
YRKNNGKDIPSYSQKEWNKNA

SEQ ID NO: 27 - protein, 21 aa, synthetic construct
YRKNNGKDIPFYSQKEWNKNA

SEQ ID NO: 28 - protein, 21 aa, synthetic construct
NIGEREAVDYLSNHLKHKELY

SEQ ID NO: 29 - protein, 21 aa, synthetic construct
NIGEREAVDYPSNHLKHKELY

SEQ ID NO: 30 - protein, 21 aa, synthetic construct
RNRNGKDLKSDFIFSEHPNAE

SEQ ID NO: 31 - protein, 21 aa, synthetic construct
RNRNGKDLKSNFIFSEHPNAE

SEQ ID NO: 32 - protein, 20 aa, synthetic construct
YETDSNGQLTVRLLKLPQTA

SEQ ID NO: 33 - protein, 20 aa, synthetic construct
YETDSNGQLMVRLLKLPQTA

SEQ ID NO: 34 - protein, 21 aa, synthetic construct
WENLLEDLKYRATPSVEYNRE

SEQ ID NO: 35 - protein, 21 aa, synthetic construct
WENLLEDLKYQATPSVEYNRE